SEQUENCE LISTING (84 sequences)

SEQ ID NO: 1 - protein, 857 aa, Glycine max
MADSVVAFLLDNLSRLLEDEHKLLSGVEDKVNSLCNELKFIHIFLKNSEGKRSHDTVKEVVSQIRDVAHKAEDVVDTYVTNITRHKQRSKLSKLFHLKEQVMVLHQVNSDIEKIRSQIDDIYKNRDRYGIGEGEFRSEEAAAEAESLLKRRREVEEEDVVGLVHDSSHVIQELMESESRLKVVSIIGMGGLGKTTLARKIHNNNQVQLRFPCLAWVSVSNDYRPKECLLSLLKCSMSSTSEFEKLSEEELKKKVAEWLKEKRYLVVLDDIWETQVWDEVKGAFPDDQTGSRILITSRNKDVAHYAGTASPYDLPILNEDESWELFTKKIFRGEECPSDLEPLGRSIVKTCGGLPLAIVVLAGLVAKKEKSQREWSRIKDVSWHLTEDKTGVMDILKLSYNNLPGRLKPCFLYFGIYPEDYEISARQLIQYWIAEGFIQPQKTGIADTTELEDVADFYLDELVDRSLVQVAERRSDGGVKTCRIHDLLRDLCLSESKSDKFLEVCTNSNIDTVSNTNPRRMSIHLKGDSGVSANTFNKSCTRSMFIFGSDDRMDLVPVLKNFELARVLDCDSFIGFSSYPVPRDLKRMIHLRYLRICVKHLPDCVCSLWNLETLDVTYETTVSSKIWTLKRLRHLYLSGGGKLPVVLPKANRMENLQTLLLDGSYGIDTYPQQVISLLKIDIFPRLRKLALRYPDGPDMLSSLHHLSNLRSLKVIRGFELPSDTNAFPSNLTKITLAAVRDLHFMKTLGQLTNLQILKLRFGKVGFHLDIGRGEFPQLQVLHMTQINVRQWRLEKDAMPRLRHLVINDCEELSELPEELWSMTALRVVHVSRPSEELANSLKNVEPRNGCKLKISNEL

SEQ ID NO: 2 - DNA, 2574 bp, Glycine max
  source 1..2574: mol_type = genomic DNA
atggcagatagtgttgttgcttttctgctagataacttgtctcggctgcttgaagatgagcacaagttgctttctggcgtggaggacaaagtcaactctctttgcaatgagctcaagtttatacacatcttcctcaagaattccgagggcaagcgcagtcatgacacagtgaaagaagtggtgagccaaatcagagatgtcgcccacaaagctgaagatgtggttgacacctacgttaccaacatcaccaggcataaacagagaagcaaactgagcaagttattccacctcaaagaacaagtcatggtgcttcaccaagtcaactctgacatagagaagatcaggagtcagatcgatgacatctataagaacagggacagatatggcattggagaaggtgaattccgaagcgaagaagctgctgcagaggcagaatcactcctcaaaagaaggagggaggtggaggaagaagatgtggtgggcttagtgcatgactcaagccatgtaattcaggaactcatggagagtgagtcacgtcttaaagttgtttccataattggaatgggagggttgggtaagaccactcttgcccgtaagatccataacaacaatcaagtgcagctgcggtttccttgccttgcatgggtttctgtgtccaatgattacagacccaaggaatgtcttctcagccttctcaaatgctcaatgtcatccacatctgaatttgaaaaattaagtgaggaagaactgaagaagaaggtagcggaatggttgaaagagaagaggtatctggtagtgcttgatgacatctgggaaacccaagtatgggatgaggttaaaggagcctttccagatgaccaaacaggtagtagaattctcataacaagtcgcaacaaagacgtggcacactatgctggaactgcgtctccctacgaccttcccatcctcaatgaagatgaaagctgggaactcttcacaaagaagatttttcgaggtgaagaatgcccgtctgatttagagcctctgggtagatccattgtgaaaacttgtgggggtttaccacttgccattgtggttttagcaggactcgttgccaagaaggagaagtcacaaagagagtggtcaagaatcaaggatgtgagttggcatcttacagaggataagactggagtgatggatatactgaagcttagctacaacaacttgcctggaagattaaagccttgctttctgtattttggaatctatccagaagactatgagatcagtgcaagacaattgatccaatattggatcgcagaagggttcatacaaccacaaaaaactggaattgcagatacaacagaactggaagatgtagctgacttttacttggatgagctggtggatcgcagcttggtgcaagtggcagaaagaaggagtgatgggggtgtcaagacatgtcggattcatgatcttctacgtgatctttgcttgtcagaaagcaagtctgataagtttctggaggtttgcacaaactccaacattgataccgtcagtaataccaatccccgtagaatgtccatccacttgaaaggagactcgggtgtctctgcaaacacatttaacaaatcatgcactcgttctatgttcatctttggtagtgatgatagaatggacctagttcctgttttgaaaaacttcgagttggcccgtgtgcttgattgtgattcgtttattgggttttcgtcttatccagtccccagagatttgaagaggatgatccatctaagatacttgagaatatgcgtcaagcaccttccagattgtgtatgcagcctttggaatctagaaacactggatgtaacatatgaaaccacagtatccagtaaaatttggacgttgaagcggctgagacatctttatttgagtgggggagggaagttaccagtcgtcctgccaaaagcaaatagaatggagaatctccaaacccttttgttagatggtagctatggtatagacacgtatccacaacaagtgatttccctgttgaaaattgacatatttcctaggttgcgaaaattagctttgcgctatcctgatggaccagatatgttatcaagtctacaccacctcagcaatcttcgtagcctaaaagtaattcgcggttttgaacttccatcagacaccaatgcgtttccatcaaatcttaccaagataaccttagcagcagtccgggatttacatttcatgaagacactggggcaacttaccaaccttcaaatcttgaaactaagattcggcaaagttggttttcatcttgatattggcaggggagagttcccgcaacttcaagtgcttcatatgacgcaaataaatgtaagacaatggagattagagaaagatgcaatgcctcgacttcgccatctggtcattaatgactgtgaagaattgtcagagcttccagaagaactttggtcaatgactgccttaagagtggtgcatgtttcacggccctccgaagaattggctaacagtctcaaaaatgtggagcccaggaatggctgtaagctcaaaatctctaatgaactgtga

SEQ ID NO: 3 - DNA, 28 bp, synthetic construct
  misc_feature 1..28: note = Artificially Synthesized
  source 1..28: mol_type = other DNA
atggcagatagtgttgttgcttttctgc

SEQ ID NO: 4 - DNA, 32 bp, synthetic construct
  misc_feature 1..32: note = Artificially Synthesized
  source 1..32: mol_type = other DNA
tcacagttcattagagattttgagcttacagc

SEQ ID NO: 5 - DNA, 22 bp, synthetic construct
  misc_feature 1..22: note = Artificially Synthesized
  source 1..22: mol_type = other DNA
tcggcaaagttggttttcatct

SEQ ID NO: 6 - DNA, 20 bp, synthetic construct
  misc_feature 1..20: note = Artificially Synthesized
  source 1..20: mol_type = other DNA
ccattcctgggctccacatt

SEQ ID NO: 7 - DNA, 2525 bp, synthetic construct
  misc_feature 1..2525: note = Artificially Synthesized
  source 1..2525: mol_type = other DNA
aaaggcttttttgttaagggaaggtgtcctaagtactaacccacgaaggaatgagggaagtgaggatggtaacatgtcaaataaatttggaatttagttcccgaaatttttgagtgttaattattgtaggaatttactcatttcatgataaaacttgacatggagtaccggacttaaaaaagaaaaacatgacatgaagtggatttggagtcaagttttctctcaaggcaaactggatttattctccgtactcacttattaaacaaggttaaaacaattaggtttgattgacattttggccttcagggttgtctttataattttttcatatacgaaaaaagttataagttttaatttctgtacacacatataagttaagttttgatctccgtgatcaacgcagctagtatgtatgtggcacctattcatttgctttactgagccttctcaagtgtttgttgtccacgcctgaatataatgatttattcaagaaaacaacggattgaggaagaactgaagaaggtagcagaatggttgaaggggaaaaattacctggtgatgcttgatgacatatgggatgaggttaaaggtgcatttccagatgaccaaaatggtagcagaatactaataacaaggaggtggcatactatgtacaagaacaattttcaataggatcatcttagaccttaagagcattgacgtaaagatagatgacgaagatcaagccatcattctgttgagctttcgtcctaagccatttgatcattttgctgatacaatgttgtatagaaaggctactctatcaatggatgaggtcaaggctgctttgaactcaaaagaaaatcaaggggagaatgactctggcagtaaaagactcttttctactttcttgtgctgatgtcagttttttgtctccttttttctttcatggtcattattgtgctgatgttttaacatcatttacatgtttattgtaaaaggctctttgctctggcagagtttgatgagatgatttgcaattcctctgcttctttctattcacttttctgctttcttgtgttccacaaccacttgaccagatttttcagttagcttagcatttaagcctgagaaattatgcatcatttcatttgtttgttgtacaaccatatttaatgttaacctctctcccttgtttttccattctaaacatggatcatgtttttgtttattggtctaaagtgttgtttcatgttatttttctttctcttttcccaatggaaggtcctttcatgatttagttagaatagataactgataacacaattagttggaaaggaagatgttagagtgtatgttgataatgggagacaaatcatagtgatcagttttagaattggagtgaaagcatacagacattgcttttgtgccttgtctctgctactctacccaaacctttgctggttttatcatggatataattcattattttatgatctttttggtagattttctaccaaaattcatttcttcttttcggtgatgctatatagtacgaattagttttgcaccaagaagtacgaatccacttggcaaaatatgatcgtacaaatattaatttgaaagaataaaatctagacatgcaagttctaactaccctctttcaaactggaatttgtggaattgttggaattttggaaaagcttgttctttacatcaatgtatatattctcccaaattgtcttacaacacaaaaaaacttgagtattttcttacatattatctgcctacatttttgcgcttaactggattatggaaaaataatttgctaatgagatatactaggacagatggatgaaaattacaccaacttttaacaaaggtttaaatacagtttttttttatttataatttatcatatttttgcctttagtctttatatttttttttgtttttaagctttgtaaattatatttattttatttttaatttttaaagcaccttaaataatattttgacaagtgaaaaaaagtgttatctaaaatgttagaacgaaaaataaaaaaatataatttataaggactaaactttttttacaaaaaaaataaaaaaaatactaaagaataaaaaaatatttaagacaataacaatttgctgaatacaacagaaaagttagttagaacacaataacaatttttaaagattattgttaaagactctgcattttaaaattctaactaatttttgtttctaaaccatggaagacttttatttcataaacacaataacaattaaacaaaactcacaatttaaaaaaaaatcagagtcatcaaatatattaatatttaaagacagaaaaaaaaataaacaaataaagttgagaaagccaacctataattaaaaaggttgacagaaacgcgggtccacgcgtatgcgcgttctttccctttattggcgtccacttgttatctttcgttctctactctgtaaattatctgtccacaaactctctagagtttccacccaagatttcctgtttcacagaa

SEQ ID NO: 8 - DNA, 20 bp, synthetic construct
  misc_feature 1..20: note = Artificially Synthesized
  source 1..20: mol_type = other DNA
attggactctggtgatggtg

SEQ ID NO: 9 - DNA, 21 bp, synthetic construct
  misc_feature 1..21: note = Artificially Synthesized
  source 1..21: mol_type = other DNA
tcagcagaggtggtgaacatt

SEQ ID NO: 10 - DNA, 22 bp, synthetic construct
  misc_feature 1..22: note = Artificially Synthesized
  source 1..22: mol_type = other DNA
agaaacccacgtcatgccagtt

SEQ ID NO: 11 - DNA, 32767 bp, synthetic construct
  misc_feature 1..32767: note = Artificially Synthesized
  source 1..32767: mol_type = other DNA
ggatccgtagacaatgtcggtggcaaggatgcagtctgttagcaaaacaaatggtaggaagaactaatgatcaacacaaatagtgaagcagcagtaaaagctttcaattaaaagcaaatttacaaaggctgcataacatttatgatgggtttggatgagtggtaaggagaagagaaaaatgacacaaacccttgtgtagaagatatttaatgagatgggatggaatgggaaagtcttgataagtacaggattgtgaatgaacaaatgcaccaatagccccatccttaatttggatatatgattttctgattcttgatgtagaacaactgctagagcctttcaattgcattactaggttcgaggtttgcttgcttgtgtaagtgagaagggaagattgaaaataagatggaatgaagaaaaaataaattaaagtcatggctcaaagtgaaaataagtatattttattttcctatttctcatgtttctgctaaaggcaagtgtacattcatctcaattcacctaccaaactatgcagatactaagaccatgcatgtccaggaaatataatcataaattcgctaactatccacctaccgaaccaagcagataccaagaccatgcatgtccaggaaatataatcataattcataacagactagcacaagtttaagtctttatttgtatatttatatagaagttacccttgatgatggcgtaacggatggtggctcaagagatagcctagcagaacctgactgtgaaccctgtataattcagattacagagtaaacataggaacaagtttcaaaggaaagtaaacaagggaaaaccttgaggggtgggggaagggggaactacaatataaaaccaaacttgactgcattcattctttggcttatagttaaataaatttactaagctaatgaacaaaatatatactattaaactaacaagacatcctataaaatagcctattatgcctaattgataataacagttctcccaaaaggcataattgcattgataaatgggagttgaggaaaatgctttaccctggaagagggtgaaactggtggaagctcaagggctggcctggaagaactggaagaagatgcctatataatataaaaatgtcttagtatattatattatatactatatacagcaaaaaaatagaaagaagataattgaaagcagatctgaaattaagactgcaaagccaaagccagaaaaaaaatatccatcgtaactaatggaagtctggttcatgcctgaatatactctgtggatctgctattctccaaagtcttggtaaattgacacaacatgttcactccttcaattgtggccacctaagcagattaaagccggtaaaaagtaaatcacagatataaacaatattcataacctaaaaaaggtctataattaaattatgcagtgactgaagtcaaattgaccatacctgcttctcttctatttctttaattttagattcctgagaagccaagtaatcaggtttaagttaaaacacccaataataaattaaagaaataactgaattgttgcaattgatttaccagaacatggactgcaacatgaaaagacttcgaatcttcacttattgtatctgctttccgttgaattacatatatctgcaacaggagatattctattgttaaacaaggaagaagacaaaattagacataaaaaatctttactcacatcctccctggtactttcagtaatggcagcacattcttctaaatttttatccagaccgttgattcttgcagataatttcttcttggtatcctgaaaccattgcagataagtaatttttcattctcaataacttcatggtaaaacaactgtcaatgtaattcttaatgtccagataaagaaagaaaagtaaactgagctgaatgcaaaatttaaaagcatcatcatttatagaacacacaaagttccttttcaaaattttgtttgaaatataagatttgttcgattcaataatggaaccataccaattttcacaatgaaagtgataaatgtaagttatgcacttaacgaggtcacaaaatatttacatgaataatattaccacaaaggcttgacagtaaaatcaagctcaaagattatttgaatgcagtcataataaagagaatgagctcaattaagtagtattttttgtctctctgttgatctaaatgacacatttatcctaacgtgcacttccagtgtttcaggggaaccacacatgagctcgatgtataaaacatgaacaaactaagatcatcatcaagttcatatgttacgtattttctagttcaaaagcatcagaataaataatacataattatgttattaaaaatatttaatgatttgctccttgacattttgttatatagctgtactctagttaacagatagttatcaacttatcattcattgtcatctttgataaagaatgtctggttttgacacagtctaagaagaacaaactatgcaataaaagggaaaaattaccccaattgattcataaagctttcccatctgattaccaattgatgtgcaagcatcagacaaaccacgccttgttgcaaacatcagatcaggaagcttccatccctgcaaacacacgagagccataaaactcattcataagagagtggggtaattgaagatttggagatagtatggtgagataaaagaaaattcatttttaaaaaaattagagaataagtgaactaacagccagctgctcaggcataaagtttagtatagctaaattcatgtactatgacagatctaagtgtaagagagaagggacaagccttcaagaatttgtgggaataatacatgtagattgttgtaatataacaagcaacaaaattgttgattttcttgcatagttttaagagttctctttttgggtgcataaattttattcccagaaactaaaattcctggatttgccactccatgcaaacctgccaactaataatcatctaacagcaagtaatggatagttgattctgctctaagttatgtggaaaatattagtaacatgagttgagctcaactcaaaccagtaaatccaactctcaaaaaagaatgataacaatttatgggaacagaaagaaactgctacaactcaaacaaacatgaatgccatatgatcaaatcatttttttaaaaaaaaaaagtcaaaaatggtttatatattctacaagattagcaagctctcaatttcacacttgacagttatcatgacaatatatagcaggaatctggatcctctgcagcctctcccagcaaggtttctctccttcccaaatctcaaccacccatcacattctctttcctcttcagctactaagttcaactacaacagtcaagatcttggctggacagtggcgggagagaaattaagtggagagtatccgtgtgcaaagagcagatggcactcgttaaaagcaccaatatttaaattcaaattagccataaagcttccaataagaatgtatcactacagcatcctgttacttaaatcaaaaattgagagagaaaagacattaccttccaccaaacgtatccatatcctaccacaacaataacaatcactgttgcatattttctcccacctgtgcatttcccaaacaagtcagaaaataaaaaaggagtaaaaaataataaggaaacattgttttgcaaattaacacgtcaacatttattaaattgctgaagaaataacaaaaggcccctataatgtgtataagaaagtagctaaccagtacctgttccacttgcatttacaatagtgattgatctatctctagcaagaagttgtagttcctgccgaagactgttaacctgtagatcaacagttaatcagaattttcaattacccctgtattgctatattggtatatatttacaaatatagcatttgttttagtagccaattcaacaggggggaaataaaaatcccacttcaactgctaagtgctaaaagatataactttaattatataaatgttttcattcaattcatagaatgtcagaactcagcattattagcactctatattgggaatctatgataaaaatgaatgcacagtgtaacttcataaattcccaaagcagcagacccagcatgcactttaactttaaataatttatgatcagatcacaacctacttcagaatgaatgttgtaacagaatcaatgctatgaattatagtacttgtgacccagttgtttgtttcaattttgaaacatgcaacagaagctatttttagttctcagacaagtacgaggttaattgttaagattagcattaacattatatacatttacttgtacgtaacatatgttcaaaatacaacttcagttgttatgaataagacatatatatgagtaaaaaaaatatgcatatgctcagaactcaagtcatgtatatgattcactaaatgatgttcagcatgatgcatgagactaaacttaactagacaaagaactattcctaattaaaataaaattaaatataatcaataatgaaaaggcagcaacaacaacaacgacatgcatacctgagccaacaaagcatcattatgcggctgctttttaacagttggagcaggatcatcgcttttaagttgcctcaaaacaacctggcaaaaaagtctacaatcagtaaaaagcctgcatacttttttttttttgctcagcaaaaatatatatattatagattgaaatagtaccagtggtactatgggtacaaaagcctgcatacttgattaccaagaataaatgacagttaccatatactcactatattaatagaaattggacccaatcacagatataaaataatagcaaccaaaatattgccaaacttgggattttaggatacaagacaagttgaaagaatttgcctaaatttctcaagtacatacaacaacaaccagtgtatcccaacaggtggaatcagccacatagatcaagcagggttctattaaaaaaccaaagttttagtaaaactatttagaacatggtctttttactattttttaatagtgtctcctaatatttccttggccttcatccaatcaaactatcttccatatgtttcctttgatccatttgtctcactactagactagagcctctattggccttcttacatgactaaactaccttaggaaagtttccatcatcttctccttataatcacatattatacccaaaatagtccccttaacaggaatcctaaatgtatgccattctcatctatattgctttgtagtgtacattgtagtaaaataaatcaaatcctatatgttgccattctcatttttgcttgcatgagttagagcattatgatagtcttcaacactatttttttttgttttataaattcgatttctgaaatacaattccccccatacccttaccttaggtctagaaattgcaccccccataccataaaccctctcctacacattcacacaacgtagctacagtacagactagctctccttgaacttctgttgttttaaatatcatctcaatcataccactttggacaccgaaaagaggataatcgcatcttaaaacatgggaggggtagcaaaatcataagcatcatttaccgagagggaaaaaaaattgcatttatcaatttgtacccgtcagcatagttcattaaatgtatacatccttcactagtgaaaaaaaaaatgcataaaaaaaataacaaaaacagcagcacccagagaaacaagttgagctttcaaaacccaaccttaaaagcaccagaggcgagaccagagacatctggcagacgcccttcttttgcaattactgaaccagctatacctaaaacacaaacaagcaaacgaacgcatttcagtgaggcattcaaaattcgagtttcgtcgaaaaacctagattaacgtgccaccacgagcaaacgaacctaaattgagatggcatcaattttgggggaaaaaaagatttggagaaaataattaaaattaaataaagagaatgacagtgtgtgtgaacctgcgccgacgagaatgaagagcttgccgagtgaaagagccatggcgccagtgagtctgagcaaacaagctagcgcactctgttgcgaagcatttagcgcgaaacgatgcgtttaggattaggctatgtgcgattgtgcgttgagattgtttatgtttgggcactggtgagaattgatcagcgagacaaagaagacaatgaaatcgaacaaacccttgcgattgcggtgctccctgctcctttgtttcctcagtggacgaccctagttttgaagctgtgaaaataaataaaaatactagtatattaatagtatattccctttctagttgtgcaacaaacaacaattacaaatatgagtttagagtgtgttttgaatgaaagatggaaatattattaaagataaacttttgtactttttttattgggacctattgattatactctatcttttatttttcacaaatggattatattctatctcaattcaaatttatctttatttttatattttttattttttatcatgtaaatcaaacataccattaatacttataaaatatagactcatatacagtataagacagttttatataattaattaataataaaatattattcataatatttttaaaataaaagaaaataagtggatatattaataatgtattatattatcattaaattataaatgattatgtaagataagtttgttattttttataataattataatgttatattcttaaataaataagtttatattattatctaaaggaagagagataacgtagaatacaaaaattgacataaaatattgcaatgataacacctctccctcattaaacaaattgacaaattttaaaaatcataccaataataataaaatattttatatgatttatacatacatgattactaaattcttaaaataattattatttatgtttataaacttatctcttttaatttgagattggattacattgtaaaattaatttattctgcaagtatatatgaactaattttctatatttatcactatgaacatgagtataaaaaaatggaggagaaaaatctttgtcatcaaatttgtaaatacaattagcagatctcatttgcctaattatctgtctggtttcatgtcagattttttttaccttatttattagaagtttggataaccaggccattcttcctttaagtggtttccaaattcttcaacattgtgtcgcccaagccatgaagatccctattttattgttattattattactattattattaagtgttaatttaattgttatctttcattattgtggctttttttaatactaatgtgcccaataatctatttaaaagaataatctgtattcaatttttgatatgtgaaaaaaattaaatcaaccacaatcatcacattccgtggtttaaagatataacttaatcaaattaagatagttaatatttatgatttcaacattcattcatttttaaatatttaattattattattattaaaaataatttattgagtgaatatcaaaattgaactgtgtcatgatgtaacaagttatgaatcgtgattatcatttgccacataatcaggtaacgtgacattaaaactgttaatagagaaaaaaatgtaggggacgaaaaacgctaaaaaaaatatttagaaattaaaaataaatttgacaaaaagtttagagataaaaaaaacaaatttaatcctaaaataatctgtaatattcctttttaaaaagtaacgaaatatgtgcaatattatgtgattttccggctagtatcttattatttctttctttttgtcttttaattattggacaaacaatcgaatatggccagaagctagccttggatagaggctttacgcacatcatcatttgaatctgattctacagaggacggtatcctactatccctagtctcccaactctgcagggatgaccatgtggcataaatctcaaaaacatctaagtgtaattaaatcaaatattatctcactcacaccgaagtggttggtcattaattattcagttttattcatctaatacattgtaaatgtcatggacttggttaaaatttgttagtgttgaccaaagtaaagataagtagtaggaaagttgacttacactcaagaatcttttaaaatctttaagattgtttttgtcaaaatattctctcaaaatcccaatccaatacatccattagcttgttattacatcaaaataggatatagttggacatgggagtgtcttttctatatattaaactatcttacatagtatttcctcaatcacaaggaaacaccagtccaccttggaagaactcctatcttagccttctaggcatcaggcatgccagagtcaatactctcaagagaaatgtgagtttgtaacttttaatggaaaagattaaactcaacactgaaaactgtattttgatcatacatatatataaacctaacgaataaaaaaaaatacatgtatgtacctaggacatcaccactttagaattgagattgaaccttcgccttaaaactgaataacaacgcaatattgttggggcaaaatacataagatatgtctattctgaaaaaaataacaacaataaacccttatcccactaggtcaaaatcaaaatgaaataaaaatgcaactaactaacaacatcatctgcctgcaaaagagtgcctttaaaatccatcttgaaaagggttatatttttttaattttatttatttaatttgtaatttcacctgagttgataatcgaacctgaggcaaaaagaaacatgagcatccttctctcctgcacgctaggaaatggtataattgacatttgtgatgttccttcttgctgcttctattctcccaagaattcccttaactgctatatcaaatgcatcttcaatggtctctaatctagaacttggataacgatatattatccttggaataagtcttacaatctcctttctcattgccaacacttcactttttggaactctagacaatttctcattgatcattactctcttctctctgacatctctttctggtatgtacacagagtagctagaaccatttctgggcaaatgccacaaatattggttatatgctgattctggatgaaagaaaacaggaatgcaacctgccaaaattgagtcaaaagttgatctcctagtgaatgaatcccctggaggctgtaaacagaaaactgagctctgaaacacctttgtaacatgcacaggatcattgcaataattatgaccgtcattgcaacttagaagtttgcagctcctagaggattggcattgtttgattatctcatttctgatggaggatgatgaattagaattaggccttggagcacctgcaaatgaaaacaagtaaggccttttcacttttatcatttttttctgccactgaaaaacctccttatccttggaggggtgaaagtaagttggatatgggattggaaactcattgacctttgagccagattcaattagcataattgacatgtttcttgcttctggaaaaagcattagcttggtgccccagtcatcattattttctgttcttctcctgaagtcccaaccaactctccctccaaccatgaaatgatctctaccccacattcttttccattcgggttgttgagccagccacttcactagttcttttggtgaagcatctctgattgaaacattgaagcctccccagaggtactgacccacatcaaggccagcatagtagggtacatatatagcagaagccagtgaggaatcattggttaagcacttgtagttcttcattgtgttatggaaaataacctcaagtgaaaattggttagttgcataccagctttccttcaatagagccttctcctttgatttctcaataaccttctgtccaaggcctaagttagacatgtaagggcacatatcatcccacttcatgagagagtgacaccccttcagcaaatcctcattgaacctgctagcaagatcataaacatagacatattgacctgaacaagaatccaaatttctagtggacaaatttgaaacaagtccaacattgtctctattcaaaggaggggaactcatgtggactctcttatccacttcaccatgtgcttcaggaactatggtctctgtgtgattctctacaatgtaccttggtatattggaggccccacttgatacactattttgattggtatgattaaattttctaggacaatcggcattttcattataattgaaagaagaatttgatggtaaaaatgtaatgccacgatcactcacagccatataatagtcccagagaagtaatgacatacaaaaaatgaaggaggtgaaaaacacaaaccggattttatcaaagtacttgctcaacggagggttgtccatgttttctttttggccttttgagattcagtaaaagtgaatgtgtttttctctgatgatatggggagagggtgacatagagagatttgtgtaatgcaaagtttgtgtgtgatttggggactttatcaaaggaatgatctgatatggaaaagaaaagtgggaaagatattattgacattgaaaatttccttttttacttgaaaagactgaataacaatggaaagtaatcaaagtattactgtttaattgaagaaagcattaaaaataaaatcgtgcaaaggatcaaaaaccaaaattaatagtactgtaattccgtacatgaatatttctcaagagatgtgcttcacaacatacattcaatgcattaagattgagattcttttattataatattgataatcatacatatatgtttatgatattatacgattgggtccagatcttgggaaatgcttccccattatcatttagtatctaaatggtcttcattgattttctatagttattccagagacaccagatagcaacaccatgcatggagaagcagcacaatgtagaccttcaggcttcagccaccttatttataactatatatagccacccactatatgaaattccaaaatccaccttttatattataaaattcgaaatctacttaattattaattaaaagttatagtttttattttacttattactaacaattaattattaaagtaatatttcattaaataaaattatttttatttttagattaaaattttcaagatctatttttaaattttaatttatattttttattatccataagttttattcttttattaaaaatttacgttttaggtatattttgaatttttacaaattactggtgtaatggactggataatttttagttataggtgatatgaatattgcaaatatatacagctgctgaagtgattaatgatataatatcatgtaaattataaaagcctgtttacaaattattcattgatttttgcatcttaagttcagtagagtgtgtaaattatgataaatccttgataccagataaaaaaataaaaagagaacagtattattgctaacttcgttatttttggggaacagatccaagaagcatgcaaaaagggtagtacacgatactatcattatcctctatcattatggcctgaacaattagacattcaactcgtagttgcaaagattaaattgcaagagccgagttctatcaactgatctatattcccctgagccaaatgaagtatgcacgaaggagttaagtcagttttctattcgttttcttgacgcagtggagcagttcactggcgccatggacaagccctggtttggaggatttgctgattgggaagtgatggaaacggggttgatagttacataattactgggttactgtgaagaggttgaaagatgctagttggaacttggaagagtgcgttcagggatcacattttcctaatggcagcagcccttctcccttattcacggtatgtatgtatgtattcttattttttaaagcttctttaatcacactgcaagtgattgctcattcattatttttcagttttaattcgtataatatataattctcctaaactcaaatattgggaaaattaaataattaatgttggcctacactcatgagtcatgatcatgaataatgggtgaatataaatgggttcaaggggagtggtttgcttgggaaatccaagtgttttaagaggtttttaatggagatcgaagtggctgtgttggattactcatggaggtggcggaaagatcaatcaggaaactacatacatagctttgtgagatcttcaattcggttcttgtattgatgacactttggcaaatacttggaatctgaaagtccctccaaagatcattttttttttcctttggagacttgtcagaaacaggctccgtagtggtgataatctgctaaggaggaacgtcgtggtaagtggtggctgctttttttttttttatctgagaataaaaaacagtgttaaggaaattataacaaatcacgcgttatattcaaccaaatgagttagactaccttaactagtggtgacatttatagttgtcattttgctatggttggttgggtggatctgaatagttacttacccgcatctgtaaaatcacacaactcacagcacgagtttttgttgcttggaacaaagattatgcaagcaagtgaagagtagtttggtgggggtgttatatgtgtactcttggaacacattagataatattgatttaaacgaaaagatctttcataggcagaacatcatacaggatattattttaatttccattcgtggacctgactcaagaatttcgatgcatccttttcatactcatatgttcagtggtgcttgaatccaggtgtgtgtttgaaggagtagcatgatcctcttatcaaattttgttattgaactgctatatgatttagtgatcctcaggtttgttgtttcctgactgatgagttctgtagtactggattagtttaaaagagatttcaatggatgtgtccctttaaaggaaaaaaaaaattatataatgagtaattaaaatataataaaggggatggggggagtacttagcaaaaatagcaagtagaaatttggcccaactctcaggcaggggctgagctaggccatttcttttgaggggggcaaatttaaaaactaaaattaattatacttacaatattaatatgttaaatttaactaataattaaaatatctataataattattatatttgaacatcataattaatcataaattatttttaaaatcaaatttatttattttcctaatgataaaaaaaccaagttttattttttaatttgagaataataagaatttaaattttatgcatgatctgttgttcatgattgttgtatggtgatgaatcagcaagcatagatcagtgtattgagacgtgtgtttgacacacctctttaaggatttattcacgtgtgtaaatttcccacaatttaattggtcctttctaagagcttaaattttataattataaatcaattaaaaattattctagatataatttttaaaataattactataaaatttagtaattttagcatacataatgcatagcaataattgaatgataatatatatataacatttatagtgctagtgtatttacattaaattataggattactatatctctactttaaaaaaaactacaatgaaataaatattttttatctttttattaaaaaaattagtgaaaaggaagatgtcttaaaaattatgaaaattgatggtcttataattttttagaagtgtatctattttgaatgatgagaaagatagaaagatagcaaaaaatacgtgagatgcaataggtcaagtgaagagaaagaaaaggaaagataaaaaaaaaaatgatggaaaaagggttataaaaaataacaaaaaaaagtatataaaatcattattgaaaaagtatatgcccatatttatgtatttctttcttctttagaaatgaatttctacagcatatacacacacatcagtcgtctaactcagaaacatcaacggtctactacttcaaacacactatacatgtccaattggacacatcactaaagacattcggatacatattaattcttccttgattagagattgattactattatcgtattggatattaacaaaacatattcagatttttgtctttgattttattcctaaacaccccgtctcattagcttcagaattcttcatttccaacctcaattctttgatttgagaaataaccctgttataggttcaaagattttcttctacaaataaatgaaccccatacaaggtagacgaataaaaaaatacatgtatgtacctagggcatcaccacattagaattgagatcgaacctttgccttaaaaatgaataacaacgcaatatttactgggggaaatacataggatatgtctattctgaaaaaataacaacaacaaagtcttgtccgggtgaaaatcaaactgaaatgaacatgcagctaagtgacaacatcttctgcctgcaagaccattccaagttcttataagagtgctttaaaatccatctcgaaaagggatatattttctgaatttttttatttaatttgtaatttcacctgactggataattgaacctgaggcaaagagaaacatagaatccttgtctcctgcacattaggaaatggtataattgacatttgtaatgttccttcttactgcttctattctcccaagaattcccttaactgctataccaaatgcatcttcaacggtcactgatctagaacttggataacgatatattattcttggaataagtcttattatctcctttctcatttccaacacttcactttttggaaccttagacaacttctcattgatcattactctcttctctctgacatctctttctggtatgtacacagagtagctagaaccatttctgggcaaatgccacaaatattggttatatgctgattctggatgaaagaaaacaggaatgcaacctgccaaaattgagtcaaaagttgatctcctagtgaatgaatcccctggaggctgtaaacagaaaactgagctctgaaacacctttgtaacatgcacaggatcattgcaataattatgaccatcattgcaacttagaagtttgcagctcctagaggattgacattgtttgattatttcatttctgatggaggatgatgaatgccttgaagcacctgcaaatgaaaacaagtaaggccttgaaacttttatcatttttttctgccactgaaaaaactctttatccttggaggggtgaaagtaagttggatatgggattggaaactcattgtcctttgaaccagattcaattagcaaaattgacatgtttcttgcttctggcaaaagcattagcttggtgccccagtcattattattttctgttcttctcctgaagtcccaaccaattctccctacaaccatgaaatgatccctaccccacattcttttccactcgggttgtcgtgccagccatttcactagttcttttggtgaagcatctctgattgaaacattgaagcctccccagaggtactgacccacatcaaggccagcatagtagggtacatatatagcagaagcctgtgaggaatcattggttaagcacttgtagttcttcagtgtgttatggaaaataacttcaagtgaaaattggttggttgcataccagctttccttcaacaaaaccttctcctttgatttctcaataaccttaggtccaaggcctaagttagacatgtaaagacacatatctatcgacttcctgagagagtgacaccctttcagcaaatcttcattgaacctgctagcaagatcataaacatagatatattgaccagaacaagaatccaaatttcttgtgggaacatttgtaacaactccaacattgtctctatccaaaggaggggaattcatgtggactctcttatccacttcaccatgtgctgcaggaactatggtctctgtgtgattctctacaatgcgccttggtatattggaggcaccacttgttacaccattttgattggtatgattagattttctaggacaatcggcagtgtcattaagattgaaagaagaatttgagggtttaggcatgtgtgcttgttttgcatcattcatgttgaaaaataaaaaggtaatgccatgaccactcacagccatgtaatagtccaagagaagcaatgacaaacataagataaaggaggtgaaaaacacaaaccggattttatcaaagtacttcctcaacggaggcttgcccatgttttctgtttggccagatgtttacattaacccacacaccatgcttaactaattgagctgaaacccttcattttgtttggccttttaaaattcagttaaagagaatgtgttttctcttatgatatggggagacatagagagatttgtgtaaagctttagtgttatttggggcctgtatcaaagttatgatctcatatggaaaagaaaagaagaaaagatattattgactttaaaatatagtaatgagtattcctttaagttgagaggaacaactaaatatttggtgatttgaactaatgttttttaaacactaattttttgttttttttatattttctttttttctcatctttaatatatttatcaaattttctaattatttttttaaataaatcataattttattattattttacataattacttcacttcagtgttaatcatatcaaaatcttataatttaacaagtcaaaatttttaagtttcaattaaatttttaagttcaagctagtttttcaactaatttgatcatttatctttattatatctttattaatatacaccatgtctaaattttcctcaaaaaaacttttaaggataattatttaatttcatattctaaaaataacgttgcaacaactatgaaatataatttcatattaatacaaaaggtcacgtaattatttataaatacttttgtattaatattaaaatacgaaggtattaattacgcaatttcatatttaaaaaaagtcatctttattttatctttattatataaattataaagtaatgtacaccaagtctaaatttttccctcaaagattttgaggataattatgtaatttcatactctacataaaaagtcacaataattatgaaacagaattaatttcacattaatacaaagtattaattatgcaatttcgtattttaaaaaagtcatctttattaaatataaaaaaatgtatatatatatatataataaagatatattaatttattatccataagtatttattaaataaataaatacagattttaagaaaaaaaaaattttgttggcataataaaaaatcatgagattaaactatgtaaaaattatcaaaataaattttatctcttccaacatatctacagttatagtaaaaaaaaaaaattaaactaatgtcaacatttgtttgtaaagaagtcaatcatctatcaattctgtttgactttgtttgtattttaaggaagtatttattagtagtttaaaaatatttgatttatgaagcatattttaagagaatatttattaatcataagtatttatatttataattactaatttaaatttattattctaggtttcaaaatagtttttatatgtaataagaatagtaaatatgaatttaattatttagttccaactaaaaaatttaaaattttaagatgataaagtttggtaattattatatctttatctttatctccaaagataggttgttgggaccctttttttttaaaaaaaaaaagggtaaatagtataattttttattacatttaatagatttatgtttttcattattattattattattattattattgttacaattttcaattgttaaaataccatatgctttgaattaattttttttgttattaaatgagttcctaaaatttggcatcattttcacattttcaaatagtagtttttgattacttactgtaaattgtgcaatttcaataattattattgaatgccctttataacccattggccaaccatacaacaggttaattttcaaataaatttcaaaattaaaagattcactatttttaaaaatttaaaaggttgaccatcatttgcttcttctcaattctcaagcatttaaaaggatcgttcctttctctttgctggaggggaccacggtggatagagtaactaactttgcgagctattcgagaaggttagtctggtctatatgaatcatattttaagaaaatgtttgttaatcataagtatttatatttataattactaatttagaaaaaaaagtatttatatataataagattaataaatatgaacttaattatttaattatagctaaaaattaattttttaagaatacattaataataaattttaacaattatgatgtctttatctttatctctatataaagatataaagatagggttggtgggagctagttttttcttcaaataagggtaaataagacatttttttctattacatctaatagatttgtctttttcaacaataataataatatattattattacaattttcaatctttaaaatactatatgctttgaatttattttttttgtttttttaatgtgatttttttattaagtgagttcctaaaatttgccatcattttcacattttcaaatagtaattttttattacttacagtaaattgtgcaatttcaacacttgaatgcccctttataacccattgaccaacgatacaacaggttaattttcaaataaatttgaaaattaaaaaaaatcaccattttcaacaatctaattctcaagcattcaaaatgaattgttccttgacttccttcatctttgctagaggggaccctgctaggcacgtagagtaattaattttccgagctttctgagaatgttagagaggtttatatgaaccatatttttaataaaatgtttgttaataataagtatttatatttataatcattaatttaacttgtgcaattttaacgattattattgaatgccctttataacccattgaccaaccctacaacaggttagctttatcttaattctcaggcatttaaaaagaattgttccttcatatttgctggaggggatcaataagacattttttcttttacatttaatagatttgtcttttcaatattattattattagacttaaatatattttttctatgataaatacatattttttatatttgatcctaataaatttcggattgagtccctaatattttaaaaaatttatcttagatacttactgttagttcgtatgaactggacacacgtaaaaatgtgatgtattatgtcggattattgtttatataagttggattttattattttttaacaaagaaaatatttttttttaataatccaatatgtataaatagaaatcacaaaggttatatttgacatgacacatcacacttctacgtgtctaattaagagtcatgtaggattacatattaatgatagagactttaagataaaaaatttaaaacattagaatccaaattcgaaagaaaaatttatcaaagaccaaacacaaaaaatatatatttatcaagaaccaaacatatatttaacccttattattacaccattttcaattttaaaaatatcatattctttgaaattattatttttattaaatgagttccaaaaatttgacatcattttcacatcttcaaataatggtttttattacttagggtaaacggtgcaatttcgacaattattattgaatgccctttataacccattgaccaaccctacaacaggttaattttcaaataaatttcaaaattaaaaaattcaccatttttaacaatttaaaaggtttatcctctcattcatgtttttttattataacttttaacaataaaaaattataatttttagagtaaaatatgtttaaagtctttgtaaaatttgaaaagtattaatttaattttcataaaacttttggtattaatttgatcatgtaaaaataaacacatatttttcatgatcctcagtagtaactctgatagataataatctgacatggttaatatatttaaatgtgtaatttaattataaatgaattaaataatttgtggcggttaaaatttcctgaatgaattgaacaatgtttttgcagctaaaaccttaaaaattacaatttttgtcacttattgtatgattttctcgcattttctgatgtgatgagaaaaataaaataacaatgagttctaatgtcttctaagtgtgcttgaaaaagaaggtaaagagagaaaatgattaagttagttagttaatttcttaaagaaaccatctaatagagttattaataaaggaccattaaaaatatactttatttttatgtggaccaaattcatgtttttttattggatcaaagatcatgttcttaatggccaaccgaaagtcattgatggtggacaattacaccccatcacattcatgatattaaaattttaccttttgcaccctctaaatttatatatggaacccattaccaagtaacatacccttatattgacatatggatgtgcaaaaatagtctctcactatgataaaattacaaataatttattatttttatctactaaataaatttcccctactagtctagggtctttccgtgggtgtttttaacctattggattaaaaactaattaaactcatgattttgttatgataacaaaaacaaaacaaaaatacattcaaaagcatgacggcaaagagtgatgagaggggagagagggaaaaaaagaaaagaaatgaatgaaagaagaaaaggataagaggaaagaagaaaggaaaagaggaaagagtggtggctggagaagggtgaggtgaaatcattcaaaaggtatcttggtctatgttcttgatttatagtgtcaagagtaattttataggaatcaatagtatcatccctttaaataagattgagcaattcaaaattaattttcagagcccataaattattgatttaaccgtttaacctattttatttttaaaacaatgatttaacctacttttacacaagttacaaatcaacgatgataatcttttttattctgaattaatgatgataatctgttaacttgagtaatataatttttttcaattttctctagtttttttatgcaaaatatggttacggtatttcattcataatcaatgattaaatccaatggggagctgaatcaaagacttagttacaagtaagaagtcctaaagtccttactaaagaataaatcacatgattaacttgtctcccgctaaaactcatcctagaatgatgacccagtattgccatgagtgggatagtcaacaatcatcttacaaccaattttctctagttttgttgcttgaaaacttttttatgatttatcactcttatttttttatatagtttcaatcttagtaaatactatcaaataaataatttttgtaacacaattaataattttacgttggagaaagtggaaaaaaaacctttacatatcatatgatttgttcataatatttttataaaataattaattagacatgtaccaagtcatatgggacagactcaagtcaaaataaaatatatttaaaacttatattctgttaatccaactctcttttttatcaagttattcctccatttattggacttaacacatttaacattcctactttcttgttgtggtacatgactcaatttgtcattgccacttggaatagttgggttccttggagagtggctctcccctcaagctatgcactttaaaaatatttcgaatactttctgccttgtgatgatggtaattttttccaaagtaatttatcttacctttgaatgcacccatcggtgaaaattttgttttttagccaagattcccaaaaaactgctttgctttacgaaaatatattaaaattctgaagttatcttacggtgatattttcacataaaaatgtttttaaaaaaatattcattaaaacatatacaaaatttttgaaacctgcattcattcacaataaaaccagtagtcgaaactcaactggcaaaattgactttgactaataaaagtatatgtatatcacaccacccaactaattgaagttaaaaaaaatgaaatgtattttacccaatcatgaaataaggataaaatgaataaaaaacaaatagatggccacgcgcaaaaaatattactagaagagctcccttgtgattcactctatgatgttgatatacacaagttaagtaagactcaataataggctcagactaagttaactaattacttctaaatgacatacttataaaggaaataaagtggatggaacaaaaactctataatgcaaagatgtgaaaaaatctgcaaaagaatgccttagtacacaagtgatgctagttaggaggagcccactttggaatatgattattatttgggagcttgtagaggaacagaagttagttatagaacccagtactaatccaacttattggcctagcaatcttaaaagcatttttatttttcatacaaattcctaaagcaggttgagttgcacagtttaaccaactaaccactctattactctaacacacccactccattatcatttatatatacacataccaaacttggattgaaaatagtttcctttccgcacgtaaatcattctaacttgctcataaagacaaacaggaacctaaatataaccccattctaggtacccttttttgatagatagatagctatataggctaataagctcttgaatattagaacttgctatagctatacacacataaaatttggggttaggaagagatgagaaaccgtattgaaagattagtagttccatggtctttcagctgtgcttcccattcaagtgtgaaattaggtgcacccaaaggaccaaaagaagattcaaaaggaactattgtttcaagtagtttctgctttctttcttatcagcatccatttctctattcaattttttttctttgttaagtgattggagtgctatcttgtaatttgagatattcgttatgcaaggaagacaagaaggacaagatagatcattcatcagggcccaaatgaagagaggcaagccttctgggtttctggttctaccaaagcctaatgtagctactagcatacagagaataatcaagggcatcaagaatttatctcaattatttggtgagtacaacaatctctatttttattaattaattttgtttgttatgaatcatggctggtcttaattacatgaaatttcaatttccattacccgataaaaattaaagaaacatataaatcattttatcaatatattctaattagcagacaatcatactactccttgagtgatcaaatatttgattattaagtttgtatgtatgtgtgaaaaaaaattcaataaaagatataagcttcttaaatatatcttcatatctcaaaaaattagtttccaaagtctttgtcctgaacagcccataaatcatttcttaaatgtatataatttatggtaggtggtgcattattctttatcctttctgatatataattctctttttcttgtggatctttctcttcttctttttttatgtgttatatgattacttttggactatgtatgacctgggctactcaacttgtccaattgaatatactggacacaacttttatgggcccataacattagaaaaatgaaacatatatatgtagctgaaatttagaggcatcgaaggagttagttagatcagaggagtagtttgaagttttgaaggccgattatgattttaggtgattatattaactcgtctcccttatttgaaatgattttatccagctgtggagtatggggacactaaattctaataatatgcatacaacataattgtatttgattttagtgtctgaatcctacctactaaagaaacggacacaacattaaataaatgagaccttagcaaaatatatgaaatcatgtataattaaaaaaacaatactcgttagttaagttaaaaagtagataatgataaaactaacgaaaatagttatgaaatcatctgtacaaccaataatgaaattagtaacctgagttctgtggcaaaaaatggaaaacaaggaatgcaataactaaacacaaatttaaaagactcttgcaagaactctcaaccctacgttgatctaaaactgtctctgtgtgtgattaagtttttttaacatctacacattaagttattaaaatttgatctatcattcaagaaaatttgtcattggtgccttttcaaaaatagaataaatgtcgttggactgggctttgcttacagcatatggactgggctagagccacatttaaggttaattttaatataattctttgcttaagttattataatattctaacttatttaaactctacatgtttatattcgctctgttgcatctttgtaatgtgtcgattgttgaatgttttcttcacttgaatttagtttacaaaaaagaggatgtcgaaaaaatggaacctgagatggaaattgggtatccaacagatgtgaagcatgtgacacatattggaattgatgggtcaacaaccattacaaataatgtcaagggttgggacaatctgaaggcacctgaattattgtctctgtctccaatttcattcaagcaatttgaattggccatggctagccaagctcaatatcctctcattaacgatcttaattcctctaaatgtggttagaacactttatgggaatgatggcacttccaagtcccttcttctaattgtagattaatgatgattgatgacgatgtatttaggagttcatatacaagtgcataaattgaatattcgtgaatgtttgtaatctctttcactctttgttttgctcggttcttgagattgcctaagcttcattacttcaagtcttccattttaaaattgagtttttctgtctataatattttatcaatcaaaagaaagaaaaaagcttagtaggactcactttttatcaactaaaaatttgttttaagttatccatcgctattcatttaactattattatttatttattacagattttacctataaatattcactggtgtggatgttgtagtcatctctacacatgtaaattttgatttgacaataaatctaccttaaaactaaataaattgattgatatatccttctatttccaggaaaaaaaagaagcaaacataatgataatacacttttttattattcttaataattttctattattctcttattactttgatcatatatgtttttatttttttatttttgtctcagtttatttcgtttgaagtccaaaaatgaaaaaaaaaacatttttagctatgtcttattttttatttaatttttggagatcattccacaacatagccaatacaattaattttttttaaaaaaatatacacaaaattagtaaaatgattttttttgggttcattaattaattaatacggtgccttggaggattgtaatatacgccgacaacaaatgtggctctttagtcatttcataaactaaattggcatgttcattcaacaaatatatttctaagattaaagcatcatttttaaaacatattccttagctttttaaacatataaaacaattcataattaaataatatatattttgtacagtaaaagttatacaaaaatttatataatcatgacagagattgaataagacagagaagtgttaaatacaacaagcaacgtgatgaaaagaactagagaaaaaaaactactaatgttatagaaaatggtgtaaatatttgttatataatataaatctataatttgatataaagagtaaaagagtgattaagttttagattaaattgcatactgatataataaaaatatatattttagattaatttacaaaatattgcttcaattttaacctattggtactttttttcccccaaaattataagtttacattattctcactcaaactttaaacatatagtaattatttcatttttttttctagaagtataactaagttccatacaactttatatgtatattctaaaaaattattagaaagaataaaagatattcaactgttaatgatgaaaaaattgtgaattattaaacatttaaaatccaaacagataattgaaaatttaagaaataatttatcaaaattataaaattatgccaagcttttgtcattaaagttaagtttatgttctagaaaagaccatttgtttatggtttagtgtgtctattgagttgacaaacaaagaagtgtgtactattgagtacaattaattatagaagataattcgttgataagtttctttccgttgccgttacttatgtcattctccagttctttctttattccttaaccatttccccccctcttcttcactctattctttccatagttaaaaacttttaatttcaaactagggagaataattttaataaaattaattttaaattagtaatgtcattacaataaataaataatatatataaactaattttaagttaaaaagtgtgttaataaaagattttaaattgtgatcacaatttcttaatccctgattacaattttcttattttttgagtattaaaataattattttaaaataatttgaattatattttgtgattgaaagtgtataaaaattattattttaattttaataataatttttcatattttaataaaagatgtacattttaacataagcttatgtcaacaattaatttcagtccatttcttttgcaataataatcacgttagaagagaatagacccgcgaatctgaatcagaaaaaatataggaataaatagacttgccaactaaaccgagtgaaggggcaataaggtcattcgaggtgtcctccttattatttgtcggtgccgtagcgtggttctttgcctcaaagccagtctgttcttacttaaacatcgaacacttcgtgtcccacctagaccttacccaccgtcgattcccctaaacgtgttcgacatttaatcacacacactaagcacacataacaccatcggagaaggaccttccatcaacccataactagtcgtagtattatttcattgattctctcccttctctttctctctgcgacaacagatgatggagacgacggcgaagcgaaacgctgtcgttgcgccaatcgcggcgttgttgttggtggtgctactcgcggcgagtgtggtggtggaggctcaggagaactcggacgtgaagacgctggtgaagcacatgcacgggaagaagtactgcgacaaaggatgggaatgtaagggctggtcaatttactgttgtaacctcactattactgattatttccagacctaccagttcgagaatctcttctccaagcggaactcgccggtggcgcacgccgtcggattttgggactaccactcgttcatcgccgctgccgcgcttttcgagccgcaagggttcggcaccaccgggaacaagacgatgcagatgatggagattgcggcgttcctcggacacgtcggcagcaagacctcttgtgagtactactctcgtatcgataatttgttgatttagttttgtcaacttcgttgagattgcgcgcgcgtgtgagagagagtttgtttaggttgagctagcggtttttgaggttgtgagatctgtgtgtgttgtagtgtttgagacttcgaatgatttggattttgttgttgtgttgcataatttgatttctgtgctgtaaaccttgttttgtttggaactggagtcggtgcagtcgatttttcatgcatttacatagtcattgaatgatttggatttgttattgtgttgtctgatttgattgctattttgtaagccttgatttgtttggaactgtattctgtgcattcaatttttttcatgcattcaggcagttgttgcatttgtaactgactattggatgaagattggacgactcggattttgaatgtgtattttatggtctggtttatcaaaataaagcatgagtcatccgatcttcattcaacggatctgagtagatgaatgtgtgaatgcgtggaaaattgtcttagatgtctttatttgtgatttacatggtgtctgatgtgtttaagcacattttgtgaggtagaatgttgttttgtgagagttgatttatcggagacatgtgaataggatttattgtttgtgttgtgggagttgcgtgtggtgcagcggttgatttagttttgctttaaatttggacaactgtgtggtgttattgttcttataatgctggtgtcaggtggttatggagtggctaccggaggacctttggcctggggtctttgttacaaccatgaaatgagtcctatgcagtcatactgtgatgactacttcaaactcacatacccctgtacacctggtgctgagtactatggtcgtggagctattcccattttctggtatgtgtatgtttgtttggctgtatgtataatagaagcatattttcctggttttggaattgcctgtttattgtcattgtttgaataaattgcgctgtaatacatagagatgcctaaaggtagctagtcttagaaagtttctttatcattcctgaaaattgccattcttctcaaatttttcttgtattgtttaacttaagtattctgcaatatccctgaccagtctgtaccaaatcaattagacttgttgaaaggttaaccaaacatcccaaggttatttgttcttaatttatagcttcccctattgtctgaaaaaatgagttaatagatagcagttaggtactagtgctctcaacaattaattcatccgaagcacctcaatggctatgatcataaacactttagcaaagaagtatatggcctgagcagtctatgaaatgtaaatgacacatttcatagaaagataactattatatggttaaaacctttaaaatatcatttagcttcccattgtgcgattataggtccttcaaatcacaataaagaaaatataagagttaagtgggcattgcaaagttttgaaagattatagtaatgaaaaagaaactgttgcaacaagagataaagtaggataagggtgggggaaatagaacaaaagcagtaattgattagttactgcagagatacagagctggatttctgggtttgtgtaaatgatttatgaaattatcagtgataccaatgaaaattaaaaataatgtatgcatgtgcagattttcagttaagaccagtcaagtcttaaacctgttcccttactttctattgttctgatatactagtttctgtttggtctgaatggtaggatatttgaccatatcagtatattggcagtgtgatcaacttaagtagcatctcagactgtcaactgacattttgtcaaagaagaaattgtttgaagatttaaactagtattttatgcatatttttaggcaagtctagttatatttcattggagccattttttacttgaaacgtatagataggctgtaagaaaactatttattaggttttatcaacatcattgtgcttgttgaatgtggaggttatacttaatggtatcatcaatataggaactacaactatggtgctgttggagaagctttgaagatcgacctactcagccatccagaatacatagagcagaatgccacccttgctttccaggctgctatttggagatggatgacaccaatgaaaaagtcacagccctctgcgcatgatgcctttgtcggcaactggaagcctaccaagaatgatactatggagaatcgtcttcctggatttggcactacaatgaacattctctatggggatggtgtttgtggacaaggtgatgtggactcgatgaacaatatcgtttcccattaccagtattatcttgaccttctaggtgttggtcgagaacaggctgggccccatgaaatccttacatgcgctgagcaggttcctttcaacccatcttccaaacctgcttcttcatcttaagattatatttattagcatatgatattgttgtaccaatccctttgaaagtgaaagttaataagggttcttgaaattattggtatgcaataattttgaactgcgtggtagtgtgggaattcattatatagcttttgttcgttgttatctagaagttttagtcatttgtaatatcatatgtcgtgaaaaatttgtatctggtatatatgtacgttatgggcgattttgttatttttatatatacattccttggttgcaatgtaaatttgaatatggtcatgtcaaacaaaatccatttcgagtcatctaggtcttcattagtggctaacaatataagttatttatgccacgtatttaaaatttcaattaaatagcattattgtacatctgccacgcagagaaaatagaggaaaatgatttgcgtgttaatttttaagatgacagggttatgttctaattttgcatttgacataaagtctgctttatgttttaacaagagtaataaatgtctttttttagaattatcctcctttcggggtgattgcctcatgttaatatcaaatctaccagaagcaacacgagatagtagtattttgatgatttttgtgaagcctataatgaaagcataaggtatgaaaatattttcaggggaaatgtgttccattctaattcaaataagggcatcttgtaggtttcaatacaaaaatttgcaaagtaatccctataaatcgtctgtaaaataagtaatgtaaatctgcaacctaataaaattatcttcttggccgaaatatgatcagttgccacatttgtccataaatctgaatctaataatatgaaaagttgagataaaaatttattgtcgtttatagaaataaataaatggaccggtctttactcatacagcagaataaataaagggacttatatccttcacctctctaaagactaaagtggcttttcttattttggaatacctatttcgggtaggttatagagtcctcttttattttagaatataatcatactctaagatagttattttaaaagagtcttattctgaaatgatactctagaatataattttttgcattccagaatagctatttcaatatatgtaagttttcgtattgtgtggaaacatatcttattctaaaataatagttttggagtactcctttggcaatggtgttttcaaagatgaactcctgcgcaaacataagcttctccagggacaaatccatggtggtggaactcccaacagaggccttcatggaggtgtggcggcaattgtcaacggtgttccgatcatatggcaaacatactgtcgtgagcgacaaatcagagtggcgttccactgtgggccaatgtggcttgcacgactataggcccaaaggctgtatctgttcataattaatcgatcgagcgcaattttacttggattacagtgatgttttctcgtttttatgattcacaacatccctgtatatatatatatatatatatatatatatatatatataatatatatatatatatatttacaatgtattagttaacttattgaattcaaatgttaatttaaatacacaatgataaaaaaaaggataacgtagtttgatcaagtctaatatttggaaagtacaaggctaagaaaacaagtacatctaaaataagcttttagaagaaaaaaattaagacggtaacgaataatcttataactcatttaaccttctccatgaaagcgttcgtgcacaagaaaataatcgtattagataatgttttctgaaaatgtaaaaaggatttctctagttatttatttttattatagattagatatctaaatttagtagttatgggcacaaatgattatggttgacatatttttcgagtgtcctccggcgtggattcggattgctattttaacggattttttaagttttcttttactgtggtttctagtttgagcttaactctcttttagcaccaaaaataaaaatgatattaaacttattttagttttattgatatgtattgatgatttaatggtatagttatcatgttttgttaaaaaaaaagaatcatttttacgatgttgacatttttttcacagatagatactgtcattttagatagttttgtattttaatacacttgtctacattatttagttgctcataagtttacaatcattaagtaaaataaataatattaattaagtattttaattagaaatatttaatcaataatgttgcattgctgcttaaatttactataaagagtgtgtgtgtgagttatgtgatgaagtagtgtttctacatttcttgttgcacaaatgttacctaaacaatttcatgttaactttagagaatgaaaggttaggcatgcaattttactcttatgactaattagtattttagactgtgcaatgacaagataattgtctaatttatataactaaaatttgtaatatattaatatattttaataaataaattatattataattaaatttaaaataaataatatttttaacttataaaatatattttagacttatgataaacaaattatatagtgataacag

SEQ ID NO: 12 - DNA, 32767 bp, synthetic construct
  misc_feature 1..32767: note = Artificially Synthesized
  source 1..32767: mol_type = other DNA
ttattaggttttatcaacatcattgtgcttgttgaatgtggaggttatacttaatggtatcatcaatataggaactacaactatggtgctgttggagaagctttgaagatcgacctactcagccatccagaatacatagagcagaatgccacccttgctttccaggctgctatttggagatggatgacaccaatgaaaaagtcacagccctctgcgcatgatgcctttgtcggcaactggaagcctaccaagaatgatactatggagaatcgtcttcctggatttggcactacaatgaacattctctatggggatggtgtttgtggacaaggtgatgtggactcgatgaacaatatcgtttcccattaccagtattatcttgaccttctaggtgttggtcgagaacaggctgggccccatgaaatccttacatgcgctgagcaggttcctttcaacccatcttccaaacctgcttcttcatcttaagattatatttattagcatatgatattgttgtaccaatccctttgaaagtgaaagttaataagggttcttgaaattattggtatgcaataattttgaactgcgtggtagtgtgggaattcattatatagcttttgttcgttgttatctagaagttttagtcatttgtaatatcatatgtcgtgaaaaatttgtatctggtatatatgtacgttatgggcgattttgttatttttatatatacattccttggttgcaatgtaaatttgaatatggtcatgtcaaacaaaatccatttcgagtcatctaggtcttcattagtggctaacaatataagttatttatgccacgtatttaaaatttcaattaaatagcattattgtacatctgccacgcagagaaaatagaggaaaatgatttgcgtgttaatttttaagatgacagggttatgttctaattttgcatttgacataaagtctgctttatgttttaacaagagtaataaatgtctttttttagaattatcctcctttcggggtgattgcctcatgttaatatcaaatctaccagaagcaacacgagatagtagtattttgatgatttttgtgaagcctataatgaaagcataaggtatgaaaatattttcaggggaaatgtgttccattctaattcaaataagggcatcttgtaggtttcaatacaaaaatttgcaaagtaatccctataaatcgtctgtaaaataagtaatgtaaatctgcaacctaataaaattatcttcttggccgaaatatgatcagttgccacatttgtccataaatctgaatctaataatatgaaaagttgagataaaaatttattgtcgtttatagaaataaataaatggaccggtctttactcatacagcagaataaataaagggacttatatccttcacctctctaaagactaaagtggcttttcttattttggaatacctatttcgggtaggttatagagtcctcttttattttagaatataatcatactctaagatagttattttaaaagagtcttattctgaaatgatactctagaatataattttttgcattccagaatagctatttcaatatatgtaagttttcgtattgtgtggaaacatatcttattctaaaataatagttttggagtactcctttggcaatggtgttttcaaagatgaactcctgcgcaaacataagcttctccagggacaaatccatggtggtggaactcccaacagaggccttcatggaggtgtggcggcaattgtcaacggtgttccgatcatatggcaaacatactgtcgtgagcgacaaatcagagtggcgttccactgtgggccaatgtggcttgcacgactataggcccaaaggctgtatctgttcataattaatcgatcgagcgcaattttacttggattacagtgatgttttctcgtttttatgattcacaacatccctgtatatatatatatatatatatatatatatatatatataatatatatatatatatatttacaatgtattagttaacttattgaattcaaatgttaatttaaatacacaatgataaaaaaaaggataacgtagtttgatcaagtctaatatttggaaagtacaaggctaagaaaacaagtacatctaaaataagcttttagaagaaaaaaattaagacggtaacgaataatcttataactcatttaaccttctccatgaaagcgttcgtgcacaagaaaataatcgtattagataatgttttctgaaaatgtaaaaaggatttctctagttatttatttttattatagattagatatctaaatttagtagttatgggcacaaatgattatggttgacatatttttcgagtgtcctccggcgtggattcggattgctattttaacggattttttaagttttcttttactgtggtttctagtttgagcttaactctcttttagcaccaaaaataaaaatgatattaaacttattttagttttattgatatgtattgatgatttaatggtatagttatcatgttttgttaaaaaaaaagaatcatttttacgatgttgacatttttttcacagatagatactgtcattttagatagttttgtattttaatacacttgtctacattatttagttgctcataagtttacaatcattaagtaaaataaataatattaattaagtattttaattagaaatatttaatcaataatgttgcattgctgcttaaatttactataaagagtgtgtgtgtgagttatgtgatgaagtagtgtttctacatttcttgttgcacaaatgttacctaaacaatttcatgttaactttagagaatgaaaggttaggcatgcaattttactcttatgactaattagtattttagactgtgcaatgacaagataattgtctaatttatataactaaaatttgtaatatattaatatattttaataaataaattatattataattaaatttaaaataaataatatttttaacttataaaatatattttagacttatgataaacaaattatatagtgataacagatttctattttgagcacatgtgactaattattgaatcatcttaaaatgcatattctagttcaatttaatttgaatcagattgatattcgagataactttttcaattttcagtttatgtagttataactttgcagttcaattatcacaatgtgcttctgattgaatatgttgatgaacacacttcaattataggaaacatggaaaacacaagtcttttctctcatttgtttctcccatgaaatctctgtagactttttccaagtcccactccttaaaaaatgttgtcgggacaaacttgttccccagatttacagtcaaataaatacgatcgagtttggacaaacaaggatgaagaccagataaactacctcaacctcatgccatgcatgatttttttcttcttaatagaaattttaataatcaaattgatgcacagactattttttcttctttttttttttaccactttagttgggatctttgattttttttaaagagattctaaaatatttttactagttttaatcgtttgatcagttatgagatttgttctcataaatttaatatgtattgtgaattgaattagacttgttgcataaacagtattcttttaaaataatagaagttgggtgattgaaataatcgactttgttgactgagtcaccaacatgttcacgttaagttttttaatcgtgaaactgacgcgcatggaagccaaacacgtgcatgtacatttacgagtaaaaaatggatgaatctagaatgtcgttaatttagacaaagaggaaatttggtagtgttttctgctattacacaatctttccaaagaaaaaccttcaacaatctattttgtcttcatcatatatataacatgagatattgattatccttatttttaatcaattaaatcacttaattttttttaaaaagtatttttacaatgttgataaaatcggcctgcccaattcctcatatttgttttttactcgattaattcattcattactggttcaagtttatttatttaagtaaataaataaatttaaacttaaaattaaacttatttaaatgaatggaacccaagctttgaaaattcaacattatataaagattgatgaatagctcgtttatgtatatataaatcatgttttttatgaaaataattttatctcttaaaaataagttaaatagattattaagtatgttttctattaatttttaacttcacttatcacatgctagactcatttttctattagtagataattattaaaaattatattgataaagtttaaaatttatttaagaaaattttcataaaaaaataattaagctataaatgagctaaataagtcaatctaaagttttaatatttcataacaaaccaaatccaaactttctacaaggctagattaaataaataaatcaagcttgagtcattaattttttttcccacaaacatagtccaagtttcaagagctcaactggatttacccttatttacatccctagtgttaaatatttgaaagaagagtttatagtctataataatcctataaagtttgaacaaaaggtggtctaaataataatttttccaatcccttataattaattaacgagtcgtttgttatgagactattttagtgattttgataaaaaaaaaatagcttatttactttttgacgcaacttatttactaagaaagataacttatgacaactacaccaattccatttataattttttttggtctgtaaatcaatcaacaatgaatgccaattagtaaaatttgacgactatgacaattccatttgattctgtcaaccaacagtgtacatcaattgttttaattgaccattaattatagtagatggaaaaagactaatattttaaatgaaaaaaaatttaaaaatggtataattttttgaataaagtctctgtcgtatacacttttttttggaagaaatgtcttatacatgcatggttaataattgagtcccaattaacgtatttaagagactctctattttaaggtaaagagatcataattatcataactttttttaatgttatttttacacaaactaattaatatatttaagggactagtattattttggactaaatttattatgatttaaaagtaactttaaatctaaaacttaacataaaatcttaaaaaaataaaataaaagttatttgtttttttaactaaaaaatatttttataactaaaaaaaagttttttttccggtcacaccaatttattcaatatgaaagtagaaagaaacaaaaaatataacttgtttttaaaatttattttccttatttaaaaaataagtatttttattttttttgctacaattttaattaacctagctttttttaaatgaaaaaaataaaaatgtgaataactattttttggtaggcgaaaaattaggaataagttgaatcaagcattactttaatatttcacaaaaaaaaaactttaatctcttcttgaaatgcaccaatctctcccttacagtgaaaaaaatatgcttaaaataaatataacatgtcttaactcttcaaataaatatgcttaatgtgaaagtttttttttccgtaaaaaaaaaaaaactcttaataaacttttcttcgtacaggcaattggaaattaaactcaaatcaatctacttaacttaaaaacatgatttattgtctttaatagatttgactgtttttcgaaccaccgacatctaacagagaagaatagatcaagtaatgataaaccattaataacattgaattaagtcattaaaagtaagtgttacgtacttttgataatttacgaaatttaaaaccgtatttgaaccgaaattatgataatggcatgaagttcatccgtcatattaggtatatccttttacatattttttgacataattttaattatttaacgattgtgtaaaattttctgacaaaaatatatttaaattattcagtaaaatattttatatgagacttatttaacattaaatattaggtataaaaaaactgtcactgtggatctattaattaattagtgattttaaatttgaaaattttaaatcaataaatattgataagtttggatggattgtttattctaaaaatctatgaaaatataatgtttttgtgtatacatttttacatctattttatttttataattgcattcataagttattattttatctttataattattttcacaaatcattcactatattatatttatttttaattaaaaaatatgaaaatgcagtgagacatgaagtgcctattttttccttataaaaataaaaattataacttgaaccgaattaatctaataagatttgagttacttaggcagagttataaagccacttaaataaaataaattaataaataaatttaacattgaattaagtcattaaaagtaggtgttacgtaaaattctatgacaaaaatacatttaaattatttagtataatattttatatgagacttatttaacgttgaaaaaaatgtcagtgtggatctattaattcattagtgattttaaatttgaaaattttaatttattgataagattgaatgaattgtttattctaaaattctataatgtgaacatgacgttttagtgtatacatatttttatatttattttatctttacaactatattagtaagttattattttatttttataattatttttacaaattattcactagtattgtcttttattttaattaaaaaaaaagcatgaaaatacagtaagataggagatacttattttctcttcataaaaacaaaaattataacctaaactgagtcaatctaattacatttgaggtagtttgattggattataaagccacccatattaaataaattaataaacacataaaaaatagaaaaataaatattgatgtaaaattataataaaagaaaattttctgaaaaaaagttgaaatgaatataatttattagtactttgttttctgattccataatcctatccatccactcccgagaaacccaatcatccgcaccgtccaatccgcgcgtcattgtttatttatgacgttgtcgttctgatccatactctcaatcaatatttaattcccttattttttgcgtggcggagaaacaaaatctgtttgcttcaaagccaccacctgccggtgacgcacccgctcgtgacggtagcgctgcccttaacgttttccgacacaagacgacgaacgcctcgttcaattcttcttcaaccgacatttttgtttgattcgaagattcgcttctcagttttcgcgggcgattctcaaggaacccaaggagatagcgctggatcgagaatctcgattcggttatttgcgatttcacgctttgatttttgcgctattcagcatacgcgttttttcagttccgattctcgcgctttccgacaagtttactgtgctgatacacggctggaatttccgatttcattgcaatcatgactcaggttccatttttaattgcttttttcttttatgcgtttgattgtgttagtttctgaatctgaatgtctagggccatggtttgtggtttctgaaatgattataattttttttttttatcaaatgttaattgttttgttagcaataggaatcgaacctgccaccttttccacctttccttctttcttaaccacccaaccaaacttatatcttcatgaaatgattataatgatggtgtttgatcatgattatgatgatgacgatgaatatgaatatgattatgatgatgacgatgaatatgaatatgaacatgattacgatgatgacgatgattgtgattatgatgatgtttgattattgttgtaatgatgatgatgattgtggcaggcggtggaggtgctgaatcggtatgggacggatctggtgggggagccgagaggttctgattctgagtgtttggcgggttttgagtgttttctgaacattgtgagggcattgggaatgggatcgtgcatctcggaggttggagctggtggaagttcccctcctcttcttcctttttcgcctgaatcaaacatggatggtgggaagaggaggaggttaaggggttcctcttcctttgatttcaaagtgcctgggaggatgttcttgaatgggtcaagcgaggttgcatccatgtattgcaagcaaggaagaaaagggatcaaccaagatgctatgcttgtttgggaggttagcctgccctcccccctctcagcatctattaatgttgtttcgggatattgtctttttcattacatttgtatgattgtatgcacattatgtggtttgcagtaatagaaatgaaggaagtttaaaaggaatcaaagttgagtaggtattctgcttttgtcataagtttttttattttattttttaaatcattgaattggttagttatgtcttattgtttagcatatctttcagcttagaacaagtttacatgatttagtgcttaaatttttattttttttgtgtgtatttggaataaatatgagctgatatgtggagccttgaagtaatgagtatgctattacctttgtctgacctttacctctgttcatttcttttccaaatactcttctcaatcttcttaggttgttagtagtgccttgtcatactgccaatgcagtataaaatataaataacatcctccctccattcttttctaatagatcattgtgttctccatctcttcccacttcagcatagtttgaatgagatacatcaattttacaaaacaatagtttttgttataatgtcctacatcggccaagaacatggccaaaatactccttataacagttgaaaaaatcttctttccttgagttaacttttggggttgagttaaacctaacccattgctatagtttctttattctctttctcttacaatttgagtcttcagatgtgtactcacagttttgttgtttcagtttgcactaacgttaatgatctagatattaataatgtgtacaaaactttgtctattttccttgtgcagaacttttgttcaaaggaagacaccattttttgtggtgtttttgatggtcatggaccttatggccacaaggttgcaaagaaagttagggattcttttcctttaaagcttaatgcccaatgggatttgcatcataagaatagagatggactcagtgatcatagcagcgccacaggaagttataaatctgaagggaatggatttagactggtggatgagaaaactagtcccactgatcatgaacttgatgaaacagatactattttgacattacgagaatcttttttgaaggcttgtaagattatggataaagaactgaagcatcatcctgatattgactgcttttgtagtgggactacagcagttactttggtcaagcaggtaagaaatataaattacaatgtaatgttctcttgattatttcttatttgctttttacatgtggtttactgatgttctttcaggggctggaccttgttattggaaatgttggtgactcgagagctgtattgggtacccgagatcatgaggattcccttattgctgttcagttgaccgttgaccttaagccaaatcttccaagtacctctctttctctcttctcccctccattcccccctccattccccccttgtgtgtatcttctcaatgaacttgataagttggagaactccatctccttattttatttcccttgaaatattgaatcaatattgccagcttcataattatgatgtgagtgacagtgcttgtctgatttagataaatgaacttgataagttgaagaactccatctctttctcttttctgttttgattttaccctgtgtctttgggagatttaaataggtttccctctgctttatggagataatctctattttttttatcaaacaatacaaattccctgtagctgaaaccttacttttttctttgtttctctgatttattatatctaacttcaatgcctttttctcttttgttttagggcctgtttggataagcttctttagaagcacttccaagagaagaaaataagaagaaaataatgatgagtttcttcataagataaaatgagttatctattagctacccattaactaatttgtagaagctctctcatatagcttctctaaaagatgagaggacgtctacaaattagctaatggttagctaatggacaactcatttttatggaaaagctcatttcatttttttcttcatatcttctcctaaaagtgcttccagagaagcttacccaaacggacccttatccaatatatttcattggttcccttttagaacaacttgacttttgtgagacaaatgttttcaggattggtgaatctatattttcattgtatgatttaataaactctgtgacttggaaattggaatttattttgatttttgatacatcagtcagtgtggcatatctatatgggtatgaatgtgtgatgtaaagttgttttcccttaggggaagaagagaggatcaagcttcgtagaggaagggttttttcccttcagaatgaacctgatgtggctcgagtttggctgcctaactctgatttccctggtcttgctatggctcgggcatttggagatttttgccttaaggactttggattgattgctgttccagatatctcatatcaccgccttaccgagaaggatgaatttgttgtgttagcaacagatggggtaaggccttatgaggctcttttcttatgatatactgtcttctatgatctgcatgtgttgaaattgattcatttcaatatcatatttgtagttagccaattctaaaatgaaaatatgaaattgactttaaatagtatttgaatgccacttgcaacagttaaaaaaaccaaatttggttctaatttcactgtatttaaattgacatcttactgtttaagctaactacttatcatgtaaaacttattattatgagttaactactaatgtaaatgatgaacacaaacctaatatcaaaagctgtatcaatatatattttttgatccagttgtatctcaaacccatgttcatatacacagatacacatgttatttctattctctttcaaactggaatttgtaaattgttgagcttttggaaaagcttgttctttacatcaatatagcgatttacccttttctttcgcctctcctttgaagttgtaactctaatttttctcttatggtccatattatgttgtaacttgaaaatttatattctctgaattgtcttatatatatatatatataatatatatacaaatacatacttgattagatggaatattgaattattttaaactgttggattcttgggggcagatttgggatgttctgtcaaatgaagaagttgtggacattgtggcatctgctccacagtctactgctgctagagcactggttgagtcagctgttcgggcatggaagacaaaatttcctttttgtaaggttgatgattgtgccgcagtttgcctcttctttgattctgattcagatttcaagtcaacagatacaaaagacaagttaataccggaggcatctattgaccaatctgagcagttagctttacttggtgaaaagggagttggaaatgaagctgaaaaaaagcagcaatagcgcaagttttgtaccatgtctctcccaagccttgttggttttatcgaaaaggttgggtaaattgtgccgcaacagtcaaaagagaaaggaggatcattattttgatgatttttcggtagttttctgccaatattcttttcattatttatctttcatttccttcgacagcatttgagggaagagggggttatatatatatatatatatatatatatatatatttcttatctggaggaaaaggggtaccgggcttttttttttgttgctggaggtcattttccttctctgtatatttggtagatttggagtttttgtaaatccaactctctagattcatattatatagtctgttctgaagtctgccgtttttcatttgcatcattcttaggttatagtttccaatttagtctgtgaaaacatgagaactggaaagaattggatttatttcttatggactttcgagtcaggtttttgtacctctgtgttgtgttgccgtcagggattaatgatattgtctgtgtgatcctattatgggttttgtgtggaaaatttactctgacggaaatttgtctttaagctatacgagtaaaactgtatttaccttttgatttaaattttattatttcatgattttaacttaattttgagaaggataaattatttttaaaaaaggcttttttgttaagggaaggtgtcctaagtactaacccacgaaggaatgagggaagtgaggatggtaacatgtcaaataaatttggaatttagttcccgaaatttttgagtgttaattattgtaggaatttactcatttcatgataaaacttgacatggagtaccggacttaaaaaagaaaaacatgacatgaagtggatttggagtcaagttttctctcaaggcaaactggatttattctccgtactcacttattaaacaaggttaaaacaattaggtttgattgacattttggccttcagggttgtctttataattttttcatatacgaaaaaagttataagttttaatttctgtacacacatataagttaagttttgatctccgtgatcaacgcagctagtatgtatgtggcacctattcatttgctttactgagccttctcaagtgtttgttgtccacgcctgaatataatgatttattcaagaaaacaacggattgaggaagaactgaagaaggtagcagaatggttgaaggggaaaaattacctggtgatgcttgatgacatatgggatgaggttaaaggtgcatttccagatgaccaaaatggtagcagaatactaataacaaggaggtggcatactatgtacaagaacaattttcaataggatcatcttagaccttaagagcattgacgtaaagatagatgacgaagatcaagccatcattctgttgagctttcgtcctaagccatttgatcattttgctgatacaatgttgtatagaaaggctactctatcaatggatgaggtcaaggctgctttgaactcaaaagaaaatcaaggggagaatgactctggcagtaaaagactcttttctactttcttgtgctgatgtcagttttttgtctccttttttctttcatggtcattattgtgctgatgttttaacatcatttacatgtttattgtaaaaggctctttgctctggcagagtttgatgagatgatttgcaattcctctgcttctttctattcacttttctgctttcttgtgttccacaaccacttgaccagatttttcagttagcttagcatttaagcctgagaaattatgcatcatttcatttgtttgttgtacaaccatatttaatgttaacctctctcccttgtttttccattctaaacatggatcatgtttttgtttattggtctaaagtgttgtttcatgttatttttctttctcttttcccaatggaaggtcctttcatgatttagttagaatagataactgataacacaattagttggaaaggaagatgttagagtgtatgttgataatgggagacaaatcatagtgatcagttttagaattggagtgaaagcatacagacattgcttttgtgccttgtctctgctactctacccaaacctttgctggttttatcatggatataattcattattttatgatctttttggtagattttctaccaaaattcatttcttcttttcggtgatgctatatagtacgaattagttttgcaccaagaagtacgaatccacttggcaaaatatgatcgtacaaatattaatttgaaagaataaaatctagacatgcaagttctaactaccctctttcaaactggaatttgtggaattgttggaattttggaaaagcttgttctttacatcaatgtatatattctcccaaattgtcttacaacacaaaaaaacttgagtattttcttacatattatctgcctacatttttgcgcttaactggattatggaaaaataatttgctaatgagatatactaggacagatggatgaaaattacaccaacttttaacaaaggtttaaatacagtttttttttatttataatttatcatatttttgcctttagtctttatatttttttttgtttttaagctttgtaaattatatttattttatttttaatttttaaagcaccttaaataatattttgacaagtgaaaaaaagtgttatctaaaatgttagaacgaaaaataaaaaaatataatttataaggactaaactttttttacaaaaaaaataaaaaaaatactaaagaataaaaaaatatttaagacaataacaatttgctgaatacaacagaaaagttagttagaacacaataacaatttttaaagattattgttaaagactctgcattttaaaattctaactaatttttgtttctaaaccatggaagacttttatttcataaacacaataacaattaaacaaaactcacaatttaaaaaaaaatcagagtcatcaaatatattaatatttaaagacagaaaaaaaaataaacaaataaagttgagaaagccaacctataattaaaaaggttgacagaaacgcgggtccacgcgtatgcgcgttctttccctttattggcgtccacttgttatctttcgttctctactctgtaaattatctgtccacaaactctctagagtttccacccaagatttcctgtttcacagaaatggcagatagtgttgttgcttttctgctagataacttgtctcggctgcttgaagatgagcacaagttgctttctggcgtggaggacaaagtcaactctctttgcaatgagctcaagtttatacacatcttcctcaagaattccgagggcaagcgcagtcatgacacagtgaaagaagtggtgagccaaatcagagatgtcgcccacaaagctgaagatgtggttgacacctacgttaccaacatcaccaggcataaacagagaagcaaactgagcaagttattccacctcaaagaacaagtcatggtgcttcaccaagtcaactctgacatagagaagatcaggagtcagatcgatgacatctataagaacagggacagatatggcattggagaaggtgaattccgaagcgaagaagctgctgcagaagcagaatcactcctcaaaagaaggagggaggtggaggaagaagatgtggtgggcttagtgcatgactcaagccatgtaattcaggaactcatggagagtgagtcacgtcttaaagttgtttccataattggaatgggagggttgggtaagaccactcttgcccgtaagatccataacaacaatcaagtgcagctgcggtttccttgccttgcatgggtttctgtgtccaatgattacagacccaaggaatgtcttctcagccttctcaaatgctcaatgtcatccacatctgaatttgaaaaattaagtgaggaagaactgaagaagaaggtagcggaatggttgaaagagaagaggtatctggtagtgcttgatgacatctgggaaacccaagtatgggatgaggttaaaggagcctttccagatgaccaaacaggtagtagaattctcataacaagtcgcaacaaagacgtggcacactatgctggaactgcgtctccctacgaccttcccatcctcaatgaagatgaaagctgggaactcttcacaaagaagatttttcgaggtgaagaatgcccttctgatttagagcctctgggtagatccattgtgaaaacttgtgggggtttaccacttgccattgtggttttagcaggactcgttgccaagaaggagaagtcacaaagagagtggtcaagaatcaaggatgtgagttggcatcttacagaggataagactggagtgatggatatactgaagcttagctacaacaacttgcctggaagattaaagccttgctttctgtattttggaatctatccagaagactatgagatcagtccaagacaattgatccaatattggatcgcagaagggttcatacaaccacaaaaaactggaattgcagatacaacagaactggaagatgtagctgacttttacttggatgagctggtggatcgcagcttggtgcaagtggcagaaagaaggagtgatgggggtgtcaagacatgtcggattcatgatcttctacgtgatctttgcttgtcagaaagcaagtctgataagtttctggaggtttgcacaaactccaacattgataccgtcagtaataccaatccccgtagaatgtccatccacttgaaaggagactcgggtgtctctgcaaacacatttaacaaatcatgcactcgttctatgttcatctttggtagtgatgatagaatggacctagttcctgttttgaaaaacttcgagttggcccgtgtgcttgattgtgattcgtttattgggttttcgtcttatccagtccccagagatttgaagaggatgatccatctaagatacttgagaatatgcgtcaagcaccttccagattgtgtatgcagcctttggaatctagaaacactggatgtaacatatgaaaccacagtatccagtaaaatttggacgttgaagcggctgagacatctttatttgagtgggggagggaagttaccagtcgtcctgccaaaagcaaatagaatggagaatctccaaacccttttgttagatggtagctatggtatagacacgtatccacaacaagtgatttccctgttgaaaattgacatatttcctaggttgcgaaaattagctttgcgctatcctgatggaccagatatgttatcaagtctacaccacctcagcaatcttcgtagcctaaaagtaattcgcggttttgaacttccatcagacaccaatgcgtttccatcaaatcttaccaagataaccttagcagcagtccgggatttacatttcatgaagacactggggcaacttaccaaccttcaaatcttgaaactaagattcggcaaagttggttttcatcttgatattggcaggggagagttcccgcaacttcaagtgcttcatatgacgcaaataaatgtaagacaatggagattagagaaagatgcaatgcctcgacttcgccatctggtcattaatgactgtgaagaattgtcagagcttccagaagaactttggtcaatgactgccttaagagtggtgcatgtttcacggccctccgaagaattggctaacagtctcaaaaatgtggagcccaggaatggctgtaagctcaaaatctctaatgaactgtgaaaggcaagtgggaccgccttcttacattgcagacataagttgtagtctttcaatatccttcataatatttgttttttaatcccgaccccatttccattttctttgtccaaataaatttccaatttaaaatagcggatattcaaattgagaatggatgttgcttccttgccgtgtatggagttgtgtctcatgattattgattacatatttataatacctagagtttcttttctatttgtgaataatatattaaattaagggggtgtcatgtaaaagagaagtttttttttccctaaaaattatatattaaaaaatttattttctcatatttgatatacatttttaaaaataaatactaatgtaaaatgatattttttgaaaatatttttaatcaatttctcatgaaaacttgaatatttatgtatatgtatatatatagtttaactatatttaatcttgaattactaatcattcctcacagttattatacactacaaaagtaatcctacgattaattaactttatcatagtaatcatagccattgaaaattaaccatattcctttacatccaatcaaaagaaggagaaaaaaaaacaaattcttttagcacgtttaaatgttaaagctaattattcaaatgactaagaagaaaaattataagctccaaagtaaaacattgattatcaaattatcctatctaaaactctaaagaagttatgttaatgttaaatgaaatattcattcggcccaattgttaaaaaaaatcatagctgtacaaattagagaaataagatgaaaagggagagaaatatgaaataaaatagttaatgcaatacaaagagataaagaaaaaaaaatgaattgtaaaaaatgtaataagtatttgttgtaacaatattataactcttgctaaaaataggtcaaacaaatggagtcaaccagcacagaaatgctctacaataactaatgttaaatatttgcaactataacttcaattccgtgcgtgacttgtatatgttaattatacatttggcattgagatccaagctaagtcaaaaatcaaagtctaataacatatacaattggtggtaagattttgcaatttacatattcaccatgggaattgagaagaaaaatttgacccaaagcgaaggttcccaatggtcagtgtcctagttttccttgaagattacatttataaatcatggaagttcctgaaaagtcaatttctcacatagcatttttttcatgttctttctattttcaaatcctttgaccataattttgactttgatatatgtatttgttgcttcatttcaatatacccatttatgaagtagagcaagtagagacaaaatcatcacccagttgatgtttaaaccacaagcaaaagaaatgtagtggaaaccaactaaatggactgttcccgttcgctcatccctcactttaatgcttgaagaacctgtgagatacagcatagaaaatattactaatgagccattatgcaaaattaaaggaaataatgcaagtcatttgaatttgatttcttataatttttttgacaattctacccaaacctttatataacacgtcaatcctatattatctttaattcatcaattaaacataagtaagaaatctgatgtgaaacaaaaagtaagtggataaaaagaaataaggacgacacttttacatcaaacacctccaggatcagcatgcagatactttaaagttttaaaatggatcgttaaactacacagaactattcctttctcagaattaagaactaagggtcaactccgtttgagaaaaatgttttattatttttcattttcaattttcacaaaaatgggagagttgagacaaatgtgaaaaaaatactaaatttacatgttctattatagatgttttcacaaaaatctacaaatgttttatattattcatttatattatttaaaatacagaagattataaagaactaataatgatacttacagttgtactcagtccatccaataacctgattttcaaggtcatagatgtattacatgttctattatagatgttttcacaaaaatctacaaatgttttatatcattcatttatattatttaaaatacagaagattataaagaactaataatgatacttacagttgtactcagtccatccaataacctgattttcaaggtcatagaagacaagtttatttgtaagcactaaatctgcaacaaataaaaatacaggtgaggctgcatagtaaggtcatggaaagcaagctttctagtgatccaaagttgtgataactccattataatttacatctagaaaaaatataaggaaatttcttccaccagtaaaaataatacatttattataatatttagcttgtaagtttcttccaaaacaagcatggggccacttctctccataaaaaatctcaagaccaataatatgctacaaaactttcaccagcaaattgcaaaaggttgtcagaacctacattcacacctttaagccccctatcaattaatcatcatatctttgaccagattaaataaaaagtaaaaaagaaaaaacagaaaaaggcccttctcagctaaatcctcgtcttttgctttagaaggaagatttaggatgtagtaatcgaaagcaaacttctactacctcttgatttaggaacttgtatcaaaaaatacagaacacagcccaatttctttcaaatctccagttaagatactcacaagatcctatacaaatgtattccaggatatgcatttataatgtatttgtcttgtttatttaagaactcagaagtaatcaggttcaaaaatatcttttatctcaaaatgccattaaaaaagtaaccctttctttcctcttataatggctacggcagtctcattctaaatctggaagcagtattcagcaaattggtggcaaatatatctatcggtacagaaaaggatttgaagagaatacctcccaataaggtcatatttttgctatctcttgattgtgtcccactattttgccaaccaatacaccagaaatctccctgcagatgcacattagtattttatttatagtatggtatataaagattatgatcatagaaatcattgacataaaaaagaccagctcagtagtagtgcacattaataaatgagtcttttgaaaaatcattttaaaactattgcgagtatctaaaattacaaaaagaataacagtttataaggttgaaaatacgaaaagataatatttggtgcatttgcaaattgcaactccagaatccattctggaactcaaaactaatttgaagcagatgggatacaaaaggtttttaaaatgcattgtgggcactttgggcagaagcaactttttattactttagcccagaatacagttaggccaagatgcagttggtctaatatatatagagagagaggagggatcaagttactttaagaggaactcttttaaagttactctccatcctcacactgttcattctcttgaaacctaatagttgtaatgagtaattaatcttaatcattagattttaaggaaatgaatggtgaagatgaggagtaactctcttagagttactcttgaaactcttgaagtaacttgattcctcttcatatatatatatatgacatagaaggccgacaacaggtcccaaaacaagccaaataaaactcggctaatcataacaaaacacacaaagatataatgagatttgcaatatacttacagacggaaataaataatcttgtggataaaccttcaacgagagcccattttcaaaataaaatgtgacagctggaaatccatcatcaacactgcaacataaaataaaataaccattacacccctacatgaagctgataatacagacataaatgcataggatatgcaaagggagcagtccagttatcaaagcactccaacaatacatactcaataagaaagttcatagaacaaaacctacaaaatattataacttaatttcaaaaagagtttttccgattatcaaattaagatttcaaatggcaaatattttttgtccacttatcaaacccatagtcaaaatggcaagatttcctagacaattcctttaacatataatgtagattacaatttacatgcaaatgtcgttattactattatcaagaaaagaatttattaagacagaaaaaaaagatatagagacaggataagaaatcctcatggattacagatgacaccactaaaataaaaggaaaaaagaaacaaaaatcaagacatttgcaacacggcaaaccaacaaagagcataaaaccataaaccagccactacagattaagtaacaacattaacatgatcaaataaaaatcataaaacaatatgtgaaccgatactgtaaaacaagcaacagaattaaaaggaaaaaatttcaaagcatacattcattactgcatactacagtgaaaatagaaaaaatatataacaggaagataagacatctaccaaaataaaaaacaaagtgagagagtgaatagtcctatcctggaaaacttgctatgccatgaaaaagttacttgaagatccaggaatataacctcaaacaccaaccactacagatgtcattgctcatgacagcaagcttaaacaatgccaacatggcattcaattgccatattgaaggatggtgactaatttaaacaaataataacaacaaaacaatgaaatcagtgagtgaagaactgagttgatactagaggagctggtctggtaaacttaatggtaatttcaccctgttcaaaaaagatttctcaccatacaattttcaaaggagatcaaagtaaaacagaatttgccaaaagtcaaaaccacttctgatgtgtttttgtgcagtcttctggtttcctttaccacacattgctgaaggacaattctacctagaacagaggagacaaacactcaccaccttgccatttttaatgtgacatatgccatgcatcatggacaacacagctatgggagtgcaactgtgccatgttcaatagcatgcaaaagaagttttagagaaagtgaccctcaacacacccagtatggaagcacatacttttaacaacattgtcaaccctgaaccaccattatcaaaaccacacactttaggtccttccttccaaaacaaactggactacaaagaattcttctaaaacctgtatgccgaagtaagctagcaaaacacttgcgcttgttccaaccaacagcaagacactatatgcatacaatatgcacgcatgcaatatgcatatttcttttcagatgtgttcaagaaagaaaagattatgttagcagttaaacccccagttatagtagcaatacagtatatggattagttaatatgaatgttatgcatcctacctctcagaatattgaaaacatgtatactcgtcatggagagtctgaacttttagatcgggttgctgagaaattatctaacaagagaaagcaattcagacaactgcaatgtaaatttagaaatttagtaatttgcaaaatttaagatttttatgtaccttatatactagcggctcataaattccttcaggcagataggccaaggttgtaccactgtcaattattgtcccttttctgtctccttgtgttgatgtatctgttgatagactaaggaaagcatggccaacttgaactgcagtcatattaacactgtagtgtggcctaaaaaaccaaattttcaaatgtcatattctctcaaaatatggatttttgtaaaccaatatctgaatagaagagagattctatcattaagatctcatagctaaggctatgtttgacccagcttattttttgcttaaaagctacattaaagttaaagctaaaataaaaactttaaaattaaagtagaagttgtttggtagtcttagcttaaaaactccttttaagataaaagatttgtttcaactagttacggaagaaaagaaggagggaagatatgtggctaattttaaaaagctactttagttagggtttttttaaatgctaattttctcccttttcatttatttaatggtcctcaaatattaagagtttttctctaaatgtgtttggctcagtttctccttttaaggagaagaaaaaggaaaaaagagccgggccaaacacctacatatgtggcctagatactacttataatgacttgggcaagcttccaaggttgattcagccccagtctgatatcaattctagtaaaagtaaacaaaactatgagtgcataaacaaaattgaccaacatccagcatatagaagcatccagcatggacatgaaaatttaacaaagatatattgttttttcatttagaatataggggaagaaagtgctgacttttatggttgaaaaccactgaaatcctaccgccagcttcaactaacatgagttcactttacattccaattacagtttacacatgacaccttcagcttcaaagaatcttttaaaccaaaattttaataccactgaaatgatgacctcgttagccagttgaggcctacattccatttagagtgtagatgtgttgagacatgtcactgatgacttgagaagaaaccattaatcaatactccccctggactcatatataagcaaaaaagtgatcttgtaagttggacttaaatataagcaaatctaactaattttgtcttatttaatgctatcattcctaaaacatccctcatttaattgtaattctattttcaatgactcttttttattcatgatagcaagttccaacgaaagatattttagaaataaccttattttttcgcttgagattagtaaaattaaatgatgtcaactaattttctaaattagtgtgaaattagttatttttgcttatatatgagtccaaagggagtatctagcaaggacaggctagagtagagcttataaaggcttgggaaatcctcacctcatgggctagttcttgggttaatttagcccaaattcaatagaagctacatgttttattatatcgtaaaacattaaaatatttgcacaaaaaataatgtagtcactaacaaaaacatgattaaaaaaaaactagaacaaacttatcttcaaaagagtaattaataacactataagaaaaactaatctcattctaaagtgtattaagtgaatggcatactggtctggtaataatggagtcatgttcactttaggttgcacaacatgcccaatagcaaatataccacctccatttactccatttaagcaatgagcaaacatctttttcactttgccagaagaagcaagttgtgaaatcattgatgaattagcttttccaaatccaagtattccaccaagtgcctcttcattcgaagaacttagatctccagattgtctagcaccacaactacaaaacagcaaaaaactcattttaaatataaaaagagggagaaatatgaaccaaaagatgcatgcaatagacaacattaaattatcatacacaataagaatcaccgagaaaccattataacctgaatgcctccaaaatcacatcaagctgtttgagtattagaaaaaggtatttctaggagtgttccatatgataaggtgttctattcactgcctcaatatttactaaaccatgttatttacagcatgtttggtaaaactgaaaactttagcattggaagattataaattcaagcgctgatattgtgctaaaggaagctattagcatttaacatacctaacaaagataaaacaaacgaaattattgtccaggaaatagttcctgttgatacatcactgactgcacaaatgctgaattattcaatattttttttacataaatttatttttcagattgagtagggttcaagcttggaacacaacaaaaggaggactgcaagaagtacaagcctatatcaatatcaacgtcagcagtctcactctcaatcttactcaaattggtagctgacccaaaccaatatcaaaatcaagtattttttcaggctccaatattagttttggtgaggagttggacgcaatttatatacttcataatgaaataaaaagcatactcgacagtttttgaaaagcaatgcctgcacaccagtgcacccactttatgttaatatcatatgcagatatgagaaattatgctattgcaagtttgcaactaaagcaacaggttaaattactatgaacaaacatgcatggtattggtatctgagttcatgacttatgagaaattaatgtccactaacagcaacagatcaaaagtaatagacatacacaatacataatagcacacattactgccaaaagaaaactcacccaaatacaatacttccatttgctgaatcagttttaaggtccccagacacttgatcataaagtacgatatcctttacaaagtaaccagcagtcgagctcccatcaccatatatctccagatatggacaggaaatattagcagtgcatccggtcagcagacccccattgatctctttgcaaaactcttgatcacagggaacaaatttaccagaggaggattcctttatgtcgtagagtgtaaggtccatcttcgatacacagaaaaagaaattcaaatgaataaaaacatcaaatgttgaaatgagaaggctgtacatgattcagccatgtgagaattgtcttgcaagggatggtctaccatggtacacgccctgatagtgtaaaggattttatacactctcaaccaatcagaaactcccatgtaaggtaattatgttgaattttatcataactcatttaaaagtcacccctagcatgatttccgattagttgacagtgcaaaagatcgctacacaatgcataaattttaaactcaggttgtaatccatcctaattttaaaatgcatacacaaagcacaaaaaaaaaaaaaaaaaactaagcaagcacgtttggcaagtaatgaagataataagggtagcttaaactaaaaagatgagaagcattcaccaaggttgcttctggtggggcattctttgcactgaatgcaattcacccacataatgtcactaccttactaattatcaggcaccgatggtattcttcttgatattggcaggggagagttcccgcaacttcaagtgcttcatatgaggggaatatatgtaagaaaatggagattaaagaaagatacaatgcctcgtcttcgacgtctggtcattgacagttgtgagttgcctgagcttccagaagagctttggtcattgactgccttactaatggtgcatgtttcgtcgccctcccaagaattggctaacagtctaaaaaatgtggtgctgaggaatggctgtaagctcataatctctaaaagttgtgttgatgaatggttttaagctcacaatcatctagtgcttcataactgttggaataatggcaatgcaagtgggcattgctgggttttgttatggtctttctatatccatcatgataacgtgcagtggttcttatcattctgtatctttctaactttctaatctcattgccacacgtgttgtgattgctagatttccattttaatctcagtttttgtctaatttctttttctttcttggacctttgttctgaagttgtaactttatttacatgttgattgtaaaaggatatttattctggcagtgtttgattaaatgatttgtaattcctctgcttctgtctattcacttttctactttcttttcttagatgacccgatatttctgttagcttagcattgaggcatgagttacatatatgcattatttcattcatttgttgttcagtgtgctcaactatatttgattgtgcgttaattttccctcttttccccttcttttcccatccaaagtaagtgtttcgtctggtccctaatataaagtatttaacttttttccattatttatcctattaaatactctaactatatgtacttttaggtaattatgcataatattgttatttatgacaagttaactatcattaataagagtaaaattgatacattaatttatttatttttattggtatgtgtaattttgttatttttatttattggttttggtgttgtctcacgagatgtctttttcctggttgaggtgtgtttctcttctcatttcttagatggaatagttacttgataacataattaattgtataatgggagataaatggttttcatcttctcttctcatttctttttttcccattcatttttctggccgagtgatacagaggagtaaaggaatgaggacttgtttcaagtaagagtaactccaaaatagcaaagagactgaaaagggaatttctgagtaataatttctctgccttctttcattccgtgcttggatacatataggatagatttttcccctaataacctgaggatacgagtaataaattcaaacagaattcagattctaataacaatatgaaaatgatcaattttaggtgataaaagtaattactcttaaaactaaatggcagagctagtgtccaacctagtccttcgtagacatggcaatggggcggatcgggtacagatattgtctccccaatctcttaccccgactcctcaacatatccccatacccgatacccgacaggtttgagtttattgtctcatccccgtacccgtcgggtatcgggtatccccaaccccaatccacacaccatttagaaaaatatttttttttttgtaaaaaaatattaaaaatttgattttagaaaaaaataaattgattgttaaacatttatttttaactacttatatatcaataaatttattataacgcgtgtgtctacataaatagttaaaaaaataatattaaattatgtaaaaattttaaaataaaattaactagtaataaaaattatgtcttttgattaaattatgcaaaaattctaaagattttaagtggcagggcgggtttgggttcggggtcggaacgggtctagtaatcccatacccgtacccgtacccgacttttggttattgaaaaaaacccgaattcatactcatacccggtcaactcggatattacccgtcaaagttgggacggacactcatttcttgtgtccttgtgtcactcttctcaagcttgggtctctttttattgggcccaacacttatttcttgtgtccttgtgtcacttgggccaactggcctatagtgataagacatatttgttatgttcgtaacactaacacccagtttctattagaatgctgttacttggaatttgagaaggaattggagtgaaaaaatcttacagaaagcagcaatgacattgcttttgatgatattttggtagtttctatgtcaaaattctttttttttctctttccttaccatttgtatccttttatagtatttttttttcactccatttaacatttgttacataaaagacatgacaggaccaatatattacatcatcagatgtttgatagtggataatcaactaaagaaagaactaaaataattgattttgaaaaaaaaaaactaattattttaatttaagaattaaaaaaaatcaaaatttgcattatacgaaactaaaactaaattttaagcctaataatataatgttcacaaggtgactaaacgaccagggtaagtggtaactcctcctttttggtcctctactatcataaattaatgagcttataactagttctcatatggtaacaaaaagacgtaaaaaatgtttacctttaaagctgcatacatcagacggtattcaacaagaaatagaaatattagcagcaacagatttgaagtggaatgttttgatttcttatatattcagaagagttgaaattaaacaacttaaagcagagttcaagctcactgaatactgatactttgtcctctgagaagttaatctccggatgctacgcactccttatttttctttcactttatgcaatttaaatacctctccatttcaatggcataattctaaaatgttcatcagataagtcctaaaagatttcccaaattattcttgattcgttttccccggcaactagtttattacaaaaaaaatcacagaaggaaaaggaagacacgtgtgaatgctaggactaaagagtgtttgaccgttgacgtcaaggtgaagtttctcttgacttgcctcgcagcttctcaataatatcacagctcgaaaaagaagacacaatcggtaaggtgaagtttctcttgccttgcctggcagcttcccaactactaccaatatgtgcttatgtactttggtctcagtataggatatatgtgctcagtgcctcctccaaagcattaatatactttcacttaccaaatcaaatggatactgaaattgatgagagcattgacgaggccgatatcagatttgatgcaaggagttatagttccagactgaacagtatgataaggagtgagactgagtcaattttgggagcatcccctgtccatgagggagcagctgcagaagaggtgcgccaaatcagagatgtcgcccacaaagctgaagatgtggttgacacttacgttgccaacatcgccaaatcagacatggtgcttcaccaagtcaactctgacatagagaagatcaggagtgtgctcaatgagatctatgagaacagcattggagaaggtgaattccgaagcgaagaagctgctgcagaagcagaatcactcctcaagagaaggagggaggtggaggaagaagatgtagtgggcttagtgcatgactcaagccatgtaattcaggaactcatggaaagtgagtcacgtcttaaagttgtgtccataattggaatgggagggttgggtaagaccactcttgcccgtaagatctataacaacaatcaagtgcagctgtggtttccttgccttgcatgggtttctgtgtccaacgattacagacccatggtatttcttctcagccttctcaaatgttcaatgtcatccatatctgaatttgaagaattaagtgaggcagaactgaagaaggtagcagaatggttgaaagggaagaagtatctggtagtgcttgatgacatctgggaaacccaagtatgggatgaggttaaaggagcctttccagatgaccaaacaggtagtagaattctcataacaagtcgcaacaaagaggtggcacactatgctggaactgcgtctccctactaccttcccatcctcaatgaagatgaaagctgggaactcttcacaaagaagatttttcgaggtgaagaatgcccatctgatttagatcctctgggtagatccattgtgaaaacttgtgggggtttaccacttgctatcgtgattttagcaggactcgttgtcaagaaggagaagtcacaaagagagtggtcaagaatcaaggaagtgagtaggcatcttatagaggataagactggagtgatggatatactgaagcttagctataacaacttgcctggaagattaaagccttgctttctgtattttggaatctatccagaagactatgacatcagtgcaaagcaattgatccaatattggatcgcagaaggtttcatacaaccacaaaaaactggaattgcagatacaacagaactggaagatgtagctgacttttacttggatgagctagtggatcgcagcttggtgcaagtggcaaaaaggaggagtgatggggtgtcaagacatgtcggattcatgatcttctacgtgttctttgcatgtcagaaagcaagtctgataagtttctggaggtttgcacaaactccaacattgataccgtcactaataccaatccccgtagaatttccatccactggaaacgagacttggatgtctctgcaaacatatttcacaaatcatgcactcgttctatgttcatctttggtgaaaatcgtagcatgaatctagttcctgttttgaaaaacttcaagttggctcgtgtgcttggttgtgatatgatgcaaggggtttggtctgattcagtctccagagatttgaagaggatgatccatctaagatacttgagaatagaagtggagcaccttccagattgtgtaagcagcctttggaatctagaaacactgcatgtaagatatcgtgggacagtatccagtaaaatttggacgttgaagcggctgagacatctttatttgatagggggaaagataccagtcgtcctgccaaaagcaaatagaatggagaatctccaaacccttttgttatcgggtaattattcacaacaaactctttcccggttgaacagtggcatatttcccaggttgcgaaaattagctttacgctttggatttcaattcgccatgttaccaagtctacaccacctcaacaatcttcatagcctaaatgtaatgtacccttttgaacttccatcagacaccaatgcgtttccatcaaatcttaccaagataaccttgaagctagatgcagactgtgaccgtgatctccaacctttaatgaagacactagggcgacttaccaaccttcaaatcttgaaacttattaattttggtttattgtcctatggggtagagcttcttgatattggcaggggagagttcccgcaacttcaattgcttcatatgggtaaaataaatgtaagacaatggagattagagaaagatgcaatgcctcgacttcgacgtctggtcattgacagttgtgagttgcctgagcttcgagaagagctttggtcattgactgccttaataatggtgcatgttgttagaaaaataaaataaaaaaatgaaggaaaataaatatgaagaagatgcacagtcttgaattaaataacaaaatagcagtaacaaattaaatttaattgacaacacataaataatgcattatatcataatgagcacaaggaaaaattaaataaagggaaagaattaaatagcctgggttgaagcgtgtaaacgctattcttagagagaaaacgcccccactgcacgggtaagaaattctgattgcgctcttctcccaagttacgacaacccgttggttccgatcgtgtgcagcgaaaggatccccgaaccttatgaacaccaatgctcttgctctcacaaaaattaaggaaaagaaagagataaatttttggcagaaagaaaccagagctctcagtctgtcatttctaggaaagaggaaagagatatatataggcattttgcaacaacaaaatattaccgttagaaatataa

SEQ ID NO: 13 - DNA, 32767 bp, synthetic construct
  misc_feature 1..32767: note = Artificially Synthesized
  source 1..32767: mol_type = other DNA
ttttatttcccttgaaatattgaatcaatattgccagcttcataattatgatgtgagtgacagtgcttgtctgatttagataaatgaacttgataagttgaagaactccatctctttctcttttctgttttgattttaccctgtgtctttgggagatttaaataggtttccctctgctttatggagataatctctattttttttatcaaacaatacaaattccctgtagctgaaaccttacttttttctttgtttctctgatttattatatctaacttcaatgcctttttctcttttgttttagggcctgtttggataagcttctttagaagcacttccaagagaagaaaataagaagaaaataatgatgagtttcttcataagataaaatgagttatctattagctacccattaactaatttgtagaagctctctcatatagcttctctaaaagatgagaggacgtctacaaattagctaatggttagctaatggacaactcatttttatggaaaagctcatttcatttttttcttcatatcttctcctaaaagtgcttccagagaagcttacccaaacggacccttatccaatatatttcattggttcccttttagaacaacttgacttttgtgagacaaatgttttcaggattggtgaatctatattttcattgtatgatttaataaactctgtgacttggaaattggaatttattttgatttttgatacatcagtcagtgtggcatatctatatgggtatgaatgtgtgatgtaaagttgttttcccttaggggaagaagagaggatcaagcttcgtagaggaagggttttttcccttcagaatgaacctgatgtggctcgagtttggctgcctaactctgatttccctggtcttgctatggctcgggcatttggagatttttgccttaaggactttggattgattgctgttccagatatctcatatcaccgccttaccgagaaggatgaatttgttgtgttagcaacagatggggtaaggccttatgaggctcttttcttatgatatactgtcttctatgatctgcatgtgttgaaattgattcatttcaatatcatatttgtagttagccaattctaaaatgaaaatatgaaattgactttaaatagtatttgaatgccacttgcaacagttaaaaaaaccaaatttggttctaatttcactgtatttaaattgacatcttactgtttaagctaactacttatcatgtaaaacttattattatgagttaactactaatgtaaatgatgaacacaaacctaatatcaaaagctgtatcaatatatattttttgatccagttgtatctcaaacccatgttcatatacacagatacacatgttatttctattctctttcaaactggaatttgtaaattgttgagcttttggaaaagcttgttctttacatcaatatagcgatttacccttttctttcgcctctcctttgaagttgtaactctaatttttctcttatggtccatattatgttgtaacttgaaaatttatattctctgaattgtcttatatatatatatatataatatatatacaaatacatacttgattagatggaatattgaattattttaaactgttggattcttgggggcagatttgggatgttctgtcaaatgaagaagttgtggacattgtggcatctgctccacagtctactgctgctagagcactggttgagtcagctgttcgggcatggaagacaaaatttcctttttgtaaggttgatgattgtgccgcagtttgcctcttctttgattctgattcagatttcaagtcaacagatacaaaagacaagttaataccggaggcatctattgaccaatctgagcagttagctttacttggtgaaaagggagttggaaatgaagctgaaaaaaagcagcaatagcgcaagttttgtaccatgtctctcccaagccttgttggttttatcgaaaaggttgggtaaattgtgccgcaacagtcaaaagagaaaggaggatcattattttgatgatttttcggtagttttctgccaatattcttttcattatttatctttcatttccttcgacagcatttgagggaagagggggttatatatatatatatatatatatatatatatatttcttatctggaggaaaaggggtaccgggcttttttttttgttgctggaggtcattttccttctctgtatatttggtagatttggagtttttgtaaatccaactctctagattcatattatatagtctgttctgaagtctgccgtttttcatttgcatcattcttaggttatagtttccaatttagtctgtgaaaacatgagaactggaaagaattggatttatttcttatggactttcgagtcaggtttttgtacctctgtgttgtgttgccgtcagggattaatgatattgtctgtgtgatcctattatgggttttgtgtggaaaatttactctgacggaaatttgtctttaagctatacgagtaaaactgtatttaccttttgatttaaattttattatttcatgattttaacttaattttgagaaggataaattatttttaaaaaaggcttttttgttaagggaaggtgtcctaagtactaacccacgaaggaatgagggaagtgaggatggtaacatgtcaaataaatttggaatttagttcccgaaatttttgagtgttaattattgtaggaatttactcatttcatgataaaacttgacatggagtaccggacttaaaaaagaaaaacatgacatgaagtggatttggagtcaagttttctctcaaggcaaactggatttattctccgtactcacttattaaacaaggttaaaacaattaggtttgattgacattttggccttcagggttgtctttataattttttcatatacgaaaaaagttataagttttaatttctgtacacacatataagttaagttttgatctccgtgatcaacgcagctagtatgtatgtggcacctattcatttgctttactgagccttctcaagtgtttgttgtccacgcctgaatataatgatttattcaagaaaacaacggattgaggaagaactgaagaaggtagcagaatggttgaaggggaaaaattacctggtgatgcttgatgacatatgggatgaggttaaaggtgcatttccagatgaccaaaatggtagcagaatactaataacaaggaggtggcatactatgtacaagaacaattttcaataggatcatcttagaccttaagagcattgacgtaaagatagatgacgaagatcaagccatcattctgttgagctttcgtcctaagccatttgatcattttgctgatacaatgttgtatagaaaggctactctatcaatggatgaggtcaaggctgctttgaactcaaaagaaaatcaaggggagaatgactctggcagtaaaagactcttttctactttcttgtgctgatgtcagttttttgtctccttttttctttcatggtcattattgtgctgatgttttaacatcatttacatgtttattgtaaaaggctctttgctctggcagagtttgatgagatgatttgcaattcctctgcttctttctattcacttttctgctttcttgtgttccacaaccacttgaccagatttttcagttagcttagcatttaagcctgagaaattatgcatcatttcatttgtttgttgtacaaccatatttaatgttaacctctctcccttgtttttccattctaaacatggatcatgtttttgtttattggtctaaagtgttgtttcatgttatttttctttctcttttcccaatggaaggtcctttcatgatttagttagaatagataactgataacacaattagttggaaaggaagatgttagagtgtatgttgataatgggagacaaatcatagtgatcagttttagaattggagtgaaagcatacagacattgcttttgtgccttgtctctgctactctacccaaacctttgctggttttatcatggatataattcattattttatgatctttttggtagattttctaccaaaattcatttcttcttttcggtgatgctatatagtacgaattagttttgcaccaagaagtacgaatccacttggcaaaatatgatcgtacaaatattaatttgaaagaataaaatctagacatgcaagttctaactaccctctttcaaactggaatttgtggaattgttggaattttggaaaagcttgttctttacatcaatgtatatattctcccaaattgtcttacaacacaaaaaaacttgagtattttcttacatattatctgcctacatttttgcgcttaactggattatggaaaaataatttgctaatgagatatactaggacagatggatgaaaattacaccaacttttaacaaaggtttaaatacagtttttttttatttataatttatcatatttttgcctttagtctttatatttttttttgtttttaagctttgtaaattatatttattttatttttaatttttaaagcaccttaaataatattttgacaagtgaaaaaaagtgttatctaaaatgttagaacgaaaaataaaaaaatataatttataaggactaaactttttttacaaaaaaaataaaaaaaatactaaagaataaaaaaatatttaagacaataacaatttgctgaatacaacagaaaagttagttagaacacaataacaatttttaaagattattgttaaagactctgcattttaaaattctaactaatttttgtttctaaaccatggaagacttttatttcataaacacaataacaattaaacaaaactcacaatttaaaaaaaaatcagagtcatcaaatatattaatatttaaagacagaaaaaaaaataaacaaataaagttgagaaagccaacctataattaaaaaggttgacagaaacgcgggtccacgcgtatgcgcgttctttccctttattggcgtccacttgttatctttcgttctctactctgtaaattatctgtccacaaactctctagagtttccacccaagatttcctgtttcacagaaatggcagatagtgttgttgcttttctgctagataacttgtctcggctgcttgaagatgagcacaagttgctttctggcgtggaggacaaagtcaactctctttgcaatgagctcaagtttatacacatcttcctcaagaattccgagggcaagcgcagtcatgacacagtgaaagaagtggtgagccaaatcagagatgtcgcccacaaagctgaagatgtggttgacacctacgttaccaacatcaccaggcataaacagagaagcaaactgagcaagttattccacctcaaagaacaagtcatggtgcttcaccaagtcaactctgacatagagaagatcaggagtcagatcgatgacatctataagaacagggacagatatggcattggagaaggtgaattccgaagcgaagaagctgctgcagaagcagaatcactcctcaaaagaaggagggaggtggaggaagaagatgtggtgggcttagtgcatgactcaagccatgtaattcaggaactcatggagagtgagtcacgtcttaaagttgtttccataattggaatgggagggttgggtaagaccactcttgcccgtaagatccataacaacaatcaagtgcagctgcggtttccttgccttgcatgggtttctgtgtccaatgattacagacccaaggaatgtcttctcagccttctcaaatgctcaatgtcatccacatctgaatttgaaaaattaagtgaggaagaactgaagaagaaggtagcggaatggttgaaagagaagaggtatctggtagtgcttgatgacatctgggaaacccaagtatgggatgaggttaaaggagcctttccagatgaccaaacaggtagtagaattctcataacaagtcgcaacaaagacgtggcacactatgctggaactgcgtctccctacgaccttcccatcctcaatgaagatgaaagctgggaactcttcacaaagaagatttttcgaggtgaagaatgcccttctgatttagagcctctgggtagatccattgtgaaaacttgtgggggtttaccacttgccattgtggttttagcaggactcgttgccaagaaggagaagtcacaaagagagtggtcaagaatcaaggatgtgagttggcatcttacagaggataagactggagtgatggatatactgaagcttagctacaacaacttgcctggaagattaaagccttgctttctgtattttggaatctatccagaagactatgagatcagtccaagacaattgatccaatattggatcgcagaagggttcatacaaccacaaaaaactggaattgcagatacaacagaactggaagatgtagctgacttttacttggatgagctggtggatcgcagcttggtgcaagtggcagaaagaaggagtgatgggggtgtcaagacatgtcggattcatgatcttctacgtgatctttgcttgtcagaaagcaagtctgataagtttctggaggtttgcacaaactccaacattgataccgtcagtaataccaatccccgtagaatgtccatccacttgaaaggagactcgggtgtctctgcaaacacatttaacaaatcatgcactcgttctatgttcatctttggtagtgatgatagaatggacctagttcctgttttgaaaaacttcgagttggcccgtgtgcttgattgtgattcgtttattgggttttcgtcttatccagtccccagagatttgaagaggatgatccatctaagatacttgagaatatgcgtcaagcaccttccagattgtgtatgcagcctttggaatctagaaacactggatgtaacatatgaaaccacagtatccagtaaaatttggacgttgaagcggctgagacatctttatttgagtgggggagggaagttaccagtcgtcctgccaaaagcaaatagaatggagaatctccaaacccttttgttagatggtagctatggtatagacacgtatccacaacaagtgatttccctgttgaaaattgacatatttcctaggttgcgaaaattagctttgcgctatcctgatggaccagatatgttatcaagtctacaccacctcagcaatcttcgtagcctaaaagtaattcgcggttttgaacttccatcagacaccaatgcgtttccatcaaatcttaccaagataaccttagcagcagtccgggatttacatttcatgaagacactggggcaacttaccaaccttcaaatcttgaaactaagattcggcaaagttggttttcatcttgatattggcaggggagagttcccgcaacttcaagtgcttcatatgacgcaaataaatgtaagacaatggagattagagaaagatgcaatgcctcgacttcgccatctggtcattaatgactgtgaagaattgtcagagcttccagaagaactttggtcaatgactgccttaagagtggtgcatgtttcacggccctccgaagaattggctaacagtctcaaaaatgtggagcccaggaatggctgtaagctcaaaatctctaatgaactgtgaaaggcaagtgggaccgccttcttacattgcagacataagttgtagtctttcaatatccttcataatatttgttttttaatcccgaccccatttccattttctttgtccaaataaatttccaatttaaaatagcggatattcaaattgagaatggatgttgcttccttgccgtgtatggagttgtgtctcatgattattgattacatatttataatacctagagtttcttttctatttgtgaataatatattaaattaagggggtgtcatgtaaaagagaagtttttttttccctaaaaattatatattaaaaaatttattttctcatatttgatatacatttttaaaaataaatactaatgtaaaatgatattttttgaaaatatttttaatcaatttctcatgaaaacttgaatatttatgtatatgtatatatatagtttaactatatttaatcttgaattactaatcattcctcacagttattatacactacaaaagtaatcctacgattaattaactttatcatagtaatcatagccattgaaaattaaccatattcctttacatccaatcaaaagaaggagaaaaaaaaacaaattcttttagcacgtttaaatgttaaagctaattattcaaatgactaagaagaaaaattataagctccaaagtaaaacattgattatcaaattatcctatctaaaactctaaagaagttatgttaatgttaaatgaaatattcattcggcccaattgttaaaaaaaatcatagctgtacaaattagagaaataagatgaaaagggagagaaatatgaaataaaatagttaatgcaatacaaagagataaagaaaaaaaaatgaattgtaaaaaatgtaataagtatttgttgtaacaatattataactcttgctaaaaataggtcaaacaaatggagtcaaccagcacagaaatgctctacaataactaatgttaaatatttgcaactataacttcaattccgtgcgtgacttgtatatgttaattatacatttggcattgagatccaagctaagtcaaaaatcaaagtctaataacatatacaattggtggtaagattttgcaatttacatattcaccatgggaattgagaagaaaaatttgacccaaagcgaaggttcccaatggtcagtgtcctagttttccttgaagattacatttataaatcatggaagttcctgaaaagtcaatttctcacatagcatttttttcatgttctttctattttcaaatcctttgaccataattttgactttgatatatgtatttgttgcttcatttcaatatacccatttatgaagtagagcaagtagagacaaaatcatcacccagttgatgtttaaaccacaagcaaaagaaatgtagtggaaaccaactaaatggactgttcccgttcgctcatccctcactttaatgcttgaagaacctgtgagatacagcatagaaaatattactaatgagccattatgcaaaattaaaggaaataatgcaagtcatttgaatttgatttcttataatttttttgacaattctacccaaacctttatataacacgtcaatcctatattatctttaattcatcaattaaacataagtaagaaatctgatgtgaaacaaaaagtaagtggataaaaagaaataaggacgacacttttacatcaaacacctccaggatcagcatgcagatactttaaagttttaaaatggatcgttaaactacacagaactattcctttctcagaattaagaactaagggtcaactccgtttgagaaaaatgttttattatttttcattttcaattttcacaaaaatgggagagttgagacaaatgtgaaaaaaatactaaatttacatgttctattatagatgttttcacaaaaatctacaaatgttttatattattcatttatattatttaaaatacagaagattataaagaactaataatgatacttacagttgtactcagtccatccaataacctgattttcaaggtcatagatgtattacatgttctattatagatgttttcacaaaaatctacaaatgttttatatcattcatttatattatttaaaatacagaagattataaagaactaataatgatacttacagttgtactcagtccatccaataacctgattttcaaggtcatagaagacaagtttatttgtaagcactaaatctgcaacaaataaaaatacaggtgaggctgcatagtaaggtcatggaaagcaagctttctagtgatccaaagttgtgataactccattataatttacatctagaaaaaatataaggaaatttcttccaccagtaaaaataatacatttattataatatttagcttgtaagtttcttccaaaacaagcatggggccacttctctccataaaaaatctcaagaccaataatatgctacaaaactttcaccagcaaattgcaaaaggttgtcagaacctacattcacacctttaagccccctatcaattaatcatcatatctttgaccagattaaataaaaagtaaaaaagaaaaaacagaaaaaggcccttctcagctaaatcctcgtcttttgctttagaaggaagatttaggatgtagtaatcgaaagcaaacttctactacctcttgatttaggaacttgtatcaaaaaatacagaacacagcccaatttctttcaaatctccagttaagatactcacaagatcctatacaaatgtattccaggatatgcatttataatgtatttgtcttgtttatttaagaactcagaagtaatcaggttcaaaaatatcttttatctcaaaatgccattaaaaaagtaaccctttctttcctcttataatggctacggcagtctcattctaaatctggaagcagtattcagcaaattggtggcaaatatatctatcggtacagaaaaggatttgaagagaatacctcccaataaggtcatatttttgctatctcttgattgtgtcccactattttgccaaccaatacaccagaaatctccctgcagatgcacattagtattttatttatagtatggtatataaagattatgatcatagaaatcattgacataaaaaagaccagctcagtagtagtgcacattaataaatgagtcttttgaaaaatcattttaaaactattgcgagtatctaaaattacaaaaagaataacagtttataaggttgaaaatacgaaaagataatatttggtgcatttgcaaattgcaactccagaatccattctggaactcaaaactaatttgaagcagatgggatacaaaaggtttttaaaatgcattgtgggcactttgggcagaagcaactttttattactttagcccagaatacagttaggccaagatgcagttggtctaatatatatagagagagaggagggatcaagttactttaagaggaactcttttaaagttactctccatcctcacactgttcattctcttgaaacctaatagttgtaatgagtaattaatcttaatcattagattttaaggaaatgaatggtgaagatgaggagtaactctcttagagttactcttgaaactcttgaagtaacttgattcctcttcatatatatatatatgacatagaaggccgacaacaggtcccaaaacaagccaaataaaactcggctaatcataacaaaacacacaaagatataatgagatttgcaatatacttacagacggaaataaataatcttgtggataaaccttcaacgagagcccattttcaaaataaaatgtgacagctggaaatccatcatcaacactgcaacataaaataaaataaccattacacccctacatgaagctgataatacagacataaatgcataggatatgcaaagggagcagtccagttatcaaagcactccaacaatacatactcaataagaaagttcatagaacaaaacctacaaaatattataacttaatttcaaaaagagtttttccgattatcaaattaagatttcaaatggcaaatattttttgtccacttatcaaacccatagtcaaaatggcaagatttcctagacaattcctttaacatataatgtagattacaatttacatgcaaatgtcgttattactattatcaagaaaagaatttattaagacagaaaaaaaagatatagagacaggataagaaatcctcatggattacagatgacaccactaaaataaaaggaaaaaagaaacaaaaatcaagacatttgcaacacggcaaaccaacaaagagcataaaaccataaaccagccactacagattaagtaacaacattaacatgatcaaataaaaatcataaaacaatatgtgaaccgatactgtaaaacaagcaacagaattaaaaggaaaaaatttcaaagcatacattcattactgcatactacagtgaaaatagaaaaaatatataacaggaagataagacatctaccaaaataaaaaacaaagtgagagagtgaatagtcctatcctggaaaacttgctatgccatgaaaaagttacttgaagatccaggaatataacctcaaacaccaaccactacagatgtcattgctcatgacagcaagcttaaacaatgccaacatggcattcaattgccatattgaaggatggtgactaatttaaacaaataataacaacaaaacaatgaaatcagtgagtgaagaactgagttgatactagaggagctggtctggtaaacttaatggtaatttcaccctgttcaaaaaagatttctcaccatacaattttcaaaggagatcaaagtaaaacagaatttgccaaaagtcaaaaccacttctgatgtgtttttgtgcagtcttctggtttcctttaccacacattgctgaaggacaattctacctagaacagaggagacaaacactcaccaccttgccatttttaatgtgacatatgccatgcatcatggacaacacagctatgggagtgcaactgtgccatgttcaatagcatgcaaaagaagttttagagaaagtgaccctcaacacacccagtatggaagcacatacttttaacaacattgtcaaccctgaaccaccattatcaaaaccacacactttaggtccttccttccaaaacaaactggactacaaagaattcttctaaaacctgtatgccgaagtaagctagcaaaacacttgcgcttgttccaaccaacagcaagacactatatgcatacaatatgcacgcatgcaatatgcatatttcttttcagatgtgttcaagaaagaaaagattatgttagcagttaaacccccagttatagtagcaatacagtatatggattagttaatatgaatgttatgcatcctacctctcagaatattgaaaacatgtatactcgtcatggagagtctgaacttttagatcgggttgctgagaaattatctaacaagagaaagcaattcagacaactgcaatgtaaatttagaaatttagtaatttgcaaaatttaagatttttatgtaccttatatactagcggctcataaattccttcaggcagataggccaaggttgtaccactgtcaattattgtcccttttctgtctccttgtgttgatgtatctgttgatagactaaggaaagcatggccaacttgaactgcagtcatattaacactgtagtgtggcctaaaaaaccaaattttcaaatgtcatattctctcaaaatatggatttttgtaaaccaatatctgaatagaagagagattctatcattaagatctcatagctaaggctatgtttgacccagcttattttttgcttaaaagctacattaaagttaaagctaaaataaaaactttaaaattaaagtagaagttgtttggtagtcttagcttaaaaactccttttaagataaaagatttgtttcaactagttacggaagaaaagaaggagggaagatatgtggctaattttaaaaagctactttagttagggtttttttaaatgctaattttctcccttttcatttatttaatggtcctcaaatattaagagtttttctctaaatgtgtttggctcagtttctccttttaaggagaagaaaaaggaaaaaagagccgggccaaacacctacatatgtggcctagatactacttataatgacttgggcaagcttccaaggttgattcagccccagtctgatatcaattctagtaaaagtaaacaaaactatgagtgcataaacaaaattgaccaacatccagcatatagaagcatccagcatggacatgaaaatttaacaaagatatattgttttttcatttagaatataggggaagaaagtgctgacttttatggttgaaaaccactgaaatcctaccgccagcttcaactaacatgagttcactttacattccaattacagtttacacatgacaccttcagcttcaaagaatcttttaaaccaaaattttaataccactgaaatgatgacctcgttagccagttgaggcctacattccatttagagtgtagatgtgttgagacatgtcactgatgacttgagaagaaaccattaatcaatactccccctggactcatatataagcaaaaaagtgatcttgtaagttggacttaaatataagcaaatctaactaattttgtcttatttaatgctatcattcctaaaacatccctcatttaattgtaattctattttcaatgactcttttttattcatgatagcaagttccaacgaaagatattttagaaataaccttattttttcgcttgagattagtaaaattaaatgatgtcaactaattttctaaattagtgtgaaattagttatttttgcttatatatgagtccaaagggagtatctagcaaggacaggctagagtagagcttataaaggcttgggaaatcctcacctcatgggctagttcttgggttaatttagcccaaattcaatagaagctacatgttttattatatcgtaaaacattaaaatatttgcacaaaaaataatgtagtcactaacaaaaacatgattaaaaaaaaactagaacaaacttatcttcaaaagagtaattaataacactataagaaaaactaatctcattctaaagtgtattaagtgaatggcatactggtctggtaataatggagtcatgttcactttaggttgcacaacatgcccaatagcaaatataccacctccatttactccatttaagcaatgagcaaacatctttttcactttgccagaagaagcaagttgtgaaatcattgatgaattagcttttccaaatccaagtattccaccaagtgcctcttcattcgaagaacttagatctccagattgtctagcaccacaactacaaaacagcaaaaaactcattttaaatataaaaagagggagaaatatgaaccaaaagatgcatgcaatagacaacattaaattatcatacacaataagaatcaccgagaaaccattataacctgaatgcctccaaaatcacatcaagctgtttgagtattagaaaaaggtatttctaggagtgttccatatgataaggtgttctattcactgcctcaatatttactaaaccatgttatttacagcatgtttggtaaaactgaaaactttagcattggaagattataaattcaagcgctgatattgtgctaaaggaagctattagcatttaacatacctaacaaagataaaacaaacgaaattattgtccaggaaatagttcctgttgatacatcactgactgcacaaatgctgaattattcaatattttttttacataaatttatttttcagattgagtagggttcaagcttggaacacaacaaaaggaggactgcaagaagtacaagcctatatcaatatcaacgtcagcagtctcactctcaatcttactcaaattggtagctgacccaaaccaatatcaaaatcaagtattttttcaggctccaatattagttttggtgaggagttggacgcaatttatatacttcataatgaaataaaaagcatactcgacagtttttgaaaagcaatgcctgcacaccagtgcacccactttatgttaatatcatatgcagatatgagaaattatgctattgcaagtttgcaactaaagcaacaggttaaattactatgaacaaacatgcatggtattggtatctgagttcatgacttatgagaaattaatgtccactaacagcaacagatcaaaagtaatagacatacacaatacataatagcacacattactgccaaaagaaaactcacccaaatacaatacttccatttgctgaatcagttttaaggtccccagacacttgatcataaagtacgatatcctttacaaagtaaccagcagtcgagctcccatcaccatatatctccagatatggacaggaaatattagcagtgcatccggtcagcagacccccattgatctctttgcaaaactcttgatcacagggaacaaatttaccagaggaggattcctttatgtcgtagagtgtaaggtccatcttcgatacacagaaaaagaaattcaaatgaataaaaacatcaaatgttgaaatgagaaggctgtacatgattcagccatgtgagaattgtcttgcaagggatggtctaccatggtacacgccctgatagtgtaaaggattttatacactctcaaccaatcagaaactcccatgtaaggtaattatgttgaattttatcataactcatttaaaagtcacccctagcatgatttccgattagttgacagtgcaaaagatcgctacacaatgcataaattttaaactcaggttgtaatccatcctaattttaaaatgcatacacaaagcacaaaaaaaaaaaaaaaaaactaagcaagcacgtttggcaagtaatgaagataataagggtagcttaaactaaaaagatgagaagcattcaccaaggttgcttctggtggggcattctttgcactgaatgcaattcacccacataatgtcactaccttactaattatcaggcaccgatggtattcttcttgatattggcaggggagagttcccgcaacttcaagtgcttcatatgaggggaatatatgtaagaaaatggagattaaagaaagatacaatgcctcgtcttcgacgtctggtcattgacagttgtgagttgcctgagcttccagaagagctttggtcattgactgccttactaatggtgcatgtttcgtcgccctcccaagaattggctaacagtctaaaaaatgtggtgctgaggaatggctgtaagctcataatctctaaaagttgtgttgatgaatggttttaagctcacaatcatctagtgcttcataactgttggaataatggcaatgcaagtgggcattgctgggttttgttatggtctttctatatccatcatgataacgtgcagtggttcttatcattctgtatctttctaactttctaatctcattgccacacgtgttgtgattgctagatttccattttaatctcagtttttgtctaatttctttttctttcttggacctttgttctgaagttgtaactttatttacatgttgattgtaaaaggatatttattctggcagtgtttgattaaatgatttgtaattcctctgcttctgtctattcacttttctactttcttttcttagatgacccgatatttctgttagcttagcattgaggcatgagttacatatatgcattatttcattcatttgttgttcagtgtgctcaactatatttgattgtgcgttaattttccctcttttccccttcttttcccatccaaagtaagtgtttcgtctggtccctaatataaagtatttaacttttttccattatttatcctattaaatactctaactatatgtacttttaggtaattatgcataatattgttatttatgacaagttaactatcattaataagagtaaaattgatacattaatttatttatttttattggtatgtgtaattttgttatttttatttattggttttggtgttgtctcacgagatgtctttttcctggttgaggtgtgtttctcttctcatttcttagatggaatagttacttgataacataattaattgtataatgggagataaatggttttcatcttctcttctcatttctttttttcccattcatttttctggccgagtgatacagaggagtaaaggaatgaggacttgtttcaagtaagagtaactccaaaatagcaaagagactgaaaagggaatttctgagtaataatttctctgccttctttcattccgtgcttggatacatataggatagatttttcccctaataacctgaggatacgagtaataaattcaaacagaattcagattctaataacaatatgaaaatgatcaattttaggtgataaaagtaattactcttaaaactaaatggcagagctagtgtccaacctagtccttcgtagacatggcaatggggcggatcgggtacagatattgtctccccaatctcttaccccgactcctcaacatatccccatacccgatacccgacaggtttgagtttattgtctcatccccgtacccgtcgggtatcgggtatccccaaccccaatccacacaccatttagaaaaatatttttttttttgtaaaaaaatattaaaaatttgattttagaaaaaaataaattgattgttaaacatttatttttaactacttatatatcaataaatttattataacgcgtgtgtctacataaatagttaaaaaaataatattaaattatgtaaaaattttaaaataaaattaactagtaataaaaattatgtcttttgattaaattatgcaaaaattctaaagattttaagtggcagggcgggtttgggttcggggtcggaacgggtctagtaatcccatacccgtacccgtacccgacttttggttattgaaaaaaacccgaattcatactcatacccggtcaactcggatattacccgtcaaagttgggacggacactcatttcttgtgtccttgtgtcactcttctcaagcttgggtctctttttattgggcccaacacttatttcttgtgtccttgtgtcacttgggccaactggcctatagtgataagacatatttgttatgttcgtaacactaacacccagtttctattagaatgctgttacttggaatttgagaaggaattggagtgaaaaaatcttacagaaagcagcaatgacattgcttttgatgatattttggtagtttctatgtcaaaattctttttttttctctttccttaccatttgtatccttttatagtatttttttttcactccatttaacatttgttacataaaagacatgacaggaccaatatattacatcatcagatgtttgatagtggataatcaactaaagaaagaactaaaataattgattttgaaaaaaaaaaactaattattttaatttaagaattaaaaaaaatcaaaatttgcattatacgaaactaaaactaaattttaagcctaataatataatgttcacaaggtgactaaacgaccagggtaagtggtaactcctcctttttggtcctctactatcataaattaatgagcttataactagttctcatatggtaacaaaaagacgtaaaaaatgtttacctttaaagctgcatacatcagacggtattcaacaagaaatagaaatattagcagcaacagatttgaagtggaatgttttgatttcttatatattcagaagagttgaaattaaacaacttaaagcagagttcaagctcactgaatactgatactttgtcctctgagaagttaatctccggatgctacgcactccttatttttctttcactttatgcaatttaaatacctctccatttcaatggcataattctaaaatgttcatcagataagtcctaaaagatttcccaaattattcttgattcgttttccccggcaactagtttattacaaaaaaaatcacagaaggaaaaggaagacacgtgtgaatgctaggactaaagagtgtttgaccgttgacgtcaaggtgaagtttctcttgacttgcctcgcagcttctcaataatatcacagctcgaaaaagaagacacaatcggtaaggtgaagtttctcttgccttgcctggcagcttcccaactactaccaatatgtgcttatgtactttggtctcagtataggatatatgtgctcagtgcctcctccaaagcattaatatactttcacttaccaaatcaaatggatactgaaattgatgagagcattgacgaggccgatatcagatttgatgcaaggagttatagttccagactgaacagtatgataaggagtgagactgagtcaattttgggagcatcccctgtccatgagggagcagctgcagaagaggtgcgccaaatcagagatgtcgcccacaaagctgaagatgtggttgacacttacgttgccaacatcgccaaatcagacatggtgcttcaccaagtcaactctgacatagagaagatcaggagtgtgctcaatgagatctatgagaacagcattggagaaggtgaattccgaagcgaagaagctgctgcagaagcagaatcactcctcaagagaaggagggaggtggaggaagaagatgtagtgggcttagtgcatgactcaagccatgtaattcaggaactcatggaaagtgagtcacgtcttaaagttgtgtccataattggaatgggagggttgggtaagaccactcttgcccgtaagatctataacaacaatcaagtgcagctgtggtttccttgccttgcatgggtttctgtgtccaacgattacagacccatggtatttcttctcagccttctcaaatgttcaatgtcatccatatctgaatttgaagaattaagtgaggcagaactgaagaaggtagcagaatggttgaaagggaagaagtatctggtagtgcttgatgacatctgggaaacccaagtatgggatgaggttaaaggagcctttccagatgaccaaacaggtagtagaattctcataacaagtcgcaacaaagaggtggcacactatgctggaactgcgtctccctactaccttcccatcctcaatgaagatgaaagctgggaactcttcacaaagaagatttttcgaggtgaagaatgcccatctgatttagatcctctgggtagatccattgtgaaaacttgtgggggtttaccacttgctatcgtgattttagcaggactcgttgtcaagaaggagaagtcacaaagagagtggtcaagaatcaaggaagtgagtaggcatcttatagaggataagactggagtgatggatatactgaagcttagctataacaacttgcctggaagattaaagccttgctttctgtattttggaatctatccagaagactatgacatcagtgcaaagcaattgatccaatattggatcgcagaaggtttcatacaaccacaaaaaactggaattgcagatacaacagaactggaagatgtagctgacttttacttggatgagctagtggatcgcagcttggtgcaagtggcaaaaaggaggagtgatggggtgtcaagacatgtcggattcatgatcttctacgtgttctttgcatgtcagaaagcaagtctgataagtttctggaggtttgcacaaactccaacattgataccgtcactaataccaatccccgtagaatttccatccactggaaacgagacttggatgtctctgcaaacatatttcacaaatcatgcactcgttctatgttcatctttggtgaaaatcgtagcatgaatctagttcctgttttgaaaaacttcaagttggctcgtgtgcttggttgtgatatgatgcaaggggtttggtctgattcagtctccagagatttgaagaggatgatccatctaagatacttgagaatagaagtggagcaccttccagattgtgtaagcagcctttggaatctagaaacactgcatgtaagatatcgtgggacagtatccagtaaaatttggacgttgaagcggctgagacatctttatttgatagggggaaagataccagtcgtcctgccaaaagcaaatagaatggagaatctccaaacccttttgttatcgggtaattattcacaacaaactctttcccggttgaacagtggcatatttcccaggttgcgaaaattagctttacgctttggatttcaattcgccatgttaccaagtctacaccacctcaacaatcttcatagcctaaatgtaatgtacccttttgaacttccatcagacaccaatgcgtttccatcaaatcttaccaagataaccttgaagctagatgcagactgtgaccgtgatctccaacctttaatgaagacactagggcgacttaccaaccttcaaatcttgaaacttattaattttggtttattgtcctatggggtagagcttcttgatattggcaggggagagttcccgcaacttcaattgcttcatatgggtaaaataaatgtaagacaatggagattagagaaagatgcaatgcctcgacttcgacgtctggtcattgacagttgtgagttgcctgagcttcgagaagagctttggtcattgactgccttaataatggtgcatgttgttagaaaaataaaataaaaaaatgaaggaaaataaatatgaagaagatgcacagtcttgaattaaataacaaaatagcagtaacaaattaaatttaattgacaacacataaataatgcattatatcataatgagcacaaggaaaaattaaataaagggaaagaattaaatagcctgggttgaagcgtgtaaacgctattcttagagagaaaacgcccccactgcacgggtaagaaattctgattgcgctcttctcccaagttacgacaacccgttggttccgatcgtgtgcagcgaaaggatccccgaaccttatgaacaccaatgctcttgctctcacaaaaattaaggaaaagaaagagataaatttttggcagaaagaaaccagagctctcagtctgtcatttctaggaaagaggaaagagatatatataggcattttgcaacaacaaaatattaccgttagaaatataaccgttggaaaaccaacctacagttgttgttaagactttggcaagtgcaccaaatcgtgacaagtagtaaagctcggaagtccgagtatcgtgtccacatggattttattgcacttattcaatacctctcaatttgtaagtgggagtaaaatagtaaaaataagaatgaaagtaggaatagagtgctattactaaaacaaataaggaaaaagggaaacaattaatagaaatgccaagaccagacaaacccaattggcctacgatgcatgtaaataggatattcattatcaatgaaatggtattagttctacccacatctgttaactacttgcccctgatgcctcacgttggcaagcctattttaactaactatctcccaaatgcctttgtgaagattcaataattaaaatgcattaagattaagttctagatgttgcttaaatgcatgggcagtgagttatcattttatgcctagcaatgctaacccaatgattcttttctttagatgttctattaggtgttaccctttcccaagcgtataacccctaaaactgatgcatgcattgaatcttaaatattattgagaattaccctctctcgagcgaataaaacccaaaaagagatctaagaatgaatgcaagaataaaaaaaaaagaaatggaacagaaaaatctggaataaattcccttgtattgataactcttgaagctcattgtacatcgttggctttttaggcctgccaggccctagctaggggtttagccactcatggccattgagggcttacaactggggatgaaagaaagaataggctaataaaacaaagaatatatagagagaaaagaaactcaggcttgaggaactgagagtagtgctctgagacgggatgatgtttccttgggactacctctctatttatagctgctgaagtgggcttatgggccttcgtaatagcgctcagcgccacacctcgcgcttagcgcgttcagatcacgcgctgggcgccccacgcacgctggacctgtgcttctgttggatcgggcgctgggcgcctagctgggcttagcgcgcgtaacggtttccaccttcgtgcttaacgccacgctcagcgtctgcagttagttgatcgcttagcgccagatgcgcgctcagcgcccctattggattgggcctgcttcagaatttcttctttttttcttcctttctgtgccacttttgctaaatgcaaccttattttttgtatctgcaaccagaaattcaattaaattaattttctcacttttaattacaaataactgctaaataattaattttaagccaaaatttgactatttaactcctataaattcacaattatttagcagttatcagttgtcacaacaaatataacaaactagtttgactcattaaaacaaaatcttaagagaatctaaaataaatattattttataaaatagaattaatatatatttataaattttaaattaaaacacaaatatttaccaacattcccccacataatttaaaattttaaaatatattttctaaaagataatttgtataaaaacataagagaaagagcatgtgatattgtatttcggtataaggaaccttctgggtttgagccttatacctagggtttatgaattttcatccgagaaaaatgtagtgatttgattgtcttgaactacatattctttaaccggactttagtacacaacccctacaatattggtgttcaattaggttctaatcagtggtagcgcgttacacggccttgcgcttgtatcttgtttcgtaagtgtcccttagagattagcccatatctcacagtggcggccccaccaccacactcactaggtgaatcctcaaagagtgggttgtgaccccacccctacaaaaattgtcatcggattcattaagaggtatttttttttccaccaaaaatacacatatataaatacctcaaccttaatattgtcacaatttataatacacctcgtcataggaatgagaaacggaacagctccgtcaaatttcattttggtgtactttagtcaacaaacaatttcgttgttacccgttgaactccattcatgggatcaccaatcacacggagacgggtgtccattgttgtaactaaataatgggctttagtctcattcccctcgacgactcaagtacttgttgacgcgccaacccttttgtaagcggatccgcaatattattttctgacctgacaaagtcaagagaaatgacaccatgagaaatcaaatttctgatagacttattcaacaatgttttagatactgctactagtgagactgagtttcttaaaaatttgttatgtgctctaccattgttgaataagcctatacctccaattccaatgggttgtgatagtccaatgggttgtgatagttaagagaatctaaaataaatattattttataaaaaaattaatatatatttataaattttaaattaaaacacaaatatttagagaaagatgcaatgcctcgacttcgacgtctggtcattgacagttgtgagttgcctgagcttccagaagagctttggtcattgactgccttactaatggtgcatgtttcgtcgccctcccaagaattggccaacagtctcaaaaatgtggagccgaggaatggctgtaagctcataatctctaattaatgcttcacaactgttggagtaaggcaaatgcaagtgggtctgtcatcttaaattgcagacataagttgtagtcattcaatatccttcatcataacttaacttggctggcagacaatctgtaatgttcggtgcttcttgtcatctctatttttctaatattaacgtcactcttcacggttatattttcattttaatctcagtttttgtgtaatttcttttatcactgtaattgcaattgctttaccccacaccacaactaaccttgctctcataacgcaccctcttcctagaccgaaagaatctgagagaaataaacattttttatataatatttgttttttaatcccgaccccatttcgattttctttgccaaataaatttccaatttaaaatagcggaaattcaaattgagaatggatgttgcttccttgccgtgtatggagttgtgtctcatgattacatatctataatacctagagtttcttatccatttatgaataatatattaaatgtctatttttctaatattaatgtcacccttcactgttatattttcatttttcgaaaatatttttaatcaatttatcatgaaaacttgaatatttaagtatatgtatatatatagtttaactatatttaattttgaattactaatcattcctcagaattattatacactacaaaagtaatcctacgattaattaactttcccatagccattggaaattaaccatattcctttacatccaatcaaaagaagaagaaaaaaaaaacaaattcatttagcacgtttaattgttaaagctaattattcaaatgactaagaagaaaaattataagctccaaagtaaaacattgattatcaaattatcctatctaaaactctaaaaaggttatgttaatgttaaatgaaatattcattcggcccatttgttaaaaaaaattatagctgtacaaattagagaaataagatggaaagggagagaaatatgaaataaaatagttaatgcaatacaaagataaagaaaaaatatgaatcgtaaaaaatgtaataagtatttgttgtaacaatattataactcttgctaaaaataggtcaaacaaatggagtcaaccagcacagaaatgctctacaataactaatgttaaatatttgcaactataacttcaattccgtgcatgagttgtatatgttaattatacatttggcgttgaaatccaagctaagtcaaaaataaaagtctaataacatatacaattggtggtaagattttgcaatttacatattcaccacgggaattcagaagaaaaattttacccacggcgaaggttcccaatggtcagtgtcctagttttccttgaagattacatttataaatcatggaagttcctgaaaagtcaatttctcacttagcatttttttcatgttctttctattttcaaatcctttgaccataattttgactttggtatatgtatttgttgcttcatttcaatatacccatttatgaagtagagcaagtagagacaaaatcatcacccagttgatgtttaaaccacaagcaaaagaaatgtagtgaaaaccaactaaatggactgttcccgttcgctcatccctcactttaatgcttgaagaacctgtgagatacagcatagaaaatattattaaatgagcaattatgcaaaattaaaggaaataatgcaagtcatttgaaaaatcattttaaaactactgcgagtatctaaaattacaaaaagaataacggttaataaggtttgaaaataccaaaagataatatttggtgcatttgcaaattcaaactccagaatccattctggaactcaaaactaatttgaagcagatgggatacaaaaggtttttaatatgcattgtgggcactttgggcagaagcaactttttgttactttagcccagaatacagttaggccatgatgcagttgggtctaatatatatatatatagagcggagggatcaagttactttactgtctatcctcacacattcttgaaatctaatagttgtaatgaatatttaatcttaatcattagattgtaagagaatgaatgatgaagatgaggagtaactctatatatatatatatatatatatatgacatagaaggcaaccaacaggtcccaaacaagccaaataaaactctgctaatcataacaaaacacaaagatataatgagatttgcaatatacttacagagggaaataaataatcatgtggataaacaatcatctagtgcttcataacttcttgataatggcaatgcaagtgggcattgttgggttttgttatggtcttttatatccatcacgataacgggcagtggttcttatcattctgtatctttctaatctcattgccacacgtgttgtgactgctagatttccattttaatctcagtttttgtctaatttctttttctttcttggtcctttgttctgagtttgtaactttatttacatgttgattgtaaaaggatctttgttctggcagtgtttgattaaatgatttgtaagaatggcagtgtttgattaaatgatttgtaagaatgtttatcacttactatgaagtaggaacaaacgacttgcatattagacctactacattcaccttatcttttataaaatgcctgataatttctacatatttcgcgggtcatgttgaatgttacggaaaatactactatatattgtcttgttttctcgtacaatatatacaatttcttggagagagttttaggaagttgcaactctttagaattctcttaagccataaaatttcacaaattttttgggtgtttgccgtatatttacttctctaagtaaccaaaatttccccatcaaaagtgccagatgttaactgatccctgctcttatgcatgtatatagataatcatctggttcccaaactctaattggcgcacaaccacagaacagaactctactttcctgtgttccacgaccactttaccagatttttctgataggttagcattgaagcctgagagaaattatgcatcatttcatttgcttgttgtacaaccatctttaattttgcattaattaatttaaacctttctcccttgtttttcgattctaaacatggatcacgtttttgtttattggtttaaagtgttgtttcatgttatttttctttcccttttcccgattgatggtcctttcatgatttagttaggatagatacctgataacacaattagttcgaaaggaagatgttagagtgtatatagatagtgggagacaaatcataatgatcagttttataattggagtgaaagcagacagatattgcttttgtgccatgtctctataactcaacccaaacctttgctggttttaccatggataagaaaattgtgctgcaacagtccaagagatatagttcattattttatgatctttttggtagatttctgccaaattcatttcttcttttcagcgatgctatatagtacgaattagttttgcaccaagaaatacgaatccacttggcaaaatattgttggacaaatattaatttgaaagaataaaatctagacatgcaagttctaactaccctctctcaaactggtgttagagtacaggtgtaaagtgaaagtaaaaaggttgaacacgtgaacaaataacttataaacctgagtttaaggtgttgggttagagtgtggtgtcagaccataacggtaaaattgttggaaatttaattagatcaactattcataaaaatttaaatatattttttatccttataatttaatattttttttgtttctaattaatgtaaagttttttttttagttttggtaaattatgtttgatactttagataatattttgacaagtgtaaaaattattatctaaaacatcacaagaacaaaaaagaaaattaaaaaaatataatttgtaagaaaaaaataatttaataaacactaaaaaatcactaaattataagcagtaaaaaatatttaaacctttgataaaaaagaaaaaaagaagagtacatttaataaaatttgctgaaccctgcagaaagattggttagaacacaataacgatgtttaaagattactgttaaagactctgcattttataattctaactaattgttgtttctaaactatggacgactttgatttcagaaacacgataacaattcaacaaaactaacaatctaaaaagaaaatcagagtcataacatatattaatatttaaaagaaagaaaaaaaggctacgcagttaaagttgaaaagtcaaattataaataaaaaaggttgataaaaacgtgggtccaagaatattcgcgcccttttcctgtattggcgtccacttgttatcgttcgttgtctactcggcatactatctgtccacaaactctctagagtttccacgcaagatttcctgtattacaaaaatggctgatagtgttgttgcttttgtgctagataacttgtctcggctgcttgaagatgagcacaagttgctttctggcgtggaggacaaaatcaactctctttgcaatgagctcaagtttatacacatcttcctcaagaattctgagggcaagcgcagtcatgagatcgtgaaagaagtggtgagccaaatcagagatgtcgcccacaaagctgaagatgtggttgacacctacgttaccaacatcaccaggcataaacagagaagcaaactgagcaagttattccacctcaaagaacaagtcatggtgcttcaccaagtcaactctgacatagagaagatcaggagtcagatcgatgacatctataagaacagggacagatatggcattggagaaggtgaattccgaagcgaagaagctgctgcagaagcagaatcactcctcaaaagaaggagggaggtggaggaagaagatgtagtgggcttagtgcatgactcaagccatgtaattcaagaactcatggaaagtgagtcacgtcttaaagttgtttccataattggaatgggagggttgggtaagaccactcttgcccgtaagatctataacaacaatcaagtgcagctgtggtttccttgccttgcatgggtttctgtgtccaacgattacagactcaaggaatttcttctcagccttctcaaatgtttaatgtcatccacctctaaatttaaaaaattaagtgaggaagaactgaagaagaaggtagcagaatggttgaaattaagtgaggaagaactgaagaagaaggtagcaaaactgttcaaagggaagaagtatctggtagtgcttgatgacatctggacaacccaagtatggtatgaggttaaaggagcctttccagatgaccaaacaggtagtagaattctcataacaagtcgcatcaaagaggtggcacactatgctggagctgtgtctccctacgaccttcccatcctcgatgaagataaaagctgggaactcttcacaaagaagatttttcgaggtgaagaatgcccgtctgatttagagcctctgggtagatccattgtgaaaacttgtgggggtttaccacttgccattgtggttttagcaggactcgttgccaagaaggagaagtcacaaagagagtggtcaagaatcaaggaagtgagttggcatctttctgaggataagactgaagtgatggatatactgaaacttagctataacaacttgcctggaagattaaagccttgctttctgtattttggaatctatccagaagactatgagatcaaagcaaggcaattgatcaaatattggatcgcagaagggttcatacaagcacaaaaaactggaattgcagatacaacagaactggaagatgtagctgacttttacttggatgagctggtggatcgcagcttggtgcaagtggcaaaaaggaggagtgatgggggtgtcaagacatgtcggattcatgatcttctacgcgatctttgcttgtcagaaagcaagtttgataagtttctggaggtttgcacaaattccaacattcataccgtcagtaataccaatccccgtagaatgtccatccactggaaacgagactcaaatgtgtctgcaaacacatttaataaatcatgcactcgttctatgttcatctttggtagtaatactacggtcctagttcctgttttgaaaaacttcaagttggcccgtgtgcttggttgtggtgtgtatcaagggttttggtcttattcagtccccagatatttgaagaggatgatccatctaagatacttgagaatagaagtcaagcaccttccagatagtgtatgcagcctttggaatctagaaacactggatgtaacatatgaaaccacagtatccagtaaaatttggacgttgaagcgactgagacatctttatttgagtgggggagggaagctaccagtcgtcctgccaaaagcaaatagaatggagaatctccaaacccttttgttatcgggtaattattcacaacaaactctttcccggttgaacagtggcatatttccgaggttgcgaaaattagctttgcgctatgctgttgaatcagacatgttaccaattctacaccacctcaacaatcttcatagcctaaaagtaatgtacgcaattgaacttctatcagacagcaatgcgtttccatcaatcttcatagtcttaagagtggtgcatgtttcacggccctccgaagaattggctaacagtcttaaaaatgtggagcccaggaatggctgtaagctcatagtctctaattaatgcttcacaactgttggagtaaggcaaatgcaagtgggtctgtactcttacattacagacataagttgtagcttttctatatccttcatcataacttaatttggcttttaggcaatggtaatgttcggtgcttcttgtcattctgtatttttctaatattaatgtcacccttcactgttatattttcattttactctgtttttgtctaatttcttttatcattgtaattacaatcgctttaccccacaccccaactaaccttgctatcgtaacctttcttttatcattgtctaatatttgttttttaatcccgaccccatttcca

SEQ ID NO: 14 - DNA, 25 bp, synthetic construct
  misc_feature 1..25: note = Artificially Synthesized
  source 1..25: mol_type = other DNA
gtatatttatatatatatatatata

SEQ ID NO: 15 - DNA, 10 bp, synthetic construct
  misc_feature 1..10: note = Artificially Synthesized
  source 1..10: mol_type = other DNA
aatatgcttt

SEQ ID NO: 16 - DNA, 34 bp, synthetic construct
  misc_feature 1..34: note = Artificially Synthesized
  source 1..34: mol_type = other DNA
tgggtcaacctgaatgagtcagggttgacccaaa

SEQ ID NO: 17 - DNA, 12 bp, synthetic construct
  misc_feature 1..12: note = Artificially Synthesized
  source 1..12: mol_type = other DNA
ctttctttaatt

SEQ ID NO: 18 - DNA, 25 bp, synthetic construct
  misc_feature 1..25: note = Artificially Synthesized
  source 1..25: mol_type = other DNA
attcaaaaatatttttttaataatt

SEQ ID NO: 19 - DNA, 24 bp, synthetic construct
  misc_feature 1..24: note = Artificially Synthesized
  source 1..24: mol_type = other DNA
attaaatttaaattgattgttaat

SEQ ID NO: 20 - DNA, 14 bp, synthetic construct
  misc_feature 1..14: note = Artificially Synthesized
  source 1..14: mol_type = other DNA
ctattatacaccga

SEQ ID NO: 21 - DNA, 11 bp, synthetic construct
  misc_feature 1..11: note = Artificially Synthesized
  source 1..11: mol_type = other DNA
cattaaggcct

SEQ ID NO: 22 - DNA, 11 bp, synthetic construct
  misc_feature 1..11: note = Artificially Synthesized
  source 1..11: mol_type = other DNA
ttaagtatatg

SEQ ID NO: 23 - DNA, 6262 bp, synthetic construct
  misc_feature 1..6262: note = Artificially Synthesized
  source 1..6262: mol_type = other DNA
atttcttctttaattgttttttttttttttttgctcagcaaaattaaatatattatagatgagtaccagaggtactaaaatatacagatttagagccatattacaagtagttttggacagacaatgatacagtagctgaaatatcccaaaaactactcaaataagacttggagctatactctaaacctatgatgctgtcctaagaaaagcatctttgagatttgaagaccattgattgaaatgtagagagaaatctttttcaaaacatctgagccatgtccataaaataaacactgcatcatccattaatttatgagtgtcaaaattagcattggagaaaatgatgctgttcctatgcttccaaatcgagtaagtgagagcgaaccaccaccactgccatcttttttccctgcaaccctacggaagctccaaagatatgctgactaaaatgctgatccggctgataagggaaaacacccatcatatttacccaagactgtgactcccaccatagaggactaactttactgcagtggaaaaataagtgacttgcagtctcttccacagatctgcataaggggcacaagtactcttgtaactcaacccgcttctttctcaaattggctttagttggcagtttgtcttgaatcaacctccacgcgaagatagccactttcaatgggaccctaagcttccataattccttgaactttccatcctgctcttctcccactgtgacatgatgtattgccttatatgcactcttagtcgaataacagccactaggctctgctgcccacttccattggtcacttaattccggcctgattgtgaacccttccagctgttggaggaaagctaccgccatatctatctcgctatcaaacaaaggtctcctccacttaagactccattcccacccaccttctttcgcagcacctatttgatgaatgaaatgatgtttttgagctgatatggtatacaatctaggatatttatcagctaagcagttgtctccgcctatccacctatcctcccaaaacttaaacttgtccccacatccgaccctccacaatatcaacctattcagctgttgaccttgattcatgctttgatttactatctttagatccctccaccatgatgattcagtactagccctcgaagctccatcaagactcctccatcctccatactttgattcaaggactctagcccaagtctcctcctgattctgaaacatctcccatttccattttcctaataatgatgtgttgaacgagatgatatccttgactcccaacccaccttgttcctttggcaaacacaccttttcccacttgatccaagggatcttattttgatcagctgcaccaccccataagaagttcctttgtaatctggttatcttgctaatcacctgcctagggaccctgaaaaaagatagaaaataaattggaatagatgttagcactgaatttatgagaatcactctccccccaaaagacaagtgtctctgtttccatcctgccagttttctctcacacttattgattaaaggttcccacgtctgacatcttctaggattagcaccaataggtatgccaagatatgtgaaaggaaaagacatcaatccacagttgagataactggatgcaccataagtccactgctcaggcaccccaaaggccccacaactactcttggcgaaatttatctttaaccccgatgacatctcaaaagcacgaagtattgcattgattgttcttacattcgctaatgttgcctctcccaaaaatagtgtcatctgcatactgaagcaggctaatctccaccttctttgagcccactaaaaatcccttatagaatcctccctcaattgctttggtcattagaccgctcaaaccttctgcaacaatattaaacaaaaggggtgataatggatccccctgtctaagacctttctgtggatagaactccttcgtagggcttccatttaccaagactgagatggaagctgattttaaacatctctgtatccactgaatccacttcggtccaaaacccaatctcctcatcatgtatatcaaaaaatcccaagacactaagtcatacgccttctcatagtccaccttgaaaaccatgcatggtttttgacatcttttggcctcttcaactacctcatttgcaatcaccacactatgaagcatgtgcctaccttgtatgaatgctgactgacgatcactaattatgaggggcataactctcttcagtctattaggcaacactttagcgaggattttataggtacatccgatgagtgaaataggtctatattcattcaacccttgagggtccgccaccttcgggattaaagctatgaatgatgcgttcaagcccctcgggaagactccgttgacatagaactcgtcaaagaatctaagaaaatctggtttgatgacctcccaaaattgcttgatgaatttgaaattgaacccgtctgggcccggacttttatcacttccacaactccaaacagccctccttatctcctcttcctgaaagtgctcgactaacatggcattctgatgggaatcaatggtattgaagctgatcccattcagagttggcctatcaaaatctgtttcctggaacctctgtgaaaaaaatctcctaacttcctctttgacttcagccggctcctccttccacatcccatcaaccatcagtccatttaggctattggatcgcctcctggaatttatcagcaaatgttaatacctcgaattacaatcaccctccttaatccacctggatcttgccttctgacgtaacagcgactcatgagtttgggctgcctcccaaaccttctgctgcaacttctttttgagtatccgctcattatcatccagtggtctgtctgctgattcctcttctagtttgttcaactcttcctctatcctcttgaaacggcttaaagtgtctccaaagtgatctttattccacgcctttatcttctgtttgagggccttgaatttgttttttaaaacatgacccccccatccactctgagtattggatgaccaggtttcggacaccaacttcttaaaagatacatcagataaccaacaatccaatagtctgaaaggttttgggccccaatcaatggttttagatctaagcaatatgggacaatggtccgaaaaattcctggctagcggtgtttggaccgatccgggccatttggaaagccactctggggaaacgaaggctctatccaacttgcttttagctgtaccgttcggtctaaaccatgtaaatctctttcccacccaaggcgcttcttctaattccaactcctcaatccagctattaaaatcccgtatgcttccatccaccatccccctctgcgaagaccccactctttctccacttatcctgatgttgttaaagtcccctataatacaccaataaccatttggattctggattttcagctgagtgactttatcccataacacccttttgtcctgcaggttacatggggagtatatatttacaacatgcactgctatggattcttgaccccactttcctgacagtagtatgaagcccgatccagctgatttgctctccaccttcagagacttttgattccaaatacagagaataccaccagctgtatttacggctggaacctcctcccagcagaaatcagaattcccccatagagccctgcacatactcttattgaccgtttccttctttgtttcttgtaagcataaaaggtcaatatgatactctttgaccatacgtctaattgctggccatttaacccccctccccaacccccttacattataagagatgatattcatgagtcccttccccttcctcccaagctatctgcctcttttccatctctatcttccatttctgtcaactgttgaacataatctctttgagtttttcctgttgtcagtcccaactccttgatcattctccatagactttcttcgtgcgagtcaaaagtttcctcctggagctgattttcctgcctattgtgagttgatgatgtttctgtttgtatttgggggttggctgatccacgtccctcgttatctcgctggaatagtccattttgaacatgctctgggcttgaaagcacttggttcgaaacaacctcacctatttgggctttaactctcttcattttccctttacgggaatacgttaacaaaggggttaggagaggtgttgtttttaaaactgggccatttttgggagaaggacataatttaggtggggtgttaatagcttggcccaaatccagcagccccatcatctccttgtcacgtgacttgggtgagggggattcaaaattatgtttgtcttcctcgtacaccatgcctccaacgtttcttgtctgcctcctcatactattttcatacaaattgcccaaaccgtcagggccgacatcccccccgaggttatcttcttcccctaaatcggtcttcccttcttcctgcgacactggtagctcacggtccctggtctgcacgcatttatcacctgtcagtgcattatctgcttgcttcacgacccatcggacaatagctttgtcctttgggtcatgtccgctcttcgtcccgatcgcctttccgttttggtccgcaccggccgcgactgtcgccggtccttctgaccgagagacctgcctgagtcgcccttcttccacaccacgcggcgacctctcctcgtcgccacccctatcctctgtgtcatccaccgtctccgactgggctggagctcccctttgggttttaaatggcacgatttcacttctccctctgcggttatcttcctcgccccccaccactccctccgtcgccatatcctccatggcttctgcctccaaggtccctcccttcgctactgttcgtatttcctccgccacgctctctattcgcgctttgtaccgccacccctgcagctcactaccagaccacgcgtcatcgttacattcgtcagaaaggatttcctctgatgagctgaagtcacgttccctttttggctgccacctagctccaaacaacccactctcctccgctatgtgcaccggaaaaatctccccctgtatatgaatatttaccgtgtgctgaatcagcggtttccatggcgttttgaccagtatcctcgcgacgtcgaacctcctttgatcttctaagctctcctcagcctccaccatctcacctattcccgcgactatctgcttgatacagttcatgtcccatgcaaccaaaggtattccccaacattggatccaagttagtctaaaaccagggcgtagacttggattccacttctctatggagtagaacaggtcgccccatccttcatcttcttcattcaccattttctctgcgttctcttctgtgagccccagtaacaaaaccatgtcatcaccgatgtactttggtgatatgttttgtccgctatcccaccatatggcttcttctatgctatcaaagctagccaagtttttcagtctacccacccaggcctccttgagccattgtttacccgtcatcgaaatgtccaggttcacttccgatgtggcattggaacttatgtattggagtttggctgttgttctcctctgtgctggtttcgggatattcgtgttgaccacctccgcatacgaccgtccttggattcccatattcgcttctggtaacctgctcaggtacacttctgttcttcccttttgtgtttgtcctgtctctgctgtccccctctcgactgtttgtgattctcccttctttggtctcccatatttagggatattaacattcatcttcattcctccgaagaacctattgtccaaatgctgctgaagttgatgcacatctctcacccccttatacctcacaaaaccatatctccttccttggttgtttcttttcctgggtatgaaaacctctctcacgtctccttgctgtttaaaatggaaccacaaatcctttgctgttgcgtcttccgggaacctcgtaaagtaaaaggatataacgtccttatggtctctccagttcgctcgcgtgtaggattgacgctgtcctgtggctaacccttcgatggtcagatgggaaccctctgactctcgaaatctgactctagctctcctctctttcctagtcctaactctctcccacccactgtttctctctctactctctctctctctccatgattggattgttct

SEQ ID NO: 24 - DNA, 17 bp, synthetic construct
  misc_feature 1..17: note = Artificially Synthesized
  source 1..17: mol_type = other DNA
acagctcgagttaatat

SEQ ID NO: 25 - DNA, 16 bp, synthetic construct
  misc_feature 1..16: note = Artificially Synthesized
  source 1..16: mol_type = other DNA
agaaatggagaaagtg

SEQ ID NO: 26 - DNA, 12 bp, synthetic construct
  misc_feature 1..12: note = Artificially Synthesized
  source 1..12: mol_type = other DNA
ccttcttcagtt

SEQ ID NO: 27 - DNA, 13 bp, synthetic construct
  misc_feature 1..13: note = Artificially Synthesized
  source 1..13: mol_type = other DNA
ttttatcatgaaa

SEQ ID NO: 28 - DNA, 16 bp, synthetic construct
  misc_feature 1..16: note = Artificially Synthesized
  source 1..16: mol_type = other DNA
atatatatatatatat

SEQ ID NO: 29 - DNA, 13 bp, synthetic construct
  misc_feature 1..13: note = Artificially Synthesized
  source 1..13: mol_type = other DNA
aaggggggaatgg

SEQ ID NO: 30 - DNA, 42 bp, synthetic construct
  misc_feature 1..42: note = Artificially Synthesized
  source 1..42: mol_type = other DNA
aaatatatatatatatatatatatatatatatatatatatat

SEQ ID NO: 31 - DNA, 19 bp, synthetic construct
  misc_feature 1..19: note = Artificially Synthesized
  source 1..19: mol_type = other DNA
ataaataaataataatagt

SEQ ID NO: 32 - DNA, 26 bp, synthetic construct
  misc_feature 1..26: note = Artificially Synthesized
  source 1..26: mol_type = other DNA
cttcttacttgtaactaagtctttga

SEQ ID NO: 33 - DNA, 289 bp, synthetic construct
  misc_feature 1..289: note = Artificially Synthesized
  source 1..289: mol_type = other DNA
tatattttttgtgtttggtctttgataaatttttctttcgaatttggattctaatgttttaaattttttatcttaaagtctctatcattaatatgtaatcctacatgactcttaattagacacgtagaagtgtgatgtgtcatgtcaaatataacctttgtgatttctatttatacatattggattattaaaaaaaaatattttctttgttaaaaaataataaaatccaacttatataaacaataatccgacataatacatcacatttttacgtgtgtccagttcatac

SEQ ID NO: 34 - DNA, 13 bp, synthetic construct
  misc_feature 1..13: note = Artificially Synthesized
  source 1..13: mol_type = other DNA
ttttgacaaaaac

SEQ ID NO: 35 - DNA, 18 bp, synthetic construct
  misc_feature 1..18: note = Artificially Synthesized
  source 1..18: mol_type = other DNA
caacactaacaaatttta

SEQ ID NO: 36 - DNA, 84 bp, synthetic construct
  misc_feature 1..84: note = Artificially Synthesized
  source 1..84: mol_type = other DNA
caagtatgcaggcttttgtacccatagtaccactggtactatttcaatctataatatatatatttttgctgagcaaaaaaaaaa

SEQ ID NO: 37 - DNA, 54 bp, synthetic construct
  misc_feature 1..54: note = Artificially Synthesized
  source 1..54: mol_type = other DNA
aaacccatgataactggttttaattgtgggctgtctccatactctacacaagct

SEQ ID NO: 38 - DNA, 332 bp, synthetic construct
  misc_feature 1..332: note = Artificially Synthesized
  source 1..332: mol_type = other DNA
ctgtccagccaagatcttgactgttgtagttgaacttagtagctgaagaggaaagagaatgtgatgggtggttgagatttgggaaggagagaaaccttgctgggagaggctgcagaggatccagattcctgctatatattgtcatgataactgtcaagtgtgaaattgagagcttgctaatcttgtagaatatataaaccatttttgactttttttttttaaaaaaatgatttgatcatatggcattcatgtttgtttgagttgtagcagtttctttctgttcccataaattgttatcattcttttttgagagttggatttactggtttgag

SEQ ID NO: 39 - DNA, 19 bp, synthetic construct
  misc_feature 1..19: note = Artificially Synthesized
  source 1..19: mol_type = other DNA
aatagccccacccttaatt

SEQ ID NO: 40 - DNA, 21 bp, synthetic construct
  misc_feature 1..21: note = Artificially Synthesized
  source 1..21: mol_type = other DNA
tcatcatgctaaaactgagta

SEQ ID NO: 41 - DNA, 25 bp, synthetic construct
  misc_feature 1..25: note = Artificially Synthesized
  source 1..25: mol_type = other DNA
ttgaggaagaactgaagaaggtagc

SEQ ID NO: 42 - DNA, 20 bp, synthetic construct
  misc_feature 1..20: note = Artificially Synthesized
  source 1..20: mol_type = other DNA
attgaaaattgttcctgtac

SEQ ID NO: 43 - DNA, 19 bp, synthetic construct
  misc_feature 1..19: note = Artificially Synthesized
  source 1..19: mol_type = other DNA
tggcttaggaagaaagctc

SEQ ID NO: 44 - DNA, 18 bp, synthetic construct
  misc_feature 1..18: note = Artificially Synthesized
  source 1..18: mol_type = other DNA
atctctgatctggctcac

SEQ ID NO: 45 - DNA, 23 bp, synthetic construct
  misc_feature 1..23: note = Artificially Synthesized
  source 1..23: mol_type = other DNA
caacaaatacatataccaaagtc

SEQ ID NO: 46 - DNA, 21 bp, synthetic construct
  misc_feature 1..21: note = Artificially Synthesized
  source 1..21: mol_type = other DNA
acaaaatcatcaccaagttga

SEQ ID NO: 47 - DNA, 18 bp, synthetic construct
  misc_feature 1..18: note = Artificially Synthesized
  source 1..18: mol_type = other DNA
atctgcatgctaatcctg

SEQ ID NO: 48 - DNA, 18 bp, synthetic construct
  misc_feature 1..18: note = Artificially Synthesized
  source 1..18: mol_type = other DNA
ttcgatctgactcctgat

SEQ ID NO: 49 - DNA, 18 bp, synthetic construct
  misc_feature 1..18: note = Artificially Synthesized
  source 1..18: mol_type = other DNA
caatagccccatccttaa

SEQ ID NO: 50 - DNA, 22 bp, synthetic construct
  misc_feature 1..22: note = Artificially Synthesized
  source 1..22: mol_type = other DNA
ctcatcatgctaaaattgagta

SEQ ID NO: 51 - DNA, 23 bp, synthetic construct
  misc_feature 1..23: note = Artificially Synthesized
  source 1..23: mol_type = other DNA
acggattgaggaaggtagcagaa

SEQ ID NO: 52 - DNA, 23 bp, synthetic construct
  misc_feature 1..23: note = Artificially Synthesized
  source 1..23: mol_type = other DNA
cctattgaaaattgttcttgtac

SEQ ID NO: 53 - DNA, 18 bp, synthetic construct
  misc_feature 1..18: note = Artificially Synthesized
  source 1..18: mol_type = other DNA
tggcttaggacgaaagct

SEQ ID NO: 54 - DNA, 17 bp, synthetic construct
  misc_feature 1..17: note = Artificially Synthesized
  source 1..17: mol_type = other DNA
tctctgatttggctcac

SEQ ID NO: 55 - DNA, 25 bp, synthetic construct
  misc_feature 1..25: note = Artificially Synthesized
  source 1..25: mol_type = other DNA
agcaacaaatacatatatcaaagtc

SEQ ID NO: 56 - DNA, 19 bp, synthetic construct
  misc_feature 1..19: note = Artificially Synthesized
  source 1..19: mol_type = other DNA
caaaatcatcacccagttg

SEQ ID NO: 57 - DNA, 16 bp, synthetic construct
  misc_feature 1..16: note = Artificially Synthesized
  source 1..16: mol_type = other DNA
ctgcatgctcatcctg

SEQ ID NO: 58 - DNA, 17 bp, synthetic construct
  misc_feature 1..17: note = Artificially Synthesized
  source 1..17: mol_type = other DNA
cgatctgagtcctgatc

SEQ ID NO: 59 - DNA, 25 bp, synthetic construct
  misc_feature 1..25: note = Artificially Synthesized
  source 1..25: mol_type = other DNA
ggatggaatgggaaagtcttgataa

SEQ ID NO: 60 - DNA, 24 bp, synthetic construct
  misc_feature 1..24: note = Artificially Synthesized
  source 1..24: mol_type = other DNA
aaaacctgcgaagctcctttacta

SEQ ID NO: 61 - DNA, 20 bp, synthetic construct
  misc_feature 1..20: note = Artificially Synthesized
  source 1..20: mol_type = other DNA
gtttgttgtccacgcctgaa

SEQ ID NO: 62 - DNA, 25 bp, synthetic construct
  misc_feature 1..25: note = Artificially Synthesized
  source 1..25: mol_type = other DNA
ttacgtcaatgctcttaaggtctaa

SEQ ID NO: 63 - DNA, 25 bp, synthetic construct
  misc_feature 1..25: note = Artificially Synthesized
  source 1..25: mol_type = other DNA
gccttgacctcatccattgatagag

SEQ ID NO: 64 - DNA, 24 bp, synthetic construct
  misc_feature 1..24: note = Artificially Synthesized
  source 1..24: mol_type = other DNA
ggtgtcaaccacatcttcagcttt

SEQ ID NO: 65 - DNA, 25 bp, synthetic construct
  misc_feature 1..25: note = Artificially Synthesized
  source 1..25: mol_type = other DNA
acttgctctacttcataaatgggta

SEQ ID NO: 66 - DNA, 25 bp, synthetic construct
  misc_feature 1..25: note = Artificially Synthesized
  source 1..25: mol_type = other DNA
acccatttatgaagtagagcaagta

SEQ ID NO: 67 - DNA, 23 bp, synthetic construct
  misc_feature 1..23: note = Artificially Synthesized
  source 1..23: mol_type = other DNA
tccgagaaaggaatagttctgtg

SEQ ID NO: 68 - DNA, 24 bp, synthetic construct
  misc_feature 1..24: note = Artificially Synthesized
  source 1..24: mol_type = other DNA
caccttctccaatgccatatctgt

SEQ ID NO: 69 - DNA, 20 bp, synthetic construct
  misc_feature 1..20: note = Artificially Synthesized
  source 1..20: mol_type = other DNA
gcaagcaaacctcgaaccta

SEQ ID NO: 70 - DNA, 24 bp, synthetic construct
  misc_feature 1..24: note = Artificially Synthesized
  source 1..24: mol_type = other DNA
cccacccgtttatcatttgcactt

SEQ ID NO: 71 - DNA, 22 bp, synthetic construct
  misc_feature 1..22: note = Artificially Synthesized
  source 1..22: mol_type = other DNA
tgtcatcaagcatcaccaggta

SEQ ID NO: 72 - DNA, 24 bp, synthetic construct
  misc_feature 1..24: note = Artificially Synthesized
  source 1..24: mol_type = other DNA
actaataacaaggaggtggcatac

SEQ ID NO: 73 - DNA, 24 bp, synthetic construct
  misc_feature 1..24: note = Artificially Synthesized
  source 1..24: mol_type = other DNA
actaataacaaggaggtggcatac

SEQ ID NO: 74 - DNA, 19 bp, synthetic construct
  misc_feature 1..19: note = Artificially Synthesized
  source 1..19: mol_type = other DNA
ttctggcgtggaggacaaa

SEQ ID NO: 75 - DNA, 23 bp, synthetic construct
  misc_feature 1..23: note = Artificially Synthesized
  source 1..23: mol_type = other DNA
gtcagtgtcctagttttccttga

SEQ ID NO: 76 - DNA, 20 bp, synthetic construct
  misc_feature 1..20: note = Artificially Synthesized
  source 1..20: mol_type = other DNA
agcgaacgggaacagtccat

SEQ ID NO: 77 - DNA, 24 bp, synthetic construct
  misc_feature 1..24: note = Artificially Synthesized
  source 1..24: mol_type = other DNA
ggacgacacttttacatcaaacac

SEQ ID NO: 78 - DNA, 21 bp, synthetic construct
  misc_feature 1..21: note = Artificially Synthesized
  source 1..21: mol_type = other DNA
gcttcaccaagtcaactctga

SEQ ID NO: 79 - DNA, 27 bp, synthetic construct
  misc_feature 1..27: note = Artificially Synthesized
  source 1..27: mol_type = other DNA
tttccctgtattggcgtccacttgtta

SEQ ID NO: 80 - DNA, 19 bp, synthetic construct
  misc_feature 1..19: note = Artificially Synthesized
  source 1..19: mol_type = other DNA
atcgtcaaccactacatcg

SEQ ID NO: 81 - DNA, 27 bp, synthetic construct
  misc_feature 1..27: note = Artificially Synthesized
  source 1..27: mol_type = other DNA
tttaacaaatgggacgaatgaatattt

SEQ ID NO: 82 - DNA, 11 bp, synthetic construct
  misc_feature 1..11: note = Artificially Synthesized
  source 1..11: mol_type = other DNA
atgtgtgtgtg

SEQ ID NO: 83 - DNA, 10 bp, synthetic construct
  misc_feature 1..10: note = Artificially Synthesized
  source 1..10: mol_type = other DNA
gaaaggtgga

SEQ ID NO: 84 - DNA, 20 bp, synthetic construct
  misc_feature 1..20: note = Artificially Synthesized
  source 1..20: mol_type = other DNA
taatataatttatttattaa

(19) United States
(12) Patent Application Publication
(10) Pub. No.: US 2026/0043043 A1
(43) Pub. Date: Feb. 12, 2026

(54) NOVEL GENETIC LOCI ASSOCIATED WITH RUST RESISTANCE IN SOYBEANS

(71) Applicants: OIL CROPS RESEARCH INSTITUTE, CHINESE ACADEMY OF AGRICULTURAL SCIENCES, Wuhan (CN); SYNGENTA CROP PROTECTION AG, Basel (CH)

(72) Inventors: Zhihui SHAN, Wuhan (CN); Qingnan HAO, Wuhan (CN); Haifeng CHEN, Wuhan (CN); Yanyan YANG, Wuhan (CN); Chanjuan ZHANG, Wuhan (CN); Limiao CHEN, Wuhan (CN); Songli YUAN, Wuhan (CN); Dong CAO, Wuhan (CN); Wei GUO, Wuhan (CN); Xiaojuan ZHANG, Wuhan (CN); Shuilian CHEN, Wuhan (CN); Zhonglu YANG, Wuhan (CN); Dezhen QIU, Wuhan (CN); Xinan ZHOU, Wuhan (CN); Qingli LIU, Durham, NC (US); Becky Welsh BREITINGER, Durham, NC (US); Shujie DONG, Durham, NC (US)

(73) Assignees: OIL CROPS RESEARCH INSTITUTE, CHINESE ACADEMY OF AGRICULTURAL SCIENCES, Wuhan (CN); SYNGENTA CROP PROTECTION AG, Basel (CH)

(21) Appl. No.: 19/324,863

(22) Filed: Sep. 10, 2025

Related U.S. Application Data
(63) Continuation of application No. 17/624,173, now Pat. No. 12,435,341.

(30) Foreign Application Priority Data
Jul. 1, 2019 (CN) 201910584420.1

(51) Int. Cl. C12N 15/82; C07K 14/415; C12Q 1/6895

(52) U.S. Cl. CPC C12N 15/8282; C07K 14/415; C12N 15/8205; C12Q 1/6895; C07K 2319/00; C12Q 2600/13; C12Q 2600/156

(57) ABSTRACT

The present invention relates to methods and compositions for identifying, selecting, and/or producing a soybean plant or germplasm resistant to Asian soybean rust using markers, genes and chromosomal intervals derived from Glycine max strain SX6907. A soybean plant or germplasm that has been identified, selected, and/or produced by any of the methods of the present invention is also provided. Asian soybean rust resistant soybean seeds, plants, and germplasms are also provided.

### CROSS REFERENCE TO RELATED APPLICATIONS

This application is a continuation of U.S. application Ser. No. 17/624,173, filed Dec. 30, 2021, which is a National Stage application of International Application No. PCT/CN2020/099619, filed Jul. 1, 2020, which claims priority to Chinese Patent Application No. 201910584420.1, filed Jul. 1, 2019, the disclosure of which is incorporated herein in its entirety.

### SEQUENCE LISTING

The instant application contains a Sequence Listing, which has been filed electronically in XML format and is hereby incorporated by reference in its entirety. Said XML copy, created Sep. 10, 2025, is named S19996_1600US.C1_ST26.xml and is 218,109 bytes in size.

### TECHNICAL FIELD

The invention relates to the field of plant genetic engineering, in particular to a protein related to rust resistance in soybeans, a coding gene and use thereof.

### BACKGROUND

Soybean (Glycine max) is one of the four major oil-bearing crops around the world and one of the most important crops for producing proteins. Rust is a major disease in soybean production around the world and the main method to control the disease is the application of foliar fungicides.

At present, there are no commercial varieties with complete resistance against rust. The pathogen of rust is Phakopsora pachyrhizi, and the hosts of the pathogen of rust include a wide range of leguminous plants (at least 31 species in 17 genera; Slaminko et al., (2008) Plant Dis., 92: 797-771; and at least 42 species in 19 genera; Frederick et al., (2002) Mycology, 92: 217-227, respectively). In addition, another 152 species have been recognized as potential hosts of Phakopsora pachyrhizi (Bonde et al., (2008) Plant Dis., 92: 30-38; Goellner et al., (2010) Molecular Plant Pathology, 11: 169-177; Ono et al., (1992) Mycol. Res., 96 (10): 825-850; and Slaminko et al., (2008) Plant Dis., 92: 797-771). The application of fungicides is currently the only available method to mitigate rust.

There are few soybean resources that are resistant to Phakopsora pachyrhizi. The resistance of soybean resources to Phakopsora pachyrhizi is specific for individual physiological races, therefore, when using resistant resources for breeding, if such resistance specificity is ignored, the resistance in the resistant resources may be lost due to the incompatibility between host resistance and the physiological races, which is not conducive to the persistent utilization of the resistant resources.

Cultivating rust-resistant varieties is the most economical and effective way to prevent rust damage. By exploring new resistance genes for diseases in soybean germplasm and other leguminous plants and transferring them as a single gene, multiple genes or in the form of multiple gene cassettes to soybean, these resistance genes may be able to provide resistance to Phakopsora pachyrhizi through homologous or heterologous expression. Accordingly, what is needed are novel resistance genes to rust that can be introduced into commercial soybean plants to control rust resistance.

### SUMMARY OF THE INVENTION

This summary lists several embodiments of the presently disclosed subject matter, and in many cases lists variations and permutations of these embodiments.

Thus, it is an object of the presently disclosed subject matter to provide methods for conveying rust resistance into non-resistant soybean germplasm or plant lines. Further the presently disclosed subject matter provides novel Glycine max lines comprising in its genome a chromosome interval, loci, and/or gene that is derived from Glycine max strains SX6907 and further confers Asian soybean rust resistance (herein, “ASR”) in said novel Glycine max line.

The present invention provides for chromosomal intervals derived from Glycine max strain SX6907 that when introduced into a plant (e.g. a soybean such as Glycine max strain Williams 82) are sufficient to confer increased rust resistance, such as e.g. Asian soybean rust (“ASR”) resistance, as compared to a control plant not comprising said chromosomal interval. The invention also provides for proteins and nucleic acids derived from Glycine max strain SX6907 that confer rust resistance.

Compositions and methods for identifying, selecting, and producing Glycine plants (including wild Glycines (e.g. Glycine tomentella and Glycine max lines)) with enhanced rust resistance are also provided. Rust resistant soybean plants and germplasms are also provided.

In some embodiments, methods of identifying a rust resistant soybean plant or germplasm are provided. Such methods may comprise detecting, in the soybean plant or germplasm, a genetic loci or molecular marker (e.g. SNP or a Quantitative Trait Loci (QTL)) associated with enhanced disease resistance, in particular ASR resistance. In some embodiments the genetic loci or molecular marker associates with the presence of a chromosomal interval comprising the nucleotide sequence or a portion thereof of SEQ ID NOs 11, 12, or 13, or a portion thereof wherein the portion thereof associates with ASR resistance. In another embodiment, the genetic loci or molecular marker associates with the presence of nucleotide of SEQ ID NO: 2 or a portion thereof associated with ASR resistance. In yet another embodiment, the genetic loci or molecular marker associates with the presence of nucleotide encoding the amino acid sequence of SEQ ID NO: 1 or a portion thereof associated with ASR resistance.

In some embodiments, methods of producing ASR resistant soybean plants are provided. Such methods may comprise detecting, in a soybean plant or germplasm, the presence of a genetic loci and/or a genetic marker associated with enhanced pathogen resistance (e.g. ASR) and producing a progeny plant from said soybean germplasm. In some embodiments, the methods are used to generate novel ASR resistant Glycine max strains.

Other embodiments include methods of selecting a disease resistant soybean plant or germplasm. Such methods may include crossing a first soybean plant or germplasm with a second soybean plant or germplasm, wherein the first soybean plant or germplasm comprises a genetic loci derived from Glycine max strains SX6907 or a progeny plant thereof comprising any one of SEQ ID NOs 2, 11, 12, or 13, or a portion thereof associated with enhanced disease and/or ASR resistance, and/or tolerance, and selecting a progeny plant or germplasm that possesses the genetic loci.

In some embodiments, methods of introgressing a genetic loci derived from soybean strains SX6907 associated with enhanced rust resistance into a soybean plant or germplasm are provided. Such methods may comprise crossing a first soybean plant or germplasm comprising a chromosomal interval (e.g. SEQ ID Nos 11, 12, or 13, or a portion thereof) derived from soybean strains SX6907 associated with enhanced rust (ASR) resistance with a second soybean plant or germplasm that lacks said genetic loci and optionally repeatedly backcrossing progeny plants comprising said genetic allele with the second soybean plant or germplasm to produce an soybean plant (e.g. Glycine max) or germplasm with enhanced pathogen resistance comprising the chromosomal interval derived from soybean strains SX6907 and/or ZRYCR1 associated with enhanced ASR resistance. In one embodiment, the chromosome interval comprises SEQ ID NO: 2. In another embodiment, the chromosome interval comprises SEQ ID NO: 1.

Progeny comprising the chromosomal interval associated with enhanced pathogen resistance may be identified by detecting, in their genomes, the presence of a marker associated with or genetically linked to said chromosomal interval derived from soybean accession number strains SX6907 and/or ZRYCR1 wherein said chromosomal interval comprises SEQ ID NOs 11, 12, or 13, or a portion thereof and the marker can be any of the favorable alleles as described in Table 1.

Soybean plants and/or germplasms identified, produced or selected by the methods of this invention are also provided, as are any progeny and/or seeds derived from a soybean plant or germplasm identified, produced or selected by these methods. In one embodiment molecular markers associating with the presence of a chromosomal intervals depicted in any one of SEQ ID NOs 11, 12, or 13 may be used to identify or select for plant lines resistant to ASR. Further said molecular markers may be located within 20 cM, 10 cM, 5 cM, 4 cM, 3 cM, 2 cM, and 1 cM of said chromosomal interval or from any respective favorable allele associated with ASR resistance as depicted in Table 1. In another embodiment, said molecular marker may be located within 20 cM, 10 cM, 5 cM, 4 cM, 3 cM, 2 cM, 1 cM of any SNP markers associated with ASR as described in Table 1.

Non-naturally occurring soybean seeds, plants and/or germplasms comprising one or genetic loci derived from strains SX6907 and/or ZRYCR1 associated with enhanced rust resistance are also provided. In specific embodiments said genetic loci comprises any one of SEQ ID NO: 11, 12, or 13, or a portion thereof and/or any favorable alleles as depicted in Table 1. In other embodiments, the genetic loci comprises the nucleic acid sequence of SEQ ID NO: 2 or a nucleic acid encoding the protein of SEQ ID NO: 1.

A marker associated with enhanced rust (ASR) resistance may comprise, consist essentially of or consist of a single allele or a combination of alleles at one or more genetic loci derived from strains SX6907 and/or ZRYCR1 that associate with enhanced pathogen (ASR) resistance. In one embodiment, the marker is within a chromosomal interval as described by SEQ ID NO: 11, 12, or 13. In another embodiment, the marker is within SEQ ID NO: 2. In another embodiment, the marker is any one of the favorable alleles as depicted in Table 1.

Additional compositions and methods for producing Glycine plants having enhanced disease resistance are also provided. In one aspect of the invention, there is provided a DNA construct that comprises a promoter that functions in plant cells operably linked to a DNA molecule encoding a protein having at least 80% 100% homology to SEQ ID NO: 1. The current disclosure is also directed to DNA molecules. Exemplary DNA molecules include (B1) a DNA molecule shown in SEQ ID NO: 2; (B2) a DNA molecule hybridizing to the DNA molecule defined in (B1) under a stringent condition and encoding the protein; (B3) a DNA molecule having more than 99%, more than 95%, more than 90%, more than 85%, or more than 80% homology with the DNA sequences defined in (B1) and (B2) and encoding the protein.

In another aspect of the invention is directed to an expression cassette, a recombinant vector, a recombinant bacterium, or a transgenic cell line comprising the nucleic acid molecule. The invention is also directed to a method of preparing a fertile transgenic plant comprising providing a plant expression cassette comprising at least one of an RG21 gene and an RG22 gene and contacting recipient plant cells with the plant expression cassette under conditions permitting the uptake of the plant expression cassette by the recipient cells; selecting the recipient plant cells that contain the plant expression cassette; regenerating plants from the selected recipient plant cells; and identifying a fertile transgenic plant that is resistant to soybean pathogens, particularly ASR.

In another aspect of the invention there is provided a fertile transgenic plant that comprises a plant expression cassette as described above wherein the plant is resistant to soybean pathogens, particularly ASR.

In another aspect of the invention there is provided a method of controlling ASR in a field comprising the step of planting the seed from a plant comprising an expression cassette as described herein.

As a further aspect are seeds that produce the transgenic plants of the invention and seeds produced by the transgenic plants of the invention.

Also provided are harvested products derived from the transgenic plants of the invention, wherein the harvested product optionally comprises a nucleotide sequence, expression cassette, vector and/or at least one of a protein or DNA molecule of the invention. Further provided are processed products derived from the harvested products of the invention, wherein the harvested product optionally comprises a nucleotide sequence, expression cassette, vector and/or at least one of a protein or DNA molecule of the invention.

Still further, the disclosure provides as an additional aspect a method of producing a transgenic plant with increased resistance to a soybean pathogen. In embodiments, the method may comprise increasing the expression level and/or activity of a protein having at least 80%-100% homology to SEQ ID NO: 1. In another, the disclosure is directed to methods for breeding a plant variety with improved resistance against rust, comprising the step of increasing the expression level and/or activity of a protein having at least 80%-100% homology to SEQ ID NO: 1 in a recipient plant.

Compositions of the invention also include probes and primer pairs for detecting the novel resistance genes disclosed herein.

The foregoing and other objects and aspects of the present invention are explained in detail in the drawings and specification set forth below.

### BRIEF DESCRIPTION OF THE DRAWINGS

FIG. 1 is a plasmid map of a recombinant vector pB2GW7-RppRC1 with rust resistance gene RppRC1.

FIG. 2 is a PCR detection picture of T1 generation of RppRC1 transgenic plants. M: marker. L1-1, L1-2, and L1-3 are partial individual plants of the T1 plants of the transformation event L1, and L2-1, L2-2 and L2-3 are partial individual plants of the T1 plants of the transformation event L2, and the negative: negative control Tianlong No. 1, positive: positive control SX6907.

FIG. 3 shows the RT-PCR identification of the expression of RppRC1 gene in T1 generation of transgenic plants. L1-2 is an individual plant of the T1 plants of transformation event L1, and L2-1 is an individual plant of the T1 plants of transformation event L2.

FIGS. 4A and 4B show the southern analysis of transgenic plants. FIG. 4A shows southern analysis of RppRC1 transgenic plants. L1-1, L1-2, L1-3, L1-4 and L1-5 are partial individual plants of the T1 plants of transformation event L1, respectively. L2-1 and L2-2 are partial individual plants of the T1 plants of transformation event L2, while Tianlong No. 1 is the negative control. FIG. 4B shows southern analysis of transgenic plants with empty vector. L3-1, L3-2, L3-3, L3-4, L3-5 and L3-6 were partial individual plants of the T1 plants of empty vector transformation event L3. CK is Tianlong No. 1.

FIG. 5 shows the phenotype for resistance identification of TO transgenic plants 12 days after inoculation. SX6907 is a resistance control and shows immunity; RppRC1 transformation event L2 shows immunity; empty vector transformation event L3 shows susceptibility; non-transgenic Tianlong No. 1 shows susceptibility.

FIG. 6 shows the phenotype for resistance identification of T1 transgenic plants 12 days after inoculation. SX6907 is a resistance control and shows immunity; the individual plant L2-1 of the T1 plants of RppRC1 transformation event L2 shows immunity; the negative control Tianlong No. 1 shows susceptibility.

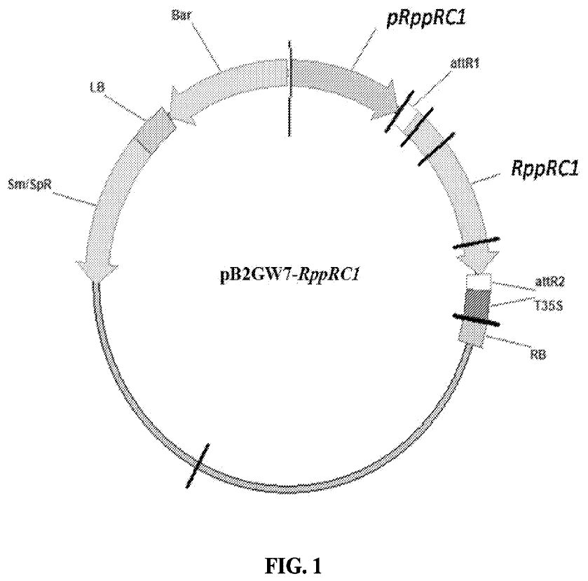

With reference to FIG. 1-6, the same transformation event is marked with the same label and the same individual plant is marked with the same label.

### DETAILED DESCRIPTION OF ILLUSTRATIVE EMBODIMENTS

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

All patents, published patent applications, and publications cited herein are incorporated by reference as if set forth fully herein.

Unless defined otherwise, all technical and scientific terms used herein have the same meaning commonly understood to one of ordinary skill in the art to which this invention pertains. Otherwise, certain terms used herein have the meanings as set forth in the specification.

The instant application is directed to new genes encoding proteins for rust resistance and their use to provide rust resistance in plants, in particular in soybeans. In one embodiment of the invention, the gene is derived from soybean (Glycine max) SX6907.

Accordingly, in one aspect, the instant application provides proteins related to rust resistance in a plant, a coding gene and use thereof. In the invention, the resistance against rust of transgenic soybean obtained by transforming RppRC1 gene into soybean variety Tianlong No. 1 is significantly higher than that of wild-type soybean, indicating that RppRC1 and the coding gene thereof can regulate and control the resistance of leguminous plants against rust, and improve the rust resistance of plants after overexpression. RppRC1 and the coding gene thereof can be used to improve the disease resistance of leguminous crops and are of great significance for breeding new varieties with disease resistance.

Other aspects of the invention include methods for conveying rust resistance into non-resistant soybean germplasm or plant lines. Further the presently disclosed subject matter provides novel Glycine max lines comprising in its genome a chromosome interval, loci, and/or gene that is derived from Glycine max SX6907 and further confers soybean rust resistance in said novel Glycine max line.

### Definitions

Unless defined otherwise, all technical and scientific terms used herein have the same meaning as commonly understood to one of ordinary skill in the art to which the presently disclosed subject matter belongs.

Although the following terms are believed to be well understood by one of ordinary skill in the art, the following definitions are set forth to facilitate understanding of the presently disclosed subject matter.

Unless explicitly stated otherwise in the context, the singular forms “a,” “an,” and “the” as used herein include multiple references. Thus, for example, references to “a cell” include a plurality of such cells, and references to “the protein” include references to one or more proteins and their equivalents known to those skilled in the art, and so on. Unless explicitly stated otherwise, all technical and scientific terms used herein have the same meanings generally understood by those of ordinary skill in the art to which the present invention belongs.

As used in the specification and claims, the term “comprise” and grammatical variations thereof may include aspects of “consist of” and “substantially consist of”. “Comprise” and grammatical variations thereof may also mean “comprise, but not limited to”.

As used herein, the word “or” refers to any member of a particular list and also comprises any combination of members of the list.

As used herein, the term “and/or” refers to and encompasses any and all possible combinations of one or more of the associated listed items, as well as the lack of combinations when interpreted in the alternative (“or”).

The range may be expressed in the present invention as being from “about” one specific value and/or to “about” another specific value. When expressing such ranges, other aspects include from one specific value and/or to other specific values. Similarly, when the value is expressed as an approximation, it should be understood that by using the antecedent “about”, the specific value forms another aspect. It should also be understood that the endpoints of each of the ranges are both significantly related to and independent of the other endpoint. It should also be understood that there are multiple values disclosed in the present invention and, in addition to the value itself, each value is also disclosed herein in the form of “about” the specific value. For example, if the value “10” is disclosed, “about 10” is also disclosed. It should also be understood that each unit between two specific units is also disclosed. For example, if 10 and 15 are disclosed, 11, 12, 13 and 14 are also disclosed.

The term “consists essentially of” (and grammatical variants thereof), as applied to a polynucleotide sequence of this invention, means a polynucleotide sequence that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to express the polynucleotide sequence of at least about 50% or more as compared to the expression level of a polynucleotide sequence consisting of the recited sequence.

The term “introduced” as used herein, in connection to a plant, means accomplished by any manner including but not limited to; introgression, transgenic, Clustered Regularly Interspaced Short Palindromic Repeats modification (CRISPR), Transcription activator-like effector nucleases (TALENs) (Feng et al. 2013, Joung & Sander 2013), meganucleases, or zinc finger nucleases (ZFNs).

As used herein, the term “soybean” refers to soybean and any plant variety bred or cultured using soybean including “wild Glycine” plants.

As used herein, the term “wild Glycine” refers to a perennial Glycine plant, for example any one of G. canescens, G. argyrea, G. clandestine, G. latrobeana, G. albicans, G. aphyonota, G. arenaria, G. curvata, G. cyrtoloba, G. dolichocarpa, G. falcate, G. gracei, G. hirticaulis, G. lactovirens, G. latifolia, G. microphylla, G. montis-douglas, G. peratosa, G. pescadrensis, G. pindanica, G. pullenii, G. rubiginosa, G. stenophita, G. syndetika, or G. tomentella.

In the present invention, “nucleic acid” refers to a deoxyribonucleotide or ribonucleotide polymer in single-stranded or double-stranded form and, unless otherwise limited, encompasses known analogues (e.g., peptide nucleic acids) that have the basic properties of natural nucleotides in the following aspects: it hybridizes to single-stranded nucleic acids in a manner similar to that of naturally occurring nucleotides.

The term “a variant” and grammatical variations thereof refer to a substantially similar sequence. For nucleic acid molecules, variants comprise deletion and/or addition of one or more nucleotides at one or more sites in the native nucleic acid molecule, and/or substitution of one or more nucleotides at one or more sites in the native nucleic acid molecule.

The term “protein” refers to a polymer of amino acid residues. The term applies to amino acid polymers in which one or more amino acid residues are artificial chemical analogues of corresponding natural amino acids, and to natural amino acid polymers.

As used herein, a “native” nucleic acid molecule or protein comprises a naturally occurring nucleotide sequence or an amino acid sequence, respectively.

As used herein, the term “coding” or “encoding” and grammatical variations thereof are used to mean that a nucleic acid comprises the desired information, which is specified by the use of codons to direct the translation of nucleotide sequences (for example, leguminous sequences) into specific proteins. A nucleic acid coding a protein may comprise an untranslated sequence (e.g., an intron) within the translation region of the nucleic acid or may lack such an intermediate untranslated sequence (e.g., as in cDNA).

As used herein, the term “allele” refers to one of two or more different nucleotides or nucleotide sequences that occur at a specific locus.

A marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with enhanced pathogen resistance” refers to a marker whose presence or absence can be used to predict whether and/or to what extent a plant will display a pathogen resistant phenotype (e.g. any favorable SNP allele as described herein are “associated with” ASR (rust) resistance in a soybean plant).

As used herein, the terms “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-53 (1994). The initial cross gives rise to the F1 generation. The term “BC1” refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval defined by and including,” used in reference to particular loci and/or alleles, refers to a chromosomal interval delimited by and encompassing the stated loci/alleles.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “cultivar” and “variety” refer to a group of similar plants that by structural or genetic features and/or performance can be distinguished from other varieties within the same species.

As used herein, the terms “desired allele”, “favorable allele” and “allele of interest” are used interchangeably to refer to an allele associated with a desired trait (e.g. ASR resistance).

As used herein, the terms “inhibit,” “reduce,” etc., and grammatical variations thereof refer to any reduction in the expression or function of a target gene product, including any relative reduction in the expression or function up to and including complete elimination of the expression or function of the target gene product.

The term “enhance” and grammatical variations thereof refer to improvement, increase, amplification, reproduction, rise and/or elevation to reduce one or more disease symptoms.

As used herein, the terms “increase”, “enhance” etc., and grammatical variations thereof are used to refer to any promotion or gain or increase in the expression, function or activity of a product of a target gene (for example, a resistance gene) as compared to a susceptible plant, thereby providing increased resistance to one or more pathogens (for example, Phakopsora) or diseases (for example, rust). Additionally, as used herein, the term “cause” or “increase” and grammatical variations thereof may refer to a higher expression of a target gene product such that the level is increased by 10% or more, 50% or more, or 100%, relative to a cell or plant lacking the target gene or protein disclosed herein.

The term “immunity” or “immune” is used in the present invention to refer to the absence of any macroscopically visible disease symptoms. The term “partial resistance” is used in the present invention to refer to the presence of macroscopically visible lesions without or with limited spore formation and/or a reduction in the scope or degree of any disease symptoms and/or a delay in the progression of any disease symptoms, and may, for example, manifest a reduction in the number of lesions or lesions with reduced spore formation. As used herein, the term “susceptibility” or the phrase “lack of resistance” in terms of rust refers to the occurrence of a lesion in the case where the spore formation level is equal to or higher than the spore formation level observed in a reference standard, such as, for example, the variety Williams or Peking.

The term “resistance” is used herein to refer to the absence or reduction of one or more disease symptoms caused by plant pathogens in plants. Resistance may mean that disease symptoms, such as the number of diseased plants, defoliation, and associated yield loss, are reduced, minimized or decreased when compared to plants susceptible to the diseases or plants that do not comprise effective resistance genes that reduce one or more disease symptoms. In addition, resistance may include prevention or delay of pathogen proliferation. Generally speaking, the term “resistance” includes immunity and partial resistance as defined above.

As used herein, the terms “enhanced pathogen resistance”, “enhanced plant pathogen resistance”, or “enhanced disease resistance” refers to an improvement, enhancement, or increase in a plant's ability to endure and/or thrive despite being infected with a disease (e.g. Asian soybean rust) as compared to one or more control plants (e.g., one or both of the parents, or a plant lacking a marker associated with enhanced pathogen resistance to respective pathogen/disease). Enhanced disease resistance includes any mechanism (other than whole-plant immunity or resistance) that reduces the expression of symptoms indicative of infection for a respective disease such as Asian soybean rust, soybean cyst nematode, Phytophthora, etc.

“A plant pathogen” and grammatical variations thereof can be used herein to refer to, for example, a fungal pathogen of the genus Phakopsora of the class Basidiomycetes (including Phakopsora pachyrhizi and Phakopsora meibomiae). The plant diseases or the diseases of leguminous crops may be, for example, rust.

The term “disease resistance gene” or “resistance gene” is used in the present invention to refer to a gene encoding a protein capable of enhancing or improving the defense or immune system response in plants.

The term “orthologue” and grammatical variations thereof refer to genes derived from common ancestral genes and present in different species due to speciation.

An “elite line” or “elite strain” is an agronomically superior line that has resulted from many cycles of breeding and selection for superior agronomic performance. Numerous elite lines are available and known to those of skill in the art of soybean breeding. An “elite population” is an assortment of elite individuals or lines that can be used to represent the state of the art in terms of agronomically superior genotypes of a given crop species, such as soybean. Similarly, an “elite germplasm” or elite strain of germplasm is an agronomically superior germplasm, typically derived from and/or capable of giving rise to a plant with superior agronomic performance, such as an existing or newly developed elite line of soybean.

An “elite” plant is any plant from an elite line, such that an elite plant is a representative plant from an elite variety. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, Ill., USA); DKB17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minnesota, USA); 90M01, 91M30, 92M33, 93M11, 94M30, 95M30, 97B52, P008T22R2; P16T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9, S78-G6, S0009-M2; S007-Y4; S04-D3; S14-A6; S20-T6; S21-M7; S26-P3; S28-N6; S30-V6; S35-C3; S36-Y6; S39-C4; S47-K5; S48-D9; S52-Y2; S58-Z4; S67-R6; S73-S8; and S78-G6 (Syngenta Seeds, Henderson, Ky., USA); Richer (Northstar Seed Ltd. Alberta, CA); 14RD62 (Stine Seed Co. Ia., USA); or Armor 4744 (Armor Seed, LLC, Ar., USA).

The terms “agronomically elite” as used herein, means a genotype that has a culmination of many distinguishable traits such as emergence, vigor, vegetative vigor, disease resistance, seed set, standability, yield and threshability which allows a producer to harvest a product of commercial significance.

As used herein, the term “commercially significant yield” or “agronomically acceptable yield” refers to a grain yield of at least 100% of a commercial check variety such as AG2703 or DKB23-51.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

The term “germplasm” is used in the present invention to refer to genetic material derived from an individual (e.g., a plant), a group of individuals (e.g., a plant germline, variety, or family), or a clone derived from a strain, variety, species, or culture. Germplasm can be part of an organism or a cell, or can be isolated from an organism or a cell. Germplasm provides genetic material having a specific molecular composition that provides the physical basis for some or all of the genetic properties of an organism or cell culture

A “genetic map” is a description of genetic linkage relationships among loci on one or more chromosomes within a given species, generally depicted in a diagrammatic or tabular form. For each genetic map, distances between loci are measured by the recombination frequencies between them. Recombinations between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm may refer to seeds, cells (including protoplasts and calli) or tissues from which new plants may be grown, as well as plant parts that can be cultured into a whole plant (e.g., stems, buds, roots, leaves, etc.).

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “hybrid” refers to a seed and/or plant produced when at least two genetically dissimilar parents are crossed.

As used herein, the term “inbred” refers to a substantially homozygous plant or variety. The term may refer to a plant or variety that is substantially homozygous throughout the entire genome or that is substantially homozygous with respect to a portion of the genome that is of particular interest.

As used herein, the term “indel” refers to an insertion or deletion in a pair of nucleotide sequences, wherein a first sequence may be referred to as having an insertion relative to a second sequence or the second sequence may be referred to as having a deletion relative to the first sequence.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be repeatedly backcrossed to a line having a desired genetic background and selected for the desired allele, with the result being that the desired allele becomes fixed in the desired genetic background. For example, a marker associated with enhanced ASR tolerance may be introgressed from a donor into a recurrent parent that is not disease resistant. The resulting offspring could then be repeatedly backcrossed and selected until the progeny possess the ASR tolerance allele(s) in the recurrent parent background.

As used herein, the term “linkage” refers to the degree with which one marker locus is associated with another marker locus or some other locus (for example, an ASR tolerance locus). The linkage relationship between a molecular marker and a phenotype may be given as a “probability” or “adjusted probability.” Linkage can be expressed as a desired limit or range. For example, in some embodiments, any marker is linked (genetically and physically) to any other marker when the markers are separated by less than about 50, 40, 30, 25, 20, or 15 map units (or cM). For example, embodiments of the invention herein, provide for marker loci closely linked to ASR resistant chromosomal intervals comprising a nucleotide sequence of any one of SEQ ID NOs 2, 11, 12, or 13.

In some aspects of the present invention, it is advantageous to define a bracketed range of linkage, for example, from about 10 cM and about 20 cM, from about 10 cM and about 30 cM, or from about 10 cM and about 40 cM. The more closely a marker is linked to a second locus, the better an indicator for the second locus that marker becomes. Thus, “closely linked loci” such as a marker locus and a second locus display an inter-locus recombination frequency of about 10%, 9%, 8%, 7%, 6%, 5%, 4%, 3%, or 2% or less. In some embodiments, the relevant loci display a recombination frequency of about 1% or less, e.g., about 0.75%, 0.5%, 0.25% or less. Two loci that are localized to the same chromosome, and at such a distance that recombination between the two loci occurs at a frequency of less than about 10% (e.g., about 9%, 8%, 7%, 6%, 5%, 4%, 3%, 2%, 1%, 0.75%, 0.5%, or 0.25%, or less) may also be said to be “proximal to” each other. Since one cM is the distance between two markers that show a 1% recombination frequency, any marker is closely linked (genetically and physically) to any other marker that is in close proximity, e.g., at or less than about 10 cM distant. Two closely linked markers on the same chromosome may be positioned about 9, 8, 7, 6, 5, 4, 3, 2, 1, 0.75, 0.5 or 0.25 cM or less from each other.

As used herein, the term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and, by definition, are separated by less than 50 cM on the same chromosome). As used herein, linkage can be between two markers, or alternatively between a marker and a phenotype. A marker locus can be “associated with” (linked to) a trait, e.g., Asian Soybean Rust. The degree of linkage of a molecular marker to a phenotypic trait is measured, e.g., as a statistical probability of co-segregation of that molecular marker with the phenotype.

Linkage disequilibrium is most commonly assessed using the measure r², which is calculated using the formula described by Hill and Robertson, Theor. Appl. Genet. 38:226 (1968). When r²=1, complete linkage disequilibrium exists between the two marker loci, meaning that the markers have not been separated by recombination and have the same allele frequency. Values for r² above ⅓ indicate sufficiently strong linkage disequilibrium to be useful for mapping. Ardlie et al., Nature Reviews Genetics 3:299 (2002). Hence, alleles are in linkage disequilibrium when r² values between pairwise marker loci are greater than or equal to about 0.33, 0.4, 0.5, 0.6, 0.7, 0.8, 0.9, or 1.0.

As used herein, the term “linkage equilibrium” describes a situation where two markers independently segregate, i.e., sort among progeny randomly. Markers that show linkage equilibrium are considered unlinked (whether or not they lie on the same chromosome).

A “locus” is a position on a chromosome where a gene or marker or allele is located. In some embodiments, a locus may encompass one or more nucleotides.

As used herein, the terms “marker” and “genetic marker” are used interchangeably to refer to a nucleotide and/or a nucleotide sequence that has been associated with a phenotype, trait, or trait form. In some embodiments, a marker may be associated with an allele or alleles of interest and may be indicative of the presence or absence of the allele or alleles of interest in a cell or organism. A marker may be, but is not limited to, an allele, a gene, a haplotype, a restriction fragment length polymorphism (RFLP), a simple sequence repeat (SSR), random amplified polymorphic DNA (RAPD), cleaved amplified polymorphic sequences (CAPS) (Rafalski and Tingey, Trends in Genetics 9:275 (1993)), an amplified fragment length polymorphism (AFLP) (Vos et al., Nucleic Acids Res. 23:4407 (1995)), a single nucleotide polymorphism (SNP) (Brookes, Gene 234:177 (1993)), a sequence-characterized amplified region (SCAR) (Paran and Michelmore, Theor. Appl. Genet. 85:985 (1993)), a sequence-tagged site (STS) (Onozaki et al., Euphytica 138:255 (2004)), a single-stranded conformation polymorphism (SSCP) (Orita et al., Proc. Natl. Acad. Sci. USA 86:2766 (1989)), an inter-simple sequence repeat (ISSR) (Blair et al., Theor. Appl. Genet. 98:780 (1999)), an inter-retrotransposon amplified polymorphism (IRAP), a retrotransposon-microsatellite amplified polymorphism (REMAP) (Kalendar et al., Theor. Appl. Genet. 98:704 (1999)), a chromosome interval, or an RNA cleavage product (such as a Lynx tag). A marker may be present in genomic or expressed nucleic acids (e.g., ESTs). The term marker may also refer to nucleic acids used as probes or primers (e.g., primer pairs) for use in amplifying, hybridizing to and/or detecting nucleic acid molecules according to methods well known in the art. A large number of soybean molecular markers are known in the art, and are published or available from various sources, such as the SoyBase internet resource.

Markers corresponding to genetic polymorphisms between members of a population can be detected by methods well-established in the art. These include, e.g., nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), and/or detection of amplified fragment length polymorphisms (AFLPs). Well established methods are also known for the detection of expressed sequence tags (ESTs) and SSR markers derived from EST sequences and randomly amplified polymorphic DNA (RAPD).

A “marker allele,” also described as an “allele of a marker locus,” can refer to one of a plurality of polymorphic nucleotide sequences found at a marker locus in a population that is polymorphic for the marker locus.

“Marker-assisted selection” (MAS) is a process by which phenotypes are selected based on marker genotypes. In some embodiments, marker genotypes are used to identify plants that will be selected for a breeding program or for planting. In some embodiments, marker genotypes are used to identify plants that will not be selected for a breeding program or for planting (i.e., counter-selected plants), allowing them to be removed from the breeding/planting population.

As used herein, the terms “marker locus” and “marker loci” refer to a specific chromosome location or locations in the genome of an organism where a specific marker or markers can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

As used herein, the terms “marker probe” and “probe” refer to a nucleotide sequence or nucleic acid molecule that can be used to detect the presence of one or more particular alleles within a marker locus (e.g., a nucleic acid probe that is complementary to all of or a portion of the marker or marker locus, through nucleic acid hybridization). Marker probes comprising about 8, 10, 15, 20, 30, 40, 50, 60, 70, 80, 90, 100 or more contiguous nucleotides may be used for nucleic acid hybridization. Alternatively, in some aspects, a marker probe refers to a probe of any type that is able to distinguish (i.e., genotype) the particular allele that is present at a marker locus.

As used herein, the terms “molecular marker” or “genetic marker” may be used to refer to a genetic marker, as defined above, or an encoded product thereof (e.g., a protein) used as a point of reference when identifying a linked locus. A molecular marker can be derived from genomic nucleotide sequences or from expressed nucleotide sequences (e.g., from a spliced RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution, e.g., according to Watson-Crick base pairing rules. Some of the markers described herein are also referred to as hybridization markers when located on an indel region. This is because the insertion region is, by definition, a polymorphism vis-ã-vis a plant without the insertion. Thus, the marker need only indicate whether the indel region is present or absent. Any suitable marker detection technology may be used to identify such a hybridization marker, e.g., SNP technology is used in the examples provided herein.

A “non-naturally occurring variety of soybean” is any variety of soybean that does not naturally exist in nature. A “non-naturally occurring variety of soybean” may be produced by any method known in the art, including, but not limited to, transforming a soybean plant or germplasm, transfecting a soybean plant or germplasm and crossing a naturally occurring variety of soybean with a non-naturally occurring variety of soybean. In some embodiments, a “non-naturally occurring variety of soybean” may comprise one of more heterologous nucleotide sequences. In some embodiments, a “non-naturally occurring variety of soybean” may comprise one or more non-naturally occurring copies of a naturally occurring nucleotide sequence (i.e., extraneous copies of a gene that naturally occurs in soybean). In some embodiments, a “non-naturally occurring variety of soybean” may comprise a non-natural combination of two or more naturally occurring nucleotide sequences (i.e., two or more naturally occurring genes that do not naturally occur in the same soybean, for instance genes not found in Glycine max lines).

The term “transformation” and grammatical variations thereof are used in the present invention to refer to, for example, the transfer of nucleic acid fragments into the genome of a host organism, thus obtaining genetically stable heredity. The host organism comprising the transformed nucleic acid fragments is called a “transgenic” organism. The term “host cell” and grammatical variations thereof refer to a cell in which transformation of a recombinant DNA construct takes place and may include yeast cells, bacterial cells and/or plant cells.

As used herein, the term “transgenic” and grammatical variations thereof refer to a plant, including any part derived from the plant, such as a cell, tissue or organ, in which an exogenous nucleic acid (for example, a recombinant construct, vector or expression cassette comprising one or more nucleic acids) is integrated into the genome by a genetic engineering method, such as Agrobacterium transformation. Through gene technology, the exogenous nucleic acid is stably integrated into chromosomes, so that the next generation can also be transgenic. As used herein, “transgenic” and grammatical variations thereof also encompass biological treatments, which include plant hybridization and/or natural recombination.

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification. As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing. Primers can be prepared by any suitable method known in the art. Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066. Primers can be labeled, if desired, by incorporating detectable moieties by for instance spectroscopic, fluorescence, photochemical, biochemical, immunochemical, or chemical moieties. Primers diagnostic (i.e. able to identify or select based on presence of ASR resistant alleles) for ASR resistance can be created to any favorable SNP as described in Table 1. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell (2001). Molecular Cloning: A Laboratory Manual, Third Edition, Cold Spring Harbor Laboratory Press, Cold Spring Harbor, New York, United States of America. Generally, lower salt concentration and higher temperature hybridization and/or washes increase the stringency of hybridization conditions.

As used herein, the terms “phenotype,” “phenotypic trait” or “trait” refer to one or more traits and/or manifestations of an organism. The phenotype can be a manifestation that is observable to the naked eye, or by any other means of evaluation known in the art, e.g., microscopy, biochemical analysis, or an electromechanical assay. In some cases, a phenotype or trait is directly controlled by a single gene or genetic locus, i.e., a “single gene trait.” In other cases, a phenotype or trait is the result of several genes. It is noted that, as used herein, the term “disease resistant phenotype” takes into account environmental conditions that might affect the respective disease such that the effect is real and reproducible.

As used herein, the term “plant” and grammatical variations thereof may refer to a whole plant, any part thereof, or a cell or tissue culture derived from a plant. Thus, the term “plant” can refer to any of: whole plants, plant components or organs (e.g., roots, stems, leaves, buds, flowers, pods, etc.), plant tissues, seeds and/or plant cells. Plant cells are cells of plants, which are obtained directly from seeds or plants, or derived from cell cultures obtained from plants. Progenies, variants, and mutants of regenerated plants are within the scope of the present invention, provided that these parts comprise the introduced resistance genes. Thus, the term “soybean plant” may refer to a whole soybean plant, one or more parts of a soybean plant (e.g., roots, root tips, stems, leaves, buds, flowers, pods, seeds, cotyledons, etc.), soybean plant cells, soybean plant protoplasts and/or soybean plant calli.

As used herein, the term “plant part” includes but is not limited to embryos, pollen, seeds, leaves, flowers (including but not limited to anthers, ovules and the like), fruit, stems or branches, roots, root tips, cells including cells that are intact in plants and/or parts of plants, protoplasts, plant cell tissue cultures, plant calli, plant clumps, and the like. Thus, a plant part includes soybean tissue culture from which soybean plants can be regenerated. Further, as used herein, “plant cell” refers to a structural and physiological unit of the plant, which comprises a cell wall and also may refer to a protoplast. A plant cell of the present invention can be in the form of an isolated single cell or can be a cultured cell or can be a part of a higher-organized unit such as, for example, a plant tissue or a plant organ.

As used herein, the term “polymorphism” refers to a variation in the nucleotide sequence at a locus, where said variation is too common to be due merely to a spontaneous mutation. A polymorphism can be a single nucleotide polymorphism (SNP) or an insertion/deletion polymorphism, also referred to herein as an “indel.” Additionally, the variation can be in a transcriptional profile or a methylation pattern. The polymorphic site or sites of a nucleotide sequence can be determined by comparing the nucleotide sequences at one or more loci in two or more germplasm entries.

As used herein, the terms “closely linked” refers to linked markers displaying a cross over frequency with a given marker of about 10% or less (e.g. the given marker is within about 10 cM of a closely linked ASR marker). Put another way, closely linked loci co-segregate at least about 90% of the time. With regard to physical position on a chromosome, closely linked markers can be separated, for example, by about 1 megabase (Mb; 1 million nucleotides), about 500 kilobases (Kb; 1000 nucleotides), about 400 Kb, about 300 Kb, about 200 Kb, about 100 Kb, about 50 Kb, about 25 Kb, about 10 Kb, about 5 Kb, about 4 Kb, about 3 Kb, about 2 Kb, about 1 Kb, about 500 nucleotides, about 250 nucleotides, or less.

As used herein, the term “population” refers to a genetically heterogeneous collection of plants sharing a common genetic derivation.

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein, the term “reference sequence” refers to a defined nucleotide sequence used as a basis for nucleotide sequence comparison. The reference sequence for a marker, for example, is obtained by genotyping a number of lines at the locus or loci of interest, aligning the nucleotide sequences in a sequence alignment program, and then obtaining the consensus sequence of the alignment. Hence, a reference sequence identifies the polymorphisms in alleles at a locus. A reference sequence may not be a copy of an actual nucleic acid sequence from any particular organism; however, it is useful for designing primers and probes for actual polymorphisms in the locus or loci.

As used herein, the terms “disease tolerance” and “disease resistant” refer to a plant's ability to endure and/or thrive despite being infected with a respective disease. When used in reference to germplasm, the terms refer to the ability of a plant that arises from that germplasm to endure and/or thrive despite being infected with a respective disease. In some embodiments, infected Disease resistant soybean plants may yield as well (or nearly as well) as uninfected soybean plants. In general, a plant or germplasm is labeled as “Disease resistant” if it displays “enhanced pathogen resistance.”

An “unfavorable allele” of a marker is a marker allele that segregates with the unfavorable plant phenotype, therefore providing the benefit of identifying plants that can be removed from a breeding program or planting. For instance, one could eliminate from a plant breeding program plant lines carrying unfavorable alleles for ASR resistance.

### Plant Rust

The present invention pertains to proteins related to rust resistance in plants, nucleic acid sequence encoding such proteins, and uses thereof. The protein and the coding gene thereof and method disclosed in the invention can be used to protect plants from rust pathogens. In certain embodiments, the rust is leguminous plant rust.

In other embodiments, the leguminous plant rust is soybean rust. The pathogen of soybean rust may be Phakopsora pachyrhizi or Phakopsora meibomiae. As used below and in the claims each reference to soybean rust includes Asian soybean rust.

In a specific embodiment of the present invention, the pathogen of soybean rust is specifically the physiological race SS4 of Phakopsora pachyrhizi.

In preferred embodiments, the present invention pertains to proteins related to rust resistance in a leguminous plant.

The leguminous plant can be Glycine plants, Cicer plants, Cajanus plants, Lablab plants, Medicago plants, Phaseolus plants, Pisum plants, Pueraria plants, Trifolium plants or Vigna plants.

The Glycine plants can be Glycine arenaria, Glycine argyrea, Glycine cyrtoloba, Glycine canescens, Glycine clandestine, Glycine curvata, Glycine falcata, Glycine latifolia, Glycine microphylla, Glycine pescadrensis, Glycine stenophita, Glycine syndetica, Glycine soja Seib. et Zucc., Glycine max (L.) Merrill., Glycine tabacina or Glycine tomentella.

The Cicer plants can be Cicer arietinum, Cicer echinospermum, Cicer reticulatum or Cicer pinnatifidum.

The Lablab plants can be Lablab purpureus.

The Medicago plants can be Medicago truncatula or Medicago sativa.

The Phaseolus plants can be Phaseolus vulgaris, Phaseolus lunatus, Phaseolus acutifolius, or Phaseolus coccineus.

The Pisum plants can be Pisum abyssinicum, Pisum sativum, Pisum elatius, Pisum fulvum, Pisum transcaucasium, or Pisumhumile.

The Pueraria plants can be Pueraria lobata.

The Trifolium plants can be Trifolium aureum or Trifolium occidentale.

The Vigna plants can be Vigna unguiculata, Vigna dalzelliana, Vigna oblongifolia, Vigna parkeri, Vigna filicaulis, Vigna kirkii, Vigna luteola, Vigna radiata, Vigna trilobata, Vigna luteola, or Vigna mungo.

Furthermore, the leguminous plant can be any of the following: soybean, alfalfa, clover, pea, common bean, lentil, lupin, ghaf tree, carob bean, soybean, peanut, or tamarind.

In a specific embodiment of the present invention, the plant is specifically soybean variety Tianlong No. 1 ( [symbol] ).

The protein and the coding gene thereof and method disclosed in the invention can be used to protect plants from rust pathogens. The interaction between hosts and pathogens can be described as a continuum of “immunity” to “partial resistance” to “susceptibility”.

The method disclosed in the invention can increase, enhance, or improve the resistance of soybean to an obligatory biotrophic parasitic fungus Phakopsora pachyrhizi (the main pathogen of rust) or to Phakopsora meibomiae. For example, increased or enhanced resistance against rust pathogens can be compared with the impact of pathogens on susceptible plants. The manifestations of increased or enhanced resistance may be at different levels, but are related to the disease symptoms (such as the color of the disease spots) and the morbidity observed on plants or plant tissues (for example, leaves). The values of immunity, resistance and susceptibility can be given. For example, the value of resistance indicates the degree of resistance of plants to plant diseases (for example, rust). The values can also be used to compare the degree of resistance between, for example, plants of interest (e.g., transgenic leguminous plants) and susceptible plants (e.g., Tianlong No. 1 ( [symbol] ) or Williams) or reference standards.

The protein and the encoding gene thereof and the methods disclosed in the present invention relate to the isolation of a resistance gene from leguminous species and subsequent transfer of the resistance gene to a recipient plant, such as soybean, to provide or enhance resistance to Phakopsora pachyrhizi. One embodiment of the application includes transferring the resistance gene to sexually compatible or incompatible species to produce disease resistance. The resistance gene of the present invention can be used alone or in superposition with other resistance genes or together with non-resistance genes to provide or enhance the resistance of the recipient species against rust.

Therefore, the transgenic method disclosed in the present invention can be used alone or in combination with other strategies to produce or confer rust resistance in plants. Other available strategies include, but are not limited to, blocking the functional activity of effectors, inhibiting the uptake of pathogens or pathogen factors (e.g., fungi) into host cells (e.g., plant cells) and/or conventional resistance breeding.

The method disclosed in the present invention can provide or enhance the rust resistance of plants, so that the pathogen of rust cannot reproduce or the reproduction coefficient of the pathogen of rust is significantly reduced. Therefore, the method of the present invention can alleviate one or more symptoms (i.e. disease symptoms) of leguminous plant rust when compared with plants susceptible or tolerant to the genus Phakopsora. The plants referred to in the present invention also include transgenic leguminous plants (e.g., soybean) into which disease resistance genes or proteins are introduced by genetic engineering methods so as to enhance their resistance to diseases when exposed to leguminous plant rust.

The plants, plant parts or plant cells of the present invention are derived from plants that include, but are not limited to soybean, alfalfa, clover, pea, common bean, lentil, lupin, ghaf tree, carob, peanut, and tamarind.

The plants of the invention belong to Leguminosae. Examples of Leguminosae include, but are not limited to, Phaseolus (for example, French bean, string bean, Phaseolus vulgaris, Phaseolus lunatus, Phaseolus acutifolius, and Phaseolus coccineus); Glycine (for example, Glycine soja, and Glycine max (L.)); Pisum (for example, de-podded pea (sometimes referred to as smooth pea or round pea, Pisum sativum ( [symbol] )), Pisum sativum ( [symbol] ), Pisum sativum ( [symbol] ) which is also known as snow pea, edible-podded pea or Pisum granda); peanut (Arachis hypogaea), clover (Trifolium spp.), alfalfa (Medicago), kudzu (Pueraria lobata), common alfalfa, alfalfa (Medicago sativa), chickpea (Cicer), lentil (Lens culinaris), and lupin (Lupinus); vetch (Vicia), broad bean (Vicia faba), vetchling (Lathyrus) (for example, Lathyrus sativus, and Lathyrus tuberosus); Vigna (for example, Vigna aconitifolia, Vigna angularis, Vigna mungo, Vigna radiata, Vigna subterrane, Vigna umbellata, Vigna vexillata, and Vigna unguiculata (also known as long cowpea or cowpea)); Cajanus cajan, Macrotyloma (for example, Macrotyloma geocarpum, and Macrotyloma uniflorum); Psophocarpus tetragonolobus, Sphenostylis stenocarpa, Egyptian black beans, Lablab purpureus, Pachyrhizus erosus, and Cyamopsis tetragonolobus; and/or Canavalia (for example, Canavalia ensiformis, and Canavalia gladiata).

### Chromosomal Intervals

One embodiment of the invention is directed to chromosomal intervals derived from Glycine max strain SX6907. These chromosomal intervals when introduced into a plant (e.g. a soybean such a Glycine max strain Williams 82) are sufficient to confer increased rust resistance, such as e.g. Asian soybean rust (ASR) resistance, as compared to a control plant not comprising said chromosomal interval.

SEQ ID NOs 11, 12, or 13 are chromosomal interval derived from Glycine max strain SX6907. Genetic mapping studies indicate that Glycine max strain SX6907 contains chromosomal intervals associated with ASR resistance (e.g. corresponding to S SEQ ID NOs 11, 12, or 13). These chromosomal intervals or portions thereof may be introduced (i.e. introgressed through use of marker assisted breeding (MAB), or through use of GM or GE introduction) into Glycine max lines to create Glycine max lines resistant to various diseases such as ASR. For example, these chromosomal intervals may be introduced into Glycine max line Williams 82.

Table 1 indicates single nucleotide polymorphisms (SNP) within SEQ ID NOs 11, 12, or 13 that are associated with ASR resistance.

TABLE 1
SNP Positions within SEQ ID NOs 11, 12, or 13 that are associated with
increased resistance to ASR
Table 1: SNP Positions

| Chromosome | Position | Reference allele | Rpp6907 allele |
|---|---|---|---|
| Gm18 | 56601565 | A | AC |
| Gm18 | 56601634 | T | C |
| Gm18 | 56602295 | G | T |
| Gm18 | 56602866 | G | GA |
| Gm18 | 56602929 | T | A |
| Gm18 | 56604482 | G | GATATATAT |
| Gm18 | 56604846 | C | T |
| Gm18 | 56605172 | GTATATTTATATATATA | G |
| | | TATATATA | |
| | | (SEQ ID NO: 14) | |
| Gm18 | 56605243 | G | T |
| Gm18 | 56605859 | A | AATATGCTTT |
| | | | (SEQ ID NO: 15) |
| Gm18 | 56605869 | ATATG | A |
| Gm18 | 56605893 | A | G |
| Gm18 | 56605895 | A | G |
| Gm18 | 56605897 | A | G |
| Gm18 | 56605899 | A | G |
| Gm18 | 56605901 | A | G |
| Gm18 | 56606593 | TA | T |
| Gm18 | 56606764 | C | T |
| Gm18 | 56607175 | T | C |
| Gm18 | 56608059 | T | C |
| Gm18 | 56609308 | C | T |
| Gm18 | 56609424 | A | G |
| Gm18 | 56609508 | A | T |
| Gm18 | 56609511 | C | G |
| Gm18 | 56609554 | T | G |
| Gm18 | 56610268 | T | C |
| Gm18 | 56610625 | T | TAAG |
| Gm18 | 56611386 | T | A |
| Gm18 | 56612772 | C | CTATATA |
| Gm18 | 56613273 | C | T |
| Gm18 | 56613352 | A | G |
| Gm18 | 56613400 | C | T |
| Gm18 | 56613421 | C | T |
| Gm18 | 56613787 | TA | T |
| Gm18 | 56614286 | T | A |
| Gm18 | 56614344 | G | GA |
| Gm18 | 56614630 | A | G |
| Gm18 | 56614906 | G | GT |
| Gm18 | 56615544 | CT | C |
| Gm18 | 56615626 | A | ATG |
| Gm18 | 56615677 | T | A |
| Gm18 | 56616069 | C | A |
| Gm18 | 56616107 | T | A |
| Gm18 | 56616637 | T | C |
| Gm18 | 56616692 | A | T |
| Gm18 | 56616778 | T | A |
| Gm18 | 56616824 | C | T |
| Gm18 | 56616825 | T | G |
| Gm18 | 56616833 | A | G |
| Gm18 | 56616839 | C | T |
| Gm18 | 56616844 | T | C |
| Gm18 | 56616894 | A | G |
| Gm18 | 56616903 | TGGGTCAACCTGAATG | T |
| | | AGTCAGGGTTGACCCA | |
| | | AA(SEQ ID NO: 16) | |
| Gm18 | 56616938 | CGA | C |
| Gm18 | 56616943 | AT | A |
| Gm18 | 56616965 | ATT | A |
| Gm18 | 56616973 | TAAAAA | T |
| Gm18 | 56616996 | T | TAA |
| Gm18 | 56616997 | T | A |
| Gm18 | 56617058 | A | G |
| Gm18 | 56617072 | AC | A |
| Gm18 | 56617119 | C | A |
| Gm18 | 56617143 | CTTTCTTTAATT (SEQ ID | C |
| | | NO: 17) | |
| Gm18 | 56617792 | A | C |
| Gm18 | 56618125 | C | G |
| Gm18 | 56618647 | TA | T |
| Gm18 | 56621101 | T | C |
| Gm18 | 56621162 | C | A |
| Gm18 | 56621217 | A | C |
| Gm18 | 56621385 | G | A |
| Gm18 | 56621559 | C | G |
| Gm18 | 56621731 | G | A |
| Gm18 | 56621822 | A | G |
| Gm18 | 56621836 | T | C |
| Gm18 | 56621857 | C | T |
| Gm18 | 56621899 | T | C |
| Gm18 | 56621936 | T | A |
| Gm18 | 56621937 | G | GAA |
| Gm18 | 56621943 | G | A |
| Gm18 | 56622058 | A | AT |
| Gm18 | 56622285 | T | C |
| Gm18 | 56622585 | A | ATGG |
| Gm18 | 56622602 | A | T |
| Gm18 | 56622637 | A | G |
| Gm18 | 56622642 | T | G |
| Gm18 | 56622853 | T | A |
| Gm18 | 56622862 | A | G |
| Gm18 | 56622906 | G | T |
| Gm18 | 56622925 | C | T |
| Gm18 | 56623192 | TC | T |
| Gm18 | 56623667 | A | G |
| Gm18 | 56623903 | C | T |
| Gm18 | 56624060 | C | T |
| Gm18 | 56624065 | T | G |
| Gm18 | 56624185 | AT | A |
| Gm18 | 56624351 | AC | A |
| Gm18 | 56624404 | A | AAAGT |
| Gm18 | 56624427 | T | C |
| Gm18 | 56624464 | A | G |
| Gm18 | 56624551 | G | T |
| Gm18 | 56624606 | C | A |
| Gm18 | 56624609 | TTAA | T |
| Gm18 | 56624664 | G | A |
| Gm18 | 56624955 | C | T |
| Gm18 | 56625057 | C | A |
| Gm18 | 56625513 | A | G |
| Gm18 | 56625553 | A | AATAT |
| Gm18 | 56625571 | A | C |
| Gm18 | 56625591 | C | G |
| Gm18 | 56625648 | C | T |
| Gm18 | 56625668 | AGAT | A |
| Gm18 | 56625720 | C | T |
| Gm18 | 56625722 | T | A |
| Gm18 | 56625799 | A | G |
| Gm18 | 56625966 | A | G |
| Gm18 | 56626008 | G | A |
| Gm18 | 56626041 | G | T |
| Gm18 | 56626052 | C | T |
| Gm18 | 56626063 | T | G |
| Gm18 | 56626080 | T | G |
| Gm18 | 56626449 | T | G |
| Gm18 | 56626566 | G | A |
| Gm18 | 56626575 | A | G |
| Gm18 | 56626903 | T | C |
| Gm18 | 56626915 | C | G |
| Gm18 | 56626974 | ACATAC | A |
| Gm18 | 56627161 | A | AATAT |
| Gm18 | 56627238 | C | T |
| Gm18 | 56627264 | A | C |
| Gm18 | 56627287 | GT | G |
| Gm18 | 56627317 | A | G |
| Gm18 | 56627338 | G | A |
| Gm18 | 56627360 | T | TA |
| Gm18 | 56627382 | C | A |
| Gm18 | 56627386 | C | T |
| Gm18 | 56627412 | C | G |
| Gm18 | 56627575 | A | T |
| Gm18 | 56627577 | T | C |
| Gm18 | 56627857 | T | G |
| Gm18 | 56627967 | A | G |
| Gm18 | 56628395 | A | G |
| Gm18 | 56628450 | C | G |
| Gm18 | 56629061 | CTG | C |
| Gm18 | 56629276 | G | A |
| Gm18 | 56629622 | T | C |
| Gm18 | 56629688 | C | T |
| Gm18 | 56629764 | T | A |
| Gm18 | 56630232 | T | A |
| Gm18 | 56630293 | AT | A |
| Gm18 | 56630337 | C | T |
| Gm18 | 56630348 | C | T |
| Gm18 | 56630383 | C | G |
| Gm18 | 56630411 | T | C |
| Gm18 | 56630490 | G | A |
| Gm18 | 56630497 | A | ATTCAAAAATATTTTTTT |
| | | | AATAATT (SEQ ID NO: 18) |
| Gm18 | 56630555 | C | T |
| Gm18 | 56630572 | A | G |
| Gm18 | 56630585 | A | C |
| Gm18 | 56630748 | T | C |
| Gm18 | 56630769 | C | T |
| Gm18 | 56630804 | T | A |
| Gm18 | 56630811 | A | G |
| Gm18 | 56630892 | T | C |
| Gm18 | 56630893 | G | A |
| Gm18 | 56630923 | C | T |
| Gm18 | 56630934 | A | T |
| Gm18 | 56630961 | T | C |
| Gm18 | 56630985 | C | T |
| Gm18 | 56631041 | T | C |
| Gm18 | 56631056 | T | C |
| Gm18 | 56631116 | T | C |
| Gm18 | 56631151 | A | T |
| Gm18 | 56631166 | C | T |
| Gm18 | 56631200 | A | T |
| Gm18 | 56631266 | C | CAT |
| Gm18 | 56631376 | A | G |
| Gm18 | 56631609 | T | G |
| Gm18 | 56631641 | G | T |
| Gm18 | 56631665 | A | T |
| Gm18 | 56632093 | C | T |
| Gm18 | 56632157 | G | A |
| Gm18 | 56632282 | C | A |
| Gm18 | 56632296 | G | C |
| Gm18 | 56633543 | C | T |
| Gm18 | 56634232 | G | A |
| Gm18 | 56635842 | A | T |
| Gm18 | 56635932 | A | G |
| Gm18 | 56636250 | C | CA |
| Gm18 | 56636303 | T | C |
| Gm18 | 56637522 | G | C |
| Gm18 | 56637889 | A | AT |
| Gm18 | 56639048 | T | C |
| Gm18 | 56639540 | G | C |
| Gm18 | 56640035 | A | C |
| Gm18 | 56640169 | C | T |
| Gm18 | 56640187 | A | T |
| Gm18 | 56640293 | C | G |
| Gm18 | 56640394 | AGGGG | A |
| Gm18 | 56640465 | T | TA |
| Gm18 | 56640696 | CAAA | C |
| Gm18 | 56640962 | A | G |
| Gm18 | 56641591 | C | A |
| Gm18 | 56641739 | G | A |
| Gm18 | 56641791 | T | C |
| Gm18 | 56641865 | G | T |
| Gm18 | 56644131 | CAA | C |
| Gm18 | 56644396 | G | A |
| Gm18 | 56645035 | A | C |
| Gm18 | 56645098 | A | ATAC |
| Gm18 | 56645223 | ATTAAATTTAAATTGAT | A |
| | | TGTTAAT | |
| | | (SEQ ID NO: 19) | |
| Gm18 | 56645355 | T | C |
| Gm18 | 56645442 | G | T |
| Gm18 | 56645465 | A | C |
| Gm18 | 56645519 | T | G |
| Gm18 | 56645639 | G | A |
| Gm18 | 56645668 | C | G |
| Gm18 | 56645752 | T | G |
| Gm18 | 56645780 | T | A |
| Gm18 | 56645880 | G | T |
| Gm18 | 56645901 | TTA | T |
| Gm18 | 56645929 | G | T |
| Gm18 | 56645937 | C | CAA |
| Gm18 | 56645953 | A | T |
| Gm18 | 56645958 | TAAA | T |
| Gm18 | 56645984 | T | C |
| Gm18 | 56646008 | G | A |
| Gm18 | 56646045 | C | A |
| Gm18 | 56646087 | A | AT |
| Gm18 | 56646107 | T | C |
| Gm18 | 56646143 | G | A |
| Gm18 | 56646148 | A | AT |
| Gm18 | 56646159 | C | G |
| Gm18 | 56646193 | G | A |
| Gm18 | 56646220 | T | TTA |
| Gm18 | 56646239 | C | CTATTATACACCGA |
| | | | (SEQ ID NO: 20) |
| Gm18 | 56646257 | G | T |
| Gm18 | 56646309 | T | C |
| Gm18 | 56646347 | C | T |
| Gm18 | 56646350 | T | TGA |
| Gm18 | 56646354 | AT | A |
| Gm18 | 56646357 | CA | C |
| Gm18 | 56646360 | T | G |
| Gm18 | 56646361 | A | G |
| Gm18 | 56646362 | T | TG |
| Gm18 | 56646364 | T | A |
| Gm18 | 56646369 | T | C |
| Gm18 | 56646371 | T | C |
| Gm18 | 56646372 | AAGTG | A |
| Gm18 | 56646382 | G | GT |
| Gm18 | 56646383 | AGG | A |
| Gm18 | 56646387 | G | T |
| Gm18 | 56646389 | C | T |
| Gm18 | 56646390 | A | T |
| Gm18 | 56646392 | A | T |
| Gm18 | 56646394 | ACAC | A |
| Gm18 | 56646398 | C | G |
| Gm18 | 56646400 | A | G |
| Gm18 | 56646401 | C | A |
| Gm18 | 56646415 | C | T |
| Gm18 | 56646471 | A | G |
| Gm18 | 56646489 | G | A |
| Gm18 | 56646513 | C | T |
| Gm18 | 56646524 | C | T |
| Gm18 | 56646536 | T | C |
| Gm18 | 56646578 | T | C |
| Gm18 | 56646638 | G | A |
| Gm18 | 56646658 | C | T |
| Gm18 | 56646659 | G | T |
| Gm18 | 56646720 | TCTAAA | T |
| Gm18 | 56646727 | CATTAAGGCCT | C |
| | | (SEQ ID NO: 21) | |
| Gm18 | 56646741 | AGATG | A |
| Gm18 | 56646799 | T | A |
| Gm18 | 56646810 | G | A |
| Gm18 | 56646825 | ACG | A |
| Gm18 | 56646842 | G | T |
| Gm18 | 56646855 | G | T |
| Gm18 | 56646871 | TAAGTTC | T |
| Gm18 | 56646931 | C | T |
| Gm18 | 56646932 | G | A |
| Gm18 | 56647024 | T | C |
| Gm18 | 56647026 | A | G |
| Gm18 | 56647039 | C | T |
| Gm18 | 56647040 | C | T |
| Gm18 | 56647041 | T | C |
| Gm18 | 56647053 | C | T |
| Gm18 | 56647057 | T | G |
| Gm18 | 56647059 | G | A |
| Gm18 | 56647065 | T | G |
| Gm18 | 56647068 | C | T |
| Gm18 | 56647069 | T | C |
| Gm18 | 56647104 | C | T |
| Gm18 | 56647112 | C | T |
| Gm18 | 56647127 | A | G |
| Gm18 | 56647145 | T | TATTA |
| Gm18 | 56647174 | A | AG |
| Gm18 | 56647186 | G | A |
| Gm18 | 56647187 | A | G |
| Gm18 | 56647196 | C | A |
| Gm18 | 56647200 | A | G |
| Gm18 | 56647209 | C | G |
| Gm18 | 56647211 | A | G |
| Gm18 | 56647214 | A | G |
| Gm18 | 56647221 | G | A |
| Gm18 | 56647239 | c | T |
| Gm18 | 56647248 | A | G |
| Gm18 | 56647294 | G | A |
| Gm18 | 56647477 | T | C |
| Gm18 | 56647482 | C | T |
| Gm18 | 56647487 | T | G |
| Gm18 | 56647498 | G | A |
| Gm18 | 56647512 | T | A |
| Gm18 | 56647543 | TTAAGTATATG | T |
| | | (SEQ ID NO: 22) | |
| Gm18 | 56647567 | G | A |
| Gm18 | 56647605 | A | G |
| Gm18 | 56647659 | G | T |
| Gm18 | 56647792 | C | T |
| Gm18 | 56647797 | A | T |
| Gm18 | 56647842 | G | A |
| Gm18 | 56647893 | T | C |
| Gm18 | 56647916 | A | G |
| Gm18 | 56647955 | C | T |
| Gm18 | 56647957 | T | G |
| Gm18 | 56648064 | T | G |
| Gm18 | 56648082 | T | A |
| Gm18 | 56648111 | A | G |
| Gm18 | 56648129 | T | C |
| Gm18 | 56648240 | C | A |
| Gm18 | 56648316 | C | T |
| Gm18 | 56648404 | T | C |
| Gm18 | 56648436 | T | C |
| Gm18 | 56648528 | G | GT |
| Gm18 | 56648611 | A | AG |
| Gm18 | 56648613 | T | A |
| Gm18 | 56648624 | C | T |
| Gm18 | 56648718 | G | GC |
| Gm18 | 56648732 | TA | T |
| Gm18 | 56648734 | A | T |
| Gm18 | 56648765 | A | ATACATAC |
| Gm18 | 56648914 | A | T |
| Gm18 | 56649033 | C | T |
| Gm18 | 56649889 | G | C |
| Gm18 | 56650186 | G | GT |
| Gm18 | 56650603 | C | G |
| Gm18 | 56650733 | C | A |
| Gm18 | 56651552 | C | T |
| Gm18 | 56651585 | T | C |
| Gm18 | 56651696 | C | A |
| Gm18 | 56651711 | T | C |
| Gm18 | 56651853 | A | AT |
| Gm18 | 56651998 | G | GA |
| Gm18 | 56652522 | T | C |
| Gm18 | 56652542 | C | CCTAA |
| Gm18 | 56652838 | AT | A |
| Gm18 | 56653055 | C | T |
| Gm18 | 56653492 | G | A |
| Gm18 | 56653844 | G | T |
| Gm18 | 56653850 | TGGGGCG | T |
| Gm18 | 56653858 | G | T |
| Gm18 | 56653860 | G | T |
| Gm18 | 56653874 | TGGG | T |
| Gm18 | 56654008 | A | C |
| Gm18 | 56654425 | A | ATATGATAG |
| Gm18 | 56654689 | G | C |
| Gm18 | 56656609 | ATTTCTTCTTTAATTGTT | A |
| | | TTTTTTTTTTTTTTGCTC | |
| | | AGCAAAATTAAATATA | |
| | | TTATAGATGAGTACCA | |
| | | GAGGTACTAAAATATA | |
| | | CAGATTTAGAGCCATAT | |
| | | TACAAGTAGTTTTGGAC | |
| | | AGACAATGATACAGTA | |
| | | GCTGAAATATCCCAAA | |
| | | AACTACTCAAATAAGA | |
| | | CTTGGAGCTATACTCTA | |
| | | AACCTATGATGCTGTCC | |
| | | TAAGAAAAGCATCTTT | |
| | | GAGATTTGAAGACCAT | |
| | | TGATTGAAATGTAGAG | |
| | | AGAAATCTTTTTCAAAA | |
| | | CATCTGAGCCATGTCCA | |
| | | TAAAATAAACACTGCA | |
| | | TCATCCATTAATTTATG | |
| | | AGTGTCAAAATTAGCA | |
| | | TTGGAGAAAATGATGC | |
| | | TGTTCCTATGCTTCCAA | |
| | | ATCGAGTAAGTGAGAG | |
| | | CGAACCACCACCACTG | |
| | | CCATCTTTTTTCCCTGC | |
| | | AACCCTACGGAAGCTC | |
| | | CAAAGATATGCTGACT | |
| | | AAAATGCTGATCCGGC | |
| | | TGATAAGGGAAAACAC | |
| | | CCATCATATTTACCCAA | |
| | | GACTGTGACTCCCACCA | |
| | | TAGAGGACTAACTTTAC | |
| | | TGCAGTGGAAAAATAA | |
| | | GTGACTTGCAGTCTCTT | |
| | | CCACAGATCTGCATAA | |
| | | GGGGCACAAGTACTCT | |
| | | TGTAACTCAACCCGCTT | |
| | | CTTTCTCAAATTGGCTT | |
| | | TAGTTGGCAGTTTGTCT | |
| | | TGAATCAACCTCCACGC | |
| | | GAAGATAGCCACTTTC | |
| | | AATGGGACCCTAAGCT | |
| | | TCCATAATTCCTTGAAC | |
| | | TTTCCATCCTGCTCTTC | |
| | | TCCCACTGTGACATGAT | |
| | | GTATTGCCTTATATGCA | |
| | | CTCTTAGTCGAATAACA | |
| | | GCCACTAGGCTCTGCTG | |
| | | CCCACTTCCATTGGTCA | |
| | | CTTAATTCCGGCCTGAT | |
| | | TGTGAACCCTTCCAGCT | |
| | | GTTGGAGGAAAGCTAC | |
| | | CGCCATATCTATCTCGC | |
| | | TATCAAACAAAGGTCT | |
| | | CCTCCACTTAAGACTCC | |
| | | ATTCCCACCCACCTTCT | |
| | | TTCGCAGCACCTATTTG | |
| | | ATGAATGAAATGATGT | |
| | | TTTTGAGCTGATATGGT | |
| | | ATACAATCTAGGATATT | |
| | | TATCAGCTAAGCAGTTG | |
| | | TCTCCGCCTATCCACCT | |
| | | ATCCTCCCAAAACTTAA | |
| | | ACTTGTCCCCACATCCG | |
| | | ACCCTCCACAATATCAA | |
| | | CCTATTCAGCTGTTGAC | |
| | | CTTGATTCATGCTTTGA | |
| | | TTTACTATCTTTAGATC | |
| | | CCTCCACCATGATGATT | |
| | | CAGTACTAGCCCTCGA | |
| | | AGCTCCATCAAGACTCC | |
| | | TCCATCCTCCATACTTT | |
| | | GATTCAAGGACTCTAG | |
| | | CCCAAGTCTCCTCCTGA | |
| | | TTCTGAAACATCTCCCA | |
| | | TTTCCATTTTCCTAATA | |
| | | ATGATGTGTTGAACGA | |
| | | GATGATATCCTTGACTC | |
| | | CCAACCCACCTTGTTCC | |
| | | TTTGGCAAACACACCTT | |
| | | TTCCCACTTGATCCAAG | |
| | | GGATCTTATTTTGATCA | |
| | | GCTGCACCACCCCATA | |
| | | AGAAGTTCCTTTGTAAT | |
| | | CTGGTTATCTTGCTAAT | |
| | | CACCTGCCTAGGGACC | |
| | | CTGAAAAAAGATAGAA | |
| | | AATAAATTGGAATAGA | |
| | | TGTTAGCACTGAATTTA | |
| | | TGAGAATCACTCTCCCC | |
| | | CCAAAAGACAAGTGTC | |
| | | TCTGTTTCCATCCTGCC | |
| | | AGTTTTCTCTCACACTT | |
| | | ATTGATTAAAGGTTCCC | |
| | | ACGTCTGACATCTTCTA | |
| | | GGATTAGCACCAATAG | |
| | | GTATGCCAAGATATGT | |
| | | GAAAGGAAAAGACATC | |
| | | AATCCACAGTTGAGAT | |
| | | AACTGGATGCACCATA | |
| | | AGTCCACTGCTCAGGC | |
| | | ACCCCAAAGGCCCCAC | |
| | | AACTACTCTTGGCGAA | |
| | | ATTTATCTTTAACCCCG | |
| | | ATGACATCTCAAAAGC | |
| | | ACGAAGTATTGCATTG | |
| | | ATTGTTCTTACATTCGC | |
| | | TAATGTTGCCTCTCCCA | |
| | | AAAATAGTGTCATCTGC | |
| | | ATACTGAAGCAGGCTA | |
| | | ATCTCCACCTTCTTTGA | |
| | | GCCCACTAAAAATCCCT | |
| | | TATAGAATCCTCCCTCA | |
| | | ATTGCTTTGGTCATTAG | |
| | | ACCGCTCAAACCTTCTG | |
| | | CAACAATATTAAACAA | |
| | | AAGGGGTGATAATGGA | |
| | | TCCCCCTGTCTAAGACC | |
| | | TTTCTGTGGATAGAACT | |
| | | CCTTCGTAGGGCTTCCA | |
| | | TTTACCAAGACTGAGAT | |
| | | GGAAGCTGATTTTAAA | |
| | | CATCTCTGTATCCACTG | |
| | | AATCCACTTCGGTCCAA | |
| | | AACCCAATCTCCTCATC | |
| | | ATGTATATCAAAAAAT | |
| | | CCCAAGACACTAAGTC | |
| | | ATACGCCTTCTCATAGT | |
| | | CCACCTTGAAAACCAT | |
| | | GCATGGTTTTTGACATC | |
| | | TTTTGGCCTCTTCAACT | |
| | | ACCTCATTTGCAATCAC | |
| | | CACACTATGAAGCATG | |
| | | TGCCTACCTTGTATGAA | |
| | | TGCTGACTGACGATCAC | |
| | | TAATTATGAGGGGCAT | |
| | | AACTCTCTTCAGTCTAT | |
| | | TAGGCAACACTTTAGC | |
| | | GAGGATTTTATAGGTAC | |
| | | ATCCGATGAGTGAAAT | |
| | | AGGTCTATATTCATTCA | |
| | | ACCCTTGAGGGTCCGCC | |
| | | ACCTTCGGGATTAAAG | |
| | | CTATGAATGATGCGTTC | |
| | | AAGCCCCTCGGGAAGA | |
| | | CTCCGTTGACATAGAAC | |
| | | TCGTCAAAGAATCTAA | |
| | | GAAAATCTGGTTTGATG | |
| | | ACCTCCCAAAATTGCTT | |
| | | GATGAATTTGAAATTG | |
| | | AACCCGTCTGGGCCCG | |
| | | GACTTTTATCACTTCCA | |
| | | CAACTCCAAACAGCCC | |
| | | TCCTTATCTCCTCTTCCT | |
| | | GAAAGTGCTCGACTAA | |
| | | CATGGCATTCTGATGGG | |
| | | AATCAATGGTATTGAA | |
| | | GCTGATCCCATTCAGAG | |
| | | TTGGCCTATCAAAATCT | |
| | | GTTTCCTGGAACCTCTG | |
| | | TGAAAAAAATCTCCTA | |
| | | ACTTCCTCTTTGACTTC | |
| | | AGCCGGCTCCTCCTTCC | |
| | | ACATCCCATCAACCATC | |
| | | AGTCCATTTAGGCTATT | |
| | | GGATCGCCTCCTGGAAT | |
| | | TTATCAGCAAATGTTAA | |
| | | TACCTCGAATTACAATC | |
| | | ACCCTCCTTAATCCACC | |
| | | TGGATCTTGCCTTCTGA | |
| | | CGTAACAGCGACTCAT | |
| | | GAGTTTGGGCTGCCTCC | |
| | | CAAACCTTCTGCTGCAA | |
| | | CTTCTTTTTGAGTATCC | |
| | | GCTCATTATCATCCAGT | |
| | | GGTCTGTCTGCTGATTC | |
| | | CTCTTCTAGTTTGTTCA | |
| | | ACTCTTCCTCTATCCTC | |
| | | TTGAAACGGCTTAAAG | |
| | | TGTCTCCAAAGTGATCT | |
| | | TTATTCCACGCCTTTAT | |
| | | CTTCTGTTTGAGGGCCT | |
| | | TGAATTTGTTTTTTAAA | |
| | | ACATGACCCCCCCATCC | |
| | | ACTCTGAGTATTGGATG | |
| | | ACCAGGTTTCGGACAC | |
| | | CAACTTCTTAAAAGATA | |
| | | CATCAGATAACCAACA | |
| | | ATCCAATAGTCTGAAA | |
| | | GGTTTTGGGCCCCAATC | |
| | | AATGGTTTTAGATCTAA | |
| | | GCAATATGGGACAATG | |
| | | GTCCGAAAAATTCCTG | |
| | | GCTAGCGGTGTTTGGAC | |
| | | CGATCCGGGCCATTTGG | |
| | | AAAGCCACTCTGGGGA | |
| | | AACGAAGGCTCTATCC | |
| | | AACTTGCTTTTAGCTGT | |
| | | ACCGTTCGGTCTAAACC | |
| | | ATGTAAATCTCTTTCCC | |
| | | ACCCAAGGCGCTTCTTC | |
| | | TAATTCCAACTCCTCAA | |
| | | TCCAGCTATTAAAATCC | |
| | | CGTATGCTTCCATCCAC | |
| | | CATCCCCCTCTGCGAAG | |
| | | ACCCCACTCTTTCTCCA | |
| | | CTTATCCTGATGTTGTT | |
| | | AAAGTCCCCTATAATAC | |
| | | ACCAATAACCATTTGG | |
| | | ATTCTGGATTTTCAGCT | |
| | | GAGTGACTTTATCCCAT | |
| | | AACACCCTTTTGTCCTG | |
| | | CAGGTTACATGGGGAG | |
| | | TATATATTTACAACATG | |
| | | CACTGCTATGGATTCTT | |
| | | GACCCCACTTTCCTGAC | |
| | | AGTAGTATGAAGCCCG | |
| | | ATCCAGCTGATTTGCTC | |
| | | TCCACCTTCAGAGACTT | |
| | | TTGATTCCAAATACAGA | |
| | | GAATACCACCAGCTGT | |
| | | ATTTACGGCTGGAACCT | |
| | | CCTCCCAGCAGAAATC | |
| | | AGAATTCCCCCATAGA | |
| | | GCCCTGCACATACTCTT | |
| | | ATTGACCGTTTCCTTCT | |
| | | TTGTTTCTTGTAAGCAT | |
| | | AAAAGGTCAATATGAT | |
| | | ACTCTTTGACCATACGT | |
| | | CTAATTGCTGGCCATTT | |
| | | AACCCCCCTCCCCAACC | |
| | | CCCTTACATTATAAGAG | |
| | | ATGATATTCATGAGTCC | |
| | | CTTCCCCTTCCTCCCAA | |
| | | GCTATCTGCCTCTTTTC | |
| | | CATCTCTATCTTCCATT | |
| | | TCTGTCAACTGTTGAAC | |
| | | ATAATCTCTTTGAGTTT | |
| | | TTCCTGTTGTCAGTCCC | |
| | | AACTCCTTGATCATTCT | |
| | | CCATAGACTTTCTTCGT | |
| | | GCGAGTCAAAAGTTTC | |
| | | CTCCTGGAGCTGATTTT | |
| | | CCTGCCTATTGTGAGTT | |
| | | GATGATGTTTCTGTTTG | |
| | | TATTTGGGGGTTGGCTG | |
| | | ATCCACGTCCCTCGTTA | |
| | | TCTCGCTGGAATAGTCC | |
| | | ATTTTGAACATGCTCTG | |
| | | GGCTTGAAAGCACTTG | |
| | | GTTCGAAACAACCTCA | |
| | | CCTATTTGGGCTTTAAC | |
| | | TCTCTTCATTTTCCCTTT | |
| | | ACGGGAATACGTTAAC | |
| | | AAAGGGGTTAGGAGAG | |
| | | GTGTTGTTTTTAAAACT | |
| | | GGGCCATTTTTGGGAG | |
| | | AAGGACATAATTTAGG | |
| | | TGGGGTGTTAATAGCTT | |
| | | GGCCCAAATCCAGCAG | |
| | | CCCCATCATCTCCTTGT | |
| | | CACGTGACTTGGGTGA | |
| | | GGGGGATTCAAAATTA | |
| | | TGTTTGTCTTCCTCGTA | |
| | | CACCATGCCTCCAACGT | |
| | | TTCTTGTCTGCCTCCTC | |
| | | ATACTATTTTCATACAA | |
| | | ATTGCCCAAACCGTCA | |
| | | GGGCCGACATCCCCCC | |
| | | CGAGGTTATCTTCTTCC | |
| | | CCTAAATCGGTCTTCCC | |
| | | TTCTTCCTGCGACACTG | |
| | | GTAGCTCACGGTCCCTG | |
| | | GTCTGCACGCATTTATC | |
| | | ACCTGTCAGTGCATTAT | |
| | | CTGCTTGCTTCACGACC | |
| | | CATCGGACAATAGCTTT | |
| | | GTCCTTTGGGTCATGTC | |
| | | CGCTCTTCGTCCCGATC | |
| | | GCCTTTCCGTTTTGGTC | |
| | | CGCACCGGCCGCGACT | |
| | | GTCGCCGGTCCTTCTGA | |
| | | CCGAGAGACCTGCCTG | |
| | | AGTCGCCCTTCTTCCAC | |
| | | ACCACGCGGCGACCTC | |
| | | TCCTCGTCGCCACCCCT | |
| | | ATCCTCTGTGTCATCCA | |
| | | CCGTCTCCGACTGGGCT | |
| | | GGAGCTCCCCTTTGGGT | |
| | | TTTAAATGGCACGATTT | |
| | | CACTTCTCCCTCTGCGG | |
| | | TTATCTTCCTCGCCCCC | |
| | | CACCACTCCCTCCGTCG | |
| | | CCATATCCTCCATGGCT | |
| | | TCTGCCTCCAAGGTCCC | |
| | | TCCCTTCGCTACTGTTC | |
| | | GTATTTCCTCCGCCACG | |
| | | CTCTCTATTCGCGCTTT | |
| | | GTACCGCCACCCCTGCA | |
| | | GCTCACTACCAGACCA | |
| | | CGCGTCATCGTTACATT | |
| | | CGTCAGAAAGGATTTC | |
| | | CTCTGATGAGCTGAAGT | |
| | | CACGTTCCCTTTTTGGC | |
| | | TGCCACCTAGCTCCAAA | |
| | | CAACCCACTCTCCTCCG | |
| | | CTATGTGCACCGGAAA | |
| | | AATCTCCCCCTGTATAT | |
| | | GAATATTTACCGTGTGC | |
| | | TGAATCAGCGGTTTCCA | |
| | | TGGCGTTTTGACCAGTA | |
| | | TCCTCGCGACGTCGAAC | |
| | | CTCCTTTGATCTTCTAA | |
| | | GCTCTCCTCAGCCTCCA | |
| | | CCATCTCACCTATTCCC | |
| | | GCGACTATCTGCTTGAT | |
| | | ACAGTTCATGTCCCATG | |
| | | CAACCAAAGGTATTCC | |
| | | CCAACATTGGATCCAA | |
| | | GTTAGTCTAAAACCAG | |
| | | GGCGTAGACTTGGATTC | |
| | | CACTTCTCTATGGAGTA | |
| | | GAACAGGTCGCCCCAT | |
| | | CCTTCATCTTCTTCATT | |
| | | CACCATTTTCTCTGCGT | |
| | | TCTCTTCTGTGAGCCCC | |
| | | AGTAACAAAACCATGT | |
| | | CATCACCGATGTACTTT | |
| | | GGTGATATGTTTTGTCC | |
| | | GCTATCCCACCATATGG | |
| | | CTTCTTCTATGCTATCA | |
| | | AAGCTAGCCAAGTTTTT | |
| | | CAGTCTACCCACCCAG | |
| | | GCCTCCTTGAGCCATTG | |
| | | TTTACCCGTCATCGAAA | |
| | | TGTCCAGGTTCACTTCC | |
| | | GATGTGGCATTGGAAC | |
| | | TTATGTATTGGAGTTTG | |
| | | GCTGTTGTTCTCCTCTG | |
| | | TGCTGGTTTCGGGATAT | |
| | | TCGTGTTGACCACCTCC | |
| | | GCATACGACCGTCCTTG | |
| | | GATTCCCATATTCGCTT | |
| | | CTGGTAACCTGCTCAGG | |
| | | TACACTTCTGTTCTTCC | |
| | | CTTTTGTGTTTGTCCTG | |
| | | TCTCTGCTGTCCCCCTC | |
| | | TCGACTGTTTGTGATTC | |
| | | TCCCTTCTTTGGTCTCC | |
| | | CATATTTAGGGATATTA | |
| | | ACATTCATCTTCATTCC | |
| | | TCCGAAGAACCTATTGT | |
| | | CCAAATGCTGCTGAAG | |
| | | TTGATGCACATCTCTCA | |
| | | CCCCCTTATACCTCACA | |
| | | AAACCATATCTCCTTCC | |
| | | TTGGTTGTTTCTTTTCCT | |
| | | GGGTATGAAAACCTCT | |
| | | CTCACGTCTCCTTGCTG | |
| | | TTTAAAATGGAACCAC | |
| | | AAATCCTTTGCTGTTGC | |
| | | GTCTTCCGGGAACCTCG | |
| | | TAAAGTAAAAGGATAT | |
| | | AACGTCCTTATGGTCTC | |
| | | TCCAGTTCGCTCGCGTG | |
| | | TAGGATTGACGCTGTCC | |
| | | TGTGGCTAACCCTTCGA | |
| | | TGGTCAGATGGGAACC | |
| | | CTCTGACTCTCGAAATC | |
| | | TGACTCTAGCTCTCCTC | |
| | | TCTTTCCTAGTCCTAAC | |
| | | TCTCTCCCACCCACTGT | |
| | | TTCTCTCTCTACTCTCTC | |
| | | TCTCTCTCCATGATTGG | |
| | | ATTGTTCT | |
| | | (SEQ ID NO: 23) | |
| Gm18 | 56662924 | C | T |
| Gm18 | 56663558 | A | T |
| Gm18 | 56663630 | T | A |
| Gm18 | 56663642 | T | C |
| Gm18 | 56663911 | T | G |
| Gm18 | 56664225 | A | G |
| Gm18 | 56664232 | C | T |
| Gm18 | 56664489 | T | C |
| Gm18 | 56664670 | C | G |
| Gm18 | 56665263 | G | T |
| Gm18 | 56665270 | TTG | T |
| Gm18 | 56665533 | T | TA |
| Gm18 | 56665602 | G | A |
| Gm18 | 56666140 | CA | C |
| Gm18 | 56666204 | T | A |
| Gm18 | 56666205 | T | A |
| Gm18 | 56666616 | G | C |
| Gm18 | 56666694 | G | A |
| Gm18 | 56666897 | G | A |
| Gm18 | 56666919 | AT | A |
| Gm18 | 56667054 | GT | G |
| Gm18 | 56667061 | C | G |
| Gm18 | 56667091 | C | T |
| Gm18 | 56667228 | A | G |
| Gm18 | 56667372 | A | T |
| Gm18 | 56667399 | C | G |
| Gm18 | 56667482 | CAG | C |
| Gm18 | 56667528 | T | C |
| Gm18 | 56667541 | A | ACAGCTCGAGTTAATAT |
| | | | (SEQ ID NO: 24) |
| Gm18 | 56667755 | G | T |
| Gm18 | 56667840 | T | A |
| Gm18 | 56667860 | T | A |
| Gm18 | 56667861 | A | AGG |
| Gm18 | 56668194 | A | AGAAATGGAGAAAGTG |
| | | | (SEQ ID NO: 25) |
| Gm18 | 56668230 | CT | C |
| Gm18 | 56668264 | T | TA |
| Gm18 | 56668288 | TA | T |
| Gm18 | 56668290 | A | T |
| Gm18 | 56668400 | A | AAAT |
| Gm18 | 56668492 | A | T |
| Gm18 | 56668671 | A | C |
| Gm18 | 56668721 | A | ATAT |
| Gm18 | 56668741 | C | T |
| Gm18 | 56668770 | A | T |
| Gm18 | 56668923 | CCG | C |
| Gm18 | 56668926 | TCACC | T |
| Gm18 | 56669216 | T | G |
| Gm18 | 56676220 | C | A |
| Gm18 | 56676566 | T | G |
| Gm18 | 56676638 | C | T |
| Gm18 | 56680021 | C | G |
| Gm18 | 56680179 | C | T |
| Gm18 | 56682143 | A | C |
| Gm18 | 56682234 | C | T |
| Gm18 | 56682368 | C | CCTTCTTCAGTT |
| | | | (SEQ ID NO: 26) |
| Gm18 | 56682411 | A | G |
| Gm18 | 56682511 | C | T |
| Gm18 | 56682528 | C | A |
| Gm18 | 56682538 | G | C |
| Gm18 | 56682658 | T | A |
| Gm18 | 56682675 | C | A |
| Gm18 | 56682700 | C | T |
| Gm18 | 56682703 | C | T |
| Gm18 | 56682726 | T | TTTTATCATGAAA |
| | | | (SEQ ID NO: 27) |
| Gm18 | 56682755 | T | C |
| Gm18 | 56682839 | T | G |
| Gm18 | 56682841 | A | C |
| Gm18 | 56682852 | G | A |
| Gm18 | 56682858 | T | A |
| Gm18 | 56682870 | T | A |
| Gm18 | 56682968 | C | A |
| Gm18 | 56683053 | T | C |
| Gm18 | 56683054 | G | A |
| Gm18 | 56683110 | TTC | T |
| Gm18 | 56683296 | A | C |
| Gm18 | 56683332 | A | ATATATATATATATAT |
| | | | (SEQ ID NO: 28) |
| Gm18 | 56683334 | C | A |
| Gm18 | 56683428 | A | ATCC |
| Gm18 | 56683643 | A | G |
| Gm18 | 56683761 | A | G |
| Gm18 | 56683817 | T | C |
| Gm18 | 56683881 | ATGTGTGTGTG | A |
| | | (SEQ ID NO: 82) | |
| Gm18 | 56683894 | TGTA | T |
| Gm18 | 56684003 | A | G |
| Gm18 | 56684101 | A | T |
| Gm18 | 56684693 | T | G |
| Gm18 | 56684788 | G | A |
| Gm18 | 56684790 | C | A |
| Gm18 | 56684820 | T | A |
| Gm18 | 56684873 | T | G |
| Gm18 | 56684957 | A | G |
| Gm18 | 56684968 | C | T |
| Gm18 | 56685074 | T | G |
| Gm18 | 56685278 | TAA | T |
| Gm18 | 56685521 | C | G |
| Gm18 | 56685535 | A | AAGGGGGGAATGG |
| | | | (SEQ ID NO: 29) |
| Gm18 | 56685566 | C | A |
| Gm18 | 56685685 | T | C |
| Gm18 | 56685835 | T | C |
| Gm18 | 56685874 | C | A |
| Gm18 | 56685895 | C | A |
| Gm18 | 56686085 | C | T |
| Gm18 | 56686209 | T | C |
| Gm18 | 56686219 | G | A |
| Gm18 | 56686290 | TA | T |
| Gm18 | 56686387 | T | C |
| Gm18 | 56686538 | C | T |
| Gm18 | 56686683 | CA | C |
| Gm18 | 56686768 | C | A |
| Gm18 | 56686773 | G | A |
| Gm18 | 56686786 | A | ATAAAAT |
| Gm18 | 56686895 | C | T |
| Gm18 | 56686900 | T | A |
| Gm18 | 56686921 | G | A |
| Gm18 | 56686940 | AG | A |
| Gm18 | 56687096 | T | C |
| Gm18 | 56687120 | C | T |
| Gm18 | 56687358 | A | C |
| Gm18 | 56687362 | T | C |
| Gm18 | 56687459 | A | C |
| Gm18 | 56687461 | G | T |
| Gm18 | 56687462 | A | C |
| Gm18 | 56687515 | GAAAGGTGGA | G |
| | | (SEQ ID NO: 83) | |
| Gm18 | 56687733 | A | C |
| Gm18 | 56687735 | C | T |
| Gm18 | 56687743 | CCTATGCG | C |
| Gm18 | 56687842 | T | C |
| Gm18 | 56688141 | T | C |
| Gm18 | 56688170 | A | G |
| Gm18 | 56688353 | C | T |
| Gm18 | 56688392 | AT | A |
| Gm18 | 56688800 | T | A |
| Gm18 | 56689257 | T | G |
| Gm18 | 56689445 | T | G |
| Gm18 | 56689598 | G | GT |
| Gm18 | 56689744 | T | G |
| Gm18 | 56689862 | CA | C |
| Gm18 | 56689878 | TAA | T |
| Gm18 | 56689972 | GAA | G |
| Gm18 | 56690034 | A | AT |
| Gm18 | 56690170 | A | G |
| Gm18 | 56690474 | CA | C |
| Gm18 | 56690559 | A | ATT |
| Gm18 | 56690747 | A | T |
| Gm18 | 56690889 | T | C |
| Gm18 | 56690976 | T | G |
| Gm18 | 56691178 | C | A |
| Gm18 | 56691193 | A | C |
| Gm18 | 56691290 | G | T |
| Gm18 | 56691305 | T | A |
| Gm18 | 56691433 | G | T |
| Gm18 | 56691440 | C | T |
| Gm18 | 56692305 | T | TAATATAATTTATTTATT |
| | | | AA (SEQ ID NO: 84) |
| Gm18 | 56692901 | G | GA |
| Gm18 | 56692988 | G | C |
| Gm18 | 56693008 | T | C |
| Gm18 | 56693110 | T | A |
| Gm18 | 56693247 | AAATATATATATATATA | A |
| | | TATATATATATATATAT | |
| | | ATATATAT | |
| | | (SEQ ID NO: 30) | |
| Gm18 | 56693306 | TA | T |
| Gm18 | 56693344 | TA | T |
| Gm18 | 56693516 | T | C |
| Gm18 | 56695064 | C | G |
| Gm18 | 56697201 | T | TCA |
| Gm18 | 56697257 | G | C |
| Gm18 | 56697691 | G | T |
| Gm18 | 56697723 | G | A |
| Gm18 | 56698131 | G | T |
| Gm18 | 56698364 | G | A |
| Gm18 | 56698379 | A | G |
| Gm18 | 56698390 | T | A |
| Gm18 | 56698424 | G | A |
| Gm18 | 56698426 | G | A |
| Gm18 | 56698434 | G | A |
| Gm18 | 56698465 | C | A |
| Gm18 | 56698489 | C | A |
| Gm18 | 56698566 | GT | G |
| Gm18 | 56698734 | A | G |
| Gm18 | 56698802 | T | C |
| Gm18 | 56698808 | G | GA |
| Gm18 | 56698810 | G | A |
| Gm18 | 56698818 | T | G |
| Gm18 | 56698825 | C | T |
| Gm18 | 56699167 | CA | C |
| Gm18 | 56699334 | G | T |
| Gm18 | 56699339 | T | C |
| Gm18 | 56699344 | A | C |
| Gm18 | 56699389 | AT | A |
| Gm18 | 56699399 | C | A |
| Gm18 | 56699401 | A | ATT |
| Gm18 | 56699504 | G | A |
| Gm18 | 56699527 | C | CAAAAATAA |
| Gm18 | 56699584 | G | A |
| Gm18 | 56699588 | TA | T |
| Gm18 | 56699607 | TTCA | T |
| Gm18 | 56699622 | C | CT |
| Gm18 | 56699653 | T | A |
| Gm18 | 56699721 | G | A |
| Gm18 | 56699759 | A | ATAAATAAATAATAATA |
| | | | GT (SEQ ID NO: 31) |
| Gm18 | 56699770 | C | G |
| Gm18 | 56699840 | A | G |
| Gm18 | 56699965 | T | A |
| Gm18 | 56700056 | G | T |
| Gm18 | 56700068 | G | A |
| Gm18 | 56700082 | G | A |
| Gm18 | 56700086 | T | A |
| Gm18 | 56700348 | T | C |
| Gm18 | 56700360 | C | G |
| Gm18 | 56700362 | T | C |
| Gm18 | 56700612 | A | C |
| Gm18 | 56700818 | T | TG |
| Gm18 | 56700967 | G | C |
| Gm18 | 56701142 | A | G |
| Gm18 | 56701149 | T | C |
| Gm18 | 56701244 | A | C |
| Gm18 | 56701322 | A | G |
| Gm18 | 56701339 | A | G |
| Gm18 | 56701342 | T | C |
| Gm18 | 56701385 | C | T |
| Gm18 | 56701428 | C | T |
| Gm18 | 56701439 | G | T |
| Gm18 | 56701455 | T | C |
| Gm18 | 56701492 | G | A |
| Gm18 | 56701673 | C | T |
| Gm18 | 56701736 | T | C |
| Gm18 | 56701824 | A | AG |
| Gm18 | 56701832 | A | ATT |
| Gm18 | 56702001 | C | T |
| Gm18 | 56702064 | T | C |
| Gm18 | 56702101 | T | G |
| Gm18 | 56702360 | C | T |
| Gm18 | 56702698 | AT | A |
| Gm18 | 56702760 | A | C |
| Gm18 | 56702767 | G | GC |
| Gm18 | 56702770 | GATTT | G |
| Gm18 | 56702777 | T | G |
| Gm18 | 56702779 | A | G |
| Gm18 | 56702780 | C | T |
| Gm18 | 56702788 | G | A |
| Gm18 | 56702824 | A | T |
| Gm18 | 56702882 | A | G |
| Gm18 | 56702888 | C | T |
| Gm18 | 56702894 | T | C |
| Gm18 | 56702927 | A | G |
| Gm18 | 56702944 | A | G |
| Gm18 | 56703048 | T | A |
| Gm18 | 56703067 | A | G |
| Gm18 | 56703127 | T | C |
| Gm18 | 56703131 | G | A |
| Gm18 | 56703135 | T | A |
| Gm18 | 56703188 | C | T |
| Gm18 | 56703195 | A | G |
| Gm18 | 56703209 | T | C |
| Gm18 | 56703284 | A | AT |
| Gm18 | 56703290 | T | A |
| Gm18 | 56703334 | T | C |
| Gm18 | 56703375 | A | T |
| Gm18 | 56703399 | T | A |
| Gm18 | 56703404 | T | G |
| Gm18 | 56703506 | A | G |
| Gm18 | 56703564 | G | A |
| Gm18 | 56703591 | C | CTTCTTACTTGTAACTAA |
| | | | GTCTTTGA |
| | | | (SEQ ID NO: 32) |
| Gm18 | 56703608 | G | A |
| Gm18 | 56703653 | T | TA |
| Gm18 | 56703658 | G | A |
| Gm18 | 56703672 | GAA | G |
| Gm18 | 56703682 | TA | T |
| Gm18 | 56703785 | T | A |
| Gm18 | 56703798 | T | G |
| Gm18 | 56703824 | T | G |
| Gm18 | 56703833 | A | AAATT |
| Gm18 | 56703842 | C | A |
| Gm18 | 56703861 | AG | A |
| Gm18 | 56703866 | G | A |
| Gm18 | 56704030 | CT | C |
| Gm18 | 56704201 | A | G |
| Gm18 | 56704273 | T | A |
| Gm18 | 56704278 | C | A |
| Gm18 | 56704284 | G | C |
| Gm18 | 56704352 | T | TG |
| Gm18 | 56704382 | G | C |
| Gm18 | 56704398 | ATACT | A |
| Gm18 | 56704417 | T | C |
| Gm18 | 56704420 | TA | T |
| Gm18 | 56704443 | T | A |
| Gm18 | 56704456 | A | G |
| Gm18 | 56704501 | T | C |
| Gm18 | 56704508 | T | A |
| Gm18 | 56704531 | C | T |
| Gm18 | 56704539 | T | C |
| Gm18 | 56704548 | A | C |
| Gm18 | 56704611 | C | G |
| Gm18 | 56704650 | C | T |
| Gm18 | 56704669 | A | T |
| Gm18 | 56704693 | T | C |
| Gm18 | 56704697 | G | A |
| Gm18 | 56704748 | A | T |
| Gm18 | 56704760 | A | ACCATG |
| Gm18 | 56704766 | A | T |
| Gm18 | 56704771 | G | A |
| Gm18 | 56704777 | T | C |
| Gm18 | 56704845 | C | T |
| Gm18 | 56704940 | TAA | T |
| Gm18 | 56704963 | G | A |
| Gm18 | 56705061 | G | A |
| Gm18 | 56705077 | T | C |
| Gm18 | 56705088 | A | C |
| Gm18 | 56705094 | G | C |
| Gm18 | 56705113 | C | T |
| Gm18 | 56705146 | A | T |
| Gm18 | 56705200 | A | G |
| Gm18 | 56705219 | A | G |
| Gm18 | 56705251 | T | TATATTTTTTGTGTTTGG |
| | | | TCTTTGATAAATTTTTCT |
| | | | TTCGAATTTGGATTCTAA |
| | | | TGTTTTAAATTTTTTATCT |
| | | | TAAAGTCTCTATCATTAA |
| | | | TATGTAATCCTACATGAC |
| | | | TCTTAATTAGACACGTAG |
| | | | AAGTGTGATGTGTCATGT |
| | | | CAAATATAACCTTTGTGA |
| | | | TTTCTATTTATACATATT |
| | | | GGATTATTAAAAAAAAA |
| | | | TATTTTCTTTGTTAAAAA |
| | | | ATAATAAAATCCAACTT |
| | | | ATATAAACAATAATCCG |
| | | | ACATAATACATCACATTT |
| | | | TTACGTGTGTCCAGTTCA |
| | | | TAC (SEQ ID NO: 33) |
| Gm18 | 56705253 | G | A |
| Gm18 | 56705254 | G | A |
| Gm18 | 56711483 | TA | T |
| Gm18 | 56711576 | G | GA |
| Gm18 | 56711711 | A | G |
| Gm18 | 56711752 | T | TA |
| Gm18 | 56712550 | T | C |
| Gm18 | 56712561 | T | C |
| Gm18 | 56712563 | A | G |
| Gm18 | 56712847 | T | A |
| Gm18 | 56712894 | CTATATA | C |
| Gm18 | 56712934 | A | G |
| Gm18 | 56712958 | C | CCA |
| Gm18 | 56712959 | T | TGC |
| Gm18 | 56712988 | C | T |
| Gm18 | 56713017 | C | A |
| Gm18 | 56713045 | G | T |
| Gm18 | 56713064 | C | T |
| Gm18 | 56713220 | T | C |
| Gm18 | 56713259 | G | A |
| Gm18 | 56713332 | T | C |
| Gm18 | 56713478 | G | C |
| Gm18 | 56713624 | C | A |
| Gm18 | 56713748 | A | G |
| Gm18 | 56713764 | C | A |
| Gm18 | 56713852 | A | T |
| Gm18 | 56713995 | C | A |
| Gm18 | 56714029 | G | A |
| Gm18 | 56714182 | G | C |
| Gm18 | 56714236 | A | G |
| Gm18 | 56714245 | G | T |
| Gm18 | 56714295 | T | G |
| Gm18 | 56714368 | G | T |
| Gm18 | 56714611 | T | C |
| Gm18 | 56714635 | T | C |
| Gm18 | 56714850 | A | C |
| Gm18 | 56714865 | A | G |
| Gm18 | 56715062 | T | A |
| Gm18 | 56715180 | C | A |
| Gm18 | 56715529 | C | CTT |
| Gm18 | 56715568 | C | T |
| Gm18 | 56715595 | C | T |
| Gm18 | 56715695 | A | G |
| Gm18 | 56715700 | T | TGGA |
| Gm18 | 56715702 | T | G |
| Gm18 | 56715704 | AC | A |
| Gm18 | 56715707 | T | TGGA |
| Gm18 | 56715709 | T | TGG |
| Gm18 | 56715710 | A | G |
| Gm18 | 56715712 | C | T |
| Gm18 | 56715714 | G | T |
| Gm18 | 56715716 | G | GAGA |
| Gm18 | 56715717 | G | GAA |
| Gm18 | 56715720 | T | TTTTGACAAAAAC |
| | | | (SEQ ID NO: 34) |
| Gm18 | 56715724 | C | CTTAAA |
| Gm18 | 56715725 | C | G |
| Gm18 | 56715727 | T | TTTTAAAAG |
| Gm18 | 56715732 | A | T |
| Gm18 | 56715747 | T | C |
| Gm18 | 56715775 | C | CAACACTAACAAATTTTA |
| | | | (SEQ ID NO: 35) |
| Gm18 | 56715781 | A | G |
| Gm18 | 56715841 | T | C |
| Gm18 | 56715854 | T | A |
| Gm18 | 56715872 | G | A |
| Gm18 | 56715929 | C | T |
| Gm18 | 56715931 | A | G |
| Gm18 | 56715950 | G | C |
| Gm18 | 56715958 | G | A |
| Gm18 | 56715971 | C | T |
| Gm18 | 56715981 | T | C |
| Gm18 | 56715982 | A | G |
| Gm18 | 56715993 | G | A |
| Gm18 | 56716009 | A | T |
| Gm18 | 56716021 | ATT | A |
| Gm18 | 56716031 | A | T |
| Gm18 | 56716262 | C | CG |
| Gm18 | 56716273 | G | A |
| Gm18 | 56716285 | G | A |
| Gm18 | 56716310 | G | A |
| Gm18 | 56716354 | TTAA | T |
| Gm18 | 56716388 | A | T |
| Gm18 | 56716452 | T | C |
| Gm18 | 56716547 | TA | T |
| Gm18 | 56716576 | T | C |
| Gm18 | 56716720 | T | TA |
| Gm18 | 56716984 | G | A |
| Gm18 | 56717056 | T | TGC |
| Gm18 | 56717086 | A | G |
| Gm18 | 56717118 | T | C |
| Gm18 | 56717139 | T | C |
| Gm18 | 56717216 | G | A |
| Gm18 | 56717267 | T | A |
| Gm18 | 56717293 | T | C |
| Gm18 | 56717338 | C | T |
| Gm18 | 56717407 | G | C |
| Gm18 | 56717408 | G | C |
| Gm18 | 56717438 | C | T |
| Gm18 | 56717695 | G | A |
| Gm18 | 56718157 | A | T |
| Gm18 | 56718193 | G | T |
| Gm18 | 56718199 | A | AT |
| Gm18 | 56718208 | C | T |
| Gm18 | 56718334 | A | G |
| Gm18 | 56718585 | A | AT |
| Gm18 | 56718665 | A | T |
| Gm18 | 56718667 | G | A |
| Gm18 | 56718831 | A | G |
| Gm18 | 56718997 | G | A |
| Gm18 | 56719142 | C | CAAGTATGCAGGCTTTTG |
| | | | TACCCATAGTACCACTGG |
| | | | TACTATTTCAATCTATAA |
| | | | TATATATATTTTTGCTGA |
| | | | GCAAAAAAAAAA |
| | | | (SEQ ID NO: 36) |
| Gm18 | 56719289 | T | C |
| Gm18 | 56719463 | C | A |
| Gm18 | 56719488 | C | T |
| Gm18 | 56719491 | T | C |
| Gm18 | 56719540 | T | C |
| Gm18 | 56719572 | C | T |
| Gm18 | 56719814 | A | G |
| Gm18 | 56719828 | G | A |
| Gm18 | 56719975 | A | G |
| Gm18 | 56720147 | AAACCCATGATAACTG | A |
| | | GTTTTAATTGTGGGCTG | |
| | | TCTCCATACTCTACACA | |
| | | AGCT (SEQ ID NO: 37) | |
| Gm18 | 56720232 | G | T |
| Gm18 | 56720354 | G | T |
| Gm18 | 56720393 | G | A |
| Gm18 | 56720436 | A | T |
| Gm18 | 56720464 | T | C |
| Gm18 | 56720492 | T | G |
| Gm18 | 56720496 | C | CTGTCCAGCCAAGATCTT |
| | | | GACTGTTGTAGTTGAACT |
| | | | TAGTAGCTGAAGAGGAA |
| | | | AGAGAATGTGATGGGTG |
| | | | GTTGAGATTTGGGAAGG |
| | | | AGAGAAACCTTGCTGGG |
| | | | AGAGGCTGCAGAGGATC |
| | | | CAGATTCCTGCTATATAT |
| | | | TGTCATGATAACTGTCAA |
| | | | GTGTGAAATTGAGAGCT |
| | | | TGCTAATCTTGTAGAATA |
| | | | TATAAACCATTTTTGACT |
| | | | TTTTTTTTTTAAAAAAAT |
| | | | GATTTGATCATATGGCAT |
| | | | TCATGTTTGTTTGAGTTG |
| | | | TAGCAGTTTCTTTCTGTT |
| | | | CCCATAAATTGTTATCAT |
| | | | TCTTTTTTGAGAGTTGGA |
| | | | TTTACTGGTTTGAG |
| | | | (SEQ ID NO: 38) |
| Gm18 | 56720499 | A | G |
| Gm18 | 56720501 | T | G |
| Gm18 | 56720502 | T | C |
| Gm18 | 56720534 | T | C |
| Gm18 | 56720672 | G | C |
| Gm18 | 56720684 | T | C |
| Gm18 | 56720710 | A | C |
| Gm18 | 56720828 | C | T |
| Gm18 | 56720902 | C | T |
| Gm18 | 56720938 | T | C |
| Gm18 | 56720942 | C | T |
| Gm18 | 56721082 | C | T |
| Gm18 | 56721119 | T | C |
| Gm18 | 56721214 | G | A |
| Gm18 | 56721542 | G | A |
| Gm18 | 56721704 | C | T |
| Gm18 | 56721865 | G | A |
| Gm18 | 56722043 | G | A |
| Gm18 | 56722203 | G | T |
| Gm18 | 56722409 | G | T |
| Gm18 | 56722545 | A | C |
| Gm18 | 56722832 | C | T |
| Gm18 | 56722859 | A | C |
| Gm18 | 56723297 | G | A |

Oligonucleotide primers (herein, ‘primers’) can be developed and used to identify plants carrying any one of the chromosomal intervals depicted in SEQ ID NO: 11, 12, or 13 found to be highly associated with ASR resistance. Primers can also be developed and used to identify plants carrying SEQ ID NO: 2. Specifically, one having ordinary skill in the art can develop primers to detect any single nucleotide polymorphism (herein ‘SNP’) as identified in Table 1 in respect to identifying or producing soybean lines having any one of or a portion of the chromosome intervals depicted in SEQ ID NOs: 11, 12, or 13 that are associated with ASR resistance. A TAQMAN® assay (e.g. generally a two-step allelic discrimination assay or similar), a KASP™ assay (generally a one-step allelic discrimination assay defined below or similar), or both can be employed to identify the SNPs that associate with increased ASR resistance as disclosed herein (e.g. favorable alleles as depicted in Table 1 above). In an exemplary two-step assay, a forward primer, a reverse primer, and two assay probes (or hybridization oligos) are employed. The forward and reverse primers are employed to amplify genetic loci that comprise SNPs that are associated with ASR resistance loci (for example, any of the favorable alleles as shown in Table 1). The particular nucleotides that are present at the SNP positions are then assayed using the assay primers (which in some embodiments are differentially labeled with, for example, fluorophores to permit distinguishing between the two assay probes in a single reaction), which in each pair differ from each other with respect to the nucleotides that are present at the SNP position (although it is noted that in any given pair, the probes can differ in their 5′ or 3′ ends without impacting their abilities to differentiate between nucleotides present at the corresponding SNP positions). In some embodiments, the assay primers and the reaction conditions are designed such that an assay primer will only hybridize to the reverse complement of a 100% perfectly matched sequence, thereby permitting identification of which allele(s) that are present based upon detection of hybridizations.

In one embodiment of the invention, the following assay can be employed to identify the SNPs that associate with increased ASR resistance as disclosed herein:

TABLE 2
Assays for detection of SNPs across interval

| | Type of | | Physical | | |
|---|---|---|---|---|---|
| | variant (SNP | Position in | position on | Favorable | Unfavorable |
| Assay | or InDel) | BAC sequence | chromosome | allele | allele |
| 1 | SNP | 282 | 56,723,297 | T | C |
| 2 | SNP | 173,304 | 56,607,175 | G | A |
| 3 | Insertion | 42631-42641 | 56,682,368 | aactgaagaag | — |
| 4 | SNP | 42,775 | 56,682,234 | A | G |
| 5 | SNP | 42,866 | 56,682,143 | G | T |
| 6 | SNP | 44,833 | 56,680,179 | A | G |
| 7 | SNP | 48,513 | 56,676,638 | A | G |
| 8 | SNP | 48,585 | 56,676,566 | C | A |
| 9 | SNP | 87,329 | 56,676,220 | T | G |
| 10 | SNP | 106,768 | 56,680,021 | C | G |

| | TAQ Allelic Discrimination Assay | | | |
|---|---|---|---|---|
| | probe 1 | probe 2 | primer 1 | primer 2 |
| Assay | sequence | sequence | sequence | sequence |
| 1 | AATAGCCCCACCC | CAATAGCCCCATC | GGATGGAATGGG | GCAAGCAAACCTC |
| | TTAATT | CTTAA | AAAGTCTTGATAA | GAACCTA |
| | (SEQ ID NO: 39) | (SEQ ID NO: 49) | (SEQ ID NO: 59) | (SEQ ID NO: 69) |
| 2 | TCATCATGCTAAA | CTCATCATGCTAA | AAAACCTGCGAA | CCCACCCGTTTAT |
| | ACTGAGTA | AATTGAGTA | GCTCCTTTACTA | CATTTGCACTT |
| | (SEQ ID NO: 40) | (SEQ ID NO: 50) | (SEQ ID NO: 60) | (SEQ ID NO: 70) |
| 3 | TTGAGGAAGAACT | ACGGATTGAGGA | GTTTGTTGTCCAC | TGTCATCAAGCAT |
| | GAAGAAGGTAGC | AGGTAGCAGAA | GCCTGAA | CACCAGGTA |
| | (SEQ ID NO: 41) | (SEQIDNO: 51) | (SEQ ID NO: 61) | (SEQ ID NO: 71) |
| 4 | ATTGAAAATTGTT | CCTATTGAAAATT | TTACGTCAATGCT | ACTAATAACAAGG |
| | CCTGTAC | GTTCTTGTAC | CTTAAGGTCTAA | AGGTGGCATAC |
| | (SEQ ID NO: 42) | (SEQ ID NO: 52) | (SEQ ID NO: 62) | (SEQ ID NO: 72) |
| 5 | TGGCTTAGGAAGA | TGGCTTAGGACGA | GCCTTGACCTCAT | ACTAATAACAAGG |
| | AAGCTC | AAGCT | CCATTGATAGAG | AGGTGGCATAC |
| | (SEQ ID NO: 43) | (SEQ ID NO: 53) | (SEQ ID NO: 63) | (SEQ ID NO: 73) |
| 6 | ATCTCTGATCTGG | TCTCTGATTTGGC | GGTGTCAACCACA | TTCTGGCGTGGAG |
| | CTCAC | TCAC | TCTTCAGCTTT | GACAAA |
| | (SEQ ID NO: 44) | (SEQ ID NO: 54) | (SEQ ID NO: 64) | (SEQ ID NO: 74) |
| 7 | CAACAAATACATA | AGCAACAAATAC | ACTTGCTCTACTT | GTCAGTGTCCTAG |
| | TACCAAAGTC | ATATATCAAAGTC | CATAAATGGGTA | TTTTCCTTGA |
| | (SEQ ID NO: 45) | (SEQ ID NO: 55) | (SEQ ID NO: 65) | (SEQ ID NO: 75) |
| 8 | ACAAAATCATCAC | CAAAATCATCACC | ACCCATTTATGAA | AGCGAACGGGAA |
| | CAAGTTGA | CAGTTG | GTAGAGCAAGTA | CAGTCCAT |
| | (SEQ ID NO: 46) | (SEQ ID NO: 56) | (SEQ ID NO: 66) | (SEQ ID NO: 76) |
| 9 | ATCTGCATGCTAA | CTGCATGCTCATC | TCCGAGAAAGGA | GGACGACACTTTT |
| | TCCTG | CTG | ATAGTTCTGTG | ACATCAAACAC |
| | (SEQ ID NO: 47) | (SEQ ID NO: 57) | (SEQ ID NO: 67) | (SEQ ID NO: 77) |
| 10 | TTCGATCTGACTC | CGATCTGAGTCCT | CACCTTCTCCAAT | GCTTCACCAAGTC |
| | CTGAT | GATC | GCCATATCTGT | AACTCTGA |
| | (SEQ ID NO: 48) | (SEQ ID NO: 50) | (SEQ ID NO: 68) | (SEQ ID NO: 78) |

### Genetic Mapping

Genetic loci correlating with particular phenotypes, such as rust resistance, can be mapped in an organism's genome. By identifying a marker or cluster of markers that co-segregate with a trait of interest, the breeder is able to rapidly select a desired phenotype by selecting for the proper marker (a process called marker-assisted selection, or “MAS”). Such markers may also be used by breeders to design genotypes in silico and to practice whole genome selection.

In certain embodiments, the present invention provides markers associated with enhanced resistance to rust (e.g. Asian soybean rust). Detection of these markers and/or other linked markers can be used to identify, select, and/or produce rust resistant, more specifically Asian soybean rust resistant (herein, “ASR”), plants and/or to eliminate plants that are not disease resistant from breeding programs or planting.

Genetic Loci Associated with Enhanced Disease Resistance

Markers associated with enhanced disease resistance are identified herein (see Table 1 indicating favorable markers associated with enhanced ASR resistance). A marker of the present invention may comprise a single allele or a combination of alleles at one or more genetic loci (for example, any combination of a favorable markers from Table 1. For example, the marker may comprise one or more marker alleles located within a first chromosomal interval (e.g. SEQ ID NO: 11) and one or more marker alleles located within a second chromosomal interval (e.g. SEQ ID NO: 12 or SEQ ID NO: 13).

### Marker-Assisted Selection

Markers can be used in a variety of plant breeding applications. See, e.g., Staub et al., Hortscience 31: 729 (1996); Tanksley, Plant Molecular Biology Reporter 1: 3 (1983). One of the main areas of interest is to increase the efficiency of backcrossing and introgressing genes using marker-assisted selection (MAS). In general, MAS takes advantage of genetic markers that have been identified as having a significant likelihood of co-segregation with a desired trait. Such markers are presumed to be in/near the gene(s) that give rise to the desired phenotype, and their presence indicates that the plant will possess the desired trait. Plants which possess the marker are expected to transfer the desired phenotype to their progeny.

A marker that demonstrates linkage with a locus affecting a desired phenotypic trait provides a useful tool for the selection of the trait in a plant population. This is particularly true where the phenotype is hard to assay or occurs at a late stage in plant development. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing or imparting the trait. Having flanking markers decreases the chances that false positive selection will occur. The ideal situation is to have a marker within the causative gene itself, so that recombination cannot occur between the marker and the gene. Such a marker is called a “perfect marker”.

When a gene is introgressed by MAS, it is not only the gene that is introduced but also the flanking regions. Gepts, Crop Sci 42:1780 (2002). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite soybean line. This is also sometimes referred to as “yield drag.” The size of the flanking region can be decreased by additional backcrossing, although this is not always successful, as breeders do not have control over the size of the region or the recombination breakpoints. Young et al., Genetics 120:579 (1998). In classical breeding, it is usually only by chance that recombinations that contribute to a reduction in the size of the donor segment are selected. Tanksley et al., Biotechnology 7: 257 (1989). Even after 20 backcrosses, one might find a sizeable piece of the donor chromosome still linked to the gene being selected. With markers, however, it is possible to select those rare individuals that have experienced recombination near the gene of interest. In 150 backcross plants, there is a 95% chance that at least one plant will have experienced a crossover within 1 cM of the gene, based on a single meiosis map distance. Markers allow for unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers. See Tanksley et al., supra. When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The availability of integrated linkage maps of the soybean genome containing increasing densities of public soybean markers has facilitated soybean genetic mapping and MAS.

Of all the molecular marker types, SNPs are the most abundant and have the potential to provide the highest genetic map resolution. Bhattramakki et al., Plant Molec. Biol. 48:539 (2002). SNPs can be assayed in a so-called “ultra-high-throughput” fashion because they do not require large amounts of nucleic acid and automation of the assay is straight-forward. SNPs also have the benefit of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing and coded spheres. Such methods have been reviewed in various publications: Gut, Hum. Mutat. 17:475 (2001); Shi, Clin. Chem. 47:164 (2001); Kwok, Pharmacogenomics 1:95 (2000); Bhattramakki and Rafalski, Discovery and application of single nucleotide polymorphism markers in plants, in PLANT GENOTYPING: THE DNA FINGERPRINTING OF PLANTS, CABI Publishing, Wallingford (2001). A wide range of commercially available technologies utilize these and other methods to interrogate SNPs, including Masscode™ (Qiagen, Germantown, MD), Invader® (Hologic, Madison, WI), SnapShot® (Applied Biosystems, Foster City, CA), Taqman® (Applied Biosystems, Foster City, CA) and Beadarrays™ (Illumina, San Diego, CA).

A number of SNP alleles together within a sequence, or across linked sequences, can be used to describe a haplotype for any particular genotype. Ching et al., BMC Genet. 3:19 (2002); Gupta et al., (2001), Rafalski, Plant Sci. 162:329 (2002b). Haplotypes can be more informative than single SNPs and can be more descriptive of any particular genotype. For example, a single SNP may be allele “T” for a specific Disease resistant line or variety, but the allele “T” might also occur in the soybean breeding population being utilized for recurrent parents. In this case, a combination of alleles at linked SNPs may be more informative. Once a unique haplotype has been assigned to a donor chromosomal region, that haplotype can be used in that population or any subset thereof to determine whether an individual has a particular gene. The use of automated high throughput marker detection platforms known to those of ordinary skill in the art makes this process highly efficient and effective.

The markers of the present invention can be used in marker-assisted selection protocols to identify and/or select progeny with enhanced Asian soybean rust resistance. Such methods can comprise, consist essentially of or consist of crossing a first soybean plant or germplasm with a second soybean plant or germplasm, wherein the first soybean plant or germplasm comprises a chromosomal interval derived from SX6907 wherein said chromosome interval comprises SEQ ID NOs 11, 12, or 13, or a portion thereof encoding ASR resistance, or wherein the chromosome interval comprises SEQ ID NO: 2 or a nucleic acid encoding SEQ ID NO: 1, and selecting a progeny plant that possesses the marker. Either of the first and second soybean plants, or both, may be of a non-naturally occurring variety of soybean. In some embodiments, the second soybean plant or germplasm is of an elite variety of soybean. In some embodiments, the genome of the second soybean plant or germplasm is at least about 50%, 55%, 60%, 65%, 70%, 75%, 80%, 85%, 90%, 95%, 97%, 99%, or 100% identical to that of an elite variety of soybean. In another embodiment, the first soybean plant comprises a chromosomal interval derived from SX6907 wherein said chromosomal interval comprises SEQ ID NOs 11, 12, or 13 and wherein the chromosome interval further comprises at least one allele as depicted in Table 1. In another embodiment, the first soybean comprises a chromosome interval comprising the nucleic acid sequence of SEQ ID NO: 2, or a portion thereof encoding ASR resistance, or the nucleic acid sequence encoding the protein of SEQ ID NO: 1, or a portion thereof encoding ASR resistance.

### Producing Disease Resistant Plants

Methods for identifying and/or selecting a disease resistant soybean plant or germplasm may comprise, consist essentially of or consist of detecting the presence of a marker associated with enhanced ASR tolerance. The marker may be detected in any sample taken from the plant or germplasm, including, but not limited to, the whole plant or germplasm, a portion of said plant or germplasm (e.g., a seed chip, a leaf punch disk or a cell from said plant or germplasm) or a nucleotide sequence from said plant or germplasm. Such a sample may be taken from the plant or germplasm using any present or future method known in the art, including, but not limited to, automated methods of removing a portion of endosperm with a sharp blade, drilling a small hole in the seed and collecting the resultant powder, cutting the seed with a laser and punching a leaf disk. The soybean plant may be of a non-naturally occurring variety of soybean. In some embodiments, the genome of the soybean plant or germplasm is at least about 50%, 55%, 60%, 65%, 70%, 75%, 80%, 85%, 90%, 95%, 97%, 99% or 100% identical to that of an elite variety of soybean. In some embodiments, the marker detected in the sample may comprise, consist essentially of or consist of one or more marker alleles located within a chromosomal interval selected from:
1) a chromosomal interval comprising SEQ ID NO: 2, or a portion thereof encoding ASR resistance;
2) a chromosomal interval encoding the protein sequence of SEQ ID NO: 1, or a portion thereof encoding ASR resistance;
3) a chromosomal interval comprising SEQ ID NOs 11, 12, or 13, or a portion thereof encoding ASR resistance; or
4) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker displayed in Table 1.

Alternatively, the one or more marker alleles may be located within SEQ ID NO: 2.

Methods for producing a disease resistant soybean plant may comprise, consist essentially of or consist of detecting, in a germplasm, a marker associated with enhanced disease resistance (e.g. ASR) wherein said marker is selected from Table 1 or wherein marker is a closely linked loci of any marker described in Table 1 and producing a soybean plant from said germplasm. Alternatively, the methods may comprise, consist essentially of or consist of detecting, in a germplasm, a marker associated with SEQ ID NO: 2. The marker may be detected in any sample taken from the germplasm, including, but not limited to, a portion of said germplasm (e.g., a seed chip or a cell from said germplasm) or a nucleotide sequence from said germplasm. Such a sample may be taken from the germplasm using any present or future method known in the art, including, but not limited to, automated methods of removing a portion of endosperm with a sharp blade, drilling a small hole in the seed and collecting the resultant powder, cutting the seed with a laser and punching a leaf disk. The germplasm may be of a non-naturally occurring variety of soybean. In some embodiments, the genome of the germplasm is at least about 50%, 55%, 60%, 65%, 70%, 75%, 80%, 85%, 90%, 95%, 97%, 99%, or 100% identical to that of an elite variety of soybean. A disease resistant soybean plant is then produced from the germplasm identified as having the marker associated with enhanced disease resistance (e.g. ASR) according to methods well known in the art for breeding and producing plants from germplasm.

In some embodiments, the marker detected in the germplasm may comprise, consist essentially of or consist of one or more marker alleles located within a chromosomal interval selected from:
1) a chromosomal interval comprising SEQ ID NO: 2, or a portion thereof encoding ASR resistance;
2) a chromosomal interval encoding the protein sequence of SEQ ID NO: 1, or a portion thereof encoding ASR resistance;
3) a chromosomal interval comprising SEQ ID NOs 11, 12, or 13, or a portion thereof encoding ASR resistance; or
4) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker displayed in Table 1.

In another embodiment, the marker is located with the nucleic acid of SEQ ID NO: 2. In some embodiments, the marker detected in the germplasm may comprise, consist essentially of or consist of one or more marker alleles selected from Table 1.

Methods for producing and/or selecting an Asian soybean rust resistant/tolerant soybean plant or germplasm may comprise crossing a first soybean plant or germplasm with a second soybean plant or germplasm, wherein said first soybean plant or germplasm comprises a chromosomal interval selected from:
1) a chromosomal interval comprising SEQ ID NO: 2, or a portion thereof encoding ASR resistance;
2) a chromosomal interval encoding the protein sequence of SEQ ID NO: 1, or a portion thereof encoding ASR resistance;
3) a chromosomal interval comprising SEQ ID NOs 11, 12, or 13, or a portion thereof encoding ASR resistance;
4) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker displayed in Table 1; or
5) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any SNP marker displayed in Table 1, and crossing with a second soybean plant not comprising the chromosome interval then producing a progeny plant with increased ASR resistance. Either the first or second soybean plant or germplasm, or both, may be of a non-naturally occurring variety of soybean. In some embodiments, the second soybean plant or germplasm is of an elite variety of soybean. In some embodiments, the genome of the second soybean plant or germplasm is at least about 50%, 55%, 60%, 65%, 70%, 75%, 80%, 85%, 90%, 95%, 97%, 99% or 100% identical to that of an elite variety of soybean.

In one embodiment, the soybean plant may be used to introduce Asian soybean rust resistance/tolerance into Glycine max strain Williams 82.

Alternatively, methods for producing and/or selecting an Asian soybean rust resistant/tolerant soybean plant or germplasm may comprise crossing a first soybean plant or germplasm with a second soybean plant or germplasm, wherein said first soybean plant or germplasm comprises the nucleic acid sequence of SEQ ID NO: 2 or a protein encoding the amino acid sequence of SEQ ID NO: 1.

Also provided herein is a method of introgressing an allele associated with enhanced Disease (e.g. ASR, SCN, SDS, RKN, Phytophthora, etc.) resistance/tolerance into a soybean plant. Such methods for introgressing an allele associated with enhanced Disease (e.g. ASR, SCN, SDS, RKN, Phytophthora, etc.) resistance/tolerance into a soybean plant or germplasm may comprise, consist essentially of or consist of crossing a first soybean plant or germplasm comprising said allele (the donor) wherein said allele is selected from any allele listed in Table 1 or a maker in “close proximity” to a marker listed in Table 1 with a second soybean plant or germplasm that lacks said allele (the recurrent parent) and repeatedly backcrossing progeny comprising said allele with the recurrent parent. Progeny comprising said allele may be identified by detecting, in their genomes, the presence of a marker associated with enhanced Disease (e.g. ASR, SCN, SDS, RKN, Phytophthora, etc.) resistance/tolerance. The marker may be detected in any sample taken from the progeny, including, but not limited to, a portion of said progeny (e.g., a seed chip, a leaf punch disk, or a cell from said plant or germplasm) or a nucleotide sequence from said progeny. Such a sample may be taken from the progeny using any present or future method known in the art, including, but not limited to, automated methods of removing a portion of endosperm with a sharp blade, drilling a small hole in the seed and collecting the resultant powder, cutting the seed with a laser and punching a leaf disk. Either the donor or the recurrent parent, or both, may be of a non-naturally occurring variety of soybean. In some embodiments, the recurrent parent is of an elite variety of soybean. In some embodiments, the genome of the recurrent parent is at least about 50%, 55%, 60%, 65%, 70%, 75%, 80%, 85%, 90%, 95%, 97%, 99% or 100% identical to that of an elite variety of soybean.

In some embodiments, the marker used to identify progeny comprising an allele associated with enhanced resistance/tolerance to rust may comprise, consist essentially of or consist of one or more marker alleles located within a chromosomal interval selected from:
1) a chromosomal interval comprising SEQ ID NO: 2, or a portion thereof encoding ASR resistance;
2) a chromosomal interval encoding the protein sequence of SEQ ID NO: 1, or a portion thereof encoding ASR resistance;
3) a chromosomal interval comprising SEQ ID NOs 11, 12, or 13, or a portion thereof encoding ASR resistance;
4) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker displayed in Table 1; or
5) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any SNP marker displayed in Table 1 or any closely linked markers in close proximity to said intervals (SEQ ID NOs 11, 12, or 13).

In some embodiments, the marker may comprise, consist essentially of or consist of marker alleles located in at least two different chromosomal intervals. For example, the marker may comprise one or more alleles located in the chromosomal interval defined by and including any two markers in SEQ ID NOs 11, 12, or 13.

### Disease Resistant Soybean Plants and Germplasms

In another embodiment, the present invention provides soybean plants and germplasms that are resistant to rust. As discussed above, the methods of the present invention may be utilized to identify, produce, and/or select a disease resistant soybean plant or germplasm (for example a soybean plant resistant or having increased tolerance to Asian Soybean Rust). In addition, to the methods described above, a soybean plant or germplasm resistant to ASR may be produced by any method whereby a marker associated with enhanced disease tolerance is introduced into the soybean plant or germplasm, including, but not limited to, transformation, protoplast transformation or fusion, a double haploid technique, embryo rescue, gene editing and/or by any other nucleic acid transfer system.

In some embodiments, the soybean plant or germplasm comprises a non-naturally occurring variety of soybean. In some embodiments, the soybean plant or germplasm is at least about 50%, 55%, 60%, 65%, 70%, 75%, 80%, 85%, 90%, 95%, 97%, 99% or 100% identical to that of an elite variety of soybean.

The disease resistant soybean plant or germplasm may be the progeny of a cross between an elite variety of soybean and a variety of soybean that comprises an allele associated with enhanced rust resistance (e.g. ASR) wherein the allele is within a chromosomal interval selected from:
1) a chromosomal interval comprising SEQ ID NO: 2, or a portion thereof encoding ASR resistance;
2) a chromosomal interval encoding the protein sequence of SEQ ID NO: 1, or a portion thereof encoding ASR resistance;
3) a chromosomal interval comprising SEQ ID NOs 11, 12, or 13, or a portion thereof encoding ASR resistance;
4) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker displayed in Table 1.

In other embodiments, the disease resistant soybean plant or germplasm may be the progeny of a cross between an elite variety of soybean and a variety of soybean that comprises an allele associated with enhanced rust resistance (e.g. ASR) wherein the allele comprises SEQ ID NO: 2 or encodes a protein of SEQ ID NO: 1.

One embodiment of the invention is a Glycine max plant that has Asian soybean rust resistance/tolerance and that comprise the nucleic acid sequence of SEQ ID NO: 2 or a chromosomal interval selected from:
1) a chromosomal interval comprising SEQ ID NO: 2, or a portion thereof encoding ASR resistance;
2) a chromosomal interval encoding the protein sequence of SEQ ID NO: 1, or a portion thereof encoding ASR resistance;
3) a chromosomal interval comprising SEQ ID NOs 11, 12, or 13, or a portion thereof encoding ASR resistance; or
4) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker displayed in Table 1.

In certain embodiments, the chromosomal interval confers increased Asian soybean rust (ASR) resistance as compared to a control plant not comprising said chromosomal interval. The Glycine max plant may be derived from strain Williams 82

The disease resistant soybean plant or germplasm may be the progeny of an introgression wherein the recurrent parent is an elite variety of soybean and the donor comprises an allele associated with enhanced disease tolerance and/or resistance wherein the donor carries a chromosomal interval or a portion thereof comprising any one of SEQ ID NOs: 11, 12, or 13, and wherein the chromosome interval comprises at least one allele selected respectively from Table 1.

The disease resistant soybean plant or germplasm may be the progeny of a cross between a first elite variety of soybean (e.g., a tester line) and the progeny of a cross between a second elite variety of soybean (e.g., a recurrent parent) and a variety of soybean that comprises an allele associated with enhanced ASR tolerance (e.g., a donor).

The disease resistant soybean plant or germplasm may be the progeny of a cross between a first elite variety of soybean and the progeny of an introgression wherein the recurrent parent is a second elite variety of soybean and the donor comprises an allele associated with enhanced ASR tolerance.

A disease resistant soybean plant and germplasm of the present invention may comprise one or more markers of the present invention (e.g. any marker described in Table 1; or any marker in close proximity to any marker as described in Table 1).

In some embodiments, the disease resistant soybean plant or germplasm may comprise within its genome, a marker associated with enhanced ASR tolerance, wherein said marker is located within a chromosomal interval selected from:
1) a chromosomal interval comprising SEQ ID NO: 2, or a portion thereof encoding ASR resistance;
2) a chromosomal interval encoding the protein sequence of SEQ ID NO: 1, or a portion thereof encoding ASR resistance;
3) a chromosomal interval comprising SEQ ID NOs 11, 12, or 13, or a portion thereof encoding ASR resistance; or
4) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker displayed in Table 1.

In other embodiments, the marker is located with the nucleic acid sequence of SEQ ID NO: 2.

In some embodiments, the disease resistant soybean plant or germplasm may comprise within its genome a marker that comprises, consists essentially of or consists of marker alleles located in at least two different chromosomal intervals. For example, the marker may comprise one or more alleles located in the chromosomal interval defined by and including any combination of two markers in Table 1 and one or more alleles located in the chromosomal interval defined by and including any combination of two markers in Table 1.

In certain embodiments, the disease resistant soybean plant is derived from Glycine max strain Williams 82.

### Disease Resistant Soybean Seeds

The present invention also provides disease resistant soybean seeds. As discussed above, the methods of the present invention may be utilized to identify, produce, and/or select a disease resistant soybean seed. In addition to the methods described above, a disease resistant soybean seed may be produced by any method whereby a marker associated with enhanced ASR tolerance is introduced into the soybean seed, including, but not limited to, transformation, protoplast transformation or fusion, a double haploid technique, embryo rescue, genetic editing (e.g. CRISPR or TALEN or MegaNucleases) and/or by any other nucleic acid transfer system.

One embodiment of the invention is a seed from Glycine max strain Williams 82 that has been modified to have Asian soybean rust resistance/tolerance i

In some embodiments, the disease resistant soybean seed comprises a non-naturally occurring variety of soybean. In some embodiments, the soybean seed is at least about 50%, 55%, 60%, 65%, 70%, 75%, 80%, 85%, 90%, 95%, 97%, 99% or 100% identical to that of an elite variety of soybean.

The disease resistant soybean seed may be produced by a disease resistant soybean plant identified, produced, or selected by the methods of the present invention. In some embodiments, the disease resistant soybean seed is produced by a disease resistant soybean or wild Glycine plant (e.g. Glycine tomentella) plant comprising any one of chromosomal intervals corresponding to SEQ ID NOs: 11-13, a chromosomal interval comprising SEQ ID NO: 2, or a chromosomal interval encoding a protein of SEQ ID NO: 1, or any portion of these intervals encoding ASR resistance.

A disease resistant soybean seed of the present invention may comprise, be selected by or produced by use of one or more markers from Table 1 of the present invention.

In some embodiments, the disease resistant soybean seed may comprise within its genome, a marker associated with enhanced ASR tolerance, wherein said marker is located within a chromosomal interval selected from the group consisting of:
1) a chromosomal interval comprising SEQ ID NO: 2, or a portion thereof encoding ASR resistance;
2) a chromosomal interval encoding the protein sequence of SEQ ID NO: 1, or a portion thereof encoding ASR resistance;
3) a chromosomal interval comprising SEQ ID NOs 11, 12, or 13, or a portion thereof encoding ASR resistance; or
4) a chromosomal interval spanning 20 cM, 15 cM, 10 cM, 5 cM, 1 cM, 0.5 cM from a SNP marker that associates with increased ASR resistance in soybean wherein the SNP marker is selected from the group consisting of any favorable SNP marker displayed in Table 1.

In other embodiments, the marker is located with the nucleic acid sequence of SEQ ID NO: 2.

### Proteins Conferring Rust Resistance

In addition to providing Glycine max plants having increased Asian soybean rust resistance, in certain embodiments, the invention provides proteins that are related to rust resistance, in particular Asian soybean rust resistance (herein, “ASR”). In particular embodiments, these proteins confer increased Asian soybean resistance. The protein and the coding gene thereof can be used to protect plants from rust pathogens.

In certain embodiments, the proteins are encoded by the nucleic acid sequence of SEQ ID NO: 2. In other embodiments, the proteins have at least 75%, at least 85%, at least 90%, at least, at least 95%, at least 97%, at least 98%, or at least 99% identical to a protein encoded by the nucleic acid sequence of SEQ ID NO: 1.

In other embodiments, the proteins are encoded by a chromosomal interval of comprising the nucleic acid sequence of SEQ NO: 11, 12, or 13. In other embodiments, the proteins have at least 75%, at least 85%, at least 90%, at least, at least 95%, at least 97%, at least 98%, or at least 99% identical to a protein encoded by a chromosomal interval of comprising the nucleic acid sequence of SEQ NO: 11, 12, or 13.

In certain embodiment, the protein of the instant disclosures is derived from soybean and named as RppRC1. In one embodiment of the invention, the protein has the amino acid sequence of SEQ ID NO: 1. In another embodiment of the invention, the protein has an amino acid sequence at least 75%, at least 85%, at least 90%, at least, at least 95%, at least 97%, at least 98%, or at least 99% identical to the amino acid sequence of SEQ ID NO: 2. In one embodiment, the protein of the instant disclosure can be any one of the following proteins:
(A1) a protein having the amino acid sequence shown in SEQ ID NO: 1;
(A2) a protein having substitution and/or deletion and/or addition of one or several amino acid residues from and having the same function as the amino acid sequence shown in SEQ ID NO: 1;
(A3) a protein having more than 99%, more than 95%, more than 90%, more than 85%, or more than 80% homology with and having the same function as the amino acid sequence defined in either (A1) or (A2); and
(A4) a fusion protein obtained by tagging at the N-terminus and/or C-terminus of the protein defined in any one of (A1) to (A3).

In another embodiment, the protein of the instant disclosure can be any one of the following proteins:
(A5) a protein having the amino acid sequence encoded by a chromosomal interval of comprising the nucleic acid sequence of SEQ NO: 11, 12, or 13;
(A6) a protein having substitution and/or deletion and/or addition of one or several amino acid residues from and having the same function as the amino acid sequence encoded by a chromosomal interval of comprising the nucleic acid sequence of SEQ NO: 11, 12, or 13;
(A7) a protein having more than 99%, more than 95%, more than 90%, more than 85%, or more than 80% homology with and having the same function as the amino acid sequence defined in either (A5) or (A6); and
(A8) a fusion protein obtained by tagging at the N-terminus and/or C-terminus of the protein defined in any one of (A5) to (A7).

In these proteins, the tag refers to a polypeptide or protein which is fused and expressed together with the protein of interest by using DNA in vitro recombination technology, so as to facilitate the expression, detection, tracing and/or purification of the protein of interest. The tag may be a FLAG tag, a His tag, an MBP tag, an HA tag, a myc tag, a GST tag, and/or a SUMO tag, etc.

In these proteins, identity refers to the identity between amino acid sequences. Homology retrieval websites on the Internet can be used to determine the identity between amino acid sequences, such as the BLAST web page on the NCBI homepage website. For example, the identity value (%) can be obtained in advanced BLAST2.1 by using blastp as the program, setting the Expect value to 10, setting all Filters to OFF, using BLOSUM62 as the Matrix, setting Gap existence cost, Per residue gap cost, and Lambda ratio to 11, 1, and 0.85 (default values), respectively, and retrieving the identity of a pair of amino acid sequences for calculation.

The proteins of the present invention can be produced from the nucleic acid molecules disclosed herein or by using standard molecular biology techniques.

The present invention encompasses an isolated or substantially purified protein. The “isolated” or “purified” protein or a biologically active portion thereof is substantially or largely free of components concomitant with or interacting with the protein that are normally present in the natural environment of the protein. The protein that is substantially free of cellular materials include protein formulations having less than about 30%, about 20%, about 10%, about 5%, or about 1% (by dry weight) of contaminating proteins. When the protein or the biologically active portion thereof in the embodiments are produced by recombinant methods, most preferably, the medium has less than about 30%, about 20%, about 10%, about 5% or about 1% (by dry weight) of chemical precursors or chemicals that are not proteins of interest. Fragments and variants related proteins are within the scope of the present disclosure.

Variant proteins encompassed by the present invention are bioactive, that is, they continue to possess the required bioactivity (i.e. the ability to enhance plant resistance (i.e. plant resistance against fungal pathogens) as described in the present invention) of native proteins. Such variants can be obtained, for example, by genetic polymorphism or by human manipulation. Bioactive variants of the native protein of the present invention may have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity with the amino acid sequence of the native protein or to SEQ ID NO: 1, as determined by sequence alignment programs known in the art. The biologically active variants of the protein disclosed in the present invention may differ from the protein by as little as about 1 to 15 amino acid residues, as little as about 1 to 10 (e.g., about 6 to 10), as little as about 5, as little as 4, 3, 2 or even 1 amino acid residue.

The proteins disclosed in the instant application may be modified, for example, by including amino acid substitution, deletion, truncation, and insertion. Methods of such manipulation are known in the art. For example, amino acid sequence variants and fragments of resistant proteins can be prepared by mutating in DNA. Methods of mutagenesis and polynucleotide modification are known in the art.

In one embodiment, the protein of the invention is a biologically active fragment of SEQ ID NO: 1, which can protect plants from rust pathogens.

The proteins disclosed in the present invention also encompass naturally occurring proteins and variants, fragments, and modified forms thereof. Such variants and fragments will still have the required ability to confer or enhance plant resistance against fungal pathogens.

### Nucleic Acid Molecules

Another embodiment of the invention is directed to nucleic acid molecules relating to rust resistance. These nucleic acid molecules encode a protein of the invention, i.e. a protein conferring increased resistance to rust such as e.g. Asian soybean rust. The nucleic acid molecule may be DNA, such as cDNA, genomic DNA, or recombinant DNA. The nucleic acid molecule may also be RNA, such as mRNA.

In certain embodiments, the nucleic acid molecule is the gene RppRC1 (named as RppRC1). In one embodiment of the invention, the nucleic acid molecule has the nucleic acid sequence of SEQ ID NO: 2. In another embodiment, the nucleic acid molecule has a nucleic acid sequence at least 75%, at least 85%, at least 90%, at least, at least 95%, at least 97%, at least 98%, or at least 99% identical to the nucleic acid sequence of SEQ ID NO: 2. Embodiments of the nucleic acid molecules of the instant disclosure include:
(B1) a DNA molecule having the nucleic acid sequence of SEQ ID NO: 2;
(B2) a DNA molecule hybridizing to the nucleic acid sequence of SEQ ID NO: 2 under a stringent condition and encoding the protein described above; and
(B3) a DNA molecule having more than 99%, more than 95%, more than 90%, more than 85%, or more than 80% homology with the DNA sequences defined in (B1) and (B2) and encoding the protein described above.

In other embodiments, the nucleic acid molecule comprises a chromosomal interval comprising the nucleic acid sequence of SEQ ID NO: 11, 12, or 13, or a portion thereof encoding ASR resistance. In another embodiment, the nucleic acid molecule has a nucleic acid sequence at least 75%, at least 85%, at least 90%, at least, at least 95%, at least 97%, at least 98%, or at least 99% identical to a chromosomal interval comprising the nucleic acid sequence of SEQ ID NO: 11, 12, or 13. In certain embodiments, the nucleic acid molecules encode ASR resistance. Embodiments of such nucleic acid molecules include:
(B4) a DNA molecule having the nucleic acid sequence of SEQ ID NO: 11, 12, or 13 or portion thereof encoding ASR resistance;
(B5) a DNA molecule hybridizing to the nucleic acid sequence of SEQ ID SEQ ID NO: 11, 12, or 13 under a stringent condition and encoding the protein described above; and
(B6) a DNA molecule having more than 99%, more than 95%, more than 90%, more than 85%, or more than 80% homology with the DNA sequences defined in (B4) and (B4) and encoding a protein conferring increased rust resistance to a plant.

As for the above genes, the stringent condition may be as follows: hybridizing at 50° C. in a mixed solution of 7% sodium dodecyl sulfate (SDS), 0.5 M Na₃PO₄ and 1 mM EDTA, and rinsing at 50° C. in 2×SSC, 0.1% SDS; the stringent condition may also be: hybridizing at 50° C. in a mixed solution of 7% SDS, 0.5 M Na₃PO₄ and 1 mM EDTA, and rinsing at 50° C. in 1×SSC, 0.1% SDS; the stringent condition may also be: hybridizing at 50° C. in a mixed solution of 7% SDS, 0.5 M Na₃PO₄ and 1 mM EDTA, and rinsing at 50° C. in 0.5×SSC, 0.1% SDS; the stringent condition may also be: hybridizing at 50° C. in a mixed solution of 7% SDS, 0.5 M Na₃PO₄ and 1 mM EDTA, and rinsing at 50° C. in 0.1×SSC, 0.1% SDS; the stringent condition may also be: hybridizing at 50° C. in a mixed solution of 7% SDS, 0.5 M Na₃PO₄ and 1 mM EDTA, and rinsing at 65° C. in 0.1×SSC, 0.1% SDS; the stringent condition may also be: hybridizing at 65° C. in a solution of 6×SSC and 0.5% SDS, and then washing the membrane once with 2×SSC and 0.1% SDS, and once with 1×SSC and 0.1% SDS, respectively.

In another embodiment, the nucleic acid molecule encodes the amino acid of SEQ ID NO: 1 or a protein having an amino acid sequence at least 75%, at least 85%, at least 90%, at least, at least 95%, at least 97%, at least 98%, or at least 99% identical to the amino acid sequence of SEQ ID NO: 1.

The present invention encompasses an isolated or substantially purified nucleic acid molecule. The “isolated” or “purified” nucleic acid molecule or a biologically active portion thereof is substantially or largely free of components concomitant with or interacting with the nucleic acid molecule that are normally present in the natural environment of the nucleic acid molecule. Thus, the isolated or purified nucleic acid molecule or protein is substantially free of other cellular materials or media when produced by recombinant techniques (such as PCR amplification), or chemical precursors or other chemicals when synthesized by chemical methods. Most preferably, the “isolated” nucleic acid molecule does not comprise sequences (e.g., protein coding sequences) that are naturally located flanking the nucleic acid molecule (i.e. sequences located at the 5′ and 3′ ends of the nucleic acid molecule) in the genomic DNA of the organism from which the nucleic acid molecule is derived. For example, in some embodiments of the present invention, the isolated nucleic acid molecule may comprise less than about 5 kb, about 4 kb, about 3 kb, about 2 kb, about 1 kb, about 0.5 kb, or about 0.1 kb of nucleotide sequences that are naturally located flanking the nucleic acid molecule in the genomic DNA of the cell from which the nucleic acid molecule is derived. Fragments and variants related to coded nucleotide sequences are within the scope of the present disclosure. “A fragment” and grammatical variations thereof refer to a portion of a nucleotide sequence or a portion of an amino acid sequence and a protein coded thereby. Fragments of the nucleotide sequence can encode protein fragments that retain the biological activity of natural proteins and have the ability to confer resistance (i.e. antifungal) in plants. Alternatively, nucleotide sequence fragments that can be used as hybridization probes do not necessarily code protein fragments that maintain biological activity. Thus, the fragment of the nucleotide sequence may be in the range of at least about 15 nucleotides, about 50 nucleotides, about 100 nucleotides and at most the full-length nucleotide sequence coding the protein disclosed herein.

The fragment of the nucleotide sequence coding the biologically active portion of the disclosed protein may code at least about 15, about 25, about 30, about 40, or 45, about 50 consecutive amino acids or at most the total number of amino acids present in the full-length protein of this embodiment (e.g., 857 amino acids for SEQ ID NO: 1). Fragments of nucleotide sequences that can be used as hybridization probes or PCR primers usually do not have to code biologically active portions of proteins.

When referring to a specified nucleic acid molecule, the term “full-length sequence” refers to the entire nucleic acid sequence of a native sequence. “A native sequence” and grammatical variations thereof are use in the present invention to refer to an endogenous sequence, i.e. an unengineered sequence present in the genome of an organism.

Therefore, the fragment of the nucleotide sequence disclosed in the present invention can code a biologically active portion of a protein, or it can be a fragment used as a hybridization probe or PCR primer. In certain embodiments, the nucleic acid molecule of the present invention comprises at least about 15, about 20, about 50, about 75, about 100, or about 150 nucleotides or at most the number of nucleotides present in the full-length nucleotide sequence disclosed herein (e.g., 2574 nucleotides for SEQ ID NO: 2).

Those skilled in the art will recognize that the nucleic acid variants of the present invention will be configured such that the open reading frame is maintained. For nucleic acid molecules, conserved variants comprise those sequences that code the amino acid sequences in the proteins of the present invention due to degeneracy of the genetic code. Native allelic variants can be identified by well-known molecular biological techniques, such as polymerase chain reaction (PCR) and hybridization techniques. Variant nucleic acid molecules also comprise synthetic nucleic acid molecules, such as those generated by using site-directed mutagenesis but still coding the proteins of the present invention. Generally, variants of a particular nucleic acid molecule disclosed herein may have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity with the particular nucleic acid molecule, as determined by sequence alignment programs well known in the art.

Variants of a particular nucleic acid molecule (i.e. a reference nucleic acid molecule) of the present invention can also be evaluated by comparing the percentage of sequence identity between the protein coded by the variant nucleic acid molecule and the protein coded by the reference nucleic acid molecule. The percentage of sequence identity between any two proteins can be calculated using sequence alignment programs known in the art. In the case where any given pair of nucleic acid molecules of the present invention is evaluated by comparing the percentage of sequence identity shared by the two proteins coded by the given pair of nucleic acid molecules, the percentage of sequence identity between the two coded proteins is at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or more sequence identity.

Variant nucleic acid molecules and proteins also encompass sequences and proteins obtained by mutagenesis and recombination procedures, including but not limited to procedures such as DNA shuffling. A library of recombinant polynucleotides can be generated from a group of related sequence polynucleotides comprising sequence regions having substantial sequence identity and capable of homologous recombination in vitro or in vivo. For example, using this method, a sequence motif coding a domain of interest can be shuffled between the protein gene disclosed in the present invention and other known protein genes to obtain a new gene coding a protein having improved properties of interest, such as an increased ability to confer or enhance resistance of plants to fungal pathogens. Such DNA shuffling strategies are known in the art.

The present disclosure encompasses sequences that are isolated based on their sequence identity with the entire sequence shown herein or the variants and fragments thereof. Such sequences include sequences that are orthologues of the disclosed sequences. Genes present in different species are considered to be orthologues when their nucleotide sequences and/or protein sequences coded thereby share at least about 60%, about 70%, about 75%, about 80%, about 85%, about 90%, about 91%, about 92%, about 93%, about 94%, about 95%, about 96%, about 97%, about 98%, about 99% or more sequence identity. The function of orthologues is often highly conserved in various species. Therefore, the present disclosure encompasses isolated nucleic acid molecules that code proteins that confer or enhance fungal plant pathogen resistance and hybridize with the sequences disclosed in the present invention or variants or fragments thereof.

In PCR methods, oligonucleotide primers can be designed for PCR reactions to amplify corresponding DNA sequences from cDNA or genomic DNA extracted from any organism of interest. Methods for designing PCR primers and for cloning by PCR are known in the art and are disclosed in the following documents: Sambrook et al., (1989) Molecular Cloning: A Laboratory Manual (2nd ed., Cold Spring Harbor Laboratory Press, Plainview, N.Y.). Known PCR methods include, but are not limited to, methods using paired primers, nested primers, single-specific primers, degenerate primers, gene-specific primers, vector-specific primers, partially mismatched primers, etc.

In hybridization techniques, all or part of a known nucleic acid molecule is used as a probe that selectively hybridizes with other corresponding polynucleotides present in a set of cloned genomic DNA fragments or cDNA fragments (i.e. a genomic or cDNA library) from a selected organism. The hybridization probe may be a genomic DNA fragment, a cDNA fragment, an RNA fragment, or other oligonucleotide and may be labeled with detectable groups such as ³²P or any other detectable markers. Therefore, for example, the hybridization probe can be prepared by labeling the synthetic oligonucleotide based on the polynucleotide of this embodiment. Methods for preparing hybridization probes and constructing cDNA libraries and genomic libraries are known in the art.

Various procedures can be used to determine the presence or absence of specific sequences of DNA, RNA, or protein. These include, for example, Southern blot, Northern blot, Western blot, and ELISA analysis. These techniques are well known in the art.

The protein and the coding gene thereof and methods disclosed in the present invention can be used to regulate the content of one or more proteins in plants. The term “regulate” and grammatical variations thereof are used in the present invention to refer to an increase or decrease in the protein content in genetically modified (i.e. transformed) plants relative to the protein content from the corresponding untransformed plants (i.e. plants that have not been genetically modified according to the methods of the present disclosure).

As used herein, the term “expression” and grammatical variations thereof refer to the biosynthesis or process whereby a polynucleotide is generated, including the transcription and/or translation of a gene product. For example, the nucleic acid molecules disclosed in the present invention may be transcribed from DNA templates (such as into mRNA or other RNA transcripts) and/or the transcribed mRNA is subsequently translated into proteins. The term “gene product” and grammatical variations thereof may refer to, for example, transcripts and coded proteins. The inhibition (or increase) of the expression or function of the gene product (i.e. the gene product of interest) may be in an environment in which comparisons are made between any two plants, For example, the expression or function of a gene product in genetically modified plants is relative to the expression or function of the gene product in corresponding but susceptible wild-type plants or other susceptible plants. The expression level of a gene product in a wild-type plant may not exist. For example, a “wild type” plant may be a plant, a plant cell or a plant part that does not express an exogenous resistance gene.

Alternatively, the inhibition (or increase) of the expression or function of the target gene product may be in an environment in which comparisons are made between plant cells, organelles, organs, tissues or plant parts within the same plant or different plants and include comparisons between developmental or temporal stages of the same plant or different plants. Any method or composition that downregulates the expression of the target gene product or downregulates the functional activity of the target gene product at the transcription or translation level can be used to achieve inhibition of the expression or function of the target gene product. Similarly, any method or composition that induces or upregulates the expression of the target gene product at the transcription or translation level, or increases or activates or upregulates the functional activity of the target gene product, may be used to achieve increased expression or function of the target gene or protein. Methods for inhibiting or enhancing gene expression are well known in the art.

Genes and nucleic acid molecules disclosed in the present invention include naturally occurring sequences and mutants or modified forms thereof. The proteins disclosed in the present invention also encompass naturally occurring proteins and variants, fragments, and modified forms thereof. Such variants and fragments will still have the required ability to confer or enhance plant resistance against fungal pathogens. In one embodiment, mutations to be made in the DNA coding the variants or fragments generally do not place the sequence outside the reading frame and, preferably, will not produce complementary regions, which may produce secondary mRNA structures.

### Expression Cassettes, Recombinant Vectors, Recombinant Bacteria, or Transgenic Cell Lines

The instant application also provides an expression cassette, a recombinant vector, a recombinant bacterium, or a transgenic cell line containing the nucleic acid molecule described above.

The expression cassette refers to DNA capable of expressing the protein in a host cell, and the DNA not only includes a promoter that initiates transcription of the gene encoding the protein, but also includes a terminator that terminates the transcription. Furthermore, the expression cassette may further include an enhancer sequence. Promoters that may be used in the present invention include, but are not limited to: constitutive promoters, tissue-, organ- and development-specific promoters and inducible promoters. Examples of promoters include, but are not limited to: constitutive promoter 35S of cauliflower mosaic virus; a trauma inducible promoter from tomato, leucine aminopeptidase (“LAP”, Chao et al., (1999) Plant Physiol 120: 979-992); a chemically inducible promoter from tobacco, pathogenesis related 1 (PR1) (induced by salicylic acid and BTH (benzothiadiazole-7-thiohydroxyacid S-methyl ester)); tomato protease inhibitor II promoter (PIN2) or LAP promoter (both induced by methyl jasmonate); heat shock promoter; tetracycline inducible promoter; seed-specific promoters, such as millet seed-specific promoter pF128, seed storage protein-specific promoters (e.g., phaseolin, napin, oleosin, and soybean beta conglycin promoters (Beachy et al., (1985) EMBO J. 4: 3047-3053)). They can be used alone or in combination with other plant promoters. Suitable transcription terminators include, but are not limited to: Agrobacterium nopaline synthase terminator (NOS terminator), cauliflower mosaic virus CaMV 35S terminator, tml terminator, pea rbcS E9 terminator and nopaline and octopine synthase terminator (see, for example: Odell et al., (I⁹⁸⁵) Nature, 313: 810; Rosenberg et al., (1987) Gene, 56: 125; Guerineau et al., (1991) Mol. Gen. Genet, 262: 141; Proudfoot (1991) Cell, 64: 671; Sanfacon et al., Genes Dev., 5: 141; Mogen et al., (1990) Plant Cell, 2: 1261; Munroe et al., (1990) Gene, 91: 151; Ballad et al., (1989) Nucleic Acids Res., 17: 7891; Joshi et al., (1987) Nucleic Acid Res., 15: 9627).

Constructing a recombinant expression vector containing the nucleic acid molecule. The plant expression vectors used can be Gateway system vectors or binary Agrobacterium vectors, such as pGWB411, pGWB412, pGWB405, pBin438, pCAMBIA1302, pCAMBIA2301, pCAMBIA1301, pCAMBIA1300, pBI121, pCAMBIA1391-Xa or pCAMBIA1391-Xb.

When using RppRC1 to construct a recombinant expression vector, any enhanced, constitutive, tissue-specific or inducible promoter can be added before the transcription initiation nucleotide, such as cauliflower mosaic virus (CAMV) 35S promoter, ubiquitin gene Ubiquitin promoter (pUbi), etc., which can be used alone or in combination with other plant promoters. In addition, when using the gene and/or chromosome intervals of the present invention to construct a plant expression vector, enhancers, including translation enhancers or transcription enhancers, can also be used, and these enhancer regions can be ATG start codons or regions adjacent to start codons, etc., but must be in the same reading frame as the coding sequence to ensure the correct translation of the entire sequence. The sources of the translation control signals and start codons are extensive and can be natural or synthetic. The translation initiation region may be from a transcription initiation region or a structural gene.

In order to facilitate identification and screening of transgenic plant cells or plants, the plant expression vector can be engineered, for example, by adding a gene (GUS gene, luciferase gene, etc.) which can be expressed in a plant and encode an enzyme which can produce a color change or a luminescent compound, an antibiotic marker having resistance (gentamicin marker, kanamycin marker, etc.), or a marker gene having resistance against a chemical reagent (such as a herbicide-resistant gene), etc.

The transgenic cell line can be either a propagating material or a non-propagating material.

In a specific embodiment, the promoter for initiating the transcription of the coding gene of the protein in the expression cassette is specifically the original endogenous promoter of the coding gene, and the nucleotide sequence of the original endogenous promoter of the coding gene is shown in SEQ ID NO: 7.

In another embodiment of the invention, the recombinant vector is specifically a recombinant plasmid obtained by cloning the nucleic acid molecule (SEQ ID NO: 2) described above between the attR1 and attR2 sites of pB2GW7 vector, and replacing the 35S promoter between the SacI and SpeI enzyme digestion sites with the endogenous promoter of RppRC1 gene shown in SEQ ID NO: 7.

The resistance gene disclosed in the present invention can be expressed as a transgene to produce a rust-resistant plant. The use of different promoters described in the present invention or known to those skilled in the art will cause gene expression to be regulated under different conditions (i.e. promoters can be selected based on desired results). For example, a higher level of expression in a particular tissue system or organ (e.g., leaves) may be required to enhance resistance. The entire gene (e.g., both the natural promoter and the coding sequence) can be inserted as a transgene, thus allowing rapid combination with other traits such as insect resistance or herbicide resistance.

In some embodiments, nucleic acid sequences can be superimposed with any combination of nucleic acid molecular sequences of interest to form plants having desired phenotypes. This superposition can be achieved by a combination of genes in a DNA construct, or by hybridizing one or more plants with transgenes with another plant strain comprising a desired combination. For example, the nucleic acid molecules or fragments thereof disclosed in the present invention can be superimposed with any other nucleic acid molecules or other genes. The resulting combination can also include multiple copies of any one of the nucleic acid molecules of interest. The nucleic acid molecules disclosed in the present invention may also be superimposed with any other gene or combination of genes to produce a plant having a desired combination of a plurality of traits. The traits include, but are not limited to, traits desired as animal feeds, such as high oil genes, balanced amino acids, increased digestibility, insect resistance, disease resistance or herbicide resistance, non-toxicity and disease resistance genes, agronomic traits (e.g., male sterility, flowering time) and/or transformation technology traits (e.g., cell cycle regulation or gene targeting).

Any method, including but not limited to cross breeding of plants by any conventional or known method or genetic transformation, can be used to gather different genes. If the traits are stacked by genetically transformed plants, the polynucleotide sequences of interest can be combined in any order at any time. For example, transgenic plants comprising one or more desired traits can be used as targets to introduce more traits through subsequent transformation. In the co-transformation scheme, traits can be introduced simultaneously with the polynucleotide of interest, which is provided by any combination of transformation cassettes. For example, if two sequences are to be introduced, the two sequences can be comprised in separate transformation cassettes (trans) or in the same transformation cassette (cis). Expression of the sequences can be driven by the same promoter or different promoters. In some cases, it is desirable to introduce a transformation cassette that can inhibit the expression of a nucleic acid molecule of interest. This can be combined with any combination of other inhibition cassettes or overexpression cassettes to generate a desired combination of traits in plants.

The constructed vector or expression cassette does not exist in the genome of the initial plant or the genome of the transgenic plant and is not located at the native locus in the genome of the initial plant.

The compositions disclosed in the present invention can be produced or maintained by a method for gene introgression. Gene introgression is sometimes referred to as “backcross” when that method is repeated two or more times. In gene introgression or backcross, “donor” parents refer to parent plants with required genes or loci to be introgressed. “Recipient” parents (used once or more) or “recurrent” parents (used twice or more) refer to parent plants in which genes or loci are introgressed. Initial hybridization produces F1 generation. The term “BC1” refers to the second use of the recurrent parents, and “BC2” refers to the third use of the recurrent parents, and so on.

The present invention may also include the described sequences which may be provided from an expression cassette or DNA construct expressed in plants of interest. The expression cassette may include 5′ and 3′ heterologous regulatory sequences operatively linked to the sequences disclosed in the present invention. The term “operatively linked” is used in the present invention to mean that a nucleic acid to be expressed is linked to a regulatory sequence, including a promoter, a terminator, an enhancer and/or other expression control elements (e.g., polyadenylation signals) in a manner that allows the expression of the nucleic acid (i.e. when a vector is introduced into a host plant cell, the nucleic acid is expressed in the host plant cell). Such regulatory sequences are well known in the art and include those nucleotide sequences that can be directly constitutively expressed in a variety of host cells and directly expressed in specific host cells or under specific conditions. The design of the vector may depend on, for example, the type of host cells to be transformed, or the desired expression level of nucleic acids. The expression cassette may comprise one or more additional genes to be co-transformed into the plants. Moreover, any additional gene can be provided in a plurality of expression cassettes.

The expression cassette of the present invention may comprise a plurality of restrictive enzyme digestion sites for insertion of the nucleotide sequence so as to be under the transcriptional regulation of a regulatory region. The expression cassette may also comprise selective marker genes.

The expression cassette may also comprise, in the 5′-3′ transcription direction, a transcription and translation initiation region, a DNA sequence of the present disclosure, and a transcription and translation termination region that function in plants. The transcription initiation region, a promoter, may be native or similar or foreign or heterologous relative to plant hosts. In addition, the promoter may be a native sequence or alternatively a synthetic sequence. The term “heterologous” means that the initial transcription region does not exist in the native plant into which the initial transcription region is introduced. As used herein, a chimeric gene comprises a coding sequence operatively linked to a transcription initiation region which is heterologous to the coding sequence. Examples of promoters include, but are not limited to, cauliflower mosaic virus 35S and soybean ubiquitin 6.

Although heterologous promoters may preferably be used to express sequences, homologous promoters or native promoter sequences may be used. Such constructs will alter the level of expression in host cells (i.e. plants or plant cells). Therefore, the phenotypes of the host cells (i.e. the plant or plant cell) are changed.

The termination region may naturally have a transcription initiation region, naturally have an operatively linked DNA sequence of interest, or originate from another source. A readily available termination region (such as octopine synthase and nopaline synthase termination regions) can be obtained from the Ti plasmid of Agrobacterium tumefaciens.

Endogenous or source gene resistant orthologue can be altered by a homologous or non-homologous recombination method, such as, for example, by genome editing. When compared to an unmodified sequence, such alteration means that the nucleotide sequence has at least one modification and includes, for example: (i) replacement of at least one nucleotide, (ii) deletion of at least one nucleotide, (iii) insertion of at least one nucleotide, or (iv) any combination of (i)-(iii).

In some embodiments, genome editing techniques may be used to introduce the resistance genes disclosed in the present invention into the genome of a plant, or genome editing techniques may be used to edit resistance genes previously introduced into the genome of a plant.

Genome editing can be implemented using any available gene editing method. For example, gene editing can be achieved by introducing a polynucleotide modification template (sometimes referred to as a gene repair oligonucleotide) into a host cell, wherein the polynucleotide modification template comprises targeted modifications of genes within the genome of the host cell. The polynucleotide modification template can be single-stranded or double-stranded.

One or more genes may be optimized as desired to increase expression in transformed plants. For example, plant-preferred codons are used to synthesize genes to improve expression. Methods for synthesizing a plant-preferred gene are known in the art.

Additional sequence modifications are known to enhance the gene expression in a cell host. These sequence modifications include the elimination of the following sequences: coded pseudo-polyadenylation signals, exon-intron splicing site signals, transposon-like repeat sequences, and other such fully characterized sequences that may be harmful to gene expression. The G-C content in a sequence can be adjusted to the average level of a given cell host, which level can be calculated from known genes expressed in the host cell. The sequence can be modified if necessary, to avoid a possible hairpin secondary mRNA structure.

An expression cassette may additionally comprise a 5′ leader sequence in a construct of the expression cassette. Such a leader sequence can enhance translation. Translation leader sequences are known in the art and include: small ribonucleic acid virus leader sequence, such as EMCV leader sequence (encephalomyocarditis 5′ non-coding region); potato y virus group leader sequence, such as TEV leader sequence (tobacco etch virus), and human immunoglobulin heavy chain binding protein (BiP); untranslated leader sequence of coat protein mRNA (AMVRNA 4) from alfalfa mosaic virus; tobacco mosaic virus (TMV) leader sequence; as well as maize chlorotic mottle virus (MCMV) leader sequence (Lommel et al., (1991) Virology 81: 382-385). Other known methods for enhancing translation, such as introns, may also be utilized.

Various DNA fragments in an expression cassette can be manipulated in appropriate reading frames according to needs to ensure that DNA sequences are in the correct direction. To this end, adapters or linkers can be used to link DNA fragments. In addition, other manipulations can also be used to provide convenient restriction sites, remove excess DNA, or remove restriction sites. For this purpose, in vitro mutagenesis, primer repair, restriction, annealing, and re-replacement (e.g., conversion and transversion) may be involved.

Generally, an expression cassette may comprise selective marker genes for selecting transformed cells. The selective marker genes are used to select transformed cells or tissues. Marker genes include genes encoding antibiotic resistance, such as genes encoding neomycin phosphotransferase II (NEO) and hygromycin phosphotransferase (HPT), as well as genes conferring resistance against herbicidal compounds such as glufosinate, phosphinothricin, bromoxynil, imidazolinone and 2,4-dichlorophenoxyacetic acid (2,4-D). The above list of selective marker genes is not meant to be limiting. Any selective marker gene can be used in the present disclosure.

In order to express the target gene and/or protein of the present invention in a plant or plant cell, the method of the present invention comprises transforming the plant or plant cell with a nucleic acid molecule coding the target protein. The nucleic acid molecule of the present invention can be operatively linked to a promoter which drives expression in a plant cell. Any promoter known in the art can be used in the method of the present invention, including, but not limited to, constitutive promoters, pathogen inducible promoters, wound inducible promoters, tissue-preferred promoters, and chemically regulated promoters. The selection of the promoter may depend on the desired expression time and location in a transformed plant, as well as other factors known to those skilled in the art. Transformed cells or plants may be planted or cultivated to form a plant comprising one or more of polynucleotides introduced, for example, into cells or plants coding R proteins.

A variety of promoters can be used to put into practice the present invention. The promoters can be selected according to the desired result. That is, a nucleic acid can be combined with a constitutive promoter, a tissue-preferred promoter, or other promoters and expressed in a host cell of interest. Such constitutive promoters include, for example, the core promoter of the Rsyn7 promoter and other constitutive promoters disclosed in WO 99/43838 and U.S. Pat. No. 6,072,050, CaMV 35S promoter, rice actin, Ubiquitin, pEMU, MAS, ALS, etc. Such constitutive promoters are known in the art and are contemplated for use in the present disclosure.

Generally, using inducible promoters, especially those from pathogens, to express the gene of the present invention is beneficial to the application of the gene of the present invention. Such promoters include the promoters from pathogenesis-related proteins (PR proteins), which proteins are induced to form by pathogen infection and are, for example, PR proteins, SAR proteins, β-1,3-glucanase, chitosanases, etc.

Promoters expressed locally at or near the pathogen infection site deserve attention. In addition, because pathogens can enter plants through wounds or insect-caused lesions, wound inducible promoters can also be used for vector construction of the present invention. Such wound inducible promoters include potato protease inhibitor (pinII) gene, wun1 and wun2, win1 and win2, systemin, WIP1, MPI gene, etc.

Chemically regulated promoters can regulate gene expression in plants by applying exogenous chemical regulatory agents. According to the purpose of utilization, the promoters may be chemically inducible promoters, such as inducing gene expression by applying chemicals, or chemically repressible promoters, such as inhibiting gene expression by applying chemicals. Chemically inducible promoters are known in the art and include, but are not limited to, the maize In2-2 promoter (which is activated by benzenesulfonamide herbicide safeners), the maize GST promoter (which is activated by hydrophobic electrophilic compounds used as pre-emergence herbicides) and the tobacco PR-1a promoter (which is activated by salicylic acid). Other chemically regulated promoters of interest include steroid responsive promoters (for example, glucocorticoid inducible promoters, tetracycline inducible promoters and tetracycline repressible promoters).

Tissue-preferred promoters can be used for targeted enhanced expression of target genes or proteins (e.g., polynucleotide sequences coding NB-LRR polypeptides derived from leguminous plants) in specific plant tissues. Preferred promoters for such tissues include, but are not limited to, leaf-specific promoters, root-specific promoters, seed-specific promoters, and stem-specific promoters. Tissue-specific promoters include Yamamoto et al., (1997) Plant J. 12 (2): 255-265; Kawamata et al., (1997) Plant Cell Physiol. 38 (7): 792-803; Hansen et al., (1997) Mol. Gen Genet. 254 (3): 337-343; Russell et al., (1997) Transgenic Res. 6 (2): 157-168; Rinehart et al., (1996) Plant Physiol. 112 (3): 1331-1341; Van Camp et al., (1996) Plant Physiol. 112 (2): 525-535; Canevascini et al., (1996) Plant Physiol. 112 (2): 513-524; Yamamoto et al., (1994) Plant Cell Physiol. 35 (5): 773-778; Lam, (1994) Results Probl. Cell Differ. 20: 181-196; Orozco et al., (1993) Plant Mol Biol. 23 (6): 1129-1138; Matsuoka et al., (1993) Proc Natl. Acad. Sci. USA, 90 (20): 9586-9590; and Guevara-Garcia et al., (1993) Plant J. 4 (3): 495-505. Such promoters may be used to modify the nucleotide sequences of the present disclosure.

Leaf-specific promoters are known in the art. See, for example, Yamamoto et al., (1997) Plant J. 12 (2): 255-265; Kwon et al., (1994) Plant Physiol. 105: 357-67; Yamamoto et al., (1994) Plant Cell Physiol. 35 (5): 773-778; Gotor et al., (1993) Plant J. 3: 509-18; Orozco et al., (1993) Plant Mol. Biol. 23 (6): 1129-1138; and Matsuoka et al., (1993) Proc. Natl. Acad. Sci. USA, 90 (20): 9586-9590.

“Seed-preferred” promoters include “seed-specific” promoters (those that are active during seed development, such as promoters of seed storage protein) and “seed germination” promoters (those that are active during seed germination). Such seed-preferred promoters include, but are not limited to, Cim1 (cytokinin induction information), mi1ps (inositol-1-phosphate synthase) and celA (cellulose synthase). Globin-1 (Glob-1) is the preferred embryo-specific promoter. For dicotyledon, seed-specific promoters include, but are not limited to, common bean β-phaseolin gene promoter, napin gene promoter, β-conglycinin gene promoter, soybean lectin gene promoter, cruciferae protein gene promoter, etc.

The expression of the nucleic acid molecules of the present invention may involve the use of complete native resistance genes, wherein the expression is driven by a homologous 5′ upstream promoter sequence or other heterologous promoters. Those skilled in the art will be able to identify the resistance genes to evaluate the expression level thereof and select a preferred promoter sequence that can be used to express the resistance genes of interest. The use of homologous or heterologous resistance gene promoter sequences provides options for regulating protein expression to avoid or minimize any potentially inappropriate or undesirable results related to plant defense activation.

Specific soybean promoters include, but are not limited to, promotors from soybean ubiquitin (subi-1), elongation factor 1A, S-adenosylmethionine synthase for constitutive expression, Rpp4, and RPG1-B, and promoters comprised in gene models, such as Glyma promoters known to those skilled in the art for more closely regulating the expression provided by NB-LRR gene promoters.

In the context of the present invention, germplasm includes cells, seeds or tissues from which new plants can be generated, or plant parts such as leaves, stems, pollen, or cells that can be cultivated into whole plants.

### Transforming Plants

Since the resistance gene disclosed in the present invention can be expressed as a transgene to produce a rust-resistant plant, certain embodiment of the invention are directed to methods for introducing a nucleic acid molecule into a plant. In addition to transforming soybean plants described above, the nucleic acids of the invention may be used in other plants as well.

As used herein, the term “introduction” and grammatical variations thereof refer to providing a plant with a nucleic acid molecule. In some embodiments, the nucleic acid molecule can exist in such a way that the sequence enters the interior of a plant cell, including their potential insertion into the genome of the plant. The method disclosed in the present invention does not depend on specific methods for introducing a sequence into a plant, as long as a nucleic acid molecule enters the interior of at least one cell of the plant. Methods for introducing a nucleic acid molecule into a plant are known in the art and include, but are not limited to stable transformation methods, transient transformation methods and virus-mediated methods.

Examples of methods for plant transformation include Agrobacterium-mediated transformation and particle bombardment, then the transformed plant can be regenerated by methods known to those skilled in the art.

A nucleic acid molecule can be transiently or stably introduced into a host cell and can remain unintegrated, for example, in plasmid form. “Stable transformation” or “stably transformed” and grammatical variations thereof mean that a nucleotide construct introduced into a plant is integrated into the genome of the plant and can be inherited by the progenies of the plant. As used herein, “transient transformation” and grammatical variations thereof mean that a nucleic acid molecule is introduced into a plant but not integrated into the genome of the plant, or a protein is introduced into a plant.

The transformation methods and the methods for introducing a nucleic acid molecular sequence into a plant may depend on the type of a plant or a plant cell to be transformed. Suitable methods for introducing a protein or a nucleic acid molecule into a plant cell include, but are not limited to, microinjection, electroporation, direct gene transfer, Lec1 transformation, and ballistic particle acceleration. As the updated methods become available, such methods can also be used in the present invention because the methods of transformation or transfection are not critical.

The transformed cells can be cultivated into plants according to conventional methods. These plants can then be grown and pollinated with the same transformation line or different lines, and then progenies with constitutive expression with required phenotypic characteristics can be identified. Two or more generations of plants can be cultivated to ensure that the expression of required phenotypic characteristics is stably maintained and inherited. The seeds are then harvested to ensure that the expression of the required phenotypic characteristics has been achieved. In some embodiments, transformed seeds or transgenic seeds have nucleotide constructs or expression cassettes stably incorporated into their genomes.

The present invention encompasses seeds comprising the nucleic acid molecular sequences disclosed in the present invention, the seeds can be developed into or used to develop one or more plants having enhanced resistance to pathogens (e.g., fungi) or infections formed by pathogens compared to, for example, the plant seeds of wild-type varieties. The present invention is characterized by seeds from transgenic leguminous plants, wherein the seeds comprise the nucleic acid molecules disclosed in the present invention.

Plants of interest include leguminous crop species, including but not limited to alfalfa (Medicago sativa); clover or trefoil (Trifolium spp.); pea, including Pisum satinum, Gajanus cajan, Vigna unguiculata and Lathyrus spp.; common bean (Fabaceae or Leguminosae); lentil (Lens culinaris); lupin (Lupinus spp.); ghaf tree (Prosopis spp.); long bean (Ceratonia siliqua), soybean (Glycine max), peanut (Arachis hypogaea) or tamarind (Tamarindus indica). The terms “leguminous species” and “leguminous crop species” are used herein to refer to plants and may, for example, be plants of interest. Leguminous species or leguminous crop species may be plants, plant parts or plant cells.

### Regulating and Enhancing Rust Resistance

The present invention also provides for use of the proteins, nucleic acids recombinant vectors, recombinant bacterium, or transgenic cell line described herein in regulating the resistance of plants against rust. In the use, the expression level and/or activity of the protein or the coding gene thereof in the plants is increased, and the resistance of the plants against rust is enhanced.

Furthermore, the invention is directed to a method for enhancing the resistance of plants to plant diseases such as rust. The method may include conferring resistance against pathogens (e.g., rust) by introgressing resistance genes from leguminous plants into germplasm in a breeding procedure (i.e. breeding procedure for rust resistance).

Methods of introgression as disclosed herein may also be used to produce soybean plants having increased resistance to any one of the following: soy cyst nematode, bacterial pustule, root knot nematode, frog eye leaf spot, Phytophthora, brown stem rot, nematode, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, or Myzus persicae.

In one embodiment, the method improves the resistance against rust. The method for improving the resistance of plants against rust may include increasing the expression level and/or activity of the above-mentioned proteins in the plants, wherein improving the resistance of plants against rust can not only confer rust resistance to the plants that are not originally resistant to rust, but can also further enhance the rust resistance of the plants that are originally resistant to rust. Increasing the expression level and/or activity of the proteins in the plants can not only make the plants that do not originally express the proteins express the proteins, but can also further increase the expression level and/or activity of the proteins in the plants that originally express the proteins. Furthermore, in certain embodiments, the increasing the expression level and/or activity of the proteins in the plants can be realized either by transgenic means or by sexual hybridization.

### Method for Breeding a Plant Variety

In another embodiment, the present invention provides a method for breeding a plant variety with improved resistance against rust.

The method for breeding a plant variety with improved resistance against rust may include the step of increasing the expression level and/or activity of the above-mentioned proteins in a recipient plant, wherein improving the resistance against rust can not only confer rust resistance to the plants that are not originally resistant to rust, but can also further enhance the rust resistance of the plants that are originally resistant to rust. Increasing the expression level and/or activity of the above-mentioned proteins in the recipient plant can not only make the plants that do not originally express the proteins express the proteins, but can also further increase the expression level and/or activity of the proteins in the plants that originally express the proteins.

Furthermore, increasing the expression level and/or activity of the proteins in the plant can be realized either by transgenic means or by sexual hybridization.

### Methods for Breeding

In another embodiment, the invention provides for method for breeding a transgenic plant with improved resistance against rust.

The method for breeding a transgenic plant with improved resistance against rust may include introducing a nucleic acid molecule as described herein to a recipient plant to obtain a transgenic plant which has improved resistance against rust compared with the recipient plant, wherein improving the resistance against rust can not only confer rust resistance to the plants that are not originally resistant to rust, but can also further enhance the rust resistance of the plants that are originally resistant to rust.

Furthermore, introducing the nucleic acid molecule to the recipient plant can be realized by introducing the expression cassette or the recombinant vector described above into the recipient plant.

In the above method, introducing the expression cassette or the recombinant vector into the recipient plant can particularly be: transforming plant cells or tissues by using conventional biological methods such as Ti plasmid, Ri plasmid, plant virus vector, direct DNA transformation, microinjection, electroporation, Agrobacterium-mediated transformation, etc., and culturing the transformed plant tissues into plants.

The invention is also directed to transgenic plants obtained by the breeding methods. Accordingly, in one embodiment, the invention provides a transgenic plant with improved resistance against rust obtained by breeding using the method described in the herein, or is soybean (Glycine max) SX6907, or a progeny plant comprising the nucleic acid molecule described in the se above obtained after sexual hybridization using the soybean (Glycine max) SX6907 as a parent. In one embodiment, a derivative of the soybean (Glycine max) SX6907 having the accession number CGMCC No. 17575 in the China General Microbiological Culture Collection Center may be used.

The plant herein may be a whole plant, or may be a plant cell, seed, or tissue, or a plant part such as a leaf, stem, pollen, or cell that can be cultivated into a whole plant.

### Assays and Kits

In certain embodiments, the nucleic acids or amino acid molecules of the invention can be used to assay plants for rust resistance. Accordingly, the invention also comprises a kit for the assay described herein. Proteins or nucleic acid molecules or expression cassettes, recombinant vectors or recombinant bacteria or transgenic cell lines comprising the nucleic acid molecules can also be packaged together with the instructions as components of the kit for completing the assay disclosed in the present invention. The kit of the present invention may include any combination of the proteins or nucleic acid molecules of the present invention or expression cassettes, recombinant vectors or recombinant bacteria or transgenic cell lines comprising the nucleic acid molecules and suitable instructions (written and/or provided as audio, visual or audio-visual materials). For example, the kit may also comprise a specific probe having a sequence corresponding to or complementary to a sequence having 80% to 100% sequence identity with a specific region of the transgenic event. The kit may comprise any reagent and material required to perform the assay or detection method.

Embodiment of the invention also include any of the following biological materials or applications:
(D1) a primer pair for amplifying the nucleic acid molecule described above. In certain embodiments, the primer pair is a primer pair 1 composed of two single-stranded DNA shown in SEQ ID NO: 3 and SEQ ID NO: 4, respectively, or a primer pair 2 composed of two single-stranded DNA shown in SEQ ID NO: 5 and SEQ ID NO: 6, respectively.
(D2) a probe for amplifying the nucleic acid molecule described above.
(D3) a kit comprising the primer pair and/or the probe.
(D4) a plant comprising the nucleic acid molecule described in the se above.
(D5) use of the primer pair or the probe or the kit in identifying whether the plant to be tested comprises the nucleic acid molecule described above.
(D6) use of the primer pair or the probe or the kit in identifying whether the plant to be tested has resistance against rust conferred by the nucleic acid molecule described above.
(D7) a DNA molecule, which is a DNA molecule shown in SEQ ID NO: 7, or a DNA molecule with promoter function obtained by the addition, deletion and/or substitution of one or more nucleotides from SEQ ID NO:7.
(D8) use of the DNA molecule in initiating the expression of a gene of interest. In certain embodiments, the gene of interest is the nucleic acid molecule described above.

Deposit information for soybean (Glycine max) SX6907 derivative useful in the claimed invention:

| | |
|---|---|
| Category naming: | Soybean |
| Latin name: | Glycine max |
| Depository: | China General Microbiological |
| | Culture Collection Center |
| Depository abbreviation: | CGMCC |
| Address: | No. 3, Yard 1, Beichen West Road, |
| | Chaoyang District, Beijing |
| Deposit date: | Friday, Jun. 28, 2019 |
| Registration number of | CGMCC No. 17575 |
| the deposit center: | |

### EXAMPLES

The following examples facilitate a better understanding of the invention, but do not limit the invention. The experimental methods in the following examples are conventional methods. The test materials used in the following examples, unless otherwise specified, were purchased from general biochemical reagent stores. For the quantitative tests in the following examples, three repeated tests were set, and the results were averaged.

### Example 1: Cloning of RppRC1 Gene

Soybean is an ancient tetraploid leguminous plant with self-pollination and has a genome size of about 1.1 Gbp. The response of soybean germplasm with different resistance to Phakopsora pachyrhizi is obviously different. Soybean (Glycine max) SX6907 is a rust resistance resource selected from Chinese soybean germplasm by the Oil Crops Research Institute, Chinese Academy of Agricultural Sciences. The variety is currently preserved in the China General Microbiological Culture Collection Center under the accession number CGMCC No. 17575, and the response of the variety to Phakopsora pachyrhizi is immunity.

The genome of soybean SX6907 (the source of RppRC1 gene) was used as a template for PCR amplification using primer F and primer R. The primer sequences are as follows:

F
(SEQ ID NO: 3)
5′-ATGGCAGATAGTGTTGTTGCTTTTCTGC-3′;
and
R
(SEQ ID NO: 4)
5′-TCACAGTTCATTAGAGATTTTGAGCTTACAGC-3′.

The obtained amplification product is the sequence shown in SEQ ID NO: 2. SEQ ID NO: 2 is the nucleotide sequence of the RppRC1 gene, which encodes the protein shown in SEQ ID NO: 1, which protein is named as RppRC1.

### Example 2: Transformation of Soybean with Soybean RppRC1 Gene

### 1. Construction of Recombinant Expression Vector

Taking the leaves of soybean variety SX6907 cultured under normal conditions to the growth stage of the first ternately compound leaf, extracting DNA, using the DNA as a template and amplifying by a conventional PCR method under the guidance of primer F and primer R (see Example 1 for primer sequences), performing 1% agarose gel electrophoresis detection on the PCR amplified product after the reaction completed, recovering and purifying about 2.5 kb of DNA fragment. At the same time, using the DNA as a template and amplifying by a conventional PCR method under the guidance of PF and PR to obtain 2525 bp promoter pRppRC1 fragment, and linking the fragment into the vector pB2GW7 (the vector comes from VIB-plant systems biology, website: gateway.psb.ugent.be/search) through an enzyme digestion and linking method to obtain recombinant vector pB2GW7-pRppRC1.

PF:
5′-GAGCTCAAAGGCTTTTTTGTTAAGGGAAGGT-3′
(underlined part is SacI recognition sequence);
PR:
5′- ACTAGTTTCTGTGAAACAGGAAATCTTGGGT-3′
(underlined part is SpeI recognition sequence).

Then, the RppRC1 gene (SEQ ID NO: 2) was cloned into the obtained recombinant vector pB2GW7-pRppRC1 by gateway method to obtain the recombinant vector pB2GW7-RppRC1. Sequencing confirmed that the recombinant vector pB2GW7-RppRC1 is a recombinant plasmid obtained by inserting the 2574 bp DNA fragment shown in SEQ ID NO: 2 between attR1 and attR2 sites of vector pB2GW7, and replacing the 35S promoter between SacI and SpeI enzyme digestion sites with the endogenous promoter of RppRC1 gene shown in SEQ ID NO: 7.

In the final recombinant expression vector pB2GW7-RppRC1, the promoter that initiates RppRC1 gene transcription is the original promoter pRppRC1. In addition to the above elements, the vector also comprises a spectinomycin resistance gene for bacterial selection and an herbicide resistance soybean Bar gene as a plant selective marker (FIG. 1).

### 2. Transformation of Soybean

The recombinant expression vector pB2GW7-RppRC1 comprising RppRC1 gene constructed in step 1 was transferred into Agrobacterium tumefaciens EHA105 by freeze-thaw method. Then positive transformants were selected for soybean genetic transformation.

Agrobacterium-mediated cotyledon node transformation (Paz, M. M., Martinez, J. C., Kalvig, A. B., et al., (2006) Plant Cell Report, 25, 206-213) was used for soybean transformation, and the transformation recipient was soybean variety Tianlong No. 1 (the variety was bred by the Oil Crops Research Institute, Chinese Academy of Agricultural Sciences (national examination number 2008023) and is available therefrom). The media used for soybean plant transformation and regeneration were as follows:

Agrobacterium culture medium: yeast powder 10 g/L, pancreatic protein powder 20 g/L, agar 20 g/L, and rifampicin 50 mg/L and spectinomycin 100 mg/L, as antibiotics. After sterilization, pouring the mixture of the regents into a petri dish with 9 cm diameter for use.

Agrobacterium liquid medium: yeast powder 10 g/L, pancreatic protein powder 20 g/L, and rifampicin, 50 mg/L and spectinomycin 100 mg/L, as antibiotics. Sterilizing for later use.

Co-cultivation medium: B5 medium 0.32 g/L, sucrose 30 g/L, ethanesulfonic acid, (2-(N-Morpholino) ethanesulfonic acid (MES)) 6.0 g/L, pH adjusted to 5.4; after sterilization, 6-Benzylaminopurine (6-BA) 1.67 mg/L, L-cysteine 400 mg/L, DL-Dithiothreitol (DTT) 150 mg/L and acetosyringone 200 g/L were added.

Regeneration shoot induction medium: MS medium 4.4 g/L, sucrose 30 g/L, MES 0.6 g/L, agar 8 g/L, pH adjusted to 5.8; after sterilization, 6-BA 1.67 mg/L, cefotoxin (Cef) 200 mg/L, vancomycin (Van) 50 mg/L, timentim (Tim) 100 mg/L and glufosinate 8 mg/L were added. Pouring the mixture of the regents into a petri dish with 9 cm diameter for use.

Regeneration shoot elongation medium: MS medium 4.4 g/L, sucrose 30 g/L, MES 0.6 g/L, agar 8 g/L, pH adjusted to 5.8; after sterilization, gibberellin acid (GA₃) 0.5 mg/L, Cef 200 mg/L, Van 50 mg/L, Tim 100 mg/L and glufosinate 8 mg/L were added. Pouring the mixture of the regents into a petri dish 9 cm in diameter for use.

Rooting solution: 30 mg of indolebutyric acid (IBA), dissolved in 10 ml of clear water; stored at 4° C. and diluted 1000 times when using.

The transformation process was as follows:

Seed disinfection: taking clean seeds, spreading the seeds 1-2 layers in a 9 cm petri dish, placing the petri dish in a 300 ml dryer, placing a beaker in the dryer, adding 10 ml of 3% sodium hypochlorite solution and then 10 ml of 15% hydrochloric acid solution into the beaker, covering the dryer cover for sealing for 16-20 hours, then taking out the petri dish filled with seeds, placing the petri dish on a super clean bench for about 30 minutes and blowing off chlorine, then adding about 40 ml of sterile water into the petri dish, with transformation after 8-12 hours. This step and the following operations were performed under aseptic conditions unless otherwise emphasized.

Agrobacterium preparation: Monoclonal Agrobacterium (transferred to pB2GW7-RppRC1) was taken, streaked on a petri dish with Agrobacterium culture medium containing corresponding antibiotics, 3 ml of liquid Agrobacterium culture medium was added, the petri dish was lightly rotated to allow the liquid Agrobacterium culture medium to cover the petri dish, and incubated overnight at 28° C. The next day, the lawn was washed with the co-culture medium to prepare a bacterial suspension with an OD value of about 0.8-1.2 for later use.

Explant preparation: The hypocotyl of a seed was cut vertically, and two cotyledons were evenly separated along the midline of the hypocotyl. The joint of cotyledon and hypocotyl was scratched, and the true leaf at the cotyledon node was removed. Each seed can be made into two explants.

Infection and co-cultivation: The explants were placed in the bacterial suspension to ensure that all explants were immersed in the co-cultivation medium. After 20-40 min, the bacterial liquid was removed with a pipette. Two pieces of sterile round filter paper were placed in the co-culture dish (15 cm in diameter), the diameter of the filter paper (about 13-14 cm) was slightly smaller than the diameter of the dish, and 10 ml of the co-cultivation medium was added to each dish. The infected explants were spread on the filter paper with the incision upward. The dish was sealed and incubated at 22° C. under an 18 h photoperiod for 5 days.

Regeneration shoot induction: After co-culture, the explants were cut off the elongated hypocotyls and placed in the regenerated shoot induction medium. The hypocotyl region was submerged into the medium, with 6-7 explants per dish (9 cm in diameter). The dish was incubated at 24° C. under an 18 h photoperiod. Two weeks later, the calli grown from the hypocotyls were cut off and transferred to a fresh regenerated shoot induction medium for further culture for two weeks.

Regeneration shoot elongation: The cotyledons were removed from the explants, and the new calli grown from the hypocotyls were cut off. The resulting explants were transferred to a regeneration shoot elongation medium and subcultured every two weeks. The new calli grown from the hypocotyls were removed at each transfer. When the shoots elongated to more than 3 cm, the elongated shoots (>3 cm) were cut off from the explants, and the remaining explants continued to be cultured in the regeneration shoot elongation medium.

Rooting of regeneration shoot (this step can be operated under open conditions): taking an empty dish (15 cm in diameter), placing a piece of filter paper having a diameter slightly smaller than the diameter of the dish in the dish, adding water to thoroughly soak the paper, immersing the end of the elongated shoot in 3 mg/L of IBA solution for 10-20 seconds, taking the shoot out and then wrapping the end of the shoot with a piece of absorbent paper, spreading the shoot on the soaked filter paper, covering the dish, and culturing the shoot at 24° C. under an 18 h photoperiod. The lid was opened every day for ventilation, and water was supplemented appropriately to keep the filter paper moist. When the new roots grew to 2-3 cm, the culture was transferred to soil and cultivated in the greenhouse until fruiting.

At the same time, a control (empty vector control) was set up in this experiment by introducing pB2GW7 empty vector into soybean variety Tianlong No. 1.

### 3. Identification of Transgenic Soybean

### A. PCR Identification

Partial T1 plants of 2 T0 RppRC1 transgenic soybean plants (called transformation event L1 and transformation event L2) were randomly selected (6 plants, respectively recorded as L1-1, L1-2, L1-3, L2-1, L2-2 and L2-3; L1-1, L1-2 and L1-3 were T1 individual plants of transformation event L1, and L2-1, L2-2 and L2-3 were T1 individual plants of transformation event L2), and a non-transgenic Tianlong No. 1 plant (negative) and a SX6907 plant (positive) were selected; the genomic DNA of these plants were extracted, respectively, and the target gene RppRC1 was amplified by PCR with primer F and primer R of example 1 and the size of the target product was 2574 bp; the amplified product was subjected to 1% agarose gel electrophoresis; and the plants from which a 2574 bp band was obtained were recorded as positive.

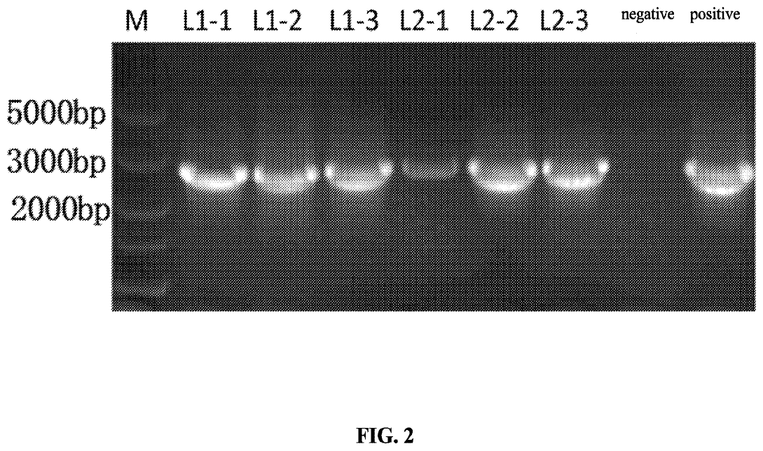

Results: L1-1, L1-2, L1-3, L2-1, L2-2 and L2-3 plants were all positive, non-transgenic plants were negative, and SX6907 plants were positive, as shown in FIG. 2.

### B. RT-PCR Identification

The T₁ RppRC1 transgenic soybean obtained in step 2 (i.e. L1-2 and L2-1 in step 1), the soybean line transformed with empty vector (CK) and the non-transgenic plant Tianlong No. 1 were taken and subjected to total RNA extraction, respectively; the resulting RNA was reverse transcribed to obtain cDNA, the resulting cDNA was used as a template to perform real-time fluorescence quantitative PCR amplification on the cDNA of the gene RppRC1 with specific primers F1 and R1, wherein soybean β-actin was used as an internal reference which was amplified with the primers FC and RC. Real-time fluorescence quantitative PCR was run on CFX Connect™ real-time fluorescence quantitative PCR instrument with 3 replicates in one parallel test. The relative expression level was calculated using the method reported by Livak KJ and Schmittgen TD (2001), i.e. 2^{-ΔΔCT}.

$$Δ Δ {C}_{T} = {( {C}_{T . Target} - {C}_{T . Actin} )}_{Time x} - {( {C}_{T . Target} - {C}_{T . Actin} )}_{Time 0}$$

Time x represents any point in time, and Time₀ represents a double amount of target gene expression after β-actin correction.

The sequences of the above primers are as follows:

F1:
(SEQ ID NO: 5)
5′-TCGGCAAAGTTGGTTTTCATCT-3′;
R1:
(SEQ ID NO: 6)
5′-CCATTCCTGGGCTCCACATT-3′;
FC
(SEQ ID NO: 8)
5′-ATTGGACTCTGGTGATGGTG-3′;
and
RC:
(SEQ ID NO: 9)
5′-TCAGCAGAGGTGGTGAACATT-3′.

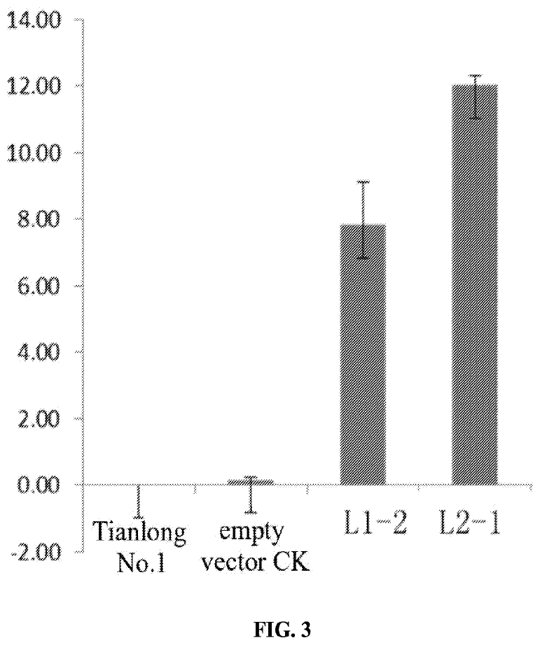

The results are shown in FIG. 3. The target gene RppRC1 was virtually not expressed in non-transgenic Tianlong No. 1 and plants with empty vector; however, the target gene RppRC1 was highly expressed in RppRC1 transgenic Tianlong No. 1 L1-2 and L2-1.

### C. Southern Detection

The T1 RppRC1 transgenic soybean plants (L1-1, L1-2, L1-3, L1-4, L1-5, L2-1, and L2-1) obtained in step 2, the non-transgenic Tianlong No. 1 plant (negative), and the T1 plants L3-1, L3-2, L3-3, L3-4 and L3-5 transformed with empty vector were taken and subjected to genomic DNA extraction, respectively; the resulting genomic DNA was digested with endonuclease HindIII, and then the enzyme-digested products were subjected to southern detection using digoxin hybridization detection kit II (chemiluminescence method), wherein BAR gene was used as a probe, and the probe primers were as follows:

F2:
(SEQ ID NO: 10)
5'-AGAAACCCACGTCATGCCAGTT-3';
and
R2:
(SEQ ID NO: 80)
5'-ATCGTCAACCACTACATCG-3'.
(421 bp)

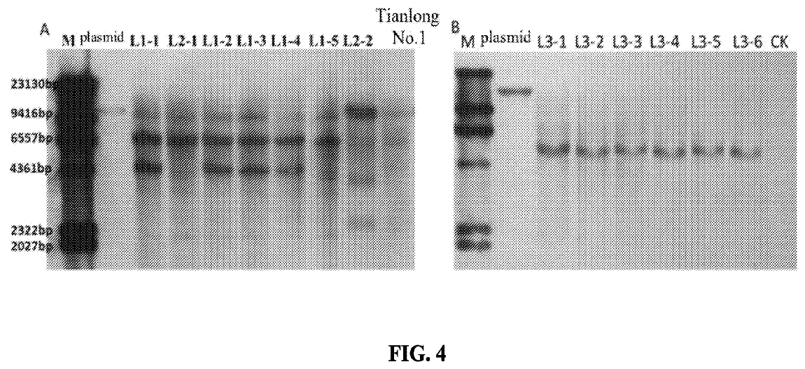

The results are shown in FIGS. 4A and 4B. The plants L1-1, L1-2, L1-3, L1-4 and L1-5 were double copies, L2-1 and L2-1 were single copies, and the plants L3-1, L3-2, L3-3, L3-4 and L3-5 with empty vector were single copies. As for RppRC1 transgenic soybean, these may represent the copy number of RppRC1 gene.

### Example 3: Detection of the Rust Resistance of Transgenic Soybean

In this example, the transgenic plants obtained in Example 2 were inoculated with the physiological race SS4 of Phakopsora pachyrhizi (described in “Shan Zhihui et al., Pathogenic responses of Phakopsora pachyrhizi in different legume hosts. Chinese Journal of Oil Crop Sciences, 2008, 30(4): 497-500”), which is available to the public from the applicant and can only be used to repeat the experiments of the present invention, but not for other purposes), and the disease symptoms of the plants were scored to determine the effect of RppRC1 gene against rust.

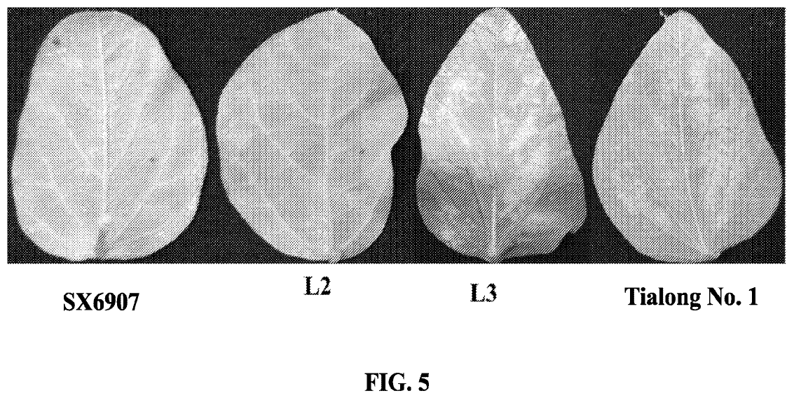

First, T0 transformation events L1 and L2 were preliminarily tested to evaluate the effect of RppRC1 transgene on rust infection. The specific operations were as follows: Fully unfolded new leaves were taken from T0 plants, and sprayed and inoculated with the suspension of the physiological race SS4 of Phakopsora pachyrhizi (1×10⁵ spores/ml) at an inoculum size of 10 μl per square centimetre. The untransformed recipient genotype Tianlong No. 1 (negative control) and the plant transformed with empty vector from the same event (empty vector control) were used as susceptible controls, and the untransformed disease resistance variety SX6907 was used as disease resistance control (positive control). After inoculation, the plants were cultured in a greenhouse at 25° C. with a photoperiod of 16 hours of light/8 hours of darkness and a relative humidity of 65%-85%. The disease symptoms were scored 12-15 days after inoculation. The disease resistance of the plants was determined according to the nature of the disease spots and the rupture of the sori. The plants were qualitatively rated as immunity (IM: no lesions), high resistance (R: reddish black disease spots, a small amount of spore formation) and susceptibility (S: tawny disease spots, a large amount of spore formation). Refer to “Bromfield, K R., Melching, J S., Kingsolver, C H. 1980, Virulence and Aggressiveness of Phakopsora pachyrhizi Isolates Causing Soybean Rust. Phytopathology. 70: 17-21”. The results showed that both positive control and transformed plants (T0 transformation events L1 and L2) showed immunity, while plants with empty vector and negative control plants showed susceptibility, as shown in FIG. 5 and Table 3-1.

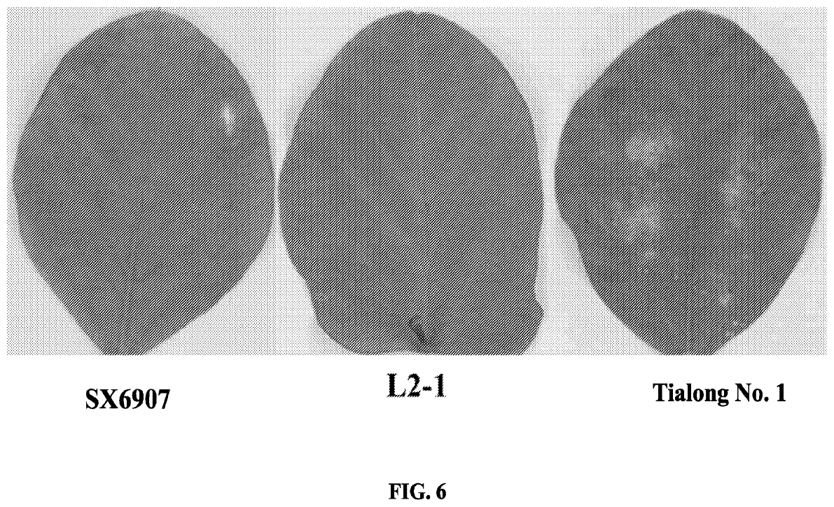

Then, the efficacy of RppRC1 gene against Phakopsora pachyrhizi was tested for T1 transgenic plants L1-1, L1-2, L1-3, L2-1 and L2-1, and the plant L3-1 transformed with empty vector. T1 seeds were planted under growth chamber conditions, and inoculation and identification were carried out when the plant grew to having two true leaves completely unfolded. Spore suspension of the physiological race SS4 of Phakopsora pachyrhizi was used for inoculation. The inoculation method was the same as above. The plant of untransformed variety Tianlong No. 1 was the susceptible control and the plant of untransformed variety SX6907 with disease resistance was the disease resistance control. Symptoms were observed 12 days later. The results showed that no disease spot formation and sorus formation were observed on the leaves of transgenic plants L1-1, L1-2, L1-3, L2-1 and L2-1 with the resistance grade of immunity. Molecular detection showed that the transgenic plants comprised the full length of and had high expression level of RppRC1 gene. The leaves of the untransformed plant and the leaves of the plant L3-1 transformed with empty vector produced tawny disease spots and produced a large amount of spores, showing a susceptible response (FIG. 6 and Table 3-2).

The results of these rust infection tests showed that RppRC1 gene can provide resistance against Phakopsora pachyrhizi though transgenesis.

TABLE 3-1
Resistance of T0 transgenic plants carrying
RppRC1 gene against rust

| Transformation Events | Resistance |
|---|---|
| L1 | Immunity |
| L2 | Immunity |
| L3 empty vector | Susceptibility |
| Negative control Tianlong No. 1 | Susceptibility |
| Positive control SX6907 | Immunity |

TABLE 3-2
Resistance of T1 transgenic plants carrying
RppRC1 gene against rust

| Transformed plants | Resistance |
|---|---|
| L1-1 | Immunity |
| L1-2 | Immunity |
| L1-3 | Immunity |
| L2-1 | Immunity |
| L2-2 | Immunity |
| L3-1 empty vector | Susceptibility |
| Negative control Tianlong No. 1 | Susceptibility |
| Positive control SX6907 | Immunity |

Examples 1-3 establish that the resistance against rust of transgenic soybean obtained by transforming RppRC1 gene into susceptible soybean variety Tianlong No. 1 is significantly higher than that of recipient parent Tianlong No. 1, indicating that RppRC1 and the coding gene thereof can regulate and control the resistance of leguminous plants against rust, and improve the rust resistance of plants after overexpression. RppRC1 and the coding gene thereof can be used to improve the disease resistance of leguminous crops and are of great significance for breeding new varieties with disease resistance.

### Example 4: Use of Gene Editing for Rust Resistance Gene Allele Replacement

In this example, gene editing is used to replace a wild type gene with an interval or gene conferring increased rust resistance to Phakopsora pachyrhizi.

First, two gRNAs are designed to target the insertion region. In this example, gRNAs were designed to target the 736 bp region and the 1642 bp region of Glycine max Williams 82. A donor DNA sequence was designed including a 6057 pb portion of the interval of SEQ ID NO: 13, further modified to include 500 bp homologous arms on each side. Other examples include other portions of the interval, but typical examples will include portions containing a nucleic acid sequence that encodes the protein of claim 1.

Second, Cas12a editing machinery, gRNAs, and donor DNA are delivered to at least one plant cell using biolistic mediated transformation.

Targeted insertion events may then be screened with PCR and sequencing for example. Phenotypic evaluation may also be used.

### NUMBERED EMBODIMENTS OF THE INVENTION

The following are all embodiments contemplated and encompassed within the invention. In certain aspects, numbered embodiment of the invention include:
1. A protein selected from the group consisting of:
   (A1) a protein having the amino acid sequence shown in SEQ ID NO: 1;
   (A2) a protein having substitution and/or deletion and/or addition of one or several amino acid residues from and having the same function as the amino acid sequence shown in SEQ ID NO: 1;
   (A3) a protein having more than 99%, more than 95%, more than 90%, more than 85%, or more than 80% homology with and having the same function as the amino acid sequence defined in either (A1) or (A2); and
   (A4) a fusion protein obtained by tagging at the N-terminus and/or C-terminus of the protein defined in any one of (A1) to (A3).
2. A nucleic acid molecule encoding the protein of embodiment 1.
3. The nucleic acid molecule of embodiment 2, wherein the nucleic acid molecule is a gene and wherein the gene is a DNA molecule of any of:
   (B1) a DNA molecule shown in SEQ ID NO: 2;
   (B2) a DNA molecule hybridizing to the DNA molecule defined in (B1) under a stringent condition and encoding the protein;
   (B3) a DNA molecule having more than 99%, more than 95%, more than 90%, more than 85%, or more than 80% homology with the DNA sequences defined in (B1) and (B2) and encoding the protein.
4. An expression cassette, a recombinant vector, a recombinant bacterium, or a transgenic cell line comprising the nucleic acid molecule of embodiment 2 or 3.
5. The expression cassette of embodiment 4, characterized in that the promoter for initiating the transcription of the nucleic acid molecule in the expression cassette is an original endogenous promoter, and the nucleotide sequence of the original endogenous promoter is shown in SEQ ID NO: 7.
6. The recombinant vector of embodiment 4, characterized in that the recombinant vector is a recombinant plasmid obtained by cloning the nucleic acid molecule between the attR1 and attR2 sites of pB2GW7 vector, and replacing the 35S promoter between the SacI and SpeI enzyme digestion sites with the endogenous promoter of RppRC1 gene shown in SEQ ID NO: 7.
7. Use of the protein of embodiment 1 or the nucleic acid molecule of embodiment 2 or 3, or the recombinant vector, recombinant bacterium, or transgenic cell line of any one of embodiments 4 to 6 in regulating the resistance of a plant against rust.
8. The use of embodiment 7, characterized in that in the use, the expression level and/or activity of the protein or the coding gene thereof in the plant is increased, and the resistance of the plant against rust is enhanced.
9. A method selected from:
   (C1) a method for improving the resistance of a plant against rust, comprising the following steps: increasing the expression level and/or activity of the protein of embodiment 1 in the plant; or
   (C2) a method for breeding a plant variety with improved resistance against rust, comprising the step of increasing the expression level and/or activity of the protein of embodiment 1 in a recipient plant.
10. The method of embodiment 9, characterized in that increasing the expression level and/or activity of the protein in the plant can be realized by transgenic means or by sexual hybridization.
11. A method for breeding a transgenic plant with improved resistance against rust, comprising the following step: introducing the nucleic acid molecule of embodiment 2 or 3 to a recipient plant to obtain a transgenic plant; the transgenic plant has improved resistance against rust compared with the recipient plant.
12. The method of embodiment 11, characterized in that introducing the nucleic acid molecule to the recipient plant is realized by introducing the expression cassette or the recombinant vector of any one of embodiments 4-6 into the recipient plant.
13. A primer pair for amplifying the nucleic acid molecule of embodiment 2 or 3.
14. The primer pair of embodiment 13, characterized in that the primer pair is a primer pair 1 composed of two single-stranded DNA shown in SEQ ID NO: 3 and SEQ ID NO: 4, respectively, or a primer pair 2 composed of two single-stranded DNA shown in SEQ ID NO: 5 and SEQ ID NO: 6, respectively.
15. A probe for amplifying the nucleic acid molecule of embodiment 2 or 3.
16. A kit comprising the primer pair of embodiment 13 or 14 and/or the probe of embodiment 15.
17. A plant comprising the nucleic acid molecule of embodiment 2 or 3.
18. The plant of embodiment 17, characterized in that the plant is a transgenic plant with improved resistance against rust obtained by breeding using the method of embodiment 11 or 12, or is soybean SX6907, or a progeny plant comprising the nucleic acid molecule of embodiment 2 or 3 obtained after sexual hybridization using the soybean SX6907 as a parent; the soybean SX6907 has the accession number CGMCC No. 17575 in the China General Microbiological Culture Collection Center.
19. Use of the primer pair of embodiment 13 or 14 or the probe of embodiment 15 or the kit of embodiment 16 in identifying whether a plant to be tested comprises the nucleic acid molecule of embodiment 2 or 3.
20. Use of the primer pair of embodiment 13 or 14 or the probe of embodiment 15 or the kit of embodiment 16 in identifying whether a plant to be tested has resistance against rust conferred by the nucleic acid molecule of embodiment 2 or 3.
21. The use or method or primer pair or probe or kit or plant of any one of embodiments 7-20, characterized in that the rust is leguminous plant rust.
22. The use or method or primer pair or probe or kit or plant of embodiment 21, characterized in that the leguminous plant rust is soybean rust.
23. The use or method or primer pair or probe or kit or plant of embodiment 22, characterized in that the pathogen of soybean rust is Phakopsora pachyrhizi or Phakopsora meibomiae.
24. The use or method or primer pair or probe or kit or plant of embodiment 23, characterized in that the Phakopsora pachyrhizi is the physiological race SS4 of Phakopsora pachyrhizi.
25. The use or method or primer pair or probe or kit or plant of any one of embodiments 7-24, characterized in that the plant is a leguminous plant.
26. The use or method or primer pair or probe or kit or plant of embodiment 25, characterized in that the leguminous plant is any of: soybean, alfalfa, clover, pea, common bean, lentil, lupin, ghaf tree, carob bean, soybean, peanut or tamarind.

In another aspect, numbered embodiments of the invention include:
1. An elite Glycine max plant having in its genome a chromosomal interval from a second Glycine plant, wherein said chromosomal interval confers increased Asian soybean rust (ASR) resistance as compared to a control plant not comprising said chromosomal interval.
2. The plant of embodiment 1, wherein the chromosome interval is derived from Glycine max strain SX6907.
3. The plant of embodiments 1 or 2, wherein the chromosome interval comprises SEQ ID NO: 2 or any portion thereof, wherein the portion confers increased ASR resistance in the plant.
4. The plant of embodiment 4, wherein the chromosome interval comprises a nucleic acid sequence that is at least 85%, at least 90%, or at least 95% identical to SEQ ID NO: 2.
5. The plant of anyone of embodiments 1-4, wherein the chromosome interval encodes a protein of SEQ ID NO: 1 and wherein protein confers increased ASR resistance in the plant.
6. The plant of any one of embodiments 1-5, wherein the chromosome interval from the second Glycine plant is inserted into chromosome 18 of the plant.
7. The plant of anyone of embodiments 1-6, wherein the chromosome interval from the second Glycine plant is inserted into the region beginning at about base pair 56,680,416 and ending at about base pair 56,677,361 on chromosome 18 of Glycine max strain Williams 82 or equivalent thereof in other Glycine max strains.
8. The plant of any one of embodiments 1-7, wherein the chromosomal interval comprises SEQ ID NOs: 11-13 or a portion of any thereof wherein said portion confers in said plant increased ASR resistance.
9. The plant of any one of embodiments 1-7, wherein the chromosomal interval comprises a SNP marker associated with increased ASR resistance wherein said SNP marker corresponds with any one of the favorable SNP markers as listed in Table 1.
10. The plant of any one of embodiments 1-9, wherein the interval is derived from chromosome 18 of the second plant.
11. The plant of any one embodiments 1-10, wherein at least one parental line of said plant was selected or identified through molecular marker selection, wherein said parental line is selected or identified based on the presence of a molecular marker located within or closely linked with said chromosome interval corresponding to any one of SEQ ID NOs: 11-13 wherein said molecular marker is associated with increased ASR resistance.
12. The plant of embodiment 11, wherein the molecular marker is a single nucleotide polymorphism (SNP), a quantitative trait locus (QTL), an amplified fragment length polymorphism (AFLP), randomly amplified polymorphic DNA (RAPD), a restriction fragment length polymorphism (RFLP) or a microsatellite.
13. The plant of embodiments 11 or 12, wherein the molecular marker is a SNP marker and the molecular marker is any favorable marker as shown in Table 1.
14. The plant of any one of embodiments 1-13, wherein the plant is an agronomically elite Glycine max plant having a commercially significant yield and/or commercially susceptible vigor, seed set, standability, or threshability.
15. The plant of any one of embodiments 1-13, wherein said interval is introduced into said plant genome by genome editing of sequences corresponding to and comprising any one of SEQ ID NOS: 2 or 11.
16. The plant of embodiment 15, wherein the interval is introduced by genome editing of a Glycine max genomic region homologous to or a ortholog to any of the intervals corresponding to SEQ ID NOs: 11-13 and further making at least one genomic edit to said Glycine max genomic region to include at least 1 allele change corresponding to any favorable allele as described in any of Table 1 wherein said Glycine max genomic region did not comprise said allele change before genome edit and further wherein said genomic edit confers in a plant increased ASR resistance.
17. The plant of embodiment 16, wherein the genomic edit is accomplished through CRISPR, TALEN, meganucleases, or through modification of genomic nucleic acids.
18. The plant of anyone one of embodiments 1-17, wherein said interval is introduced into said plant genome by transgenic expression of sequences corresponding to and comprising any one of SEQ ID NOS: 11-13.
19. The plant of any one of embodiments 1-18, wherein the chromosome interval comprises SEQ ID NO: 11, or a portion thereof conferring ASR resistance.
20. The plant of any one of embodiments 1-18, wherein the chromosome interval comprises SEQ ID NO: 12, or a portion thereof conferring ASR resistance.
21. The plant of any one of embodiments 1-18, wherein the chromosome interval comprises SEQ ID NO: 13, or a portion thereof conferring ASR resistance.
22. An agronomically elite Glycine max plant having commercially significant yield, comprising a chromosomal interval derived from Glycine max SX6907, a chromosomal interval comprising SEQ ID NO: 2, a chromosomal interval comprising SEQ ID NO: 11-13, a chromosomal interval encoding the protein of SEQ ID NO: 1, or a portion thereof wherein said chromosomal interval or portion thereof confers increased ASR resistance in said plant as compared to a control plant not comprising said chromosomal interval.
23. A plant cell, seed, or plant part derived from the plant of any one of embodiments 1-22.
24. A progeny plant from the plants of any one of embodiments 1-23.
25. A method of producing a Glycine max plant having increased resistance to Asian soybean rust (ASR), the method comprising the steps of:
   a) providing a first Glycine max plant comprising in its genome a chromosomal interval corresponding to SEQ ID NOs: 11, 12 or 13, or a chromosome interval encoding the protein of SEQ ID NO: 1 or a chromosome interval comprising SEQ ID NO: 2, wherein said first Glycine max plant has increased resistance to ASR;
   b) crossing the Glycine max plant of a) with a second Glycine max plant not comprising said chromosomal interval; and
   c) selecting a progeny plant from the cross of b) by isolating a nucleic acid from said progeny plant and detecting within said nucleic acid an allele that associates with increased ASR resistance and further wherein said allele is closely linked with or located within the chromosome intervals corresponding to SEQ ID NOs: 11, 12 or 13, or a chromosome interval encoding the protein of SEQ ID NO: 1 or chromosome comprising SEQ ID NO: 2, thereby producing a Glycine max plant having increased resistance to ASR.
26. The method of embodiment 25, wherein the allele corresponds to any of the favorable alleles as depicted in Table 1.
27. The method of embodiments 25 or 26, wherein the either first or second Glycine max plant is an elite Glycine max plant.
28. A method of producing a Glycine max plant with increased resistance to increased resistance to Asian soybean rust (ASR), the method comprising the steps of:
   a) isolating a nucleic acid from a Glycine max plant;
   b) detecting in the nucleic acid of a) at least one molecular marker associated with increased ASR wherein said molecular marker is located within 20 cM, 10 cM, 5 cM, 1 cM 0.5 cM, or closely linked with a chromosomal interval corresponding to a genomic region from Glycine max chromosome 18 comprising SEQ ID NO: 2 or SEQ ID NO: 11, or a portion thereof, wherein said portion confers to a plant increased ASR resistance;
   c) selecting a plant based on the presence of the molecular marker detected in b); and
   d) producing a Glycine max progeny plant from the plant of c) identified as having said allele associated with increased ASR resistance.
29. The method of embodiment 28, wherein the molecular marker is closely linked with or consists of any one of the favorable alleles as depicted in Table 1.
30. A method of identifying or selecting a Glycine max plant having increased ASR resistance, the method comprising the steps of
   a) isolating a nucleic acid from a Glycine max plant;
   b) detecting in the nucleic acid the presence of a molecular marker that associates with increased ASR resistance wherein the molecular marker is located within 20 cM, 10 cM, 5 cM, 1 cM, 0.5 cM of a marker as described in Table 1; and
   c) identifying or selecting a Glycine max plant having increased ASR resistance on the basis of the molecular marker detected in b).
31. The method of embodiment 30, wherein the allele detected in b) consists of any favorable marker as described in Table 1.
32. The method of any one of embodiments 28-31, wherein the molecular marker is a single nucleotide polymorphism (SNP), a quantitative trait locus (QTL), an amplified fragment length polymorphism (AFLP), randomly amplified polymorphic DNA (RAPD), a restriction fragment length polymorphism (RFLP) or a microsatellite.
33. The method of any one of embodiments 28-32, wherein the detecting comprises amplifying a marker locus or a portion of the marker locus and detecting the resulting amplified marker amplicon.
34. The method of embodiment 33, wherein the amplifying comprises: a) admixing an amplification primer or amplification primer pair with a nucleic acid isolated from the first Glycine max plant or germplasm, wherein the primer or primer pair is complementary or partially complementary to at least a portion of the marker locus, and is capable of initiating DNA polymerization by a DNA polymerase using the Glycine max nucleic acid as a template; and, b) extending the primer or primer pair in a DNA polymerization reaction comprising a DNA polymerase and a template nucleic acid to generate at least one amplicon.
35. The method of embodiments 33 or 34, wherein the amplifying comprises employing a polymerase chain reaction (PCR) or ligase chain reaction (LCR) using a nucleic acid isolated from a soybean plant or germplasm as a template in the PCR or LCR.
36. The method of any one of embodiments 28-35, wherein the nucleic acid is selected from DNA or RNA.
37. A primer diagnostic for ASR resistance, wherein said primer can be used in a PCR reaction to indicate the presence of an allele associated with ASR resistance, wherein said allele is any favorable allele as described in Table 1.
38. A method of conferring ASR resistance to Glycine max plants comprising:
   a) providing a nucleic acid molecule from chromosome 18 of a Glycine max plant having ASR resistance, wherein said nucleic acid encodes ASR resistance; and
   b) inserting the nucleic acid molecule into chromosome 18 of a Glycine max strain lacking ASR resistance to thereby produce a plant having increased ASR resistance compared to a control plant not comprising the nucleic acid,
wherein the nucleic acid molecule is selected from the group consisting of:
   a nucleic acid encoding the protein of SEQ ID NO: 1;
   a nucleic acid comprising the nucleic acid sequence of SEQ ID NO: 2 or any portion thereof, wherein the portion confers increased ASR resistance in the plant; and
   a chromosome interval comprising SEQ ID NO: 11.
39. The method of embodiment 38, wherein the nucleic acid molecule is derived from Glycine max strain SX6907.
40. The method of embodiment 38 or embodiment 39, wherein the nucleic acid molecule is inserted into chromosome 18 of a Glycine max plant.
41. The method of any one of embodiments 38-40, wherein the nucleic acid molecule is inserted a region beginning at about base pair 56,680,416 and ending at about base pair 56,677,361.
42. The method of any one of embodiments 39-41, wherein the method comprises Cas12a mediated gene replacement.
43. The method of any one of embodiments 38-42, wherein the method comprises 2 gRNAs.
44. The method of embodiment 43, wherein the method comprises a gRNA of SEQ ID NO: 79 and/or SEQ ID NO: 81.
45. The method of any one of embodiments 38-44, wherein the method comprises screening for the targeted insertion with PCR and/or sequencing.
46. A protein selected from:
   (A1) a protein having the amino acid sequence shown in SEQ ID NO: 1;
   (A2) a protein having substitution and/or deletion and/or addition of one or several amino acid residues from and having the same function as the amino acid sequence shown in SEQ ID NO: 1;
   (A3) a protein having more than 99%, more than 95%, more than 90%, more than 85%, or more than 80% homology with and having the same function as the amino acid sequence defined in either (A1) or (A2); or
   (A4) a fusion protein obtained by tagging at the N-terminus and/or C-terminus of the protein defined in any one of (A1) to (A3).
47. A nucleic acid molecule encoding the protein of embodiment 46.
48. The nucleic acid molecule of embodiment 47, wherein the nucleic acid molecule is any of:
   (B1) a DNA molecule shown in SEQ ID NO: 2;
   (B2) a DNA molecule hybridizing to the DNA molecule defined in (B1) under a stringent condition and encoding the protein; or
   (B3) a DNA molecule having more than 99%, more than 95%, more than 90%, more than 85%, or more than 80% homology with the DNA sequences defined in (B1) and (B2) and encoding the protein.
49. An expression cassette, a recombinant vector, a recombinant bacterium, or a transgenic cell line comprising the nucleic acid molecule of embodiment 47 or 48.
50. The expression cassette of embodiment 49, characterized in that the promoter for initiating the transcription of the nucleic acid molecule in the expression cassette is an original endogenous promoter, and the nucleotide sequence of the original endogenous promoter is shown in SEQ ID NO: 7.
51. The recombinant vector of embodiment 50, characterized in that the recombinant vector is a recombinant plasmid obtained by cloning the nucleic acid molecule between the attR1 and attR2 sites of pB2GW7 vector, and replacing the 35S promoter between the SacI and SpeI enzyme digestion sites with the endogenous promoter of RppRC1 gene shown in SEQ ID NO: 7.
52. Use of the protein of embodiment 46 or the nucleic acid molecule of embodiment 47 or 48, or the recombinant vector, recombinant bacterium, or transgenic cell line of any one of embodiments 49 to 51 in regulating the resistance of a plant against rust.
53. The use of embodiment 47, wherein the expression level and/or activity of the protein or the coding gene thereof in the plant is increased, and the resistance of the plant against rust is enhanced.
54. A method for improving the resistance of a plant against rust, comprising increasing the expression level and/or activity of the protein of embodiment 46 in the plant;
55. A method for breeding a plant variety with improved resistance against rust, comprising increasing the expression level and/or activity of the protein of embodiment 46 in a recipient plant.
56. The method of embodiments 54 or 55, wherein the increasing the expression level and/or activity of the protein in the plant can be realized by transgenic means or by sexual hybridization.
57. A method for breeding a transgenic plant with improved resistance against rust, comprising the following step: introducing the nucleic acid molecule of embodiment 47 or 48 to a recipient plant to obtain a transgenic plant; the transgenic plant has improved resistance against rust compared with the recipient plant.
58. The method of embodiment 56, wherein the introducing the nucleic acid molecule to the recipient plant is realized by introducing the expression cassette or the recombinant vector of any one of embodiments 49-51 into the recipient plant.
59. A primer pair for amplifying the nucleic acid molecule of embodiment 47 or 48.
60. The primer pair of embodiment 59, wherein the primer pair is a primer pair 1 composed of two single-stranded DNA shown in SEQ ID NO: 3 and SEQ ID NO: 4, respectively, or a primer pair 2 composed of two single-stranded DNA shown in SEQ ID NO: 5 and SEQ ID NO: 6, respectively.
61. A probe for amplifying the nucleic acid molecule of embodiment 47 or 48.
62. A kit comprising the primer pair of embodiment 59 or 60 and/or the probe of embodiment 61.
63. A plant comprising the nucleic acid molecule of embodiment 47 or 48.
64. The plant of embodiment 63, wherein the plant is a transgenic plant with improved resistance against rust obtained by breeding using the method of embodiment 56 or 57, or is soybean SX6907, or a progeny plant comprising the nucleic acid molecule of embodiment 47 or 48 obtained after sexual hybridization using the soybean SX6907 as a parent; the soybean SX6907 has the accession number CGMCC No. 17575 in the China General Microbiological Culture Collection Center.
65. Use of the primer pair of embodiment 59 or 60 or the probe of embodiment 61 or the kit of embodiment 62 in identifying whether a plant to be tested comprises the nucleic acid molecule of embodiment 45 or 46.
66. Use of the primer pair of embodiment 59 or 60 or the probe of embodiment 61 or the kit of embodiment 62 in identifying whether a plant to be tested has resistance against rust conferred by the nucleic acid molecule of embodiment 47 or 48.
67. The use or method or primer pair or probe or kit or plant of any one of embodiments 52-66, wherein the rust is leguminous plant rust.
68. The use or method or primer pair or probe or kit or plant of embodiment 67, wherein the leguminous plant rust is soybean rust.
69. The use or method or primer pair or probe or kit or plant of embodiment 68, wherein the pathogen of soybean rust is Phakopsora pachyrhizi or Phakopsora meibomiae.
70. The use or method or primer pair or probe or kit or plant of embodiment 69, wherein the Phakopsora pachyrhizi is the physiological race SS4 of Phakopsora pachyrhizi.
71. The use or method or primer pair or probe or kit or plant of any one of embodiments 52-70, wherein the plant is a leguminous plant.
72. The use or method or primer pair or probe or kit or plant of embodiment 73, wherein the leguminous plant is any of: soybean, alfalfa, clover, pea, common bean, lentil, lupin, ghaf tree, carob bean, soybean, peanut, or tamarind.

What is claimed:

1. A soybean plant or a soybean plant cell having resistance to Asian soybean rust (ASR), wherein said soybean plant or said soybean plant cell comprises an expression cassette located at a non-native locus of the genome of said soybean plant or said soybean plant cell, wherein said expression cassette comprises a promoter which drives expression in said soybean plant or said soybean plant cell operatively linked to a nucleotide sequence encoding a protein selected from:
(A1) a protein comprising an amino acid sequence that is at least 95% identical to SEQ ID NO: 1 and when expressed in a soybean plant provides increased resistance to Asian soybean rust (ASR); and
(A2) a fusion protein obtained by tagging at the N-terminus and/or C-terminus of the protein defined in (A1).

2. The soybean plant or soybean plant cell of claim 1, wherein the protein comprises an amino acid sequence that is at least 99% identical to SEQ ID NO: 1.

3. The soybean plant or soybean plant cell of claim 1, wherein the protein has the amino acid sequence shown in SEQ ID NO: 1.

4. The soybean plant or soybean plant cell of claim 1, wherein the nucleotide sequence is at least 95% identical to SEQ ID NO: 2.

5. The soybean plant or soybean plant cell of claim 1, wherein the nucleotide sequence is at least 99% identical to SEQ ID NO: 2.

6. The soybean plant or soybean plant cell of claim 1, wherein the nucleotide sequence is the nucleotide sequence shown in SEQ ID NO: 2.

7. The soybean plant or soybean plant cell of claim 1, wherein said soybean plant is an elite Glycine max plant or said soybean plant cell is an elite Glycine max plant cell.

8. The soybean plant or soybean plant cell of claim 1, wherein said expression cassette is not located on chromosome 18 of said soybean plant or said soybean plant cell.

9. The soybean plant or soybean plant cell of claim 1, wherein said expression cassette is not located at a region of said soybean plant or said soybean plant cell equivalent to a region of Glycine max strain Williams 82 beginning at about base pair 56,680,416 and ending at about base pair 56,677,361 of chromosome 18.

10. The soybean plant or soybean plant cell of claim 1, wherein said nucleotide sequence is operatively linked to a promoter having the sequence set forth as SEQ ID NO: 7.

11. A seed of said soybean plant or soybean plant cell of claim 1, wherein said seed has resistance to ASR and comprises said expression cassette located at said non-native locus of the genome of said seed.

12. A method for improving the resistance of a soybean plant against Asian soybean rust (ASR), comprising introducing an expression cassette into a non-native locus of the genome of said soybean plant, wherein said expression cassette comprises a promoter which drives expression in said soybean plant operatively linked to a nucleotide sequence encoding a protein selected from:
(A1) a protein comprising an amino acid sequence that is at least 95% identical to SEQ ID NO: 1 and when expressed in a soybean plant provides increased resistance to ASR; and
(A2) a fusion protein obtained by tagging at the N-terminus and/or C-terminus of the protein defined in (A1).

13. The method of claim 12, wherein the protein comprises an amino acid sequence that is at least 99% identical to SEQ ID NO: 1.

14. The method of claim 12, wherein the protein has the amino acid sequence shown in SEQ ID NO: 1.

15. The method of claim 12, wherein the nucleotide sequence is at least 95% identical to SEQ ID NO: 2.

16. The method of claim 12, wherein the nucleotide sequence is at least 99% identical to SEQ ID NO: 2.

17. The method of claim 12, wherein the nucleotide sequence has the nucleic acid sequence shown in SEQ ID NO: 2.

18. The method of claim 12, wherein said soybean plant is an elite Glycine max plant.

19. The method of claim 12, wherein the method comprises gene editing.

20. The method of claim 19, wherein said gene editing comprises Cas12a mediated gene replacement.

21. The method of claim 12, wherein said expression cassette is not introduced into chromosome 18 of said soybean plant.

22. The method of claim 12, wherein said expression cassette is not located at a region of said soybean plant equivalent to a region of Glycine max strain Williams 82 beginning at about base pair 56,680,416 and ending at about base pair 56,677,361 of chromosome 18.

23. The method of claim 12, wherein said nucleotide sequence is operatively linked to a promoter having the sequence set forth as SEQ ID NO: 7.

24. The method of claim 12, wherein said method further comprises crossing said soybean plant comprising said expression cassette located at said non-native locus of the genome of said soybean plant with a second soybean plant not comprising said expression cassette to produce a progeny soybean plant comprising said expression cassette.

25. A method for controlling Asian soybean rust (ASR) in a field by planting in said field a soybean seed or a soybean plant having resistance to Asian soybean rust (ASR), wherein said soybean seed or said soybean plant comprises an expression cassette located at a non-native locus of the genome of said soybean seed or said soybean plant, wherein said expression cassette comprises a promoter which drives expression in said soybean seed or said soybean plant operatively linked to a nucleotide sequence encoding a protein selected from:
(A1) a protein comprising an amino acid sequence that is at least 95% identical to SEQ ID NO: 1 and when expressed in a soybean plant provides increased resistance to Asian soybean rust (ASR); and
(A2) a fusion protein obtained by tagging at the N-terminus and/or C-terminus of the protein defined in (A1).

26. The method of claim 25, wherein the protein comprises an amino acid sequence that is at least 99% identical to SEQ ID NO: 1.

27. The method of claim 25, wherein the protein has the amino acid sequence shown in SEQ ID NO: 1.

28. The method of claim 25, wherein the nucleotide sequence is at least 95% identical to SEQ ID NO: 2.

29. The method of claim 25, wherein the nucleotide sequence is at least 99% identical to SEQ ID NO: 2.

30. The method of claim 25, wherein the nucleotide sequence is the nucleotide sequence shown in SEQ ID NO: 2.

31. The method of claim 25, wherein said soybean seed is an elite Glycine max seed or plant or said soybean plant is an elite Glycine max plant.

32. The method of claim 25, wherein said expression cassette is not located on chromosome 18 of said soybean seed or said soybean plant.

33. The method of claim 25, wherein said expression cassette is not located at a region of said soybean seed or said soybean plant equivalent to a region of Glycine max strain Williams 82 beginning at about base pair 56,680,416 and ending at about base pair 56,677,361 of chromosome 18.

34. The method of claim 25, wherein said nucleotide sequence is operatively linked to a promoter having the sequence set forth as SEQ ID NO: 7.
